(19) United States
(12) Patent Application Publication
(10) Pub. No.: US 2026/0371921 A1
(43) Pub. Date: Nov. 19, 2026

(54) MATERIALS FOR ORGANIC ELECTROLUMINESCENT DEVICES

(71) Applicant: Merck Patent GmbH, Darmstadt (DE)

(72) Inventors: Amir Hossain Parham, Frankfurt Am Main (DE); Sebastian Stolz, Heidelberg (DE); Christian Eickhoff, Heppenheim (DE); Anna Hayer, Darmstadt (DE); Klaus Omoregbee, Mannheim (DE)

(21) Appl. No.: 19/490,053

(22) PCT Filed: Jun. 4, 2024
(86) PCT No.: PCT/EP2024/065262

§ 371 Date: Dec. 4, 2025

(30) Foreign Application Priority Data
Jun. 7, 2023 (EP) 23178055.2

(51) Int. Cl. H10K 85/60; C07B 59/00; C07D 491/048; C07D 495/04; C09K 11/02; H10K 50/11

(52) U.S. Cl. CPC H10K 85/657; C07B 59/002; C07D 491/048; C07D 495/04; C09K 11/02; H10K 85/622; H10K 85/633; H10K 85/636; H10K 85/6572; H10K 85/6576; H10K 50/11; H10K 85/615; H10K 85/6574

(57) ABSTRACT

The present invention relates to OLED materials that contain 4H-naphtho [1,2,3,4-def] carbazole, to mixtures and formulations containing these materials, and to electronic devices containing these materials, in particular organic electroluminescent devices containing these materials as matrix materials, electronic-transport materials, or hole-blocking materials.

### TECHNICAL FIELD

The present invention relates to 4H-naphtho[1,2,3,4-def]carbazoles, to mixtures and formulations comprising these, and to electronic devices comprising these compounds, in particular organic electroluminescence devices comprising these compounds as matrix materials, electron transport materials or hole blocker materials.

### STATE OF THE ART

Phosphorescent organometallic complexes are frequently used in organic electroluminescent devices (OLEDs). In general terms, there is still a need for improvement in OLEDs, for example with regard to efficiency, operating voltage and lifetime. The properties of phosphorescent OLEDs are determined not only by the triplet emitters used. More particularly, the other materials used, for example matrix materials, are also of particular significance here. Improvements to these materials can thus also lead to distinct improvements in the OLED properties.

According to the prior art, carbazole derivatives, dibenzofuran derivatives, indenocarbazole derivatives, indolocarbazole derivatives, benzofurocarbazole derivatives and benzothienocarbazole derivatives are among the matrix materials used for phosphorescent emitters.

WO2018060218 A1, KR20220135761 A, KR20220063428 A and WO22207678 A1 describe specific diazadibenzofuran-carbazole derivatives and diazadibenzothiophene-carbazole derivatives as matrix materials inter alia.

WO2012048781 A1, CN110437241 A and describe specific 4H-naphtho[1,2,3,4-def]carbazole derivatives inter alia as matrix materials.

KR20210036304 A, KR20210034528 A, WO22038065 A1, CN115073356 A, CN112062753 A and US2022263031 A1 describe complex carbazole derivatives inter alia as matrix materials.

There is generally still a need for improvement in these materials, in particular for use as matrix materials. The problem addressed by the present invention is that of providing compounds which are especially suitable for use as matrix material, electron transport material or hole blocker material in a phosphorescent OLED. More particularly, it is an object of the present invention to provide matrix materials that lead to an improved lifetime. This is especially true of the use of a low to moderate emitter concentration, i.e. emitter concentrations in the order of magnitude of 3% to 20%, especially of 3% to 15%, since, in particular, device lifetime is limited here.

It has now been found that electroluminescent devices containing compounds of the formula (1) below have improvements over the prior art, especially when the compounds are used as matrix material for phosphorescent dopants.

It has also been found that this problem is solved, and the disadvantages from the prior art are eliminated, by the combination of at least one compound of the formula (1) as first host material and at least one hole-transporting compound, for example in combination with one or more compounds of the formulae (HH-1), (HH-2), (HH-3), (HH-4), (HH-5) or (HH-6), as further host material or further host materials in a light-emitting layer of an organic electronic device, especially an organic electroluminescent device.

### SUMMARY OF THE INVENTION

The present invention firstly provides a compound of formula (1)

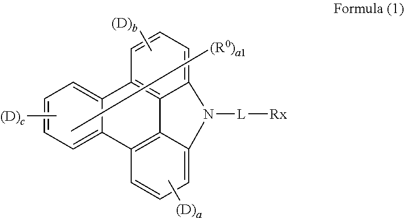

where the symbols and indices used are as follows:
L is the same or different at each instance and is a single bond or an aromatic or heteroaromatic ring system which has 5 to 40 ring atoms and may be substituted by one or more R¹ radicals;
R⁰ is the same or different at each instance and is selected from the group consisting of F, Cl, Br, I, CN, NO₂, C(═O)R², P(═O)(Ar)₂, P(Ar)₂, B(Ar)₂, Si(Ar)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR², and where one or more hydrogen atoms may be replaced by D, F, Cl, Br, I, CN or NO₂, an aromatic or heteroaromatic ring system which has 5 to 40 ring atoms and may be substituted in each case by one or more R² radicals, an aryloxy or heteroaryloxy group which has 5 to 40 ring atoms and may be substituted by one or more R² radicals, or an aralkyl or heteroaralkyl group which has 5 to 40 ring atoms and may be substituted by one or more R² radicals;
R¹ is the same or different at each instance and is selected from the group consisting of D, F, Cl, Br, I, CN, NO₂, C(═O)R², P(═O)(Ar)₂, P(Ar)₂, B(Ar)₂, Si(Ar)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR², and where one or more hydrogen atoms may be replaced by D, F, Cl, Br, I, CN or NO₂, an aromatic or heteroaromatic ring system which has 5 to 40 aromatic ring atoms and may be substituted in each case by one or more R² radicals, an aryloxy or heteroaryloxy group which has 5 to 40 ring atoms and may be substituted by one or more R² radicals, or an aralkyl or heteroaralkyl group which has 5 to 40 ring atoms and may be substituted by one or more R² radicals;
Rx conforms to one of the formulae (1-1), (1-2), (1-3) or (1-4)

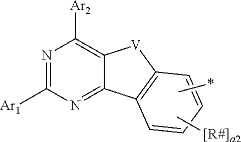

Formula (1-1) 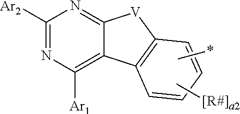
Formula (1-2) 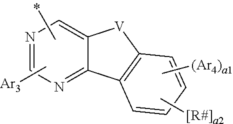
Formula (1-3) 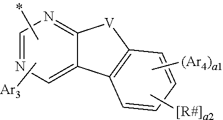
Formula (1-4)

* denotes the bond to L-Rx,
V is O or S;
R# at each instance is independently D, F, CN or phenyl that may be substituted by one or more R² radicals;
[R#]ₐ₂ represents a monosubstitution, a disubstitution, the maximum permissible substitution or no substitution by R#;
Ar is the same or different at each instance and is an aromatic or heteroaromatic ring system which has 5 to 40 ring atoms and may be substituted by one or more R² radicals;
Ar₁, Ar₂ are independently an aromatic or heteroaromatic ring system which has 5 to 40 ring atoms and may be substituted by one or more R² radicals;
Ar₃ is an aromatic ring system which has 6 to 40 ring atoms and may be substituted by one or more R² radicals, or a heteroaromatic ring system which has 5 to 40 ring atoms and may be substituted by one or more R² radicals, and where the heteroaromatic ring system contains one or more heteroatoms selected from O, S, Se or Si;
Ar₄ is the same or different at each instance and is an aromatic or heteroaromatic ring system which has 5 to 40 ring atoms and may be substituted by one or more R² radicals;
R² is the same or different at each instance and is selected from the group consisting of D, F, CN, a straight-chain alkyl group having 1 to 20 carbon atoms or a branched or cyclic alkyl group having 3 to 20 carbon atoms, where one or more nonadjacent CH₂ groups may be replaced by O or S and where one or more hydrogen atoms may be replaced by D, F, or CN;
(D)ₐ, (D)_{b}, (D)_{c} represent a monosubstitution, a disubstitution, a trisubstitution, the maximum permissible substitution or no substitution by deuterium;
a1 is 0, 1 or 2.

The invention further provides a mixture comprising at least one compound of formula (1) as described above or described as preferred later on, and at least one further compound selected from the group of the matrix materials, phosphorescent emitters, fluorescent emitters and/or emitters that exhibit TADF (thermally activated delayed fluorescence).

The invention further provides a formulation comprising at least one compound of formula (1) as described above or described as preferred later on, or a mixture as described above, and at least one solvent.

The invention further provides for the use of a compound of formula (1) in an organic electronic device.

The invention further provides an organic electronic, preferably electroluminescent, device comprising an anode, a cathode and at least one organic layer comprising at least one compound of formula (1) as described above or described as preferred later on.

The invention further provides a process for producing an organic electronic, preferably electroluminescent, device as described above or as described as preferred hereinafter, characterized in that the organic layer is applied by gas phase deposition or from solution.

### DESCRIPTION OF THE INVENTION

In the present patent application, “D” or “D atom” means deuterium.

An aryl group in the context of this invention contains 6 to 40 ring atoms, preferably carbon atoms. A heteroaryl group in the context of this invention contains 5 to 40 ring atoms, where the ring atoms include carbon atoms and at least one heteroatom, with the proviso that the sum total of carbon atoms and heteroatoms adds up to at least 5. The heteroatoms are preferably selected from N, O and/or S. What is meant here by an aryl group or heteroaryl group is either a simple aromatic cycle, i.e. phenyl, derived from benzene, or a simple heteroaromatic cycle, for example derived from pyridine, pyrimidine or thiophene, or a fused aryl or heteroaryl group, for example derived from naphthalene, anthracene, phenanthrene, quinoline or isoquinoline. An aryl group having 6 to 18 carbon atoms is therefore preferably phenyl, naphthyl, phenanthryl or triphenylenyl, with no restriction in the attachment of the aryl group as substituent. The aryl or heteroaryl group in the context of this invention may bear one or more radicals, where the suitable radical is described below. If no such radical is described, the aryl group or heteroaryl group is unsubstituted.

An aromatic ring system in the context of this invention contains 6 to 40 carbon atoms in the ring system. The aromatic ring system also includes aryl groups as described above. An aromatic ring system having 6 to 18 carbon atoms is preferably selected from phenyl, fully deuterated phenyl, biphenyl, naphthyl, phenanthryl and triphenylenyl.

A heteroaromatic ring system in the context of this invention contains 5 to 40 ring atoms and at least one heteroatom. A preferred heteroaromatic ring system has 9 to 40 ring atoms and at least one heteroatom. The heteroaromatic ring system also includes heteroaryl groups as described above. The heteroatoms in the heteroaromatic ring system are preferably selected from N, O and/or S.

What is meant by an aromatic or heteroaromatic ring system in the context of this invention is a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon or oxygen atom or a carbonyl group. For example, systems such as 9,9′-spirobifluorene, 9,9-diarylfluorene, 9,9-dialkylfluorene, diaryl ethers, stilbene, etc. shall thus also be regarded as aromatic or heteroaromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, are likewise encompassed by the definition of the aromatic or heteroaromatic ring system.

An aromatic or heteroaromatic ring system which has 5 to 40 ring atoms and may be joined to the aromatic or heteroaromatic via any position is understood to mean, for example, groups which are derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazine imidazole, quinoxaline imidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

The abbreviations Ar, Ar₁, Ar₂ and Ar₄ are the same or different at each instance and denote an aromatic or heteroaromatic ring system which has 5 to 40 ring atoms and may be substituted by one or more R² radicals, where the R² radical or the substituents R² has/have a definition as described above or hereinafter. A preferred definition of Ar and Ar and Ar₂ and Ar₄ is described hereinafter.

The abbreviation Ar₃ is the same or different at each instance and represents an aromatic ring system which has 6 to 40 ring atoms and may be substituted by one or more R² radicals, or a heteroaromatic ring system which has 5 to 40 ring atoms and may be substituted by one or more R² radicals, where the heteroaromatic ring system contains one or more heteroatoms selected from O, S, Se or Si, where the R² radical or the substituents R² has/have a definition as described above or hereinafter. A preferred definition of Ar₃ is described hereinafter.

The abbreviation Ar₅ is the same or different at each instance and is an aromatic or heteroaromatic ring system which has 5 to 40 ring atoms and may be substituted by one or more R⁷ radicals, where the R⁷ radical or the substituents R⁷ is/are defined as described above or hereinafter. A preferred definition of Ar₅ is described hereinafter.

The wording that two or more radicals may form a ring with one another, in the context of the present description, shall mean, inter alia, that the two radicals are joined to one another by a chemical bond with formal elimination of two hydrogen atoms. This is illustrated by the following scheme:

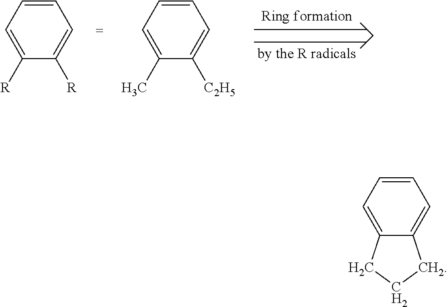

In addition, however, the abovementioned wording shall also mean that, if one of the two radicals is hydrogen, the second radical is bonded to the position to which the hydrogen atom was bonded, forming a ring. This will be illustrated by the following scheme:

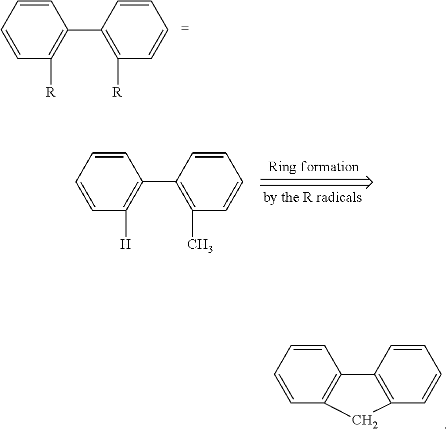

What is meant by a cyclic alkyl, alkoxy or thioalkyl group in the context of this invention is a monocyclic, a bicyclic or a polycyclic group.

In the context of the present invention, a straight-chain, branched or cyclic C₁- to C₂₀-alkyl group means, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals.

There follows a description of the compounds of the formula (1) and preferred embodiments thereof. The preferred embodiments are also applicable to the mixture of the invention, formulation of the invention and organic electronic or electroluminescent device of the invention.

The index a1 means 0, 1 or 2, where the position of the substituents R⁰, when they occur, is not restricted. Preferred compounds of the formula (1) are compounds of the formulae (1a) in which a1=0, compounds of formula (1b) in which a1=1, and compounds of formula (1c) in which a1=2:

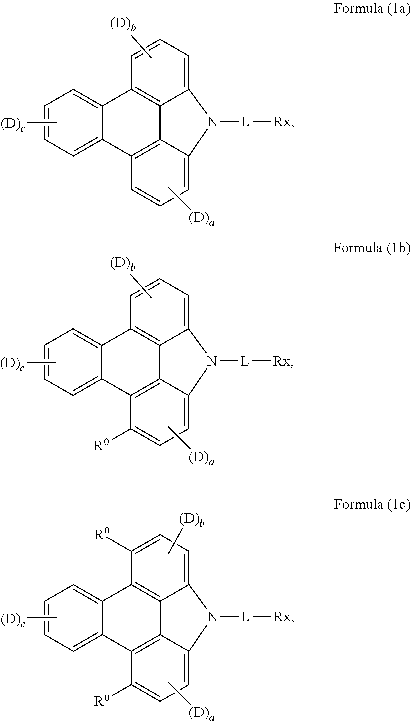

where R⁰, (D)ₐ, (D)_{b}, (D)_{c}, L and Rx have a definition given above or given with preference hereinafter.

The R⁰ radical is the same or different at each instance and is preferably F, CN, a straight-chain alkyl group having 1 to 20 carbon atoms or a branched or cyclic alkyl group having 3 to 20 carbon atoms or an alkenyl group having 2 to 20 carbon atoms, each of which may be substituted by one or more R² radicals, an aromatic ring system which has 6 to 40 ring atoms and may be substituted in each case by one or more R² radicals, dibenzofuranyl or dibenzothiophenyl, each of which may be substituted by one or more R² radicals, or conforms to one of the formulae (1-1), (1-2), (1-3) or (1-4), where Ar₁, Ar₂, Ar₃, Ar₄, a1, V, R# and [R#]ₐ₂ have a definition given above or given with preference hereinafter.

The R⁰ radical is the same or different at each instance and is more preferably F, CN, phenyl, 1,2-biphenyl, 1,3-biphenyl or 1,4-biphenyl, where phenyl, 1,2-biphenyl, 1,3-biphenyl or 1,4-biphenyl may be substituted by one or more R² radicals.

Particularly preferred compounds of the formula (1) are the compounds of the formulae (1a), (1b) and (1c), where Rx conforms to a formula (1-1) or (1-2), where R⁰, a1, (D)ₐ, (D)_{b}, (D)_{c}, L, [R#]ₐ₂, R#, V, Ar₁, Ar₂, Ar₃ and Ar₄ have a definition given above or given with preference hereinafter.

Preferred compounds of the formula (1) are furthermore the compounds of the formulae (1d), (1e), (1f) and (1g):

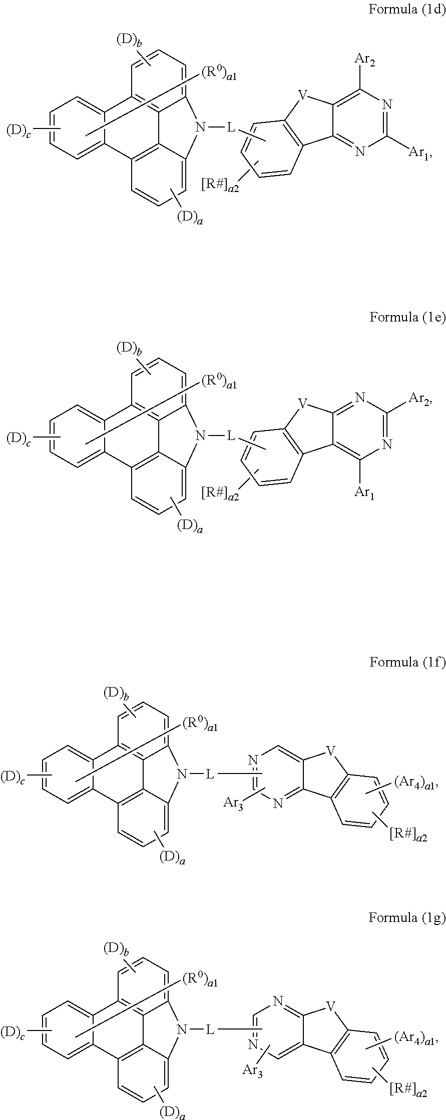

where R⁰, a1, (D)ₐ, (D)_{b}, (D)_{c}, L, [R#]ₐ₂, R#, V, Ar₁, Ar₂, Ar₃ and Ar₄ have a definition given above or given with preference hereinafter.

Particularly preferred compounds of the formula (1) are the compounds of the formulae (1d) and (1e), i.e. compounds of the formula (1) in which Rx conforms to a formula (1-1) or (1-2), where R⁰, a1, (D)ₐ, (D)_{b}, (D)_{c}, L, [R#]ₐ₂, R#, V, Ar₁, Ar₂, Ar₃ and Ar₄ have a definition given above or given with preference hereinafter.

Particularly preferred compounds of the formulae (1d) and (1e) are compounds of the formulae (1h) to (1m):

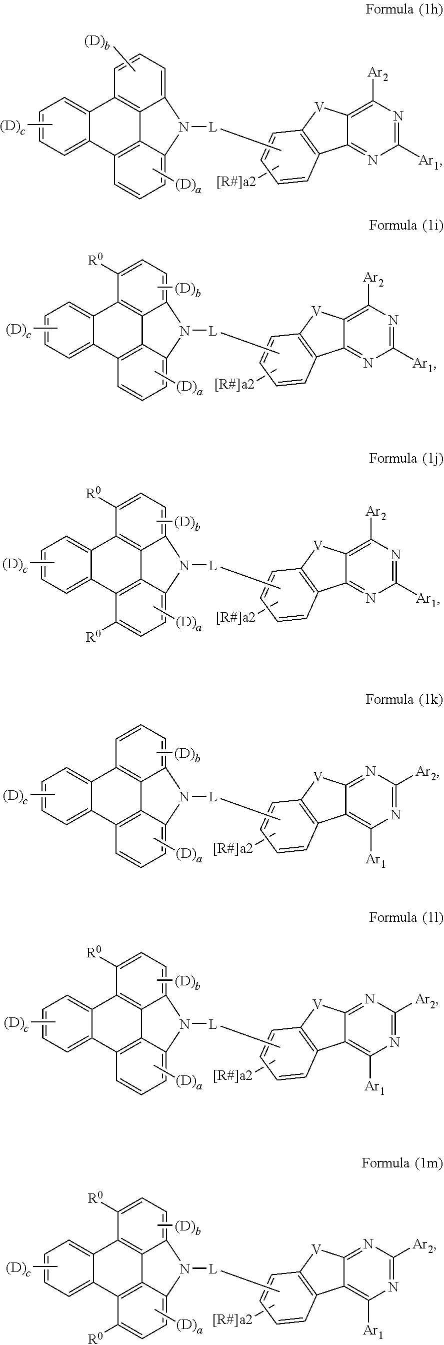

where R⁰, (D)ₐ, (D)_{b}, (D)_{c}, L, [R#]ₐ₂, R#, V, Ar₁, Ar₂, Ar₃ and Ar₄ have a definition given above or given with preference hereinafter.

In one embodiment of the invention, the linker L in compounds of the formulae (1), (1a), (1b), (1c), (1d), (1e), (1f), (1g), (1h), (1i), (1j), (1k), (1l) and (1m) is a bond or an aromatic or heteroaromatic ring system of the formulae L-1 to L-34, which may be substituted by one or more R¹ radicals:

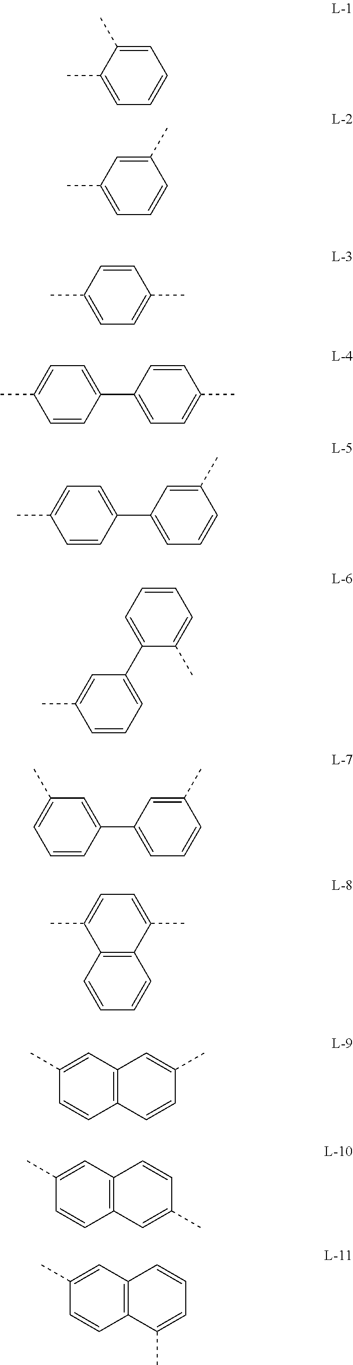

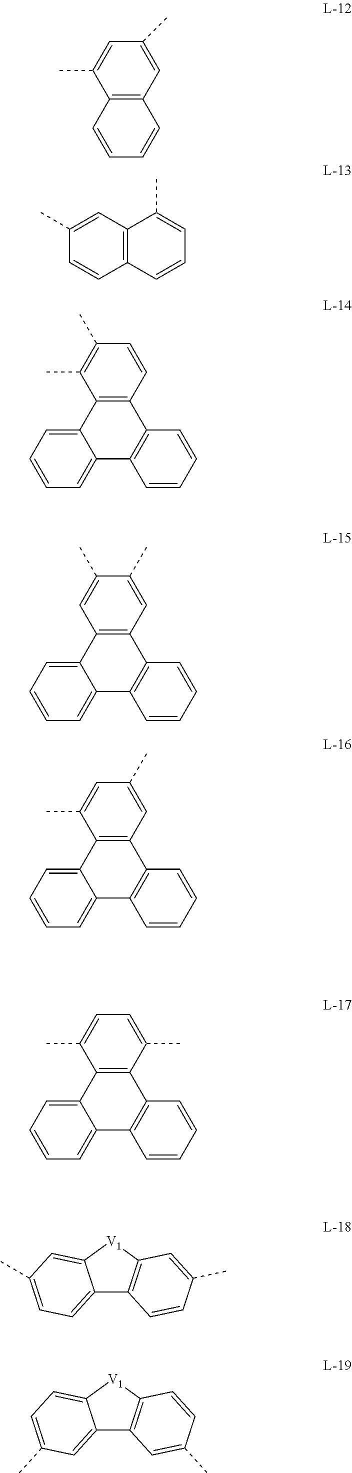

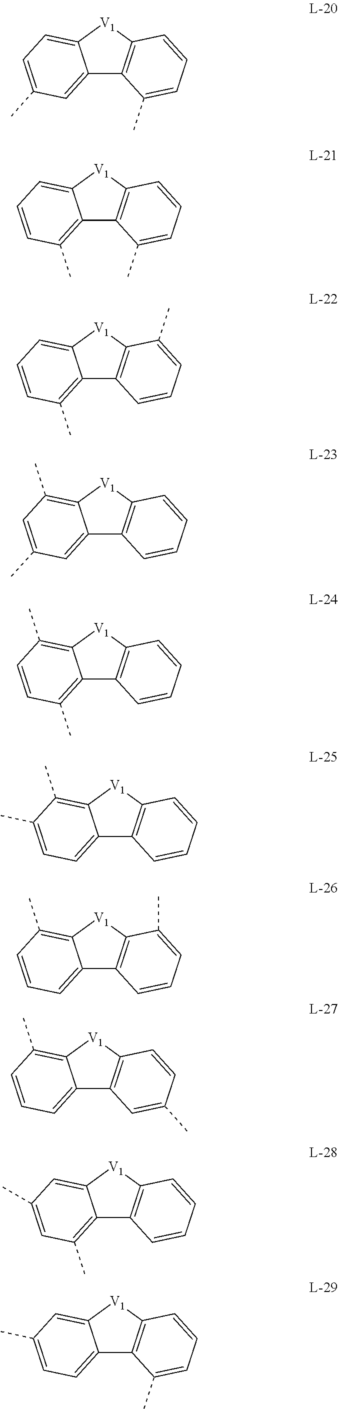

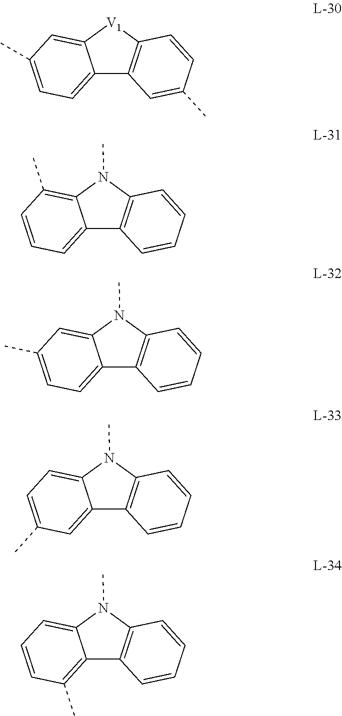

where the dashed lines denote the bond to the rest of the formula (1) or to the rest of the formulae (1a), (1b), (1c), (1d), (1e), (1f), (1g), (1h), (1i), (1j), (1k), (1l) or (1m);
V₁ is O, S or Se, and where R¹ has a definition given above or given hereinafter.

In a preferred embodiment of the invention, L in compounds of the formulae (1), (1a), (1b), (1c), (1d), (1e), (1f), (1g), (1h), (1i), (1j), (1k), (1l) and (1m) is a single bond.

In a preferred embodiment of the invention, the linker L in compounds of the formulae (1), (1a), (1b), (1c), (1d), (1e), (1f), (1g), (1h), (1i), (1j), (1k), (1l) and (1m) is a single bond or is selected from the group of linkers L-1 to L-30, which may be substituted by one or more R¹ radicals, where R¹ has a definition given above or given hereinafter. In the linkers L-18 to L-30, V₁ is preferably O or S, more preferably O.

From the group of linkers L-1 to L-30 that may be substituted by one or more R¹ radicals, the following linkers should be selected with preference: L-2, L-3, L-4, L-7, L-8, L-12, L-15, L-20, L-22, L-26, which may be substituted by one or more R¹ radicals, where R¹ has a definition given above or given below.

In a preferred embodiment of the invention, L in compounds of the formulae (1), (1a), (1b), (1c), (1d), (1e), (1f), (1g), (1h), (1i), (1j), (1k), (1l) and (1m) is linker L-15 which may be substituted by one or more R¹ radicals, where R¹ has a definition given above or given hereinafter.

The substituent R¹ where it occurs is the same or different and is preferably selected from the group of D, F, CN, Si(Ar)₃ or an aromatic or heteroaromatic ring system having 5 to 40 ring atoms, where Ar has a definition given above. The substituent R¹ where it occurs is preferably D, phenyl or dibenzofuranyl. Ar in Si(Ar)₃ is preferably the same and an aromatic ring system which has 6 to 20 ring atoms and may be substituted by one or more R² radicals. R² in Ar is preferably D, F or CN, more preferably D. In Si(Ar)₃, Ar is more preferably selected from undeuterated, partly deuterated or fully deuterated phenyl, 1,4-biphenyl, 1,3-biphenyl or 1,2-biphenyl. The substituent R¹ where it occurs is more preferably D.

The symbol V in the formulae (1-1) to (1-4) or in compounds of the formulae (1d) to (1m), as described above, preferably denotes O.

The invention accordingly further provides compounds of the formulae (1), (1a), (1b), (1c), (1d), (1e), (1f) or (1g), as described above with linkers L, as described above or described with preference, in which V is O.

In compounds of the formulae (1), (1a), (1b), (1c), (1d), (1e), (1f), (1g), (1h), (1i), (1j), (1k), (1l) and (1m) or compounds of the formulae (1), (1a), (1b), (1c), (1d), (1e), (1f), (1g), (1h), (1i), (1j), (1k), (1l) and (1m) that are mentioned with preference, (D)ₐ, (D)_{b} and (D)_{c} represent a monosubstitution, a disubstitution, a trisubstitution, the maximum permissible substitution or no substitution by deuterium.

When the compounds of the formulae (1), (1a), 1b), (1c), (1d), (1e), (1f), (1g), (1h), (1i), (1j), (1k), (1l) and (1m) are deuterated compounds, it is possible in the preparation thereof, if preparation by reaction of an undeuterated compound of one of the formulae (1), (1a), 1b), (1c), (1d), (1e), (1f), (1g), (1h), (1i), (1j), (1k), (1l) and (1m) with a deuteration source is chosen, or if deuterated starting compounds are chosen in the preparation that are a mixture of deuterated starting compounds, to form a mixture of deuterated products of the same chemical base structure that differ solely by the degree of deuteration and/or the deuteration patterns.

Such mixtures of deuterated compounds of the same chemical base structure of the formula (1) or of the base structure of the preferred embodiments that differ solely by the degree of deuteration and/or the deuteration patterns are considered to be covered by the expressions “compound of formula (1)” or “at least one compound of formula (1)” in the context of the invention.

In a preferred embodiment of the invention, the compounds of the formulae (1), (1a), (1b), (1c), (1d), (1e), (1f), (1g), (1h), (1i), (1j), (1k), (1l) and (1m) are deuterated, where the (average) degree of deuteration of the compounds of the formulae (1), (1a), (1b), (1c), (1d), (1e), (1f), (1g), (1h), (1i), (1j), (1k), (1l) and (1m) is preferably at least 10 mol % to 100 mol %, more preferably 50 mol % to 95 mol % and most preferably 70 mol % to 90 mol %.

Corresponding deuteration methods are known to the person skilled in the art and are described, for example, in KR2016041014, WO2017/122988, KR202005282, KR101978651 and WO2018/110887 or in Bulletin of the Chemical Society of Japan, 2021, 94(2), 600-605 or Asian Journal of Organic Chemistry, 2017, 6(8), 1063-1071.

A suitable method of deuterating a compound by exchange of one or more hydrogen atoms for deuterium atoms is a treatment of the compound to be deuterated in the presence of a platinum catalyst or palladium catalyst and a deuterium source. The term “deuterium source” means any compound that contains one or more deuterium atoms and is able to release them under suitable conditions.

The platinum catalyst is preferably dry platinum on charcoal, preferably 5% dry platinum on charcoal. The palladium catalyst is preferably dry palladium on charcoal, preferably 5% dry palladium on charcoal. A suitable deuterium source is D₂O, benzene-d6, chloroform-d, acetonitrile-d3, acetone-d6, acetic acid-d4, methanol-d4 or toluene-d8. A preferred deuterium source is D₂O or a combination of D₂O and a fully deuterated organic solvent. A particularly preferred deuterium source is the combination of D₂O with a fully deuterated organic solvent, where the fully deuterated solvent here is not restricted. Particularly suitable fully deuterated solvents are benzene-d6 and toluene-d8. A particularly preferred deuterium source is a combination of D₂O and toluene-d8. The reaction is preferably conducted with heating, more preferably with heating to temperatures between 100 °C and 200 °C. In addition, the reaction is preferably conducted under pressure.

In one embodiment of the invention, (D)ₐ, (D)_{b} and (D)_{c} represent no substitution.

In one embodiment of the invention, (D)ₐ, (D)_{b} and (D)_{c} represent maximum substitution.

In compounds of the formulae (1), (1a), (1b), (1c), (1d), (1e), (1f), (1g), (1h), (1i), (1j), (1k), (1l) and (1m) or compounds of the formulae (1), (1a), (1b), (1c), (1d), (1e), (1f), (1g), (1h), (1i), (1j), (1k), (1l) and (1m) that are mentioned with preference, R# at each instance is preferably independently D, F, CN or an undeuterated, a partly deuterated or a fully deuterated phenyl, more preferably for D. If R# is D, [R#]ₐ₂ preferably represents the maximum permissible substitution. If R# is F or CN, [R#]ₐ₂ preferably represents monosubstitution or disubstitution.

If R# is an undeuterated, a partly deuterated or a fully deuterated phenyl, [R#]ₐ₂ preferably represents monosubstitution.

In one embodiment of the invention, [R#]ₐ₂ in compounds of the formulae (1), (1a), (1b), (1c), (1d), (1e), (1f), (1g), (1h), (1i), (1j), (1k), (1l) and (1m) or in compounds of the formulae (1), (1a), (1b), (1c), (1d), (1e), (1f), (1g), (1h), (1i), (1j), (1k), (1l) and (1m) that are mentioned with preference represents no substitution.

In one embodiment of the invention, [R#]ₐ₂ in compounds of the formulae (1), (1a), (1b), (1c), (1d), (1e), (1f), (1g), (1h), (1i), (1j), (1k), (1l) and (1m) or in compounds of the formulae (1), (1a), (1b), (1c), (1d), (1e), (1f), (1g), (1h), (1i), (1j), (1k), (1l) and (1m) that are mentioned with preference represents no substitution or a monosubstitution, a disubstitution or the maximum permissible substitution by R#=D.

In compounds of the formulae (1), (1a), (1b), (1c), (1d), (1e), (1f), (1g), (1h), (1i), (1j), (1k), (1l) and (1m) or compounds of the formulae (1), (1a), (1b), (1c), (1d), (1e), (1f), (1g), (1h), (1i), (1j), (1k), (1l) and (1m) that are mentioned with preference, Ar, Ar₁, Ar₂ and Ar₄ are the same or different at each instance and are preferably an aromatic or heteroaromatic ring system which has 5 to 40 ring atoms and may be substituted by one or more R² radicals; where R² has a definition given above or given with preference.

In compounds of the formulae (1), (1a), (1b), (1c), (1d), (1e), (1f), (1g), (1h), (1i), (1j), (1k), (1l) and (1m) or compounds of the formulae (1), (1a), (1b), (1c), (1d), (1e), (1f), (1g), (1h), (1i), (1j), (1k), (1l) and (1m) that are mentioned with preference, Ar₁, Ar₂ and Ar₄ are the same or different at each instance and are preferably an aromatic or heteroaromatic ring system having 5 to 40 ring atoms from the group of Ar-1 to Ar-35:

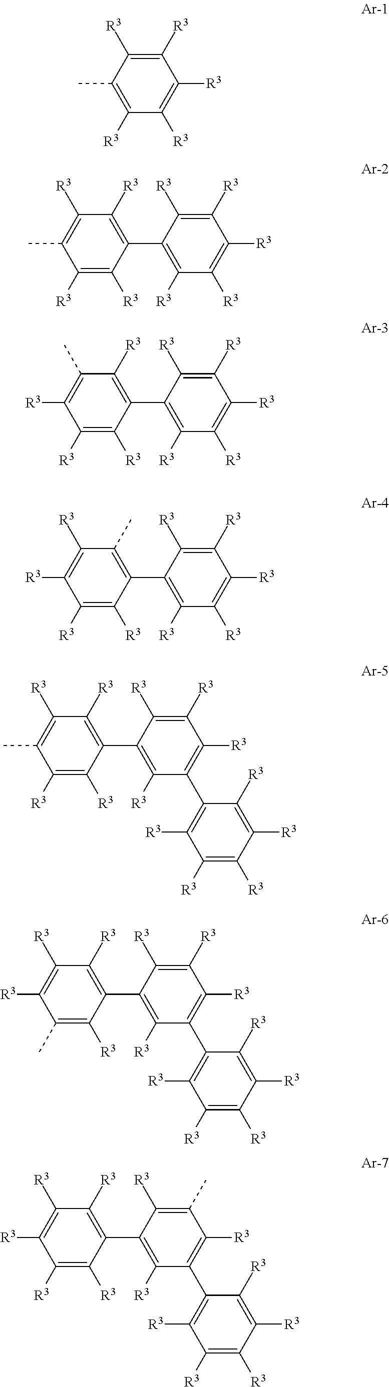

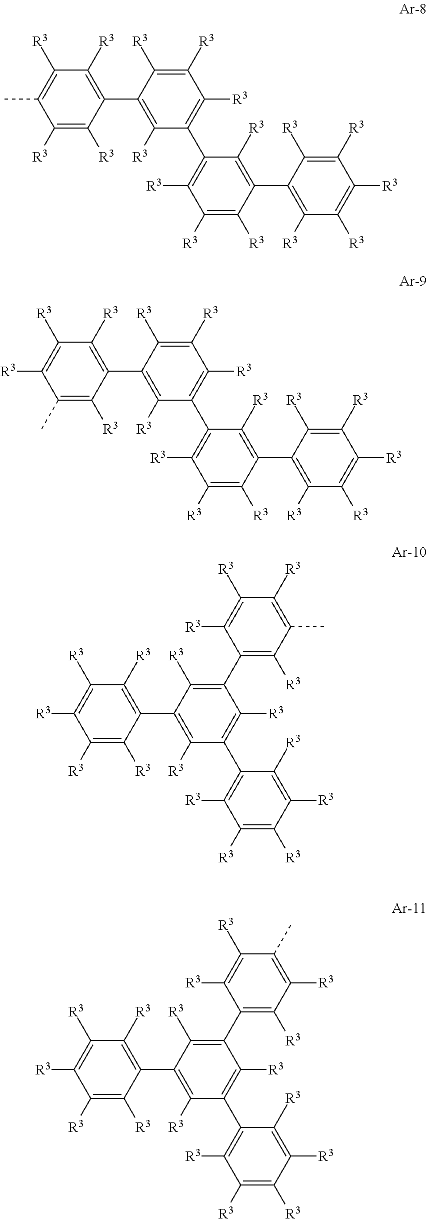

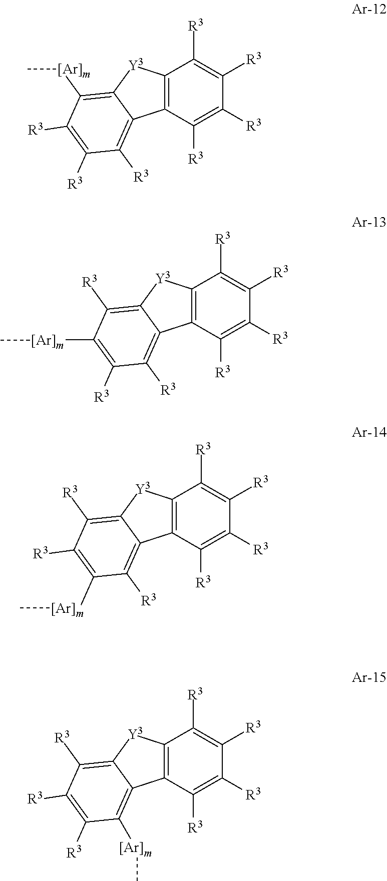

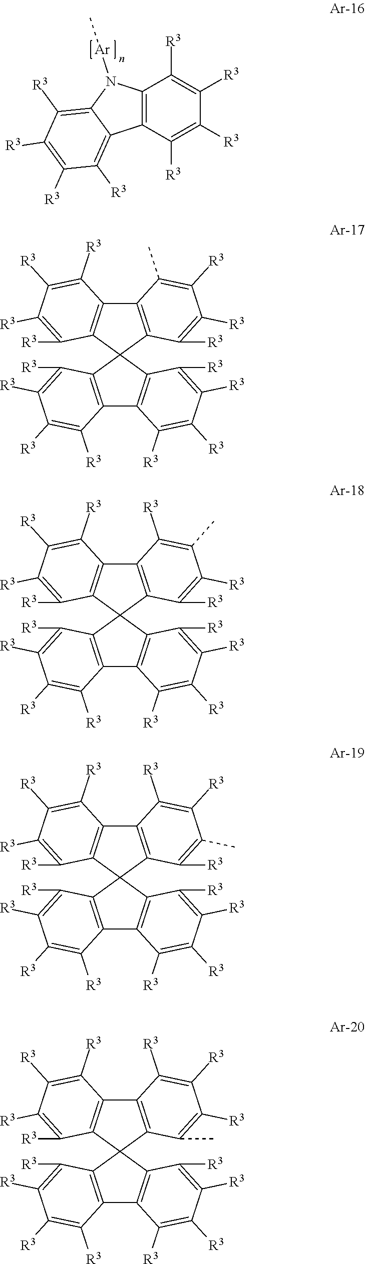

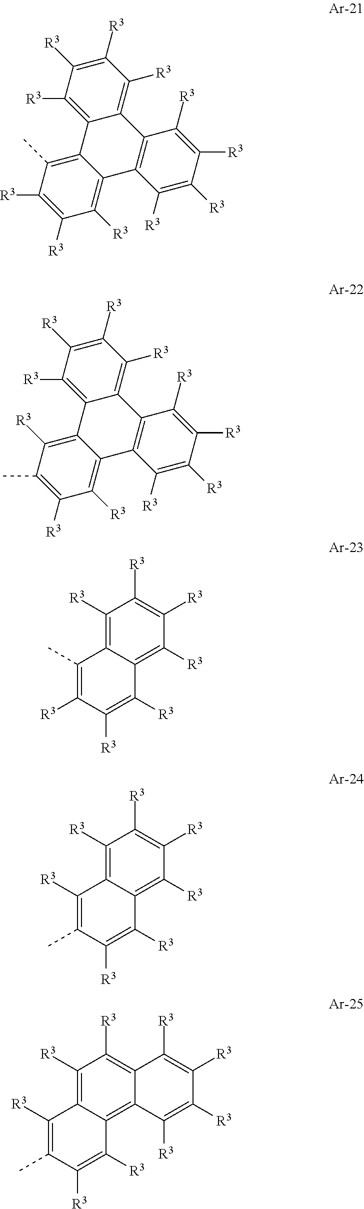

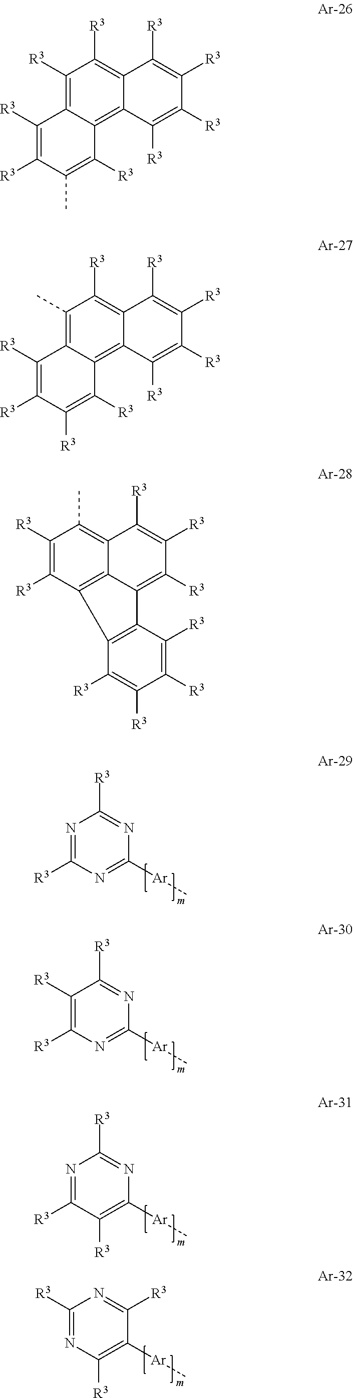

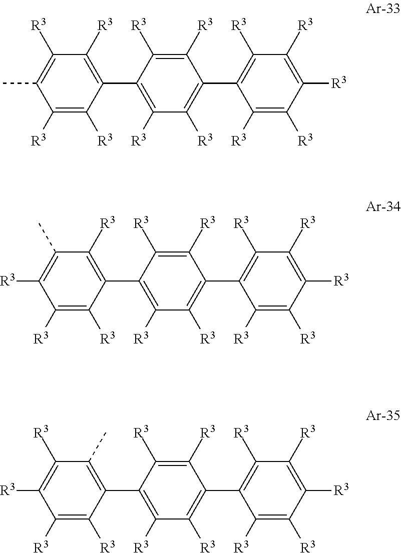

where
Y³ is the same or different at each instance and is O, S, NAr or C(R)₂,
R is methyl or phenyl,
R³ is H, R² or an aromatic or heteroaromatic ring system which has 5 to 40 ring atoms and may be substituted by one or more R² radicals;
the dashed bond represents the bond to the rest of the formulae (1), (1a), (1b), (1c), (1d), (1e), (1f), (1g), (1h), (1i), (1j), (1k), (1l) and (1m);
m is 0 or 1, where m=0 means that the ar group is absent, and
R² and Ar have a definition given above or a preferred given definition.

Y³ is preferably O, S, NAr or C(CH₃)₂. Y³ is most preferably O.

Ar is preferably phenyl, 1,2-biphenyl, 1,3-biphenyl, 1,4-biphenyl, triphenylenyl, dibenzofuranyl or dibenzothiophenyl, which may be substituted by one or more R² radicals, where R² has a definition given above or given with preference. Ar is more preferably phenyl, 1,2-biphenyl, 1,3-biphenyl or 1,4-biphenyl.

The substituent R² where it occurs is the same or different and is preferably selected from the group of D, F, CN or a straight-chain or branched alkyl group having 1 to 10 carbon atoms, where one or more hydrogen atoms of the alkyl group may be replaced by D, F, or CN. The substituent R² where it occurs is preferably D or F, more preferably D. R² in Ar is preferably D, F or CN, more preferably D.

In the structures Ar-1 to Ar-35, the substituent R³ is preferably the same or different at each instance and is selected from the group consisting of H, D, F, CN or an aromatic ring system which has 6 to 30 ring atoms and may be substituted in each case by one or more R² radicals. In the structures Ar-1 to Ar-35, the substituent R³ is more preferably the same or different at each instance and is selected from the group consisting of H, D, undeuterated or partly or fully deuterated phenyl, 1,4-biphenyl, 1,3-biphenyl or 1,2-biphenyl.

In compounds of the formulae (1), (1a), (1b), (1c), (1d), (1e), (1f), (1g), (1h), (1i), (1j), (1k), (1l) and (1m) or compounds of the formulae (1), (1a), (1b), (1c), (1d), (1e), (1f), (1g), (1h), (1i), (1j), (1k), (1l) and (1m) that are mentioned with preference, Ar₁, Ar₂ and Ar₄ are the same or different at each instance and are preferably a group selected from Ar-1 to Ar-28 and Ar-33 to Ar-35, as described above, or more preferably a group selected from Ar-1 to Ar-4, Ar-12 to Ar-16, Ar-21 to Ar-27 and Ar-33 to Ar-35, as described above.

In one embodiment of the invention, it is preferable when Ar₂ in the formula (1-1) or in compounds of the formulae (1d), (1h), (1i) and (1j) is the Ar-2 group, where R³ has a definition given above or given with preference.

In one embodiment of the invention, it is preferable when at least Ar or at least Ar₂ in the formula (1-2) or in compounds of the formulae (1e), (1k), (1l) and (1m), is the Ar-2 group, where R³ has a definition given above or given with preference.

In one embodiment of the invention, it is preferable when Ar₄ in the formula (1-3), the formula (1-4), or in compounds of the formulae (1f) and (1g), is the Ar-1 or Ar-2 group, where R³ has a definition given above or given with preference and a1 is 1.

In compounds of the formulae (1), (1a), (1b), (1c), (1f) and (1g) or compounds of the formulae (1), (1a), (1b), (1c), (1f) and (1g) that are mentioned with preference, Ar₃ is preferably a group selected from Ar-1 to Ar-15, Ar-17 to Ar-28 and Ar-33 to Ar-35, as described above, with the condition that Y³ is O, S or C(R)₂, or more preferably a group selected from Ar-1 to Ar-4, Ar-12 to Ar-15, Ar-21 to Ar-27, and Ar-33 to Ar-35, as described above, with the condition that Y³ is O, S, or C(R)₂.

In one embodiment of the invention, it is preferred when Ar₃ in the formula (1-3), the formula (1-4), or in compounds of the formulae (1f) and (1g), is the Ar-2 group, where R³ has a definition given above or given with preference.

Examples of suitable compounds of the formulae (1), (1a), (1b), (1c), (1d), (1e), (1f), (1g), (1h), (1i), (1j), (1k), (1l) and (1m) are the structures shown below in table 1.

TABLE 1

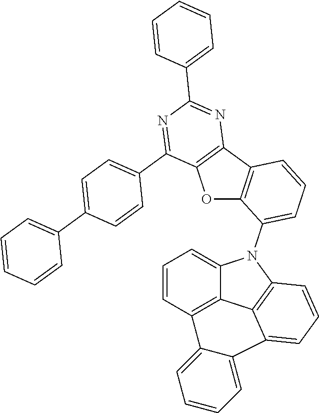 [SMILES: c1ccc(-c2ccc(-c3nc(-c4ccccc4)nc4c3oc3c(-n5c6cccc7c8ccccc8c8cccc5c8c76)cccc34)cc2)cc1]

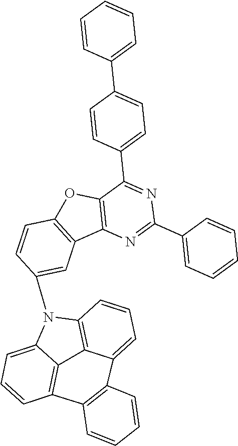 [SMILES: c1ccc(-c2ccc(-c3nc(-c4ccccc4)nc4c3oc3ccc(-n5c6cccc7c8ccccc8c8cccc5c8c76)cc34)cc2)cc1]

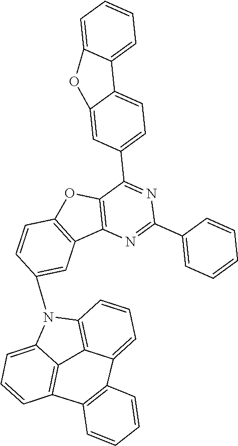 [SMILES: c1ccc(-c2nc(-c3ccc4c(c3)oc3ccccc34)c3oc4ccc(-n5c6cccc7c8ccccc8c8cccc5c8c76)cc4c3n2)cc1]

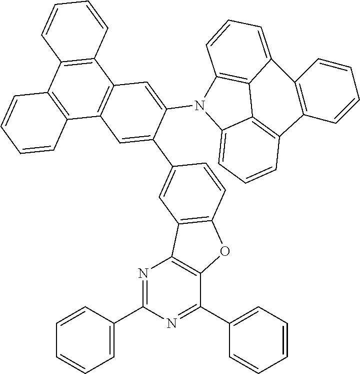 [SMILES: c1ccc(-c2nc(-c3ccccc3)c3oc4ccc(-c5cc6c7ccccc7c7ccccc7c6cc5-n5c6cccc7c8ccccc8c8cccc5c8c76)cc4c3n2)cc1]

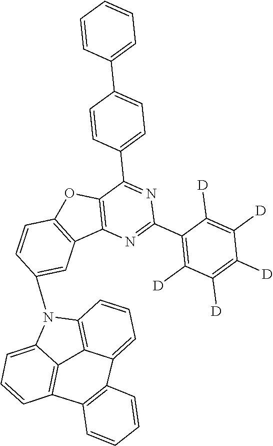 [SMILES: [2H]c1c([2H])c([2H])c(-c2nc(-c3ccc(-c4ccccc4)cc3)c3oc4ccc(-n5c6cccc7c8ccccc8c8cccc5c8c76)cc4c3n2)c([2H])c1[2H]]

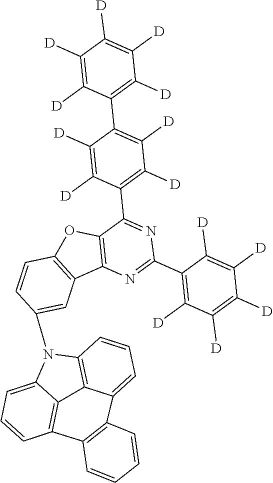 [SMILES: [2H]c1c([2H])c([2H])c(-c2nc(-c3c([2H])c([2H])c(-c4c([2H])c([2H])c([2H])c([2H])c4[2H])c([2H])c3[2H])c3oc4ccc(-n5c6cccc7c8ccccc8c8cccc5c8c76)cc4c3n2)c([2H])c1[2H]]

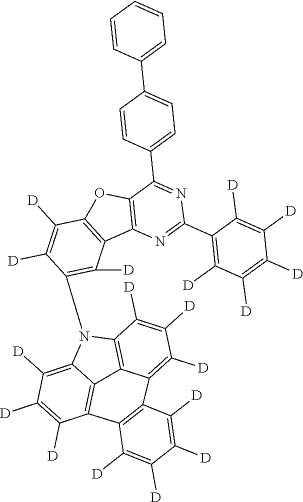 [SMILES: [2H]c1c([2H])c([2H])c(-c2nc(-c3ccc(-c4ccccc4)cc3)c3oc4c([2H])c([2H])c(-n5c6c([2H])c([2H])c([2H])c7c8c([2H])c([2H])c([2H])c([2H])c8c8c([2H])c([2H])c([2H])c5c8c76)c([2H])c4c3n2)c([2H])c1[2H]]

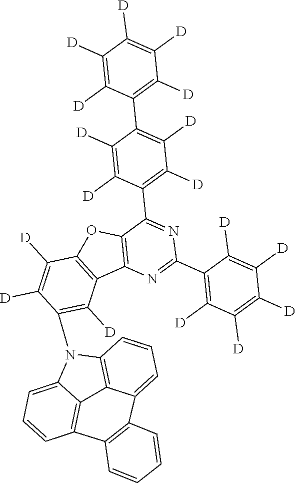 [SMILES: [2H]c1c([2H])c([2H])c(-c2nc(-c3c([2H])c([2H])c(-c4c([2H])c([2H])c([2H])c([2H])c4[2H])c([2H])c3[2H])c3oc4c([2H])c([2H])c(-n5c6cccc7c8ccccc8c8cccc5c8c76)c([2H])c4c3n2)c([2H])c1[2H]]

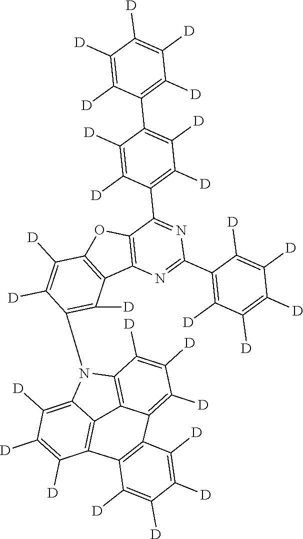 [SMILES: [2H]c1c([2H])c([2H])c(-c2nc(-c3c([2H])c([2H])c(-c4c([2H])c([2H])c([2H])c([2H])c4[2H])c([2H])c3[2H])c3oc4c([2H])c([2H])c(-n5c6c([2H])c([2H])c([2H])c7c8c([2H])c([2H])c([2H])c([2H])c8c8c([2H])c([2H])c([2H])c5c8c76)c([2H])c4c3n2)c([2H])c1[2H]]

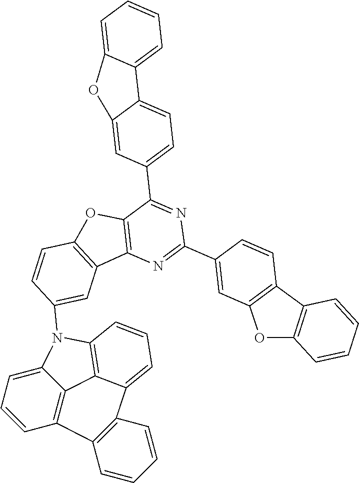 [SMILES: c1ccc2c(c1)oc1cc(-c3nc(-c4ccc5c(c4)oc4ccccc45)c4oc5ccc(-n6c7cccc8c9ccccc9c9cccc6c9c87)cc5c4n3)ccc12]

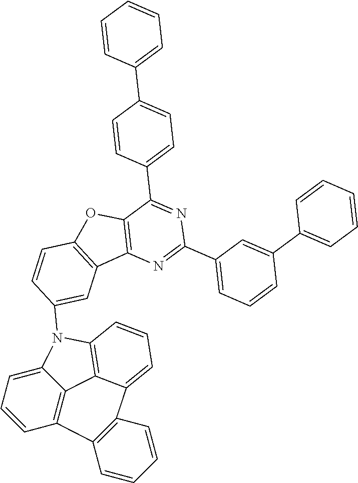 [SMILES: c1ccc(-c2ccc(-c3nc(-c4cccc(-c5ccccc5)c4)nc4c3oc3ccc(-n5c6cccc7c8ccccc8c8cccc5c8c76)cc34)cc2)cc1]

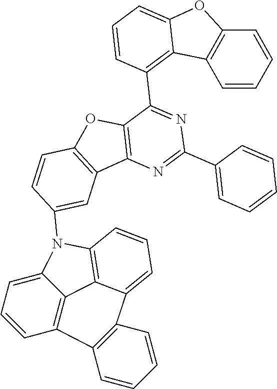 [SMILES: c1ccc(-c2nc(-c3cccc4oc5ccccc5c34)c3oc4ccc(-n5c6cccc7c8ccccc8c8cccc5c8c76)cc4c3n2)cc1]

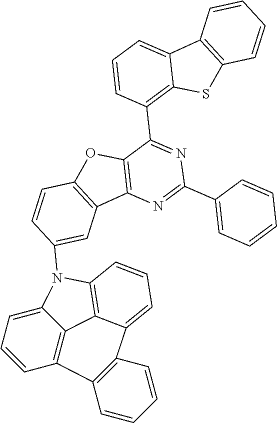 [SMILES: c1ccc(-c2nc(-c3cccc4c3sc3ccccc34)c3oc4ccc(-n5c6cccc7c8ccccc8c8cccc5c8c76)cc4c3n2)cc1]

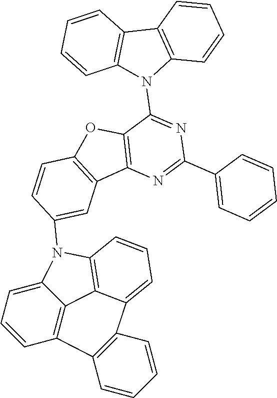 [SMILES: c1ccc(-c2nc(-n3c4ccccc4c4ccccc43)c3oc4ccc(-n5c6cccc7c8ccccc8c8cccc5c8c76)cc4c3n2)cc1]

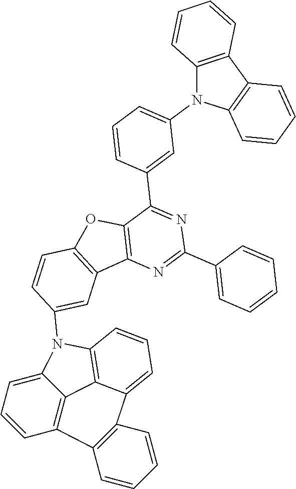 [SMILES: c1ccc(-c2nc(-c3cccc(-n4c5ccccc5c5ccccc54)c3)c3oc4ccc(-n5c6cccc7c8ccccc8c8cccc5c8c76)cc4c3n2)cc1]

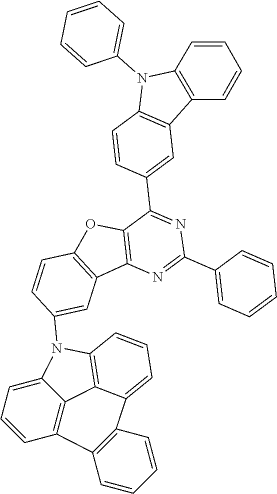 [SMILES: c1ccc(-c2nc(-c3ccc4c(c3)c3ccccc3n4-c3ccccc3)c3oc4ccc(-n5c6cccc7c8ccccc8c8cccc5c8c76)cc4c3n2)cc1]

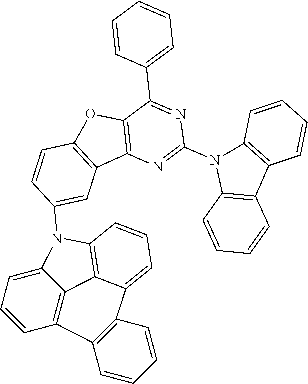 [SMILES: c1ccc(-c2nc(-n3c4ccccc4c4ccccc43)nc3c2oc2ccc(-n4c5cccc6c7ccccc7c7cccc4c7c65)cc23)cc1]

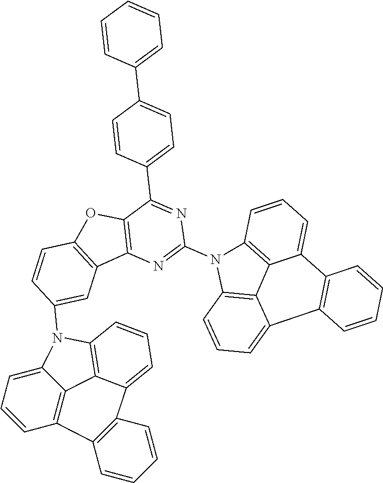 [SMILES: c1ccc(-c2ccc(-c3nc(-n4c5cccc6c7ccccc7c7cccc4c7c65)nc4c3oc3ccc(-n5c6cccc7c8ccccc8c8cccc5c8c76)cc34)cc2)cc1]

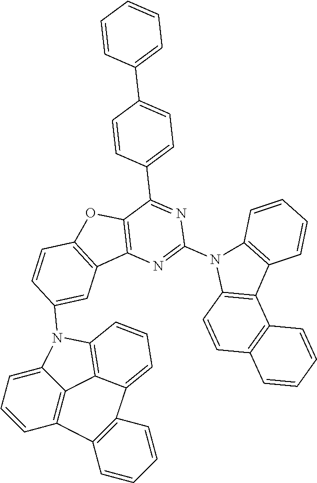 [SMILES: c1ccc(-c2ccc(-c3nc(-n4c5ccccc5c5c6ccccc6ccc54)nc4c3oc3ccc(-n5c6cccc7c8ccccc8c8cccc5c8c76)cc34)cc2)cc1]

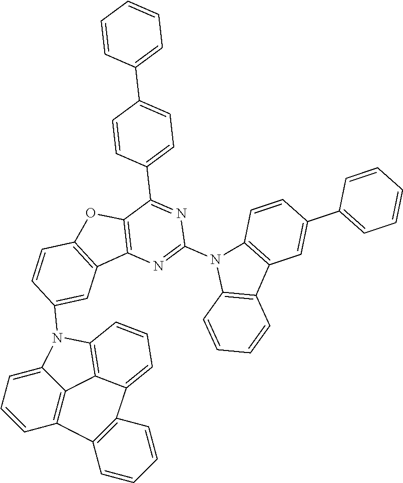 [SMILES: c1ccc(-c2ccc(-c3nc(-n4c5ccccc5c5cc(-c6ccccc6)ccc54)nc4c3oc3ccc(-n5c6cccc7c8ccccc8c8cccc5c8c76)cc34)cc2)cc1]

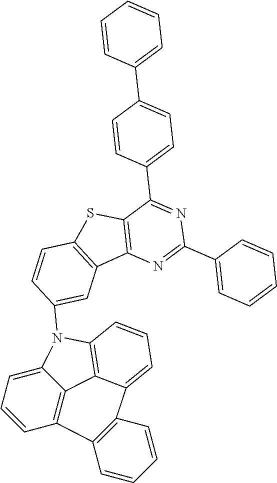 [SMILES: c1ccc(-c2ccc(-c3nc(-c4ccccc4)nc4c3sc3ccc(-n5c6cccc7c8ccccc8c8cccc5c8c76)cc34)cc2)cc1]

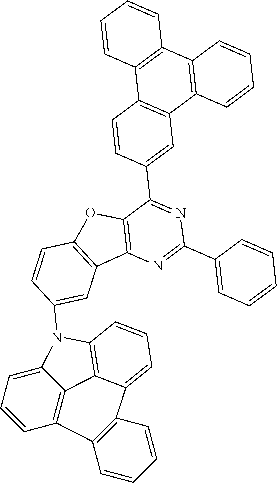 [SMILES: c1ccc(-c2nc(-c3ccc4c5ccccc5c5ccccc5c4c3)c3oc4ccc(-n5c6cccc7c8ccccc8c8cccc5c8c76)cc4c3n2)cc1]

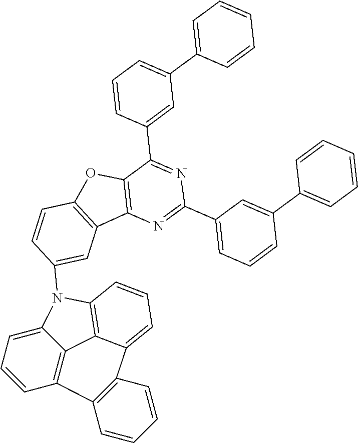 [SMILES: c1ccc(-c2cccc(-c3nc(-c4cccc(-c5ccccc5)c4)c4oc5ccc(-n6c7cccc8c9ccccc9c9cccc6c9c87)cc5c4n3)c2)cc1]

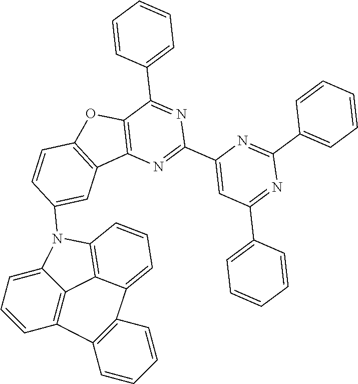 [SMILES: c1ccc(-c2cc(-c3nc(-c4ccccc4)c4oc5ccc(-n6c7cccc8c9ccccc9c9cccc6c9c87)cc5c4n3)nc(-c3ccccc3)n2)cc1]

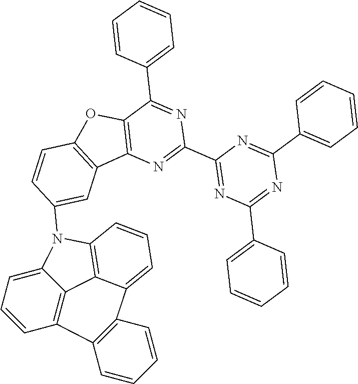 [SMILES: c1ccc(-c2nc(-c3ccccc3)nc(-c3nc(-c4ccccc4)c4oc5ccc(-n6c7cccc8c9ccccc9c9cccc6c9c87)cc5c4n3)n2)cc1]

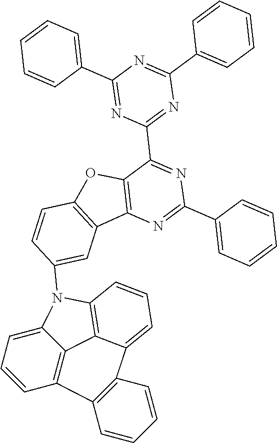 [SMILES: c1ccc(-c2nc(-c3ccccc3)nc(-c3nc(-c4ccccc4)nc4c3oc3ccc(-n5c6cccc7c8ccccc8c8cccc5c8c76)cc34)n2)cc1]

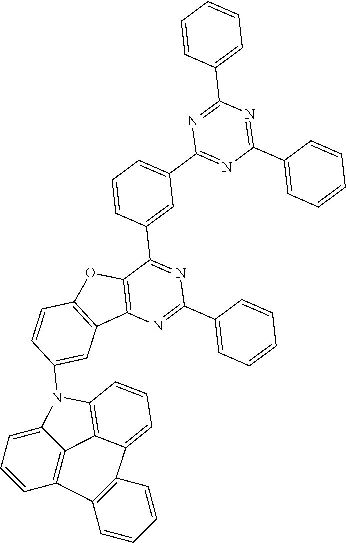 [SMILES: c1ccc(-c2nc(-c3ccccc3)nc(-c3cccc(-c4nc(-c5ccccc5)nc5c4oc4ccc(-n6c7cccc8c9ccccc9c9cccc6c9c87)cc45)c3)n2)cc1]

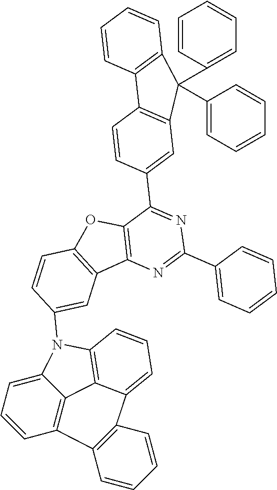 [SMILES: c1ccc(-c2nc(-c3ccc4c(c3)C(c3ccccc3)(c3ccccc3)c3ccccc3-4)c3oc4ccc(-n5c6cccc7c8ccccc8c8cccc5c8c76)cc4c3n2)cc1]

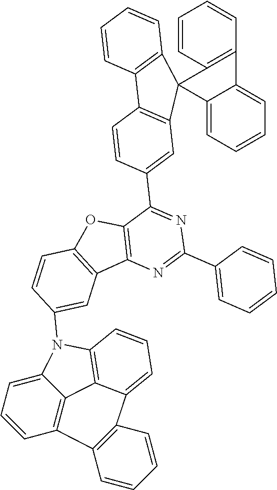 [SMILES: c1ccc(-c2nc(-c3ccc4c(c3)C3(c5ccccc5-c5ccccc53)c3ccccc3-4)c3oc4ccc(-n5c6cccc7c8ccccc8c8cccc5c8c76)cc4c3n2)cc1]

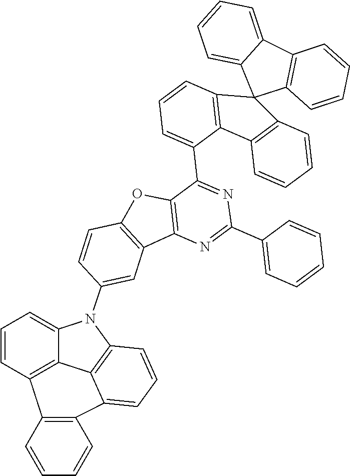 [SMILES: c1ccc(-c2nc(-c3cccc4c3-c3ccccc3C43c4ccccc4-c4ccccc43)c3oc4ccc(-n5c6cccc7c8ccccc8c8cccc5c8c76)cc4c3n2)cc1]

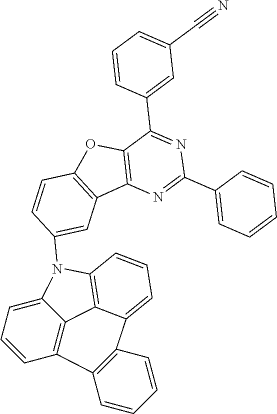 [SMILES: N#Cc1cccc(-c2nc(-c3ccccc3)nc3c2oc2ccc(-n4c5cccc6c7ccccc7c7cccc4c7c65)cc23)c1]

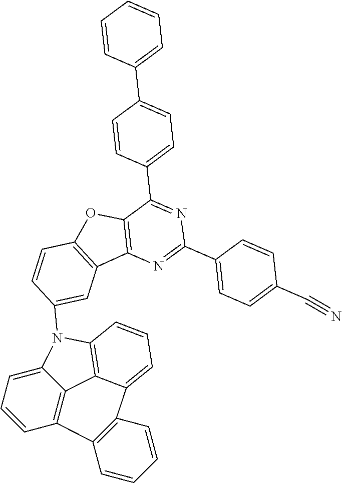 [SMILES: N#Cc1ccc(-c2nc(-c3ccc(-c4ccccc4)cc3)c3oc4ccc(-n5c6cccc7c8ccccc8c8cccc5c8c76)cc4c3n2)cc1]

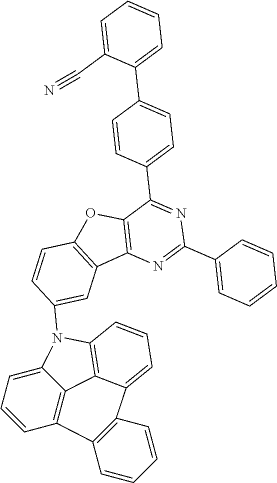 [SMILES: N#Cc1ccccc1-c1ccc(-c2nc(-c3ccccc3)nc3c2oc2ccc(-n4c5cccc6c7ccccc7c7cccc4c7c65)cc23)cc1]

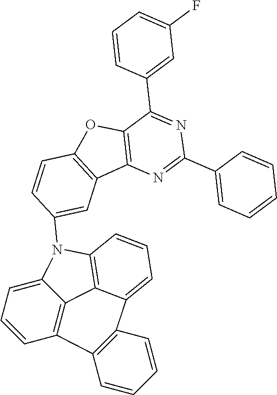 [SMILES: Fc1cccc(-c2nc(-c3ccccc3)nc3c2oc2ccc(-n4c5cccc6c7ccccc7c7cccc4c7c65)cc23)c1]

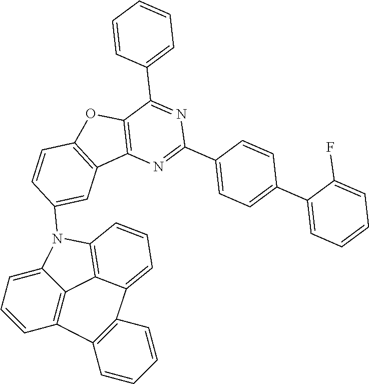 [SMILES: Fc1ccccc1-c1ccc(-c2nc(-c3ccccc3)c3oc4ccc(-n5c6cccc7c8ccccc8c8cccc5c8c76)cc4c3n2)cc1]

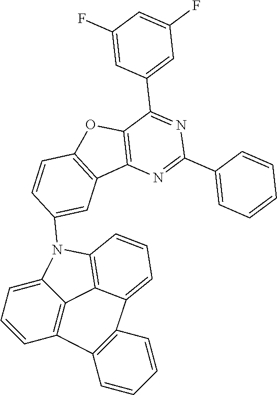 [SMILES: Fc1cc(F)cc(-c2nc(-c3ccccc3)nc3c2oc2ccc(-n4c5cccc6c7ccccc7c7cccc4c7c65)cc23)c1]

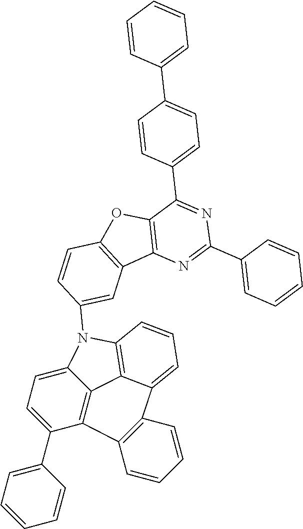 [SMILES: c1ccc(-c2ccc(-c3nc(-c4ccccc4)nc4c3oc3ccc(-n5c6cccc7c8ccccc8c8c(-c9ccccc9)ccc5c8c76)cc34)cc2)cc1]

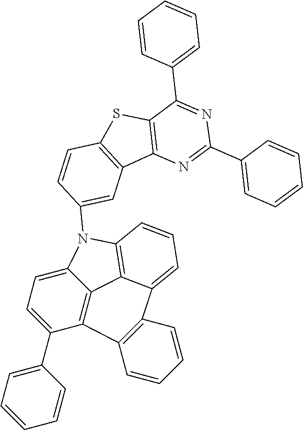 [SMILES: c1ccc(-c2nc(-c3ccccc3)c3sc4ccc(-n5c6cccc7c8ccccc8c8c(-c9ccccc9)ccc5c8c76)cc4c3n2)cc1]

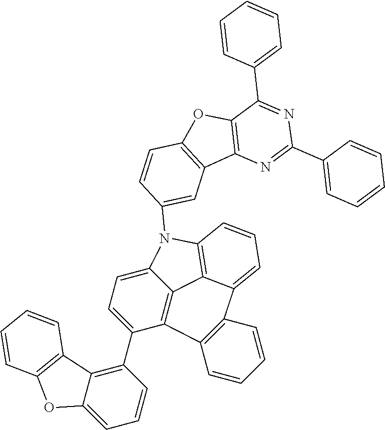 [SMILES: c1ccc(-c2nc(-c3ccccc3)c3oc4ccc(-n5c6cccc7c8ccccc8c8c(-c9cccc%10oc%11ccccc%11c9%10)ccc5c8c76)cc4c3n2)cc1]

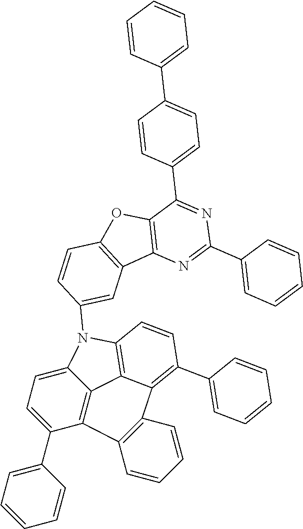 [SMILES: c1ccc(-c2ccc(-c3nc(-c4ccccc4)nc4c3oc3ccc(-n5c6ccc(-c7ccccc7)c7c8ccccc8c8c(-c9ccccc9)ccc5c8c76)cc34)cc2)cc1]

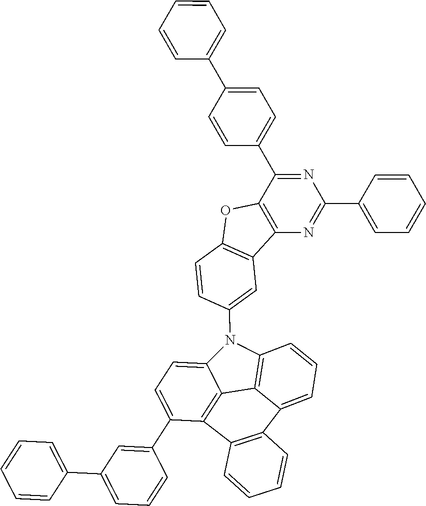 [SMILES: c1ccc(-c2ccc(-c3nc(-c4ccccc4)nc4c3oc3ccc(-n5c6cccc7c8ccccc8c8c(-c9cccc(-c%10ccccc%10)c9)ccc5c8c76)cc34)cc2)cc1]

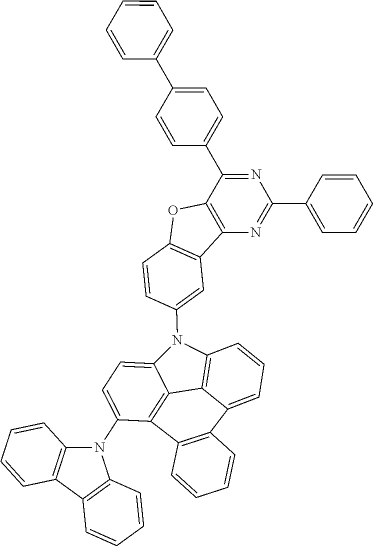 [SMILES: c1ccc(-c2ccc(-c3nc(-c4ccccc4)nc4c3oc3ccc(-n5c6cccc7c8ccccc8c8c(-n9c%10ccccc%10c%10ccccc%109)ccc5c8c76)cc34)cc2)cc1]

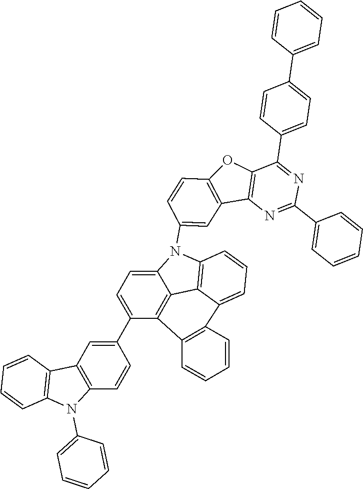 [SMILES: c1ccc(-c2ccc(-c3nc(-c4ccccc4)nc4c3oc3ccc(-n5c6cccc7c8ccccc8c8c(-c9ccc%10c(c9)c9ccccc9n%10-c9ccccc9)ccc5c8c76)cc34)cc2)cc1]

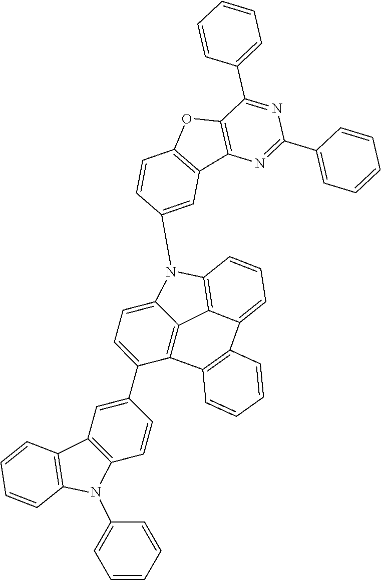 [SMILES: c1ccc(-c2nc(-c3ccccc3)c3oc4ccc(-n5c6cccc7c8ccccc8c8c(-c9ccc%10c(c9)c9ccccc9n%10-c9ccccc9)ccc5c8c76)cc4c3n2)cc1]

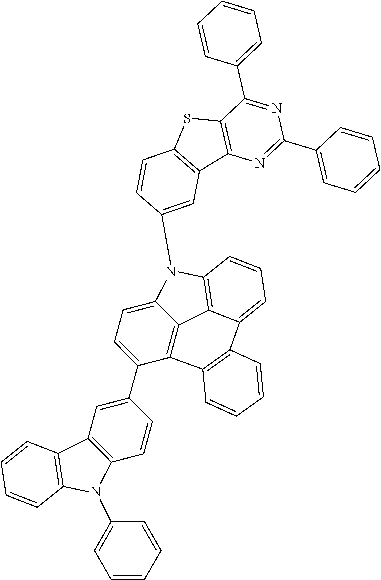 [SMILES: c1ccc(-c2nc(-c3ccccc3)c3sc4ccc(-n5c6cccc7c8ccccc8c8c(-c9ccc%10c(c9)c9ccccc9n%10-c9ccccc9)ccc5c8c76)cc4c3n2)cc1]

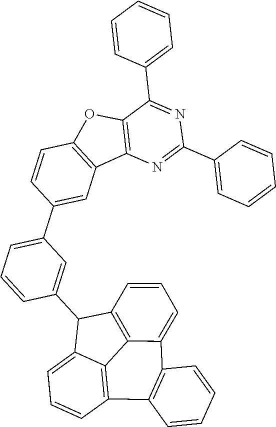 [SMILES: c1ccc(-c2nc(-c3ccccc3)c3oc4ccc(-c5cccc(C6c7cccc8c9ccccc9c9cccc6c9c78)c5)cc4c3n2)cc1]

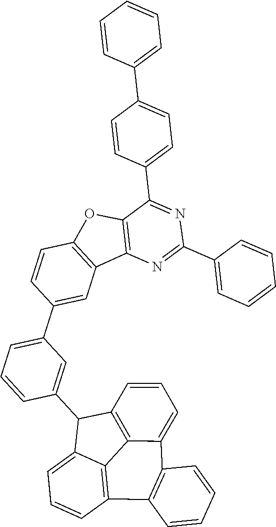 [SMILES: c1ccc(-c2ccc(-c3nc(-c4ccccc4)nc4c3oc3ccc(-c5cccc(C6c7cccc8c9ccccc9c9cccc6c9c78)c5)cc34)cc2)cc1]

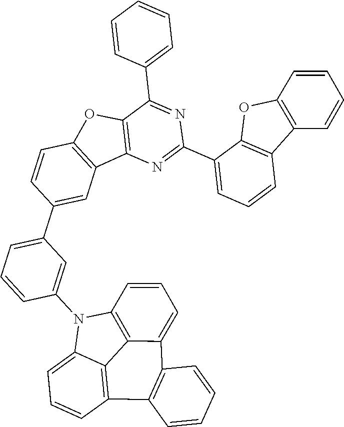 [SMILES: c1ccc(-c2nc(-c3cccc4c3oc3ccccc34)nc3c2oc2ccc(-c4cccc(-n5c6cccc7c8ccccc8c8cccc5c8c76)c4)cc23)cc1]

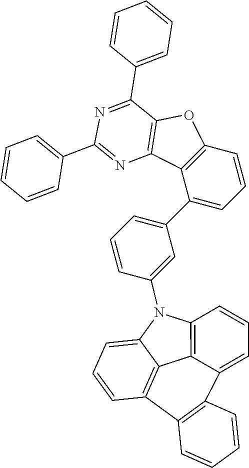 [SMILES: c1ccc(-c2nc(-c3ccccc3)c3oc4cccc(-c5cccc(-n6c7cccc8c9ccccc9c9cccc6c9c87)c5)c4c3n2)cc1]

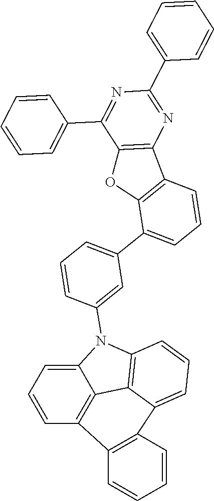 [SMILES: c1ccc(-c2nc(-c3ccccc3)c3oc4c(-c5cccc(-n6c7cccc8c9ccccc9c9cccc6c9c87)c5)cccc4c3n2)cc1]

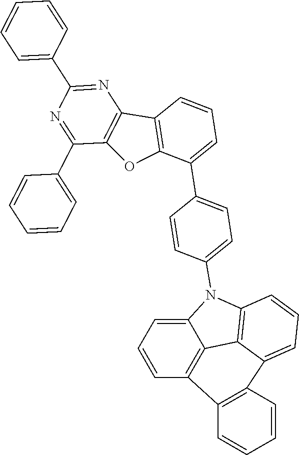 [SMILES: c1ccc(-c2nc(-c3ccccc3)c3oc4c(-c5ccc(-n6c7cccc8c9ccccc9c9cccc6c9c87)cc5)cccc4c3n2)cc1]

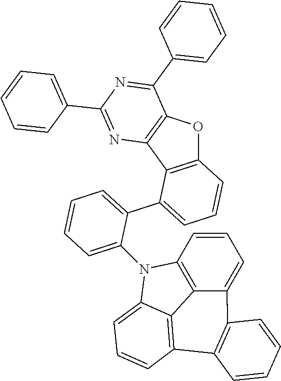 [SMILES: c1ccc(-c2nc(-c3ccccc3)c3oc4cccc(-c5ccccc5-n5c6cccc7c8ccccc8c8cccc5c8c76)c4c3n2)cc1]

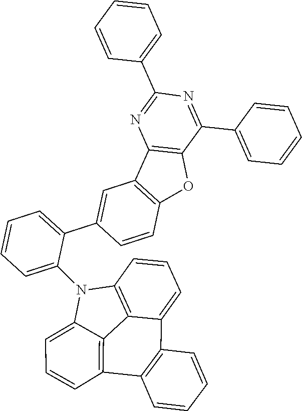 [SMILES: c1ccc(-c2nc(-c3ccccc3)c3oc4ccc(-c5ccccc5-n5c6cccc7c8ccccc8c8cccc5c8c76)cc4c3n2)cc1]

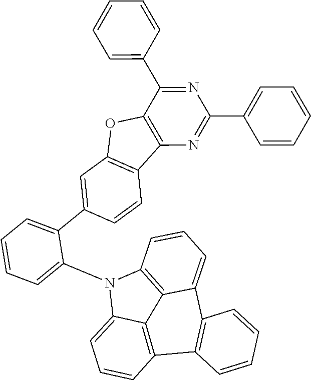 [SMILES: c1ccc(-c2nc(-c3ccccc3)c3oc4cc(-c5ccccc5-n5c6cccc7c8ccccc8c8cccc5c8c76)ccc4c3n2)cc1]

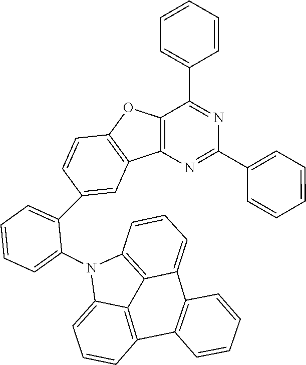 [SMILES: c1ccc(-c2nc(-c3ccccc3)c3oc4ccc(-c5ccccc5-n5c6cccc7c8ccccc8c8cccc5c8c76)cc4c3n2)cc1]

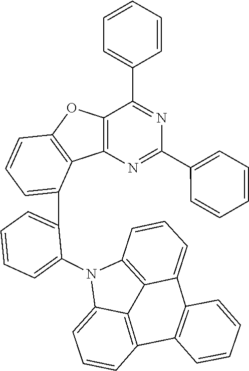 [SMILES: c1ccc(-c2nc(-c3ccccc3)c3oc4cccc(-c5ccccc5-n5c6cccc7c8ccccc8c8cccc5c8c76)c4c3n2)cc1]

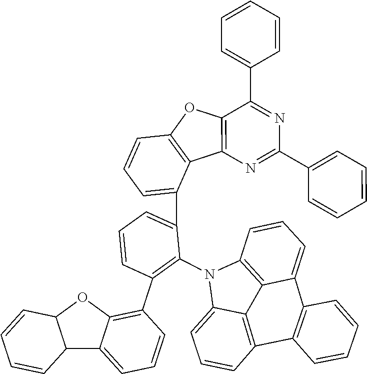 [SMILES: C1=CC2Oc3c(-c4cccc(-c5cccc6oc7c(-c8ccccc8)nc(-c8ccccc8)nc7c56)c4-n4c5cccc6c7ccccc7c7cccc4c7c65)cccc3C2C=C1]

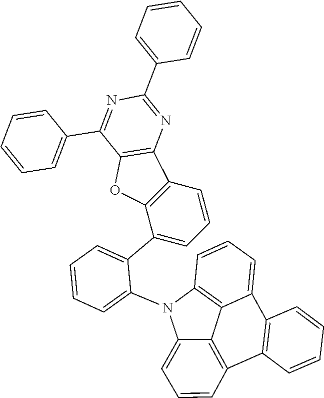 [SMILES: c1ccc(-c2nc(-c3ccccc3)c3oc4c(-c5ccccc5-n5c6cccc7c8ccccc8c8cccc5c8c76)cccc4c3n2)cc1]

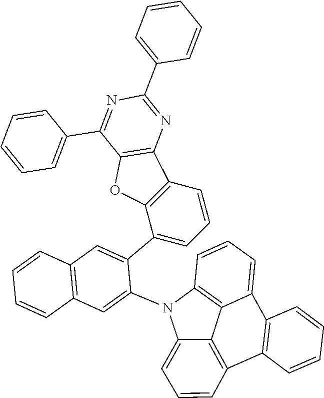 [SMILES: c1ccc(-c2nc(-c3ccccc3)c3oc4c(-c5cc6ccccc6cc5-n5c6cccc7c8ccccc8c8cccc5c8c76)cccc4c3n2)cc1]

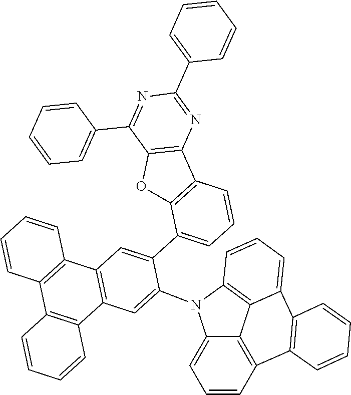 [SMILES: c1ccc(-c2nc(-c3ccccc3)c3oc4c(-c5cc6c7ccccc7c7ccccc7c6cc5-n5c6cccc7c8ccccc8c8cccc5c8c76)cccc4c3n2)cc1]

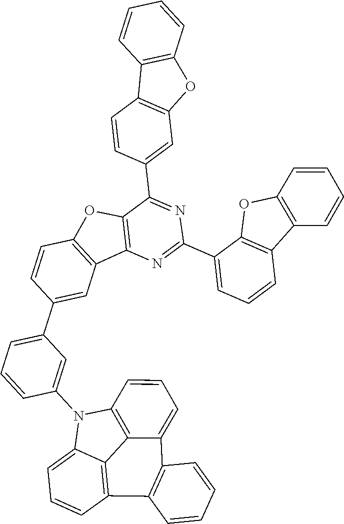 [SMILES: c1cc(-c2ccc3oc4c(-c5ccc6c(c5)oc5ccccc56)nc(-c5cccc6c5oc5ccccc56)nc4c3c2)cc(-n2c3cccc4c5ccccc5c5cccc2c5c43)c1]

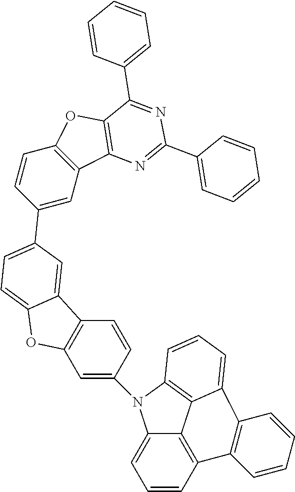 [SMILES: c1ccc(-c2nc(-c3ccccc3)c3oc4ccc(-c5ccc6oc7cc(-n8c9cccc%10c%11ccccc%11c%11cccc8c%11c%109)ccc7c6c5)cc4c3n2)cc1]

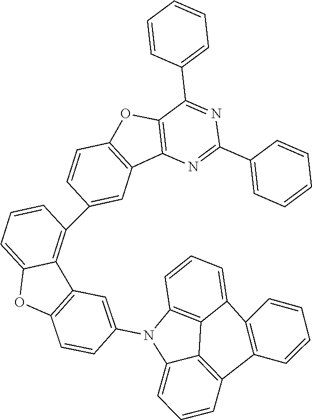 [SMILES: c1ccc(-c2nc(-c3ccccc3)c3oc4ccc(-c5cccc6oc7ccc(-n8c9cccc%10c%11ccccc%11c%11cccc8c%11c%109)cc7c56)cc4c3n2)cc1]

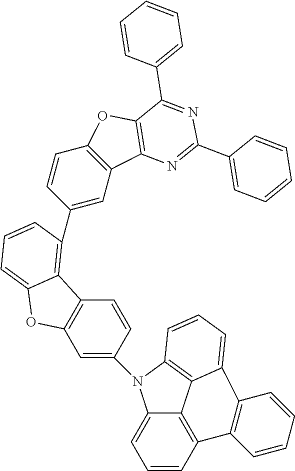 [SMILES: c1ccc(-c2nc(-c3ccccc3)c3oc4ccc(-c5cccc6oc7cc(-n8c9cccc%10c%11ccccc%11c%11cccc8c%11c%109)ccc7c56)cc4c3n2)cc1]

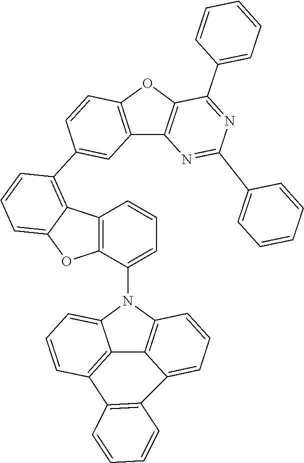 [SMILES: c1ccc(-c2nc(-c3ccccc3)c3oc4ccc(-c5cccc6oc7c(-n8c9cccc%10c%11ccccc%11c%11cccc8c%11c%109)cccc7c56)cc4c3n2)cc1]

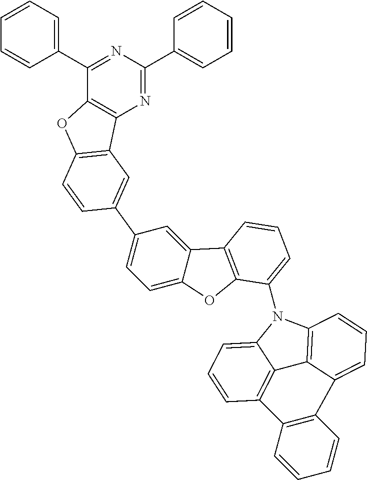 [SMILES: c1ccc(-c2nc(-c3ccccc3)c3oc4ccc(-c5ccc6oc7c(-n8c9cccc%10c%11ccccc%11c%11cccc8c%11c%109)cccc7c6c5)cc4c3n2)cc1]

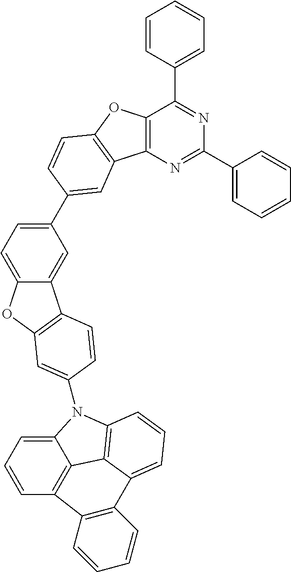 [SMILES: c1ccc(-c2nc(-c3ccccc3)c3oc4ccc(-c5ccc6oc7cc(-n8c9cccc%10c%11ccccc%11c%11cccc8c%11c%109)ccc7c6c5)cc4c3n2)cc1]

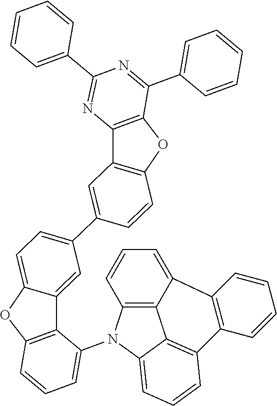 [SMILES: c1ccc(-c2nc(-c3ccccc3)c3oc4ccc(-c5ccc6oc7cccc(-n8c9cccc%10c%11ccccc%11c%11cccc8c%11c%109)c7c6c5)cc4c3n2)cc1]

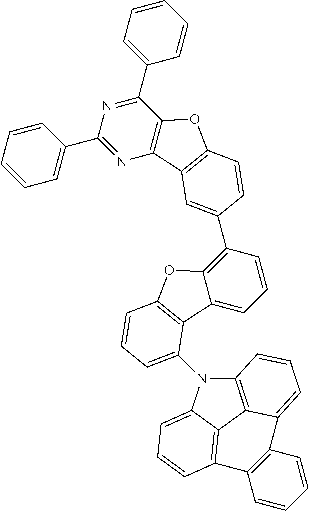 [SMILES: c1ccc(-c2nc(-c3ccccc3)c3oc4ccc(-c5cccc6c5oc5cccc(-n7c8cccc9c%10ccccc%10c%10cccc7c%10c98)c56)cc4c3n2)cc1]

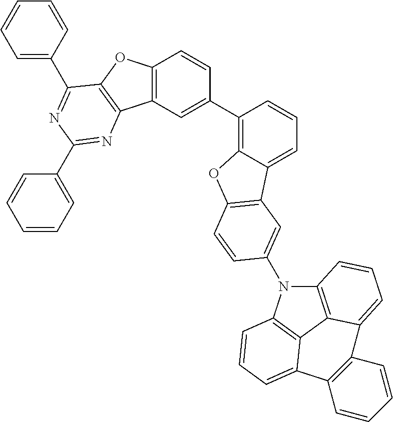 [SMILES: c1ccc(-c2nc(-c3ccccc3)c3oc4ccc(-c5cccc6c5oc5ccc(-n7c8cccc9c%10ccccc%10c%10cccc7c%10c98)cc56)cc4c3n2)cc1]

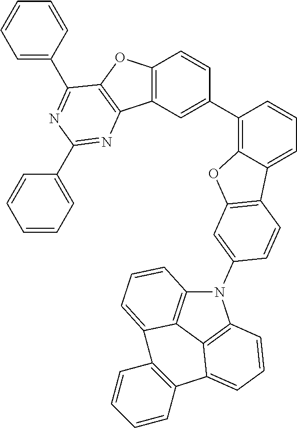 [SMILES: c1ccc(-c2nc(-c3ccccc3)c3oc4ccc(-c5cccc6c5oc5cc(-n7c8cccc9c%10ccccc%10c%10cccc7c%10c98)ccc56)cc4c3n2)cc1]

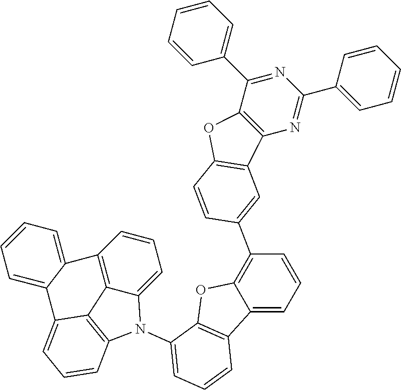 [SMILES: c1ccc(-c2nc(-c3ccccc3)c3oc4ccc(-c5cccc6c5oc5c(-n7c8cccc9c%10ccccc%10c%10cccc7c%10c98)cccc56)cc4c3n2)cc1]

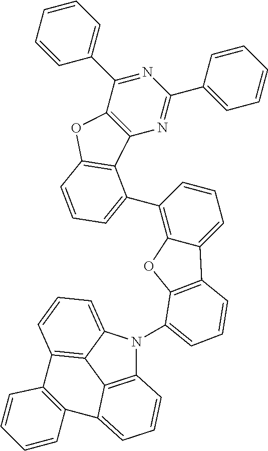 [SMILES: c1ccc(-c2nc(-c3ccccc3)c3oc4cccc(-c5cccc6c5oc5c(-n7c8cccc9c%10ccccc%10c%10cccc7c%10c98)cccc56)c4c3n2)cc1]

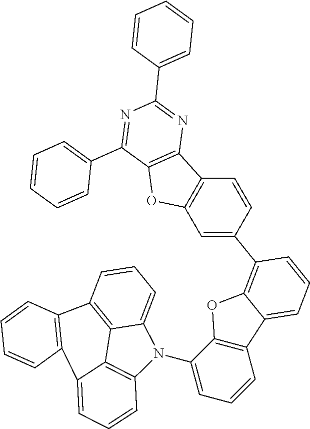 [SMILES: c1ccc(-c2nc(-c3ccccc3)c3oc4cc(-c5cccc6c5oc5c(-n7c8cccc9c%10ccccc%10c%10cccc7c%10c98)cccc56)ccc4c3n2)cc1]

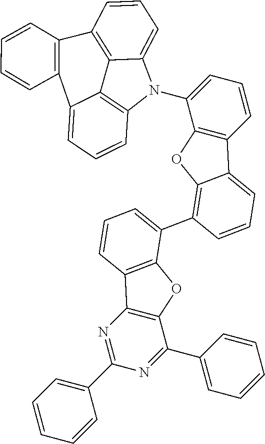 [SMILES: c1ccc(-c2nc(-c3ccccc3)c3oc4c(-c5cccc6c5oc5c(-n7c8cccc9c%10ccccc%10c%10cccc7c%10c98)cccc56)cccc4c3n2)cc1]

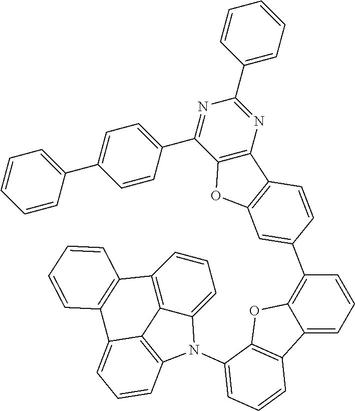 [SMILES: c1ccc(-c2ccc(-c3nc(-c4ccccc4)nc4c3oc3cc(-c5cccc6c5oc5c(-n7c8cccc9c%10ccccc%10c%10cccc7c%10c98)cccc56)ccc34)cc2)cc1]

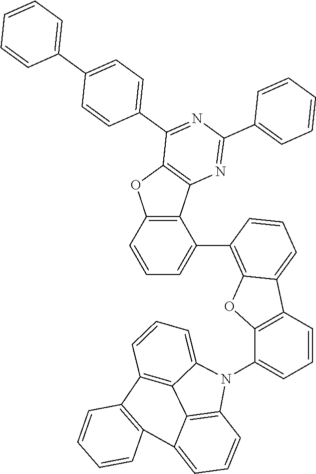 [SMILES: c1ccc(-c2ccc(-c3nc(-c4ccccc4)nc4c3oc3cccc(-c5cccc6c5oc5c(-n7c8cccc9c%10ccccc%10c%10cccc7c%10c98)cccc56)c34)cc2)cc1]

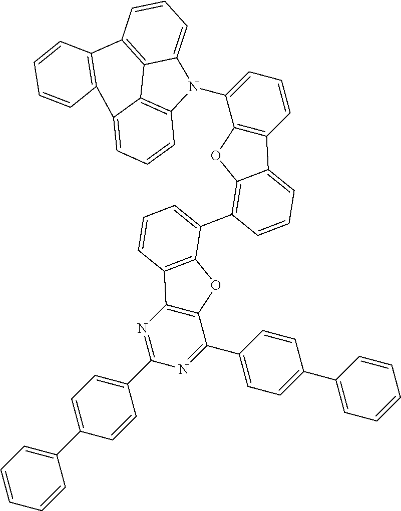 [SMILES: c1ccc(-c2ccc(-c3nc(-c4ccc(-c5ccccc5)cc4)c4oc5c(-c6cccc7c6oc6c(-n8c9cccc%10c%11ccccc%11c%11cccc8c%11c%109)cccc67)cccc5c4n3)cc2)cc1]

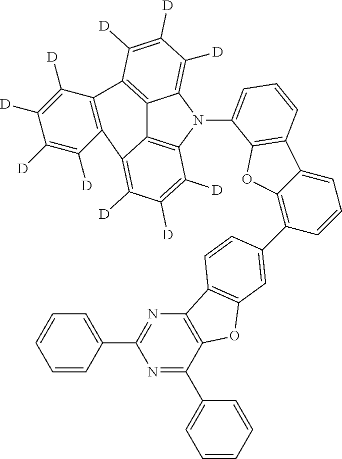 [SMILES: [2H]c1c([2H])c([2H])c2c(c1[2H])c1c([2H])c([2H])c([2H])c3c1c1c2c([2H])c([2H])c([2H])c1n3-c1cccc2c1oc1c(-c3ccc4c(c3)oc3c(-c5ccccc5)nc(-c5ccccc5)nc34)cccc12]

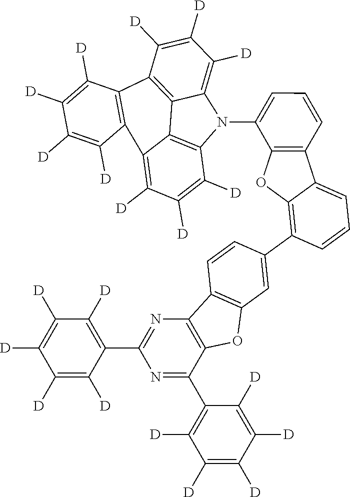 [SMILES: [2H]c1c([2H])c([2H])c(-c2nc(-c3c([2H])c([2H])c([2H])c([2H])c3[2H])c3oc4cc(-c5cccc6c5oc5c(-n7c8c([2H])c([2H])c([2H])c9c%10c([2H])c([2H])c([2H])c([2H])c%10c%10c([2H])c([2H])c([2H])c7c%10c98)cccc56)ccc4c3n2)c([2H])c1[2H]]

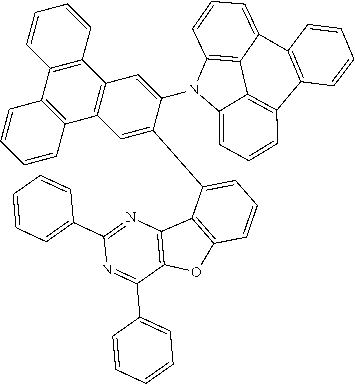 [SMILES: c1ccc(-c2nc(-c3ccccc3)c3oc4cccc(-c5cc6c7ccccc7c7ccccc7c6cc5-n5c6cccc7c8ccccc8c8cccc5c8c76)c4c3n2)cc1]

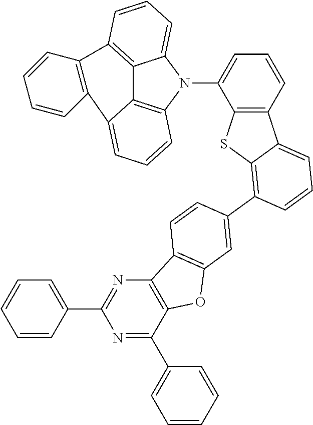 [SMILES: c1ccc(-c2nc(-c3ccccc3)c3oc4cc(-c5cccc6c5sc5c(-n7c8cccc9c%10ccccc%10c%10cccc7c%10c98)cccc56)ccc4c3n2)cc1]

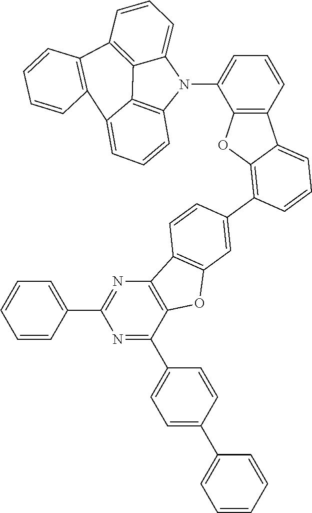 [SMILES: c1ccc(-c2ccc(-c3nc(-c4ccccc4)nc4c3oc3cc(-c5cccc6c5oc5c(-n7c8cccc9c%10ccccc%10c%10cccc7c%10c98)cccc56)ccc34)cc2)cc1]

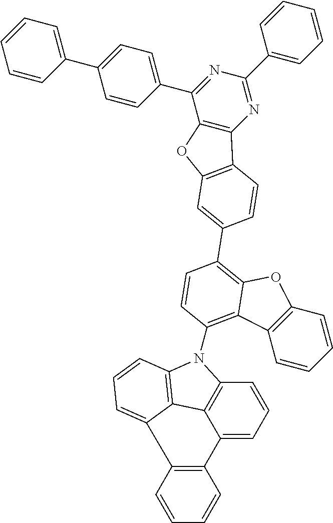 [SMILES: c1ccc(-c2ccc(-c3nc(-c4ccccc4)nc4c3oc3cc(-c5ccc(-n6c7cccc8c9ccccc9c9cccc6c9c87)c6c5oc5ccccc56)ccc34)cc2)cc1]

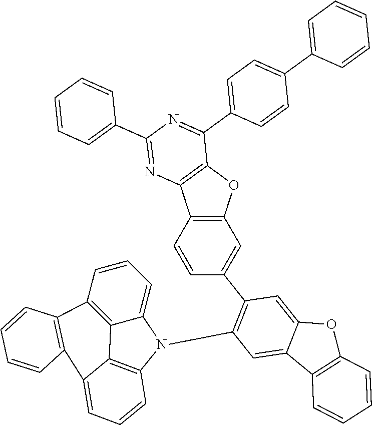 [SMILES: c1ccc(-c2ccc(-c3nc(-c4ccccc4)nc4c3oc3cc(-c5cc6oc7ccccc7c6cc5-n5c6cccc7c8ccccc8c8cccc5c8c76)ccc34)cc2)cc1]

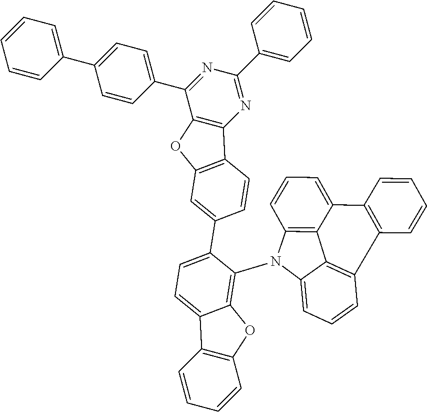 [SMILES: c1ccc(-c2ccc(-c3nc(-c4ccccc4)nc4c3oc3cc(-c5ccc6c(oc7ccccc76)c5-n5c6cccc7c8ccccc8c8cccc5c8c76)ccc34)cc2)cc1]

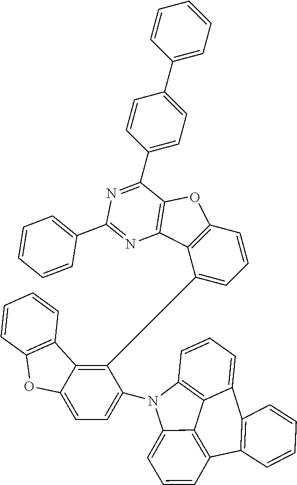 [SMILES: c1ccc(-c2ccc(-c3nc(-c4ccccc4)nc4c3oc3cccc(CCCc5c(-n6c7cccc8c9ccccc9c9cccc6c9c87)ccc6oc7ccccc7c56)c34)cc2)cc1]

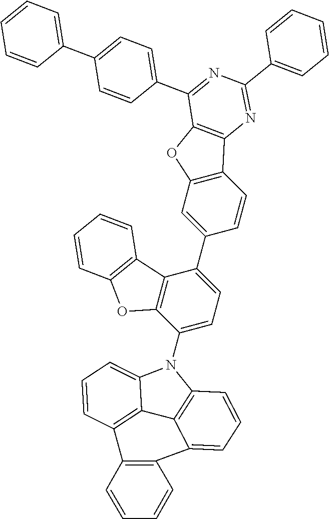 [SMILES: c1ccc(-c2ccc(-c3nc(-c4ccccc4)nc4c3oc3cc(-c5ccc(-n6c7cccc8c9ccccc9c9cccc6c9c87)c6oc7ccccc7c56)ccc34)cc2)cc1]

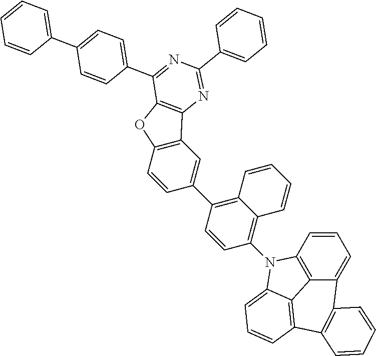 [SMILES: c1ccc(-c2ccc(-c3nc(-c4ccccc4)nc4c3oc3ccc(-c5ccc(-n6c7cccc8c9ccccc9c9cccc6c9c87)c6ccccc56)cc34)cc2)cc1]

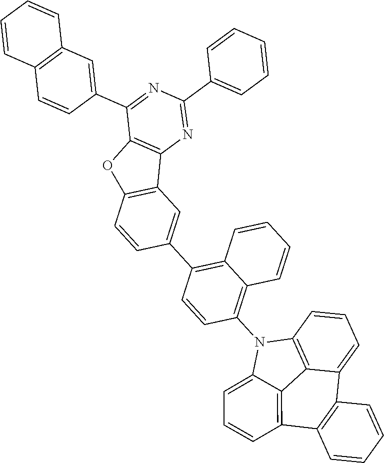 [SMILES: c1ccc(-c2nc(-c3ccc4ccccc4c3)c3oc4ccc(-c5ccc(-n6c7cccc8c9ccccc9c9cccc6c9c87)c6ccccc56)cc4c3n2)cc1]

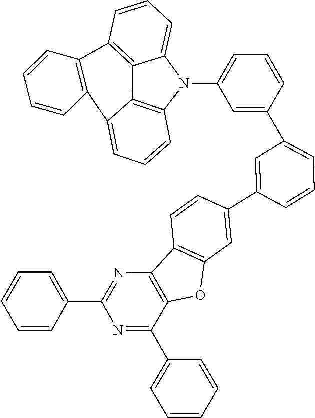 [SMILES: c1ccc(-c2nc(-c3ccccc3)c3oc4cc(-c5cccc(-c6cccc(-n7c8cccc9c%10ccccc%10c%10cccc7c%10c98)c6)c5)ccc4c3n2)cc1]

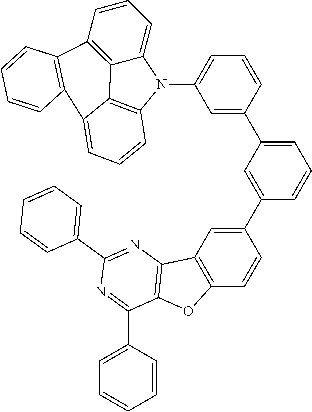 [SMILES: c1ccc(-c2nc(-c3ccccc3)c3oc4ccc(-c5cccc(-c6cccc(-n7c8cccc9c%10ccccc%10c%10cccc7c%10c98)c6)c5)cc4c3n2)cc1]

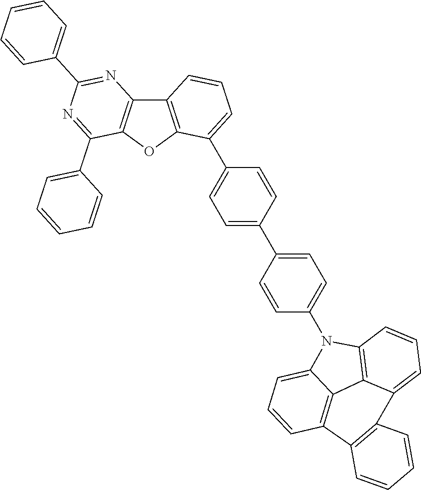 [SMILES: c1ccc(-c2nc(-c3ccccc3)c3oc4c(-c5ccc(-c6ccc(-n7c8cccc9c%10ccccc%10c%10cccc7c%10c98)cc6)cc5)cccc4c3n2)cc1]

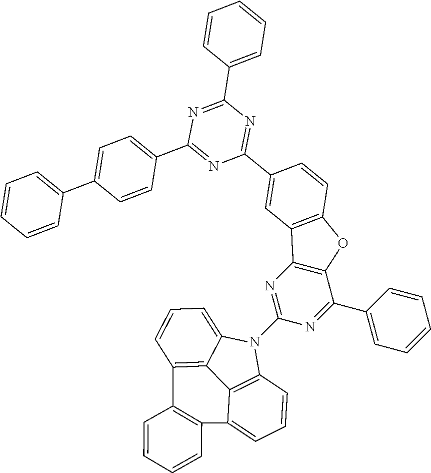 [SMILES: c1ccc(-c2ccc(-c3nc(-c4ccccc4)nc(-c4ccc5oc6c(-c7ccccc7)nc(-n7c8cccc9c%10ccccc%10c%10cccc7c%10c98)nc6c5c4)n3)cc2)cc1]

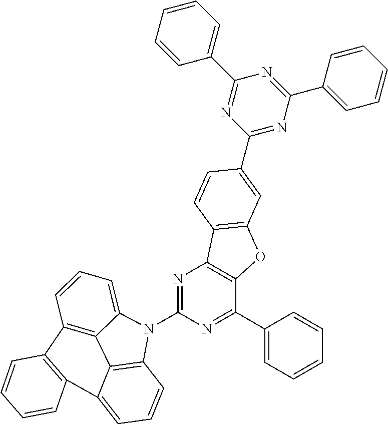 [SMILES: c1ccc(-c2nc(-c3ccccc3)nc(-c3ccc4c(c3)oc3c(-c5ccccc5)nc(-n5c6cccc7c8ccccc8c8cccc5c8c76)nc34)n2)cc1]

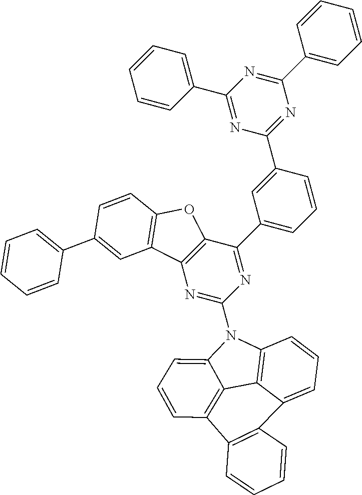 [SMILES: c1ccc(-c2ccc3oc4c(-c5cccc(-c6nc(-c7ccccc7)nc(-c7ccccc7)n6)c5)nc(-n5c6cccc7c8ccccc8c8cccc5c8c76)nc4c3c2)cc1]

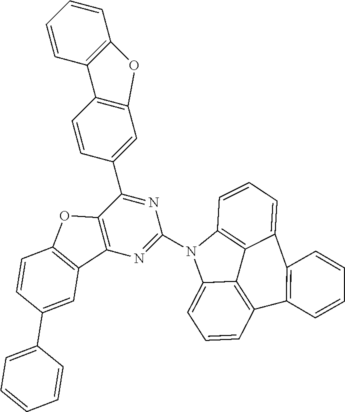 [SMILES: c1ccc(-c2ccc3oc4c(-c5ccc6c(c5)oc5ccccc56)nc(-n5c6cccc7c8ccccc8c8cccc5c8c76)nc4c3c2)cc1]

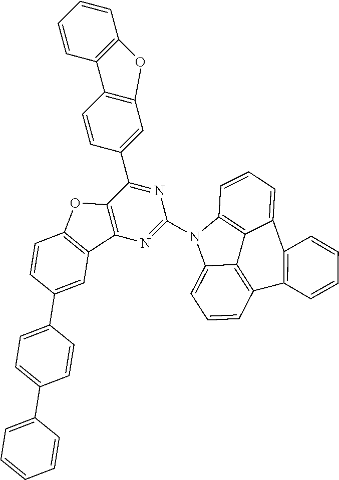 [SMILES: c1ccc(-c2ccc(-c3ccc4oc5c(-c6ccc7c(c6)oc6ccccc67)nc(-n6c7cccc8c9ccccc9c9cccc6c9c87)nc5c4c3)cc2)cc1]

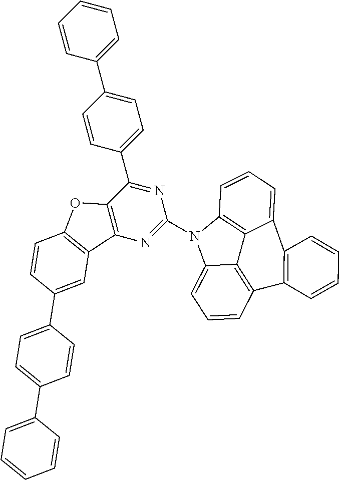 [SMILES: c1ccc(-c2ccc(-c3ccc4oc5c(-c6ccc(-c7ccccc7)cc6)nc(-n6c7cccc8c9ccccc9c9cccc6c9c87)nc5c4c3)cc2)cc1]

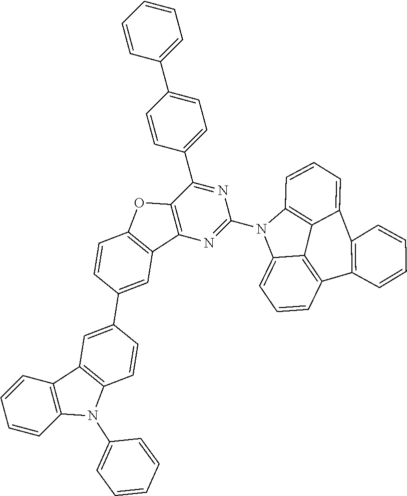 [SMILES: c1ccc(-c2ccc(-c3nc(-n4c5cccc6c7ccccc7c7cccc4c7c65)nc4c3oc3ccc(-c5ccc6c(c5)c5ccccc5n6-c5ccccc5)cc34)cc2)cc1]

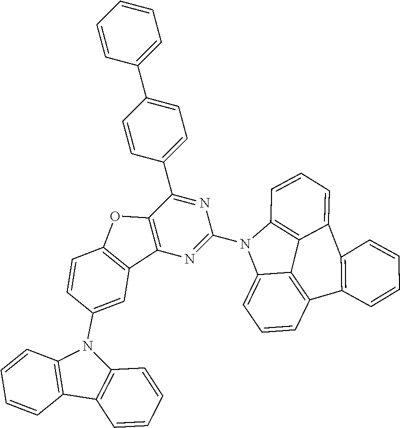 [SMILES: c1ccc(-c2ccc(-c3nc(-n4c5cccc6c7ccccc7c7cccc4c7c65)nc4c3oc3ccc(-n5c6ccccc6c6ccccc65)cc34)cc2)cc1]

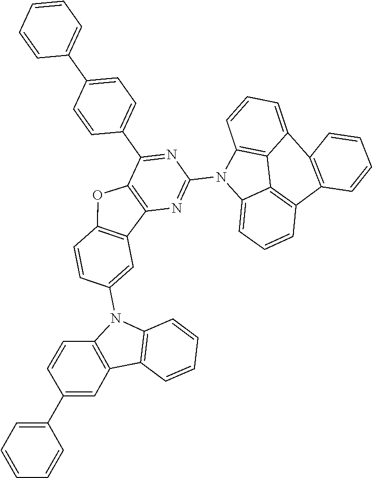 [SMILES: c1ccc(-c2ccc(-c3nc(-n4c5cccc6c7ccccc7c7cccc4c7c65)nc4c3oc3ccc(-n5c6ccccc6c6cc(-c7ccccc7)ccc65)cc34)cc2)cc1]

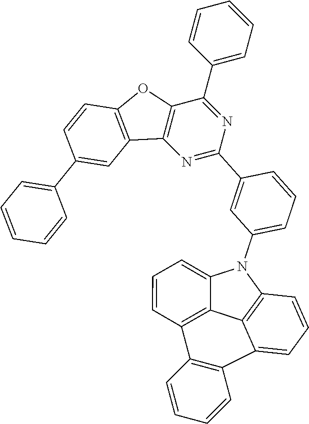 [SMILES: c1ccc(-c2ccc3oc4c(-c5ccccc5)nc(-c5cccc(-n6c7cccc8c9ccccc9c9cccc6c9c87)c5)nc4c3c2)cc1]

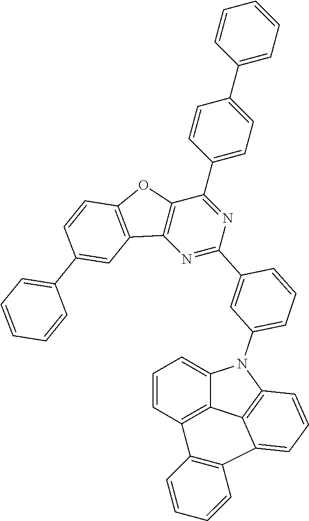 [SMILES: c1ccc(-c2ccc(-c3nc(-c4cccc(-n5c6cccc7c8ccccc8c8cccc5c8c76)c4)nc4c3oc3ccc(-c5ccccc5)cc34)cc2)cc1]

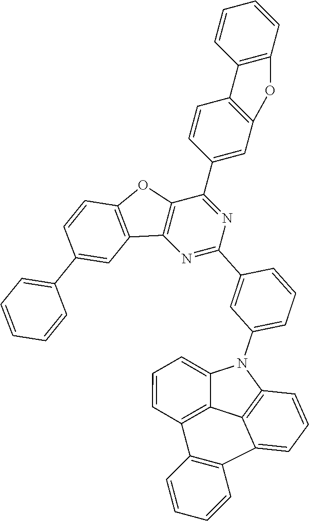 [SMILES: c1ccc(-c2ccc3oc4c(-c5ccc6c(c5)oc5ccccc56)nc(-c5cccc(-n6c7cccc8c9ccccc9c9cccc6c9c87)c5)nc4c3c2)cc1]

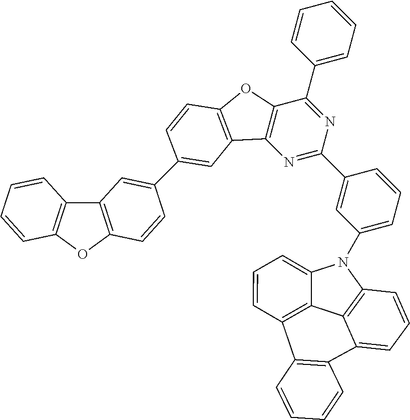 [SMILES: c1ccc(-c2nc(-c3cccc(-n4c5cccc6c7ccccc7c7cccc4c7c65)c3)nc3c2oc2ccc(-c4ccc5oc6ccccc6c5c4)cc23)cc1]

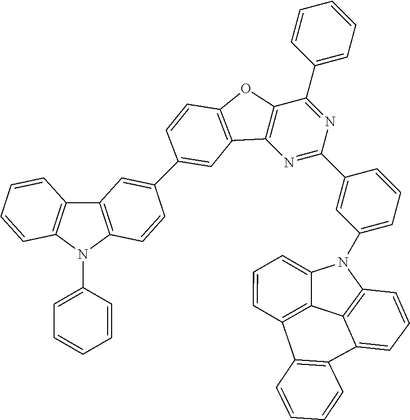 [SMILES: c1ccc(-c2nc(-c3cccc(-n4c5cccc6c7ccccc7c7cccc4c7c65)c3)nc3c2oc2ccc(-c4ccc5c(c4)c4ccccc4n5-c4ccccc4)cc23)cc1]

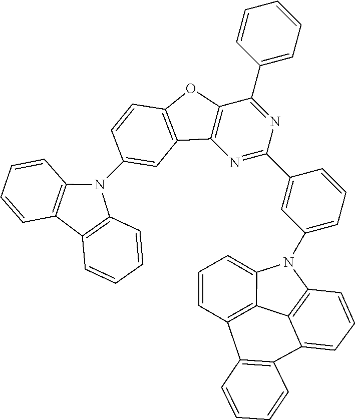 [SMILES: c1ccc(-c2nc(-c3cccc(-n4c5cccc6c7ccccc7c7cccc4c7c65)c3)nc3c2oc2ccc(-n4c5ccccc5c5ccccc54)cc23)cc1]

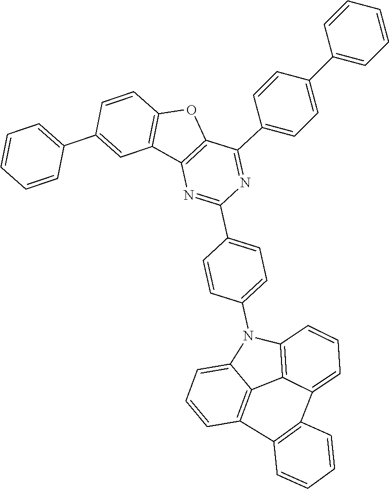 [SMILES: c1ccc(-c2ccc(-c3nc(-c4ccc(-n5c6cccc7c8ccccc8c8cccc5c8c76)cc4)nc4c3oc3ccc(-c5ccccc5)cc34)cc2)cc1]

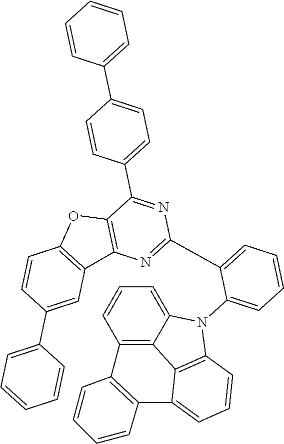 [SMILES: c1ccc(-c2ccc(-c3nc(-c4ccccc4-n4c5cccc6c7ccccc7c7cccc4c7c65)nc4c3oc3ccc(-c5ccccc5)cc34)cc2)cc1]

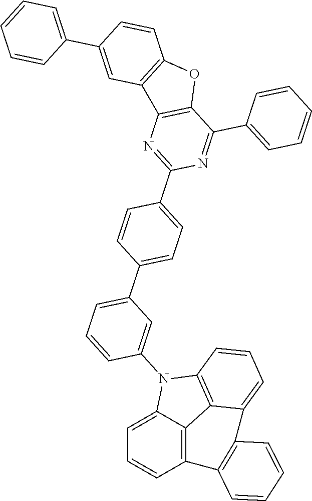 [SMILES: c1ccc(-c2ccc3oc4c(-c5ccccc5)nc(-c5ccc(-c6cccc(-n7c8cccc9c%10ccccc%10c%10cccc7c%10c98)c6)cc5)nc4c3c2)cc1]

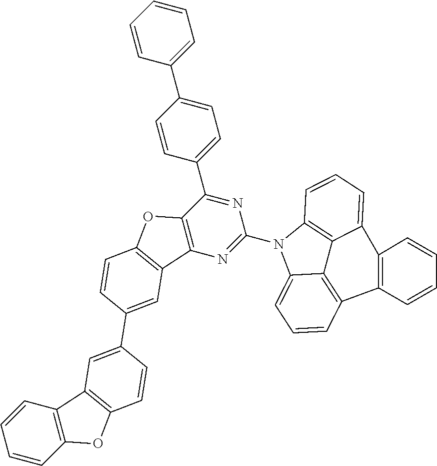 [SMILES: c1ccc(-c2ccc(-c3nc(-n4c5cccc6c7ccccc7c7cccc4c7c65)nc4c3oc3ccc(-c5ccc6oc7ccccc7c6c5)cc34)cc2)cc1]

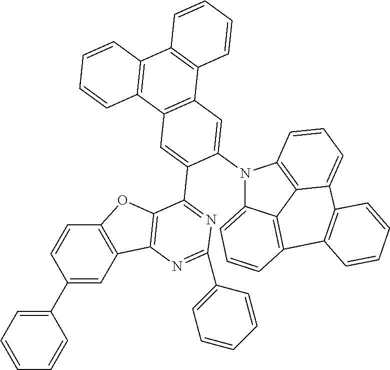 [SMILES: c1ccc(-c2ccc3oc4c(-c5cc6c7ccccc7c7ccccc7c6cc5-n5c6cccc7c8ccccc8c8cccc5c8c76)nc(-c5ccccc5)nc4c3c2)cc1]

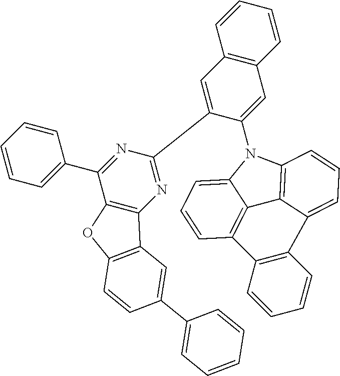 [SMILES: c1ccc(-c2ccc3oc4c(-c5ccccc5)nc(-c5cc6ccccc6cc5-n5c6cccc7c8ccccc8c8cccc5c8c76)nc4c3c2)cc1]

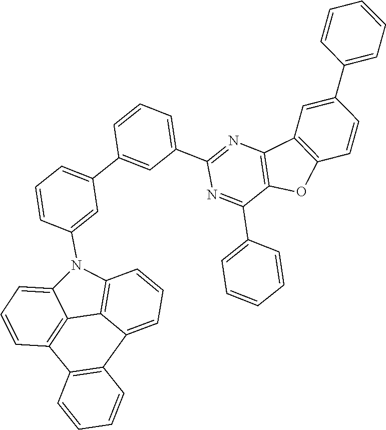 [SMILES: c1ccc(-c2ccc3oc4c(-c5ccccc5)nc(-c5cccc(-c6cccc(-n7c8cccc9c%10ccccc%10c%10cccc7c%10c98)c6)c5)nc4c3c2)cc1]

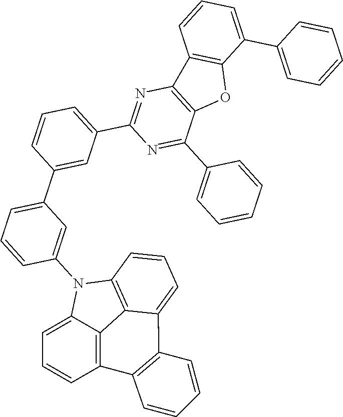 [SMILES: c1ccc(-c2cccc3c2oc2c(-c4ccccc4)nc(-c4cccc(-c5cccc(-n6c7cccc8c9ccccc9c9cccc6c9c87)c5)c4)nc23)cc1]

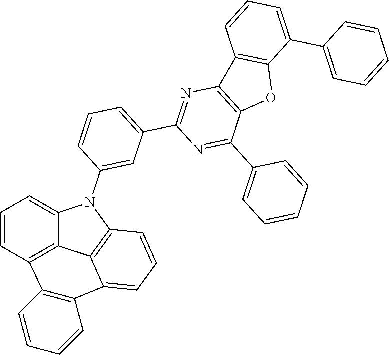 [SMILES: c1ccc(-c2cccc3c2oc2c(-c4ccccc4)nc(-c4cccc(-n5c6cccc7c8ccccc8c8cccc5c8c76)c4)nc23)cc1]

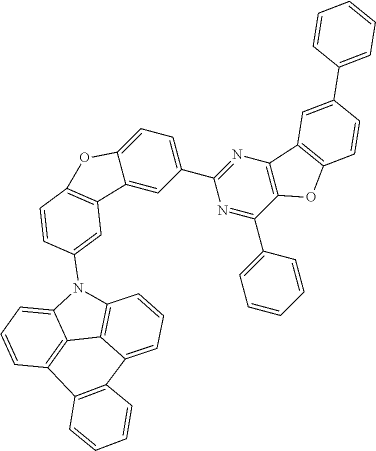 [SMILES: c1ccc(-c2ccc3oc4c(-c5ccccc5)nc(-c5ccc6oc7ccc(-n8c9cccc%10c%11ccccc%11c%11cccc8c%11c%109)cc7c6c5)nc4c3c2)cc1]

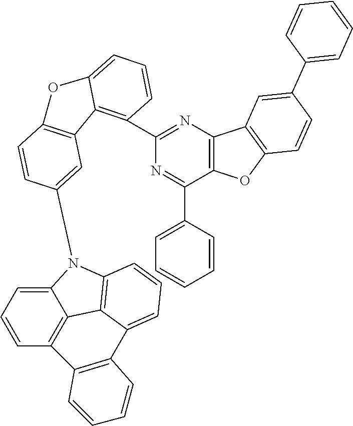 [SMILES: c1ccc(-c2ccc3oc4c(-c5ccccc5)nc(-c5cccc6oc7ccc(-n8c9cccc%10c%11ccccc%11c%11cccc8c%11c%109)cc7c56)nc4c3c2)cc1]

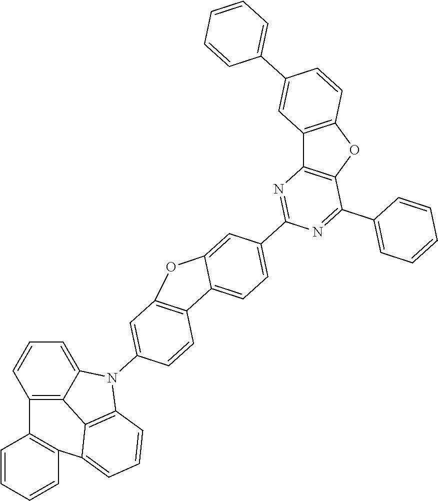 [SMILES: c1ccc(-c2ccc3oc4c(-c5ccccc5)nc(-c5ccc6c(c5)oc5cc(-n7c8cccc9c%10ccccc%10c%10cccc7c%10c98)ccc56)nc4c3c2)cc1]

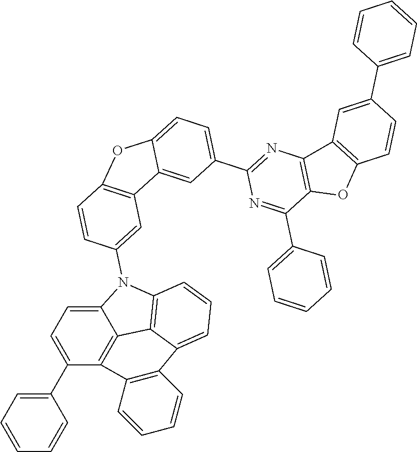 [SMILES: c1ccc(-c2ccc3oc4c(-c5ccccc5)nc(-c5ccc6oc7ccc(-n8c9cccc%10c%11ccccc%11c%11c(-c%12ccccc%12)ccc8c%11c%109)cc7c6c5)nc4c3c2)cc1]

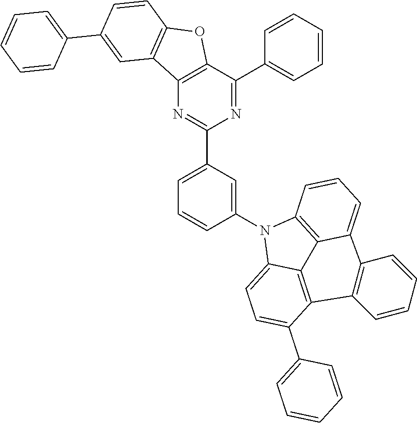 [SMILES: c1ccc(-c2ccc3oc4c(-c5ccccc5)nc(-c5cccc(-n6c7cccc8c9ccccc9c9c(-c%10ccccc%10)ccc6c9c87)c5)nc4c3c2)cc1]

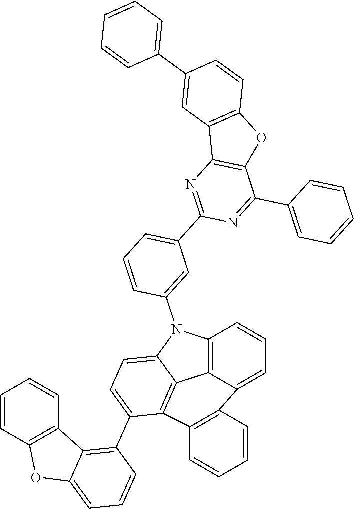 [SMILES: c1ccc(-c2ccc3oc4c(-c5ccccc5)nc(-c5cccc(-n6c7cccc8c9ccccc9c9c(-c%10cccc%11oc%12ccccc%12c%10%11)ccc6c9c87)c5)nc4c3c2)cc1]

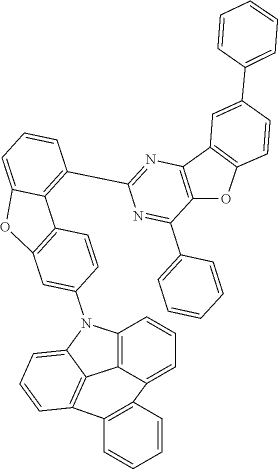 [SMILES: c1ccc(-c2ccc3oc4c(-c5ccccc5)nc(-c5cccc6oc7cc(-n8c9cccc%10c%11ccccc%11c%11cccc8c%11c%109)ccc7c56)nc4c3c2)cc1]

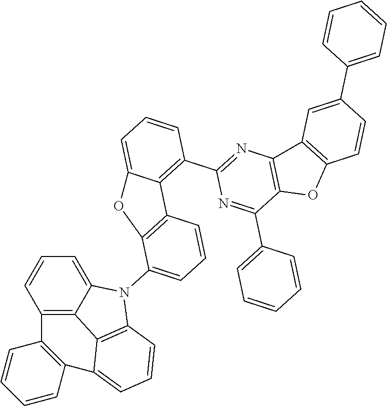 [SMILES: c1ccc(-c2ccc3oc4c(-c5ccccc5)nc(-c5cccc6oc7c(-n8c9cccc%10c%11ccccc%11c%11cccc8c%11c%109)cccc7c56)nc4c3c2)cc1]

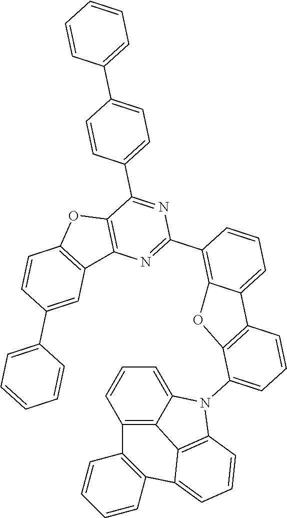 [SMILES: c1ccc(-c2ccc(-c3nc(-c4cccc5c4oc4c(-n6c7cccc8c9ccccc9c9cccc6c9c87)cccc45)nc4c3oc3ccc(-c5ccccc5)cc34)cc2)cc1]

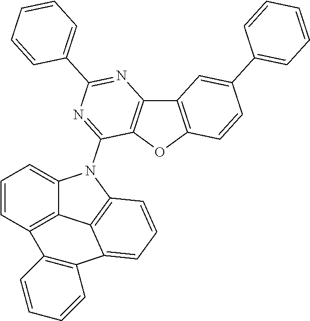 [SMILES: c1ccc(-c2ccc3oc4c(-n5c6cccc7c8ccccc8c8cccc5c8c76)nc(-c5ccccc5)nc4c3c2)cc1]

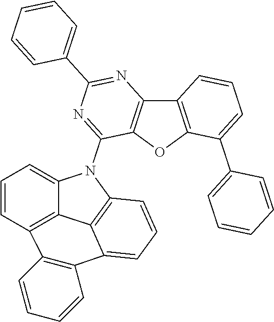 [SMILES: c1ccc(-c2nc(-n3c4cccc5c6ccccc6c6cccc3c6c54)c3oc4c(-c5ccccc5)cccc4c3n2)cc1]

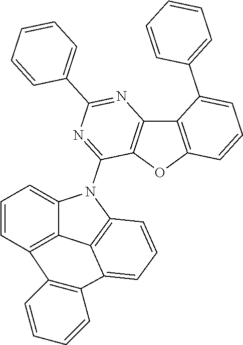 [SMILES: c1ccc(-c2nc(-n3c4cccc5c6ccccc6c6cccc3c6c54)c3oc4cccc(-c5ccccc5)c4c3n2)cc1]

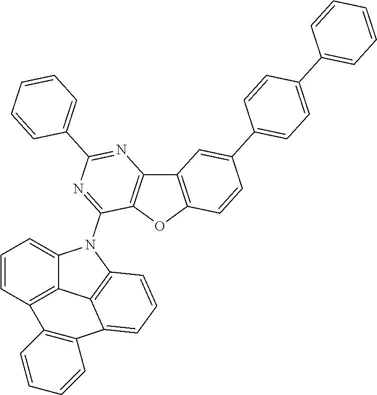 [SMILES: c1ccc(-c2ccc(-c3ccc4oc5c(-n6c7cccc8c9ccccc9c9cccc6c9c87)nc(-c6ccccc6)nc5c4c3)cc2)cc1]

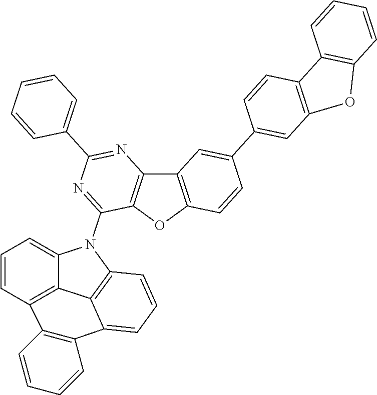 [SMILES: c1ccc(-c2nc(-n3c4cccc5c6ccccc6c6cccc3c6c54)c3oc4ccc(-c5ccc6c(c5)oc5ccccc56)cc4c3n2)cc1]

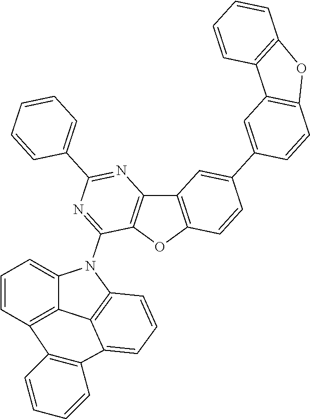 [SMILES: c1ccc(-c2nc(-n3c4cccc5c6ccccc6c6cccc3c6c54)c3oc4ccc(-c5ccc6oc7ccccc7c6c5)cc4c3n2)cc1]

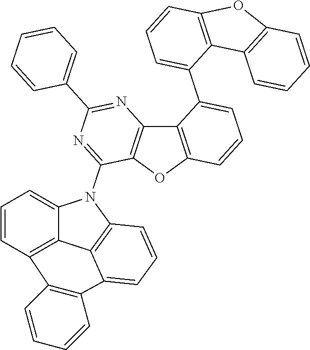 [SMILES: c1ccc(-c2nc(-n3c4cccc5c6ccccc6c6cccc3c6c54)c3oc4cccc(-c5cccc6oc7ccccc7c56)c4c3n2)cc1]

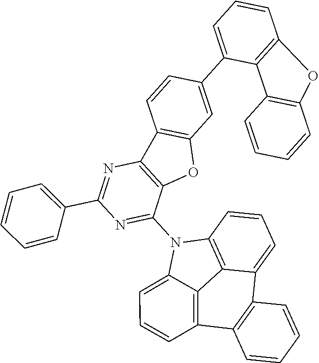 [SMILES: c1ccc(-c2nc(-n3c4cccc5c6ccccc6c6cccc3c6c54)c3oc4cc(-c5cccc6oc7ccccc7c56)ccc4c3n2)cc1]

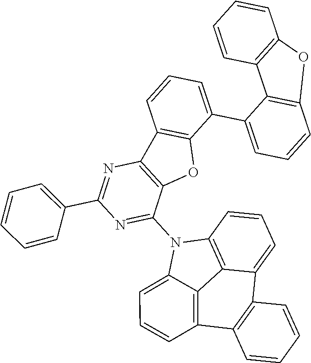 [SMILES: c1ccc(-c2nc(-n3c4cccc5c6ccccc6c6cccc3c6c54)c3oc4c(-c5cccc6oc7ccccc7c56)cccc4c3n2)cc1]

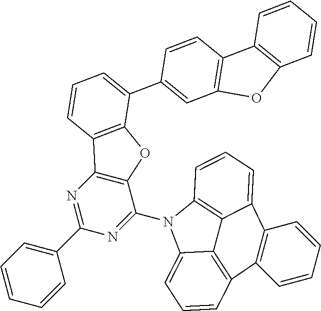 [SMILES: c1ccc(-c2nc(-n3c4cccc5c6ccccc6c6cccc3c6c54)c3oc4c(-c5ccc6c(c5)oc5ccccc56)cccc4c3n2)cc1]

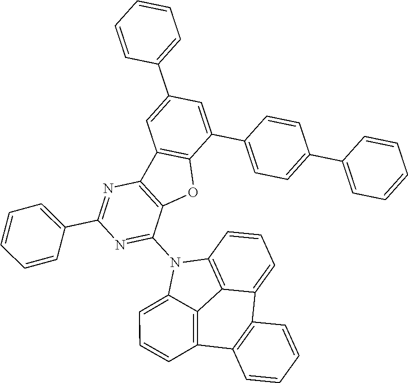 [SMILES: c1ccc(-c2ccc(-c3cc(-c4ccccc4)cc4c3oc3c(-n5c6cccc7c8ccccc8c8cccc5c8c76)nc(-c5ccccc5)nc34)cc2)cc1]

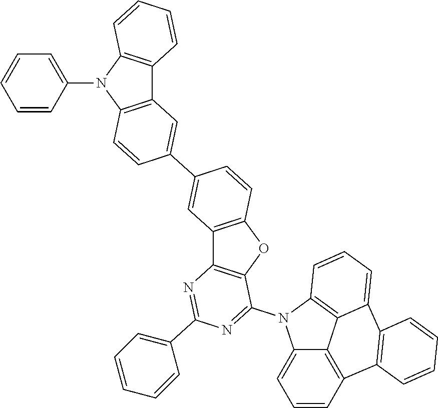 [SMILES: c1ccc(-c2nc(-n3c4cccc5c6ccccc6c6cccc3c6c54)c3oc4ccc(-c5ccc6c(c5)c5ccccc5n6-c5ccccc5)cc4c3n2)cc1]

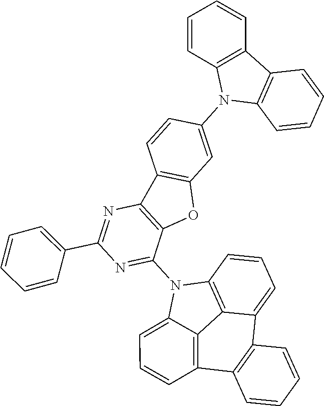 [SMILES: c1ccc(-c2nc(-n3c4cccc5c6ccccc6c6cccc3c6c54)c3oc4cc(-n5c6ccccc6c6ccccc65)ccc4c3n2)cc1]

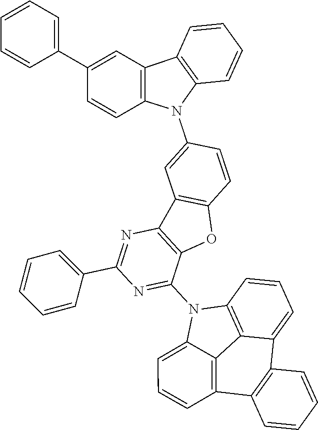 [SMILES: c1ccc(-c2ccc3c(c2)c2ccccc2n3-c2ccc3oc4c(-n5c6cccc7c8ccccc8c8cccc5c8c76)nc(-c5ccccc5)nc4c3c2)cc1]

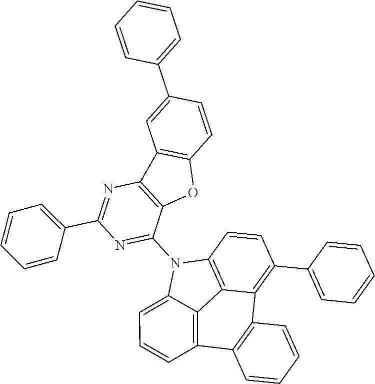 [SMILES: c1ccc(-c2ccc3oc4c(-n5c6cccc7c8ccccc8c8c(-c9ccccc9)ccc5c8c76)nc(-c5ccccc5)nc4c3c2)cc1]

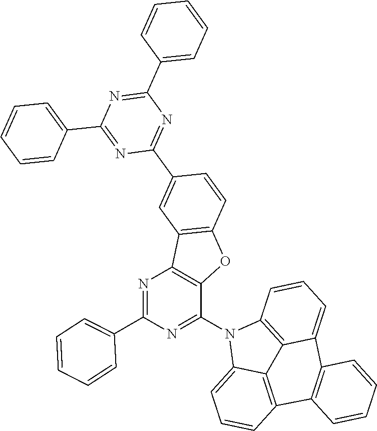 [SMILES: c1ccc(-c2nc(-c3ccccc3)nc(-c3ccc4oc5c(-n6c7cccc8c9ccccc9c9cccc6c9c87)nc(-c6ccccc6)nc5c4c3)n2)cc1]

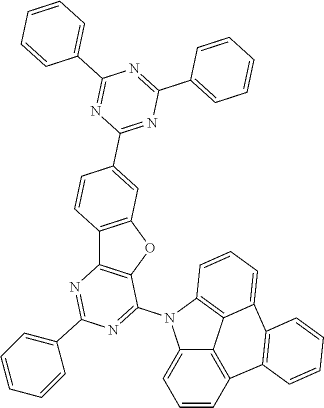 [SMILES: c1ccc(-c2nc(-c3ccccc3)nc(-c3ccc4c(c3)oc3c(-n5c6cccc7c8ccccc8c8cccc5c8c76)nc(-c5ccccc5)nc34)n2)cc1]

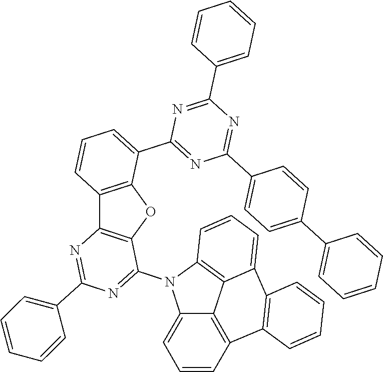 [SMILES: c1ccc(-c2ccc(-c3nc(-c4ccccc4)nc(-c4cccc5c4oc4c(-n6c7cccc8c9ccccc9c9cccc6c9c87)nc(-c6ccccc6)nc45)n3)cc2)cc1]

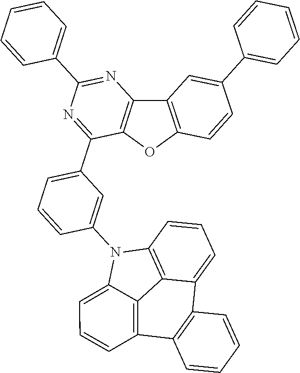 [SMILES: c1ccc(-c2ccc3oc4c(-c5cccc(-n6c7cccc8c9ccccc9c9cccc6c9c87)c5)nc(-c5ccccc5)nc4c3c2)cc1]

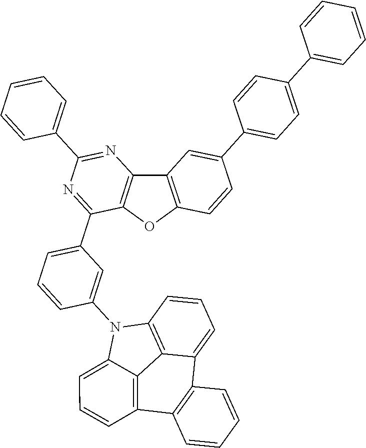 [SMILES: c1ccc(-c2ccc(-c3ccc4oc5c(-c6cccc(-n7c8cccc9c%10ccccc%10c%10cccc7c%10c98)c6)nc(-c6ccccc6)nc5c4c3)cc2)cc1]

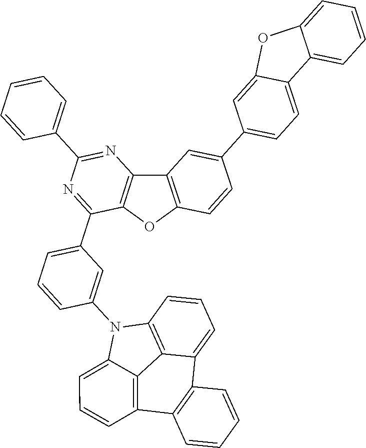 [SMILES: c1ccc(-c2nc(-c3cccc(-n4c5cccc6c7ccccc7c7cccc4c7c65)c3)c3oc4ccc(-c5ccc6c(c5)oc5ccccc56)cc4c3n2)cc1]

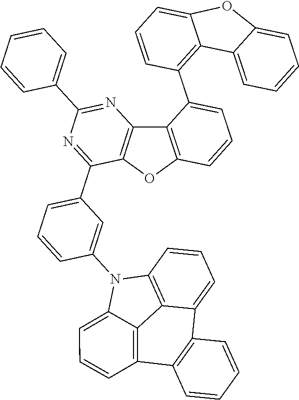 [SMILES: c1ccc(-c2nc(-c3cccc(-n4c5cccc6c7ccccc7c7cccc4c7c65)c3)c3oc4cccc(-c5cccc6oc7ccccc7c56)c4c3n2)cc1]

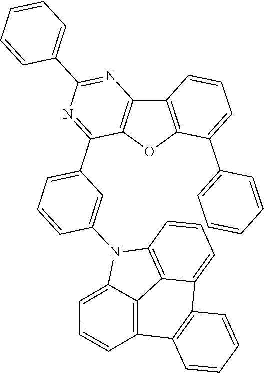 [SMILES: c1ccc(-c2nc(-c3cccc(-n4c5cccc6c7ccccc7c7cccc4c7c65)c3)c3oc4c(-c5ccccc5)cccc4c3n2)cc1]

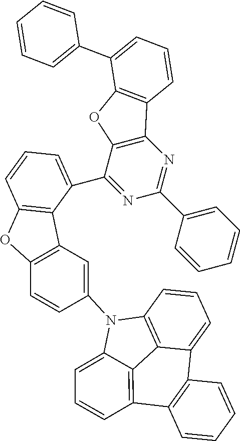 [SMILES: c1ccc(-c2nc(-c3cccc4oc5ccc(-n6c7cccc8c9ccccc9c9cccc6c9c87)cc5c34)c3oc4c(-c5ccccc5)cccc4c3n2)cc1]

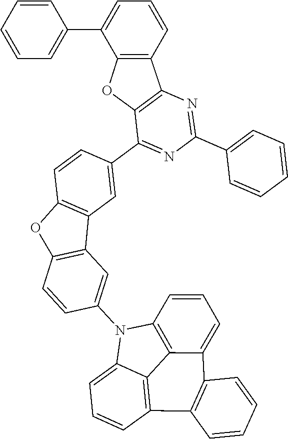 [SMILES: c1ccc(-c2nc(-c3ccc4oc5ccc(-n6c7cccc8c9ccccc9c9cccc6c9c87)cc5c4c3)c3oc4c(-c5ccccc5)cccc4c3n2)cc1]

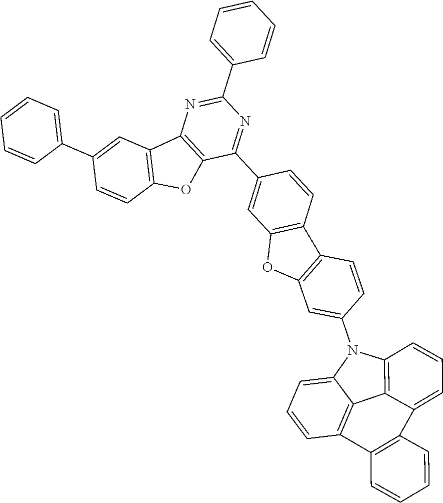 [SMILES: c1ccc(-c2ccc3oc4c(-c5ccc6c(c5)oc5cc(-n7c8cccc9c%10ccccc%10c%10cccc7c%10c98)ccc56)nc(-c5ccccc5)nc4c3c2)cc1]

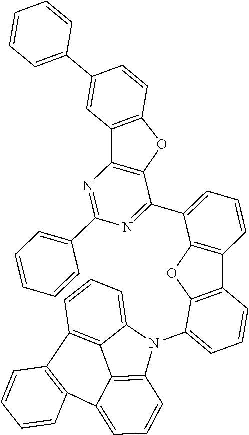 [SMILES: c1ccc(-c2ccc3oc4c(-c5cccc6c5oc5c(-n7c8cccc9c%10ccccc%10c%10cccc7c%10c98)cccc56)nc(-c5ccccc5)nc4c3c2)cc1]

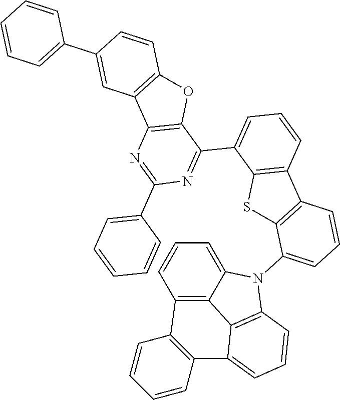 [SMILES: c1ccc(-c2ccc3oc4c(-c5cccc6c5sc5c(-n7c8cccc9c%10ccccc%10c%10cccc7c%10c98)cccc56)nc(-c5ccccc5)nc4c3c2)cc1]

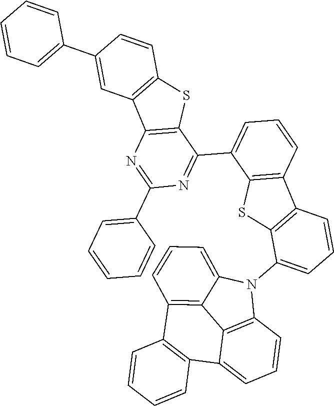 [SMILES: c1ccc(-c2ccc3sc4c(-c5cccc6c5sc5c(-n7c8cccc9c%10ccccc%10c%10cccc7c%10c98)cccc56)nc(-c5ccccc5)nc4c3c2)cc1]

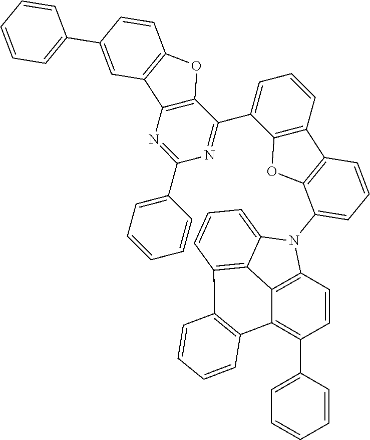 [SMILES: c1ccc(-c2ccc3oc4c(-c5cccc6c5oc5c(-n7c8cccc9c%10ccccc%10c%10c(-c%11ccccc%11)ccc7c%10c98)cccc56)nc(-c5ccccc5)nc4c3c2)cc1]

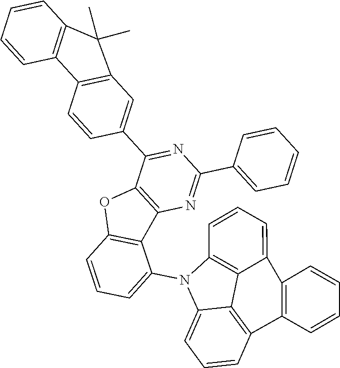 [SMILES: CC1(C)c2ccccc2-c2ccc(-c3nc(-c4ccccc4)nc4c3oc3cccc(-n5c6cccc7c8ccccc8c8cccc5c8c76)c34)cc21]

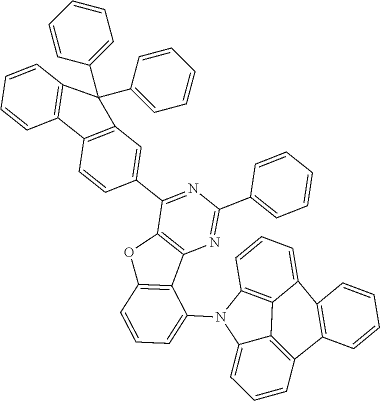 [SMILES: c1ccc(-c2nc(-c3ccc4c(c3)C(c3ccccc3)(c3ccccc3)c3ccccc3-4)c3oc4cccc(-n5c6cccc7c8ccccc8c8cccc5c8c76)c4c3n2)cc1]

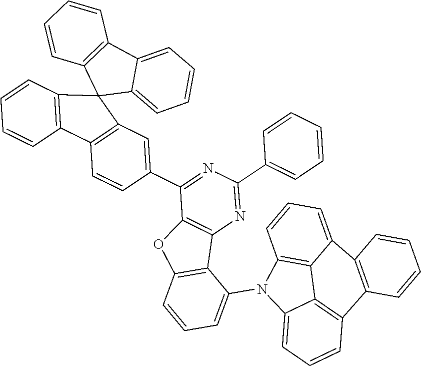 [SMILES: c1ccc(-c2nc(-c3ccc4c(c3)C3(c5ccccc5-c5ccccc53)c3ccccc3-4)c3oc4cccc(-n5c6cccc7c8ccccc8c8cccc5c8c76)c4c3n2)cc1]

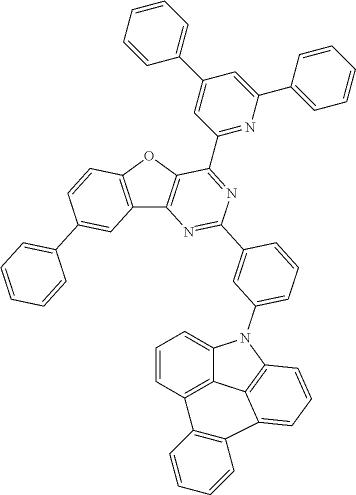 [SMILES: c1ccc(-c2cc(-c3ccccc3)nc(-c3nc(-c4cccc(-n5c6cccc7c8ccccc8c8cccc5c8c76)c4)nc4c3oc3ccc(-c5ccccc5)cc34)c2)cc1]

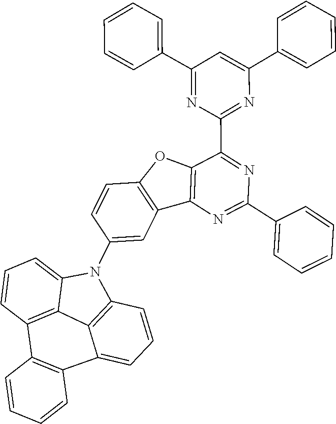 [SMILES: c1ccc(-c2cc(-c3ccccc3)nc(-c3nc(-c4ccccc4)nc4c3oc3ccc(-n5c6cccc7c8ccccc8c8cccc5c8c76)cc34)n2)cc1]

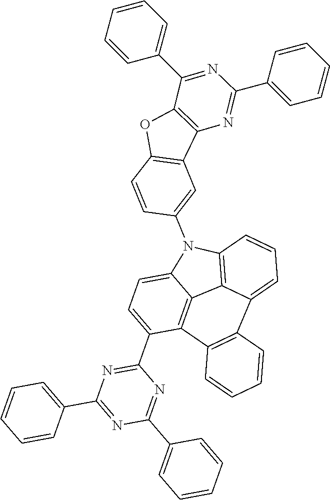 [SMILES: c1ccc(-c2nc(-c3ccccc3)nc(-c3ccc4c5c3c3ccccc3c3cccc(c35)n4-c3ccc4oc5c(-c6ccccc6)nc(-c6ccccc6)nc5c4c3)n2)cc1]

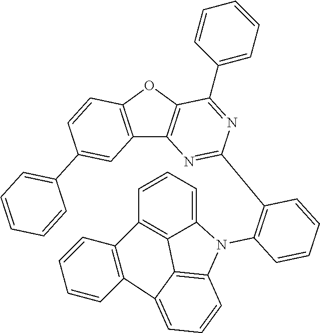 [SMILES: c1ccc(-c2ccc3oc4c(-c5ccccc5)nc(-c5ccccc5-n5c6cccc7c8ccccc8c8cccc5c8c76)nc4c3c2)cc1]

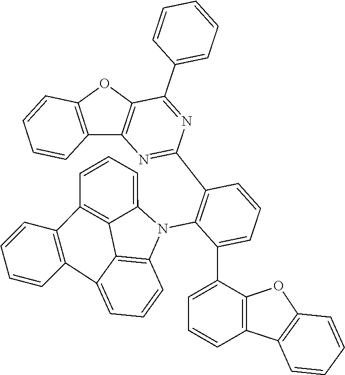 [SMILES: c1ccc(-c2nc(-c3cccc(-c4cccc5c4oc4ccccc45)c3-n3c4cccc5c6ccccc6c6cccc3c6c54)nc3c2oc2ccccc23)cc1]

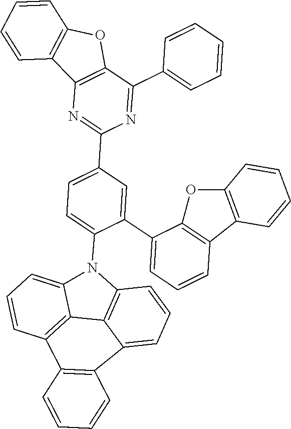 [SMILES: c1ccc(-c2nc(-c3ccc(-n4c5cccc6c7ccccc7c7cccc4c7c65)c(-c4cccc5c4oc4ccccc45)c3)nc3c2oc2ccccc23)cc1]

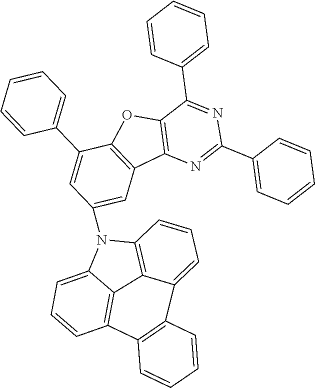 [SMILES: c1ccc(-c2nc(-c3ccccc3)c3oc4c(-c5ccccc5)cc(-n5c6cccc7c8ccccc8c8cccc5c8c76)cc4c3n2)cc1]

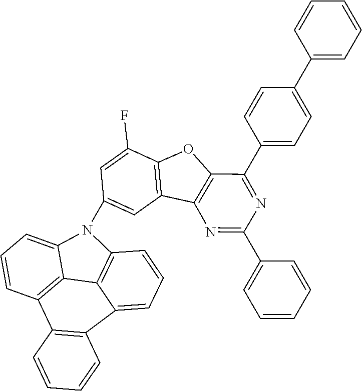 [SMILES: Fc1cc(-n2c3cccc4c5ccccc5c5cccc2c5c43)cc2c1oc1c(-c3ccc(-c4ccccc4)cc3)nc(-c3ccccc3)nc12]

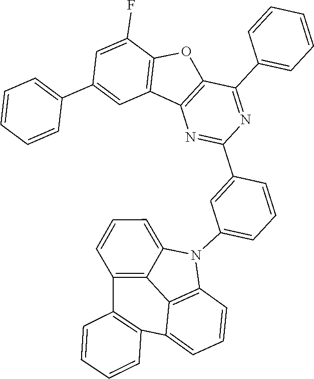 [SMILES: Fc1cc(-c2ccccc2)cc2c1oc1c(-c3ccccc3)nc(-c3cccc(-n4c5cccc6c7ccccc7c7cccc4c7c65)c3)nc12]

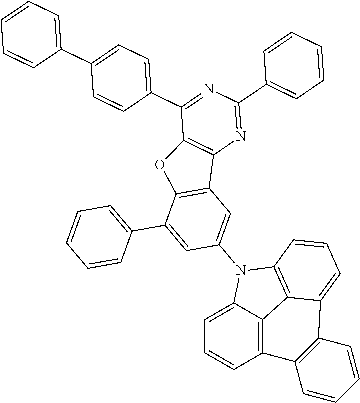 [SMILES: c1ccc(-c2ccc(-c3nc(-c4ccccc4)nc4c3oc3c(-c5ccccc5)cc(-n5c6cccc7c8ccccc8c8cccc5c8c76)cc34)cc2)cc1]

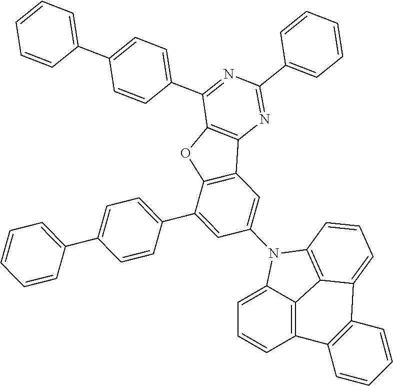 [SMILES: c1ccc(-c2ccc(-c3cc(-n4c5cccc6c7ccccc7c7cccc4c7c65)cc4c3oc3c(-c5ccc(-c6ccccc6)cc5)nc(-c5ccccc5)nc34)cc2)cc1]

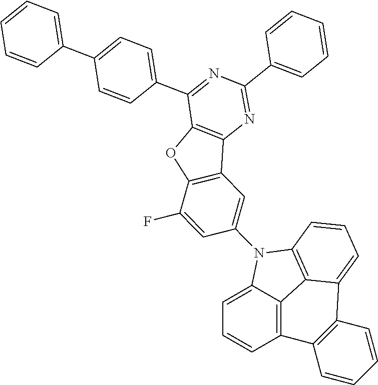 [SMILES: Fc1cc(-n2c3cccc4c5ccccc5c5cccc2c5c43)cc2c1oc1c(-c3ccc(-c4ccccc4)cc3)nc(-c3ccccc3)nc12]

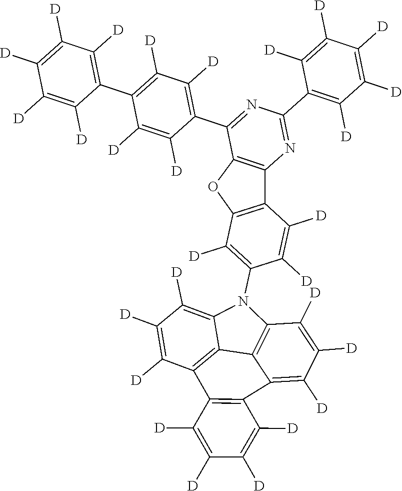 [SMILES: [2H]c1c([2H])c([2H])c(-c2nc(-c3c([2H])c([2H])c(-c4c([2H])c([2H])c([2H])c([2H])c4[2H])c([2H])c3[2H])c3oc4c([2H])c(-n5c6c([2H])c([2H])c([2H])c7c8c([2H])c([2H])c([2H])c([2H])c8c8c([2H])c([2H])c([2H])c5c8c76)c([2H])c([2H])c4c3n2)c([2H])c1[2H]]

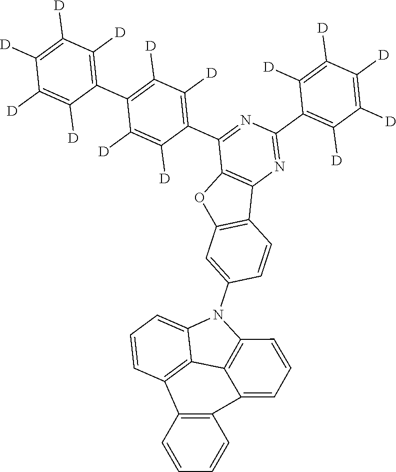 [SMILES: [2H]c1c([2H])c([2H])c(-c2nc(-c3c([2H])c([2H])c(-c4c([2H])c([2H])c([2H])c([2H])c4[2H])c([2H])c3[2H])c3oc4cc(-n5c6cccc7c8ccccc8c8cccc5c8c76)ccc4c3n2)c([2H])c1[2H]]

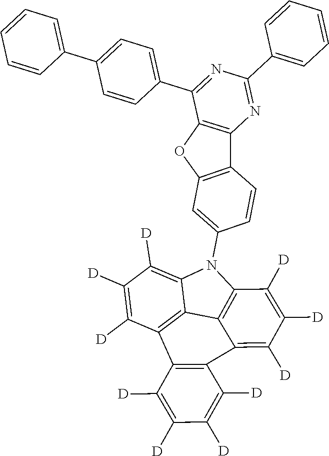 [SMILES: [2H]c1c([2H])c([2H])c2c(c1[2H])c1c([2H])c([2H])c([2H])c3c1c1c2c([2H])c([2H])c([2H])c1n3-c1ccc2c(c1)oc1c(-c3ccc(-c4ccccc4)cc3)nc(-c3ccccc3)nc12]

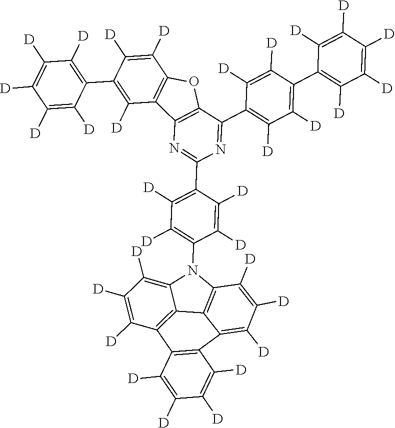 [SMILES: [2H]c1c([2H])c([2H])c(-c2c([2H])c([2H])c(-c3nc(-c4c([2H])c([2H])c(-n5c6c([2H])c([2H])c([2H])c7c8c([2H])c([2H])c([2H])c([2H])c8c8c([2H])c([2H])c([2H])c5c8c76)c([2H])c4[2H])nc4c3oc3c([2H])c([2H])c(-c5c([2H])c([2H])c([2H])c([2H])c5[2H])c([2H])c34)c([2H])c2[2H])c([2H])c1[2H]]

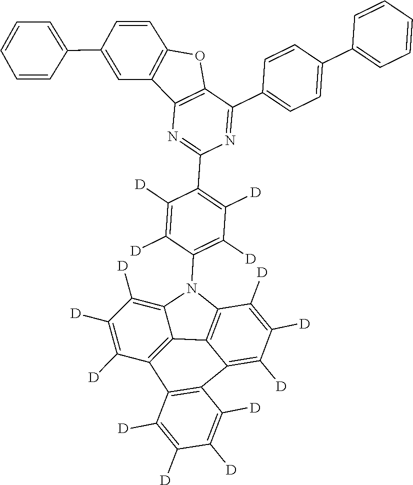 [SMILES: [2H]c1c([2H])c(-n2c3c([2H])c([2H])c([2H])c4c5c([2H])c([2H])c([2H])c([2H])c5c5c([2H])c([2H])c([2H])c2c5c43)c([2H])c([2H])c1-c1nc(-c2ccc(-c3ccccc3)cc2)c2oc3ccc(-c4ccccc4)cc3c2n1]

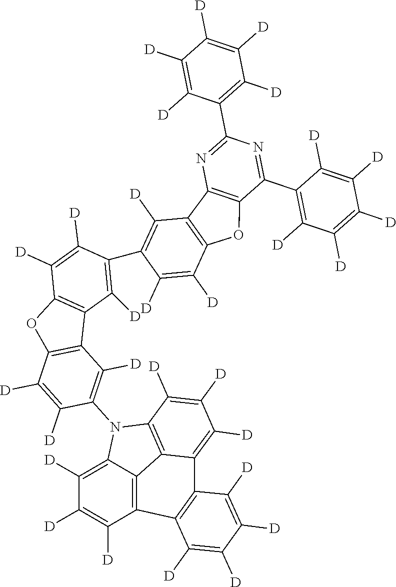 [SMILES: [2H]c1c([2H])c([2H])c(-c2nc(-c3c([2H])c([2H])c([2H])c([2H])c3[2H])c3oc4c([2H])c([2H])c(-c5c([2H])c([2H])c6oc7c([2H])c([2H])c(-n8c9c([2H])c([2H])c([2H])c%10c%11c([2H])c([2H])c([2H])c([2H])c%11c%11c([2H])c([2H])c([2H])c8c%11c%109)c([2H])c7c6c5[2H])c([2H])c4c3n2)c([2H])c1[2H]]

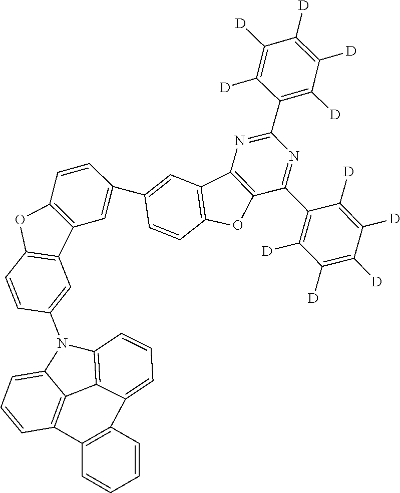 [SMILES: [2H]c1c([2H])c([2H])c(-c2nc(-c3c([2H])c([2H])c([2H])c([2H])c3[2H])c3oc4ccc(-c5ccc6oc7ccc(-n8c9cccc%10c%11ccccc%11c%11cccc8c%11c%109)cc7c6c5)cc4c3n2)c([2H])c1[2H]]

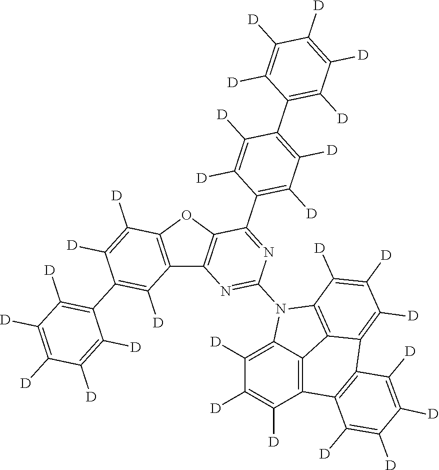 [SMILES: [2H]c1c([2H])c([2H])c(-c2c([2H])c([2H])c(-c3nc(-n4c5c([2H])c([2H])c([2H])c6c7c([2H])c([2H])c([2H])c([2H])c7c7c([2H])c([2H])c([2H])c4c7c65)nc4c3oc3c([2H])c([2H])c(-c5c([2H])c([2H])c([2H])c([2H])c5[2H])c([2H])c34)c([2H])c2[2H])c([2H])c1[2H]]

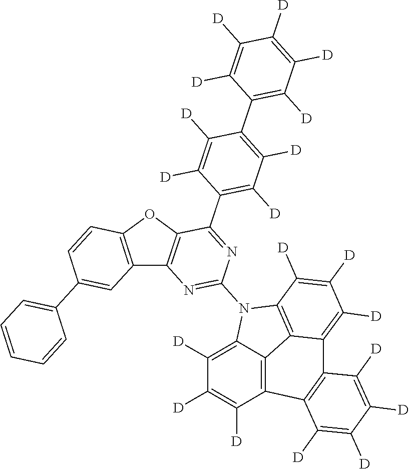 [SMILES: [2H]c1c([2H])c([2H])c(-c2c([2H])c([2H])c(-c3nc(-n4c5c([2H])c([2H])c([2H])c6c7c([2H])c([2H])c([2H])c([2H])c7c7c([2H])c([2H])c([2H])c4c7c65)nc4c3oc3ccc(-c5ccccc5)cc34)c([2H])c2[2H])c([2H])c1[2H]]

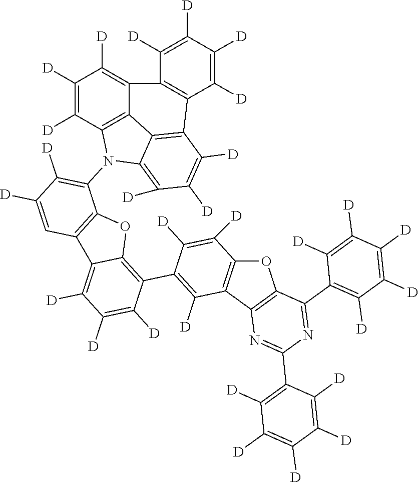 [SMILES: [2H]c1cc2c(oc3c(-c4c([2H])c([2H])c5oc6c(-c7c([2H])c([2H])c([2H])c([2H])c7[2H])nc(-c7c([2H])c([2H])c([2H])c([2H])c7[2H])nc6c5c4[2H])c([2H])c([2H])c([2H])c32)c(-n2c3c([2H])c([2H])c([2H])c4c5c([2H])c([2H])c([2H])c([2H])c5c5c([2H])c([2H])c([2H])c2c5c43)c1[2H]]

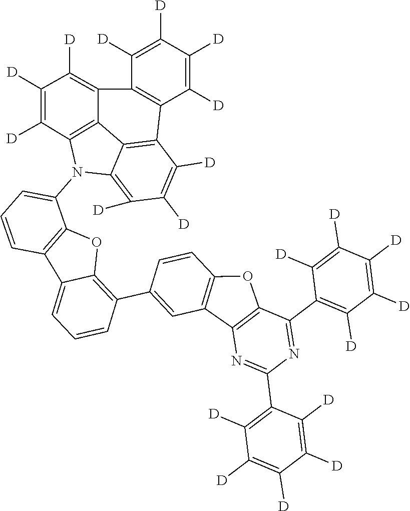 [SMILES: [2H]c1c([2H])c([2H])c(-c2nc(-c3c([2H])c([2H])c([2H])c([2H])c3[2H])c3oc4ccc(-c5cccc6c5oc5c(-n7c8c([2H])c([2H])c([2H])c9c%10c([2H])c([2H])c([2H])c([2H])c%10c%10c([2H])c([2H])c([2H])c7c%10c98)cccc56)cc4c3n2)c([2H])c1[2H]]

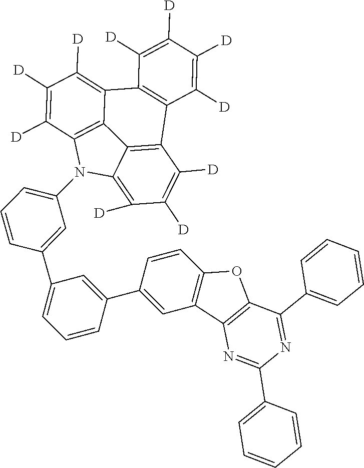 [SMILES: [2H]c1c([2H])c([2H])c2c(c1[2H])c1c([2H])c([2H])c([2H])c3c1c1c2c([2H])c([2H])c([2H])c1n3-c1cccc(-c2cccc(-c3ccc4oc5c(-c6ccccc6)nc(-c6ccccc6)nc5c4c3)c2)c1]

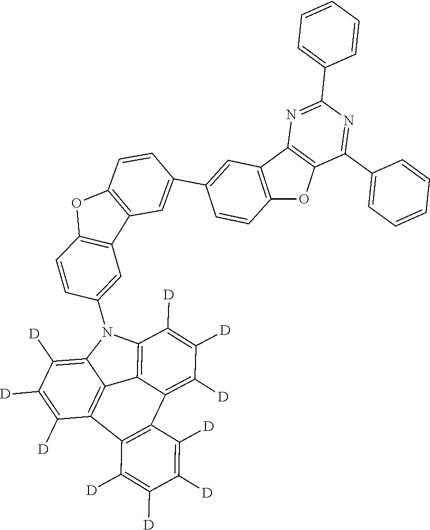 [SMILES: [2H]c1c([2H])c([2H])c2c(c1[2H])c1c([2H])c([2H])c([2H])c3c1c1c2c([2H])c([2H])c([2H])c1n3-c1ccc2oc3ccc(-c4ccc5oc6c(-c7ccccc7)nc(-c7ccccc7)nc6c5c4)cc3c2c1]

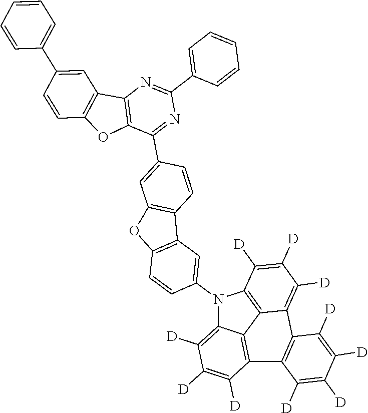 [SMILES: [2H]c1c([2H])c([2H])c2c(c1[2H])c1c([2H])c([2H])c([2H])c3c1c1c2c([2H])c([2H])c([2H])c1n3-c1ccc2oc3cc(-c4nc(-c5ccccc5)nc5c4oc4ccc(-c6ccccc6)cc45)ccc3c2c1]

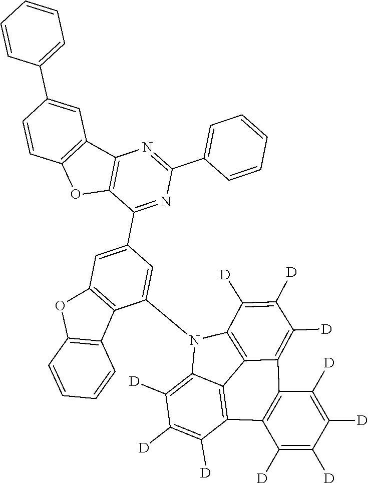 [SMILES: [2H]c1c([2H])c([2H])c2c(c1[2H])c1c([2H])c([2H])c([2H])c3c1c1c2c([2H])c([2H])c([2H])c1n3-c1cc(-c2nc(-c3ccccc3)nc3c2oc2ccc(-c4ccccc4)cc23)cc2oc3ccccc3c12]

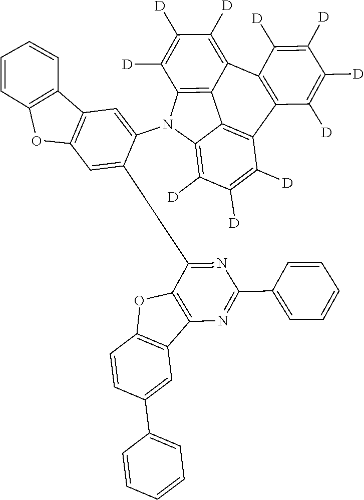 [SMILES: [2H]c1c([2H])c([2H])c2c(c1[2H])c1c([2H])c([2H])c([2H])c3c1c1c2c([2H])c([2H])c([2H])c1n3-c1cc2c(cc1-c1nc(-c3ccccc3)nc3c1oc1ccc(-c4ccccc4)cc13)oc1ccccc12]

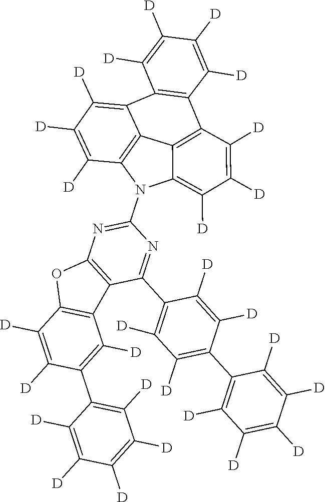 [SMILES: [2H]c1c([2H])c([2H])c(-c2c([2H])c([2H])c(-c3nc(-n4c5c([2H])c([2H])c([2H])c6c7c([2H])c([2H])c([2H])c([2H])c7c7c([2H])c([2H])c([2H])c4c7c65)nc4oc5c([2H])c([2H])c(-c6c([2H])c([2H])c([2H])c([2H])c6[2H])c([2H])c5c34)c([2H])c2[2H])c([2H])c1[2H]]

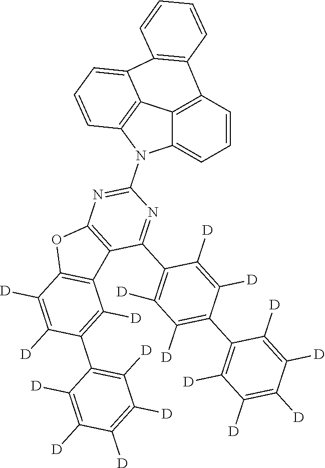 [SMILES: [2H]c1c([2H])c([2H])c(-c2c([2H])c([2H])c(-c3nc(-n4c5cccc6c7ccccc7c7cccc4c7c65)nc4oc5c([2H])c([2H])c(-c6c([2H])c([2H])c([2H])c([2H])c6[2H])c([2H])c5c34)c([2H])c2[2H])c([2H])c1[2H]]

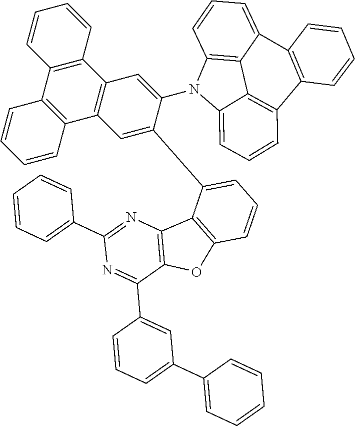 [SMILES: c1ccc(-c2cccc(-c3nc(-c4ccccc4)nc4c3oc3cccc(-c5cc6c7ccccc7c7ccccc7c6cc5-n5c6cccc7c8ccccc8c8cccc5c8c76)c34)c2)cc1]

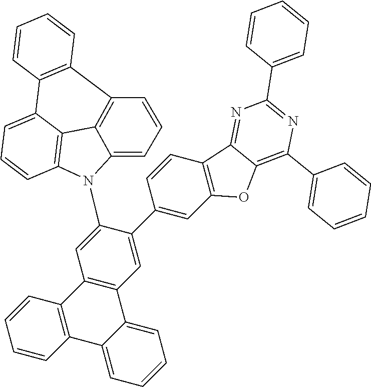 [SMILES: c1ccc(-c2nc(-c3ccccc3)c3oc4cc(-c5cc6c7ccccc7c7ccccc7c6cc5-n5c6cccc7c8ccccc8c8cccc5c8c76)ccc4c3n2)cc1]

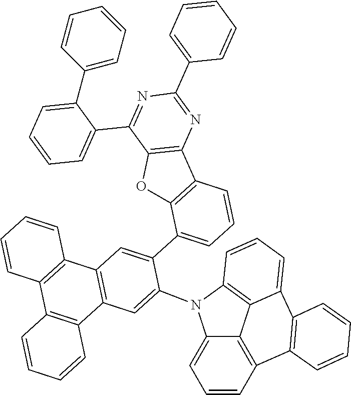 [SMILES: c1ccc(-c2nc(-c3ccccc3-c3ccccc3)c3oc4c(-c5cc6c7ccccc7c7ccccc7c6cc5-n5c6cccc7c8ccccc8c8cccc5c8c76)cccc4c3n2)cc1]

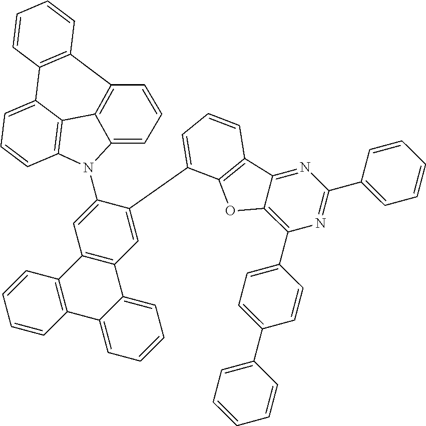 [SMILES: c1ccc(-c2ccc(-c3nc(-c4ccccc4)nc4c3oc3c(-c5cc6c7ccccc7c7ccccc7c6cc5-n5c6cccc7c8ccccc8c8cccc5c8c76)cccc34)cc2)cc1]

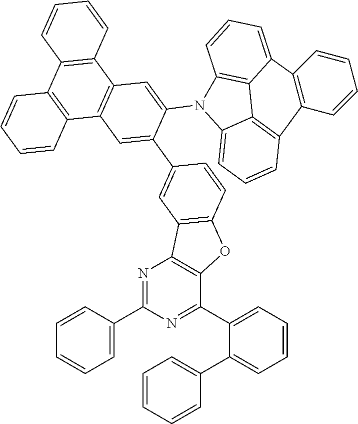 [SMILES: c1ccc(-c2nc(-c3ccccc3-c3ccccc3)c3oc4ccc(-c5cc6c7ccccc7c7ccccc7c6cc5-n5c6cccc7c8ccccc8c8cccc5c8c76)cc4c3n2)cc1]

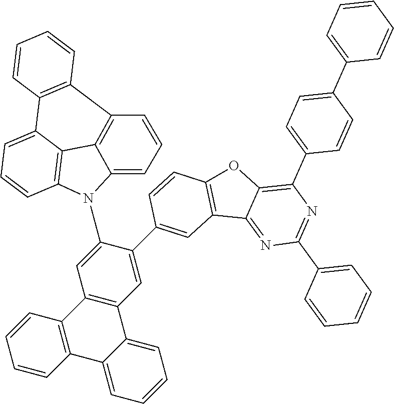 [SMILES: c1ccc(-c2ccc(-c3nc(-c4ccccc4)nc4c3oc3ccc(-c5cc6c7ccccc7c7ccccc7c6cc5-n5c6cccc7c8ccccc8c8cccc5c8c76)cc34)cc2)cc1]

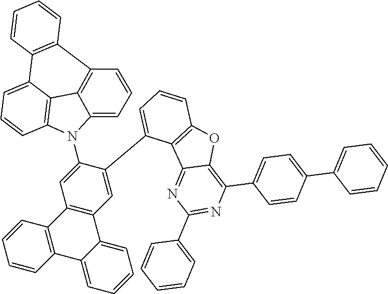 [SMILES: c1ccc(-c2ccc(-c3nc(-c4ccccc4)nc4c3oc3cccc(-c5cc6c7ccccc7c7ccccc7c6cc5-n5c6cccc7c8ccccc8c8cccc5c8c76)c34)cc2)cc1]

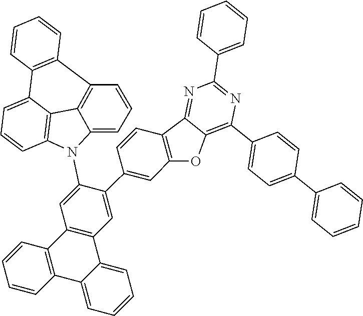 [SMILES: c1ccc(-c2ccc(-c3nc(-c4ccccc4)nc4c3oc3cc(-c5cc6c7ccccc7c7ccccc7c6cc5-n5c6cccc7c8ccccc8c8cccc5c8c76)ccc34)cc2)cc1]

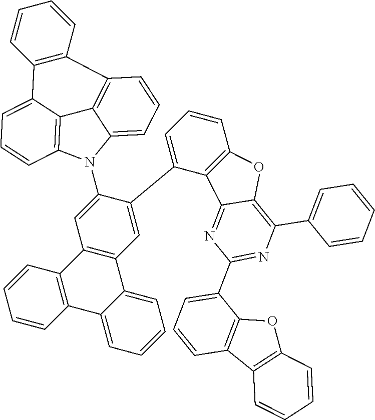 [SMILES: c1ccc(-c2nc(-c3cccc4c3oc3ccccc34)nc3c2oc2cccc(-c4cc5c6ccccc6c6ccccc6c5cc4-n4c5cccc6c7ccccc7c7cccc4c7c65)c23)cc1]

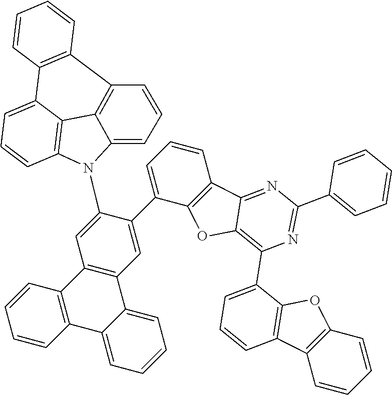 [SMILES: c1ccc(-c2nc(-c3cccc4c3oc3ccccc34)c3oc4c(-c5cc6c7ccccc7c7ccccc7c6cc5-n5c6cccc7c8ccccc8c8cccc5c8c76)cccc4c3n2)cc1]

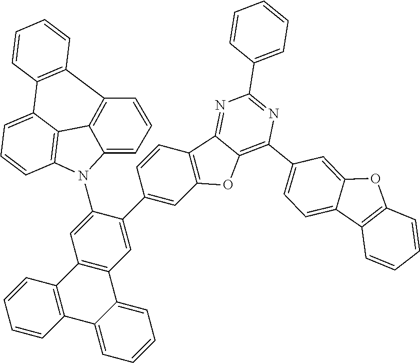 [SMILES: c1ccc(-c2nc(-c3ccc4c(c3)oc3ccccc34)c3oc4cc(-c5cc6c7ccccc7c7ccccc7c6cc5-n5c6cccc7c8ccccc8c8cccc5c8c76)ccc4c3n2)cc1]

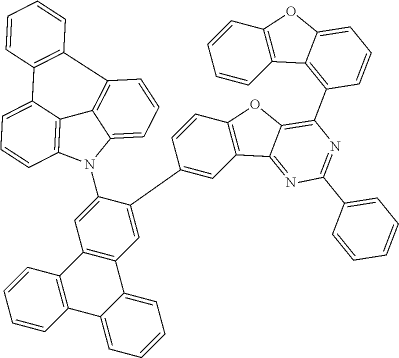 [SMILES: c1ccc(-c2nc(-c3cccc4oc5ccccc5c34)c3oc4ccc(-c5cc6c7ccccc7c7ccccc7c6cc5-n5c6cccc7c8ccccc8c8cccc5c8c76)cc4c3n2)cc1]

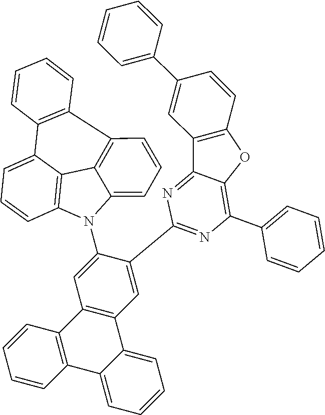 [SMILES: c1ccc(-c2ccc3oc4c(-c5ccccc5)nc(-c5cc6c7ccccc7c7ccccc7c6cc5-n5c6cccc7c8ccccc8c8cccc5c8c76)nc4c3c2)cc1]

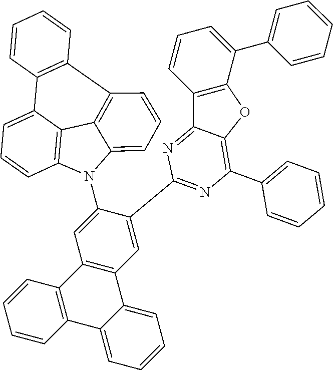 [SMILES: c1ccc(-c2cccc3c2oc2c(-c4ccccc4)nc(-c4cc5c6ccccc6c6ccccc6c5cc4-n4c5cccc6c7ccccc7c7cccc4c7c65)nc23)cc1]

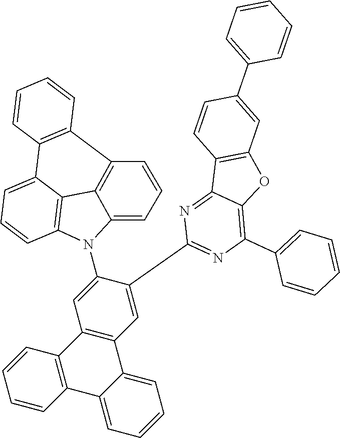 [SMILES: c1ccc(-c2ccc3c(c2)oc2c(-c4ccccc4)nc(-c4cc5c6ccccc6c6ccccc6c5cc4-n4c5cccc6c7ccccc7c7cccc4c7c65)nc23)cc1]

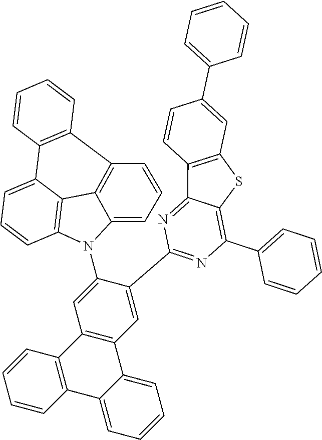 [SMILES: c1ccc(-c2ccc3c(c2)sc2c(-c4ccccc4)nc(-c4cc5c6ccccc6c6ccccc6c5cc4-n4c5cccc6c7ccccc7c7cccc4c7c65)nc23)cc1]

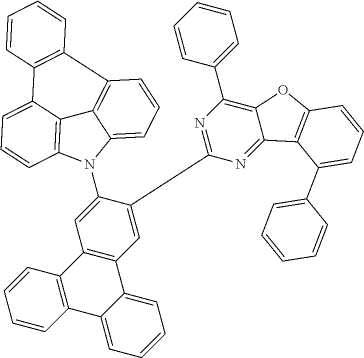 [SMILES: c1ccc(-c2nc(-c3cc4c5ccccc5c5ccccc5c4cc3-n3c4cccc5c6ccccc6c6cccc3c6c54)nc3c2oc2cccc(-c4ccccc4)c23)cc1]

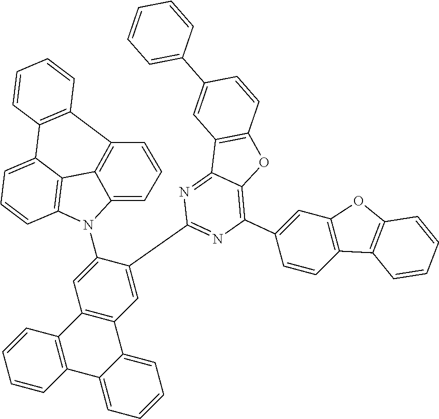 [SMILES: c1ccc(-c2ccc3oc4c(-c5ccc6c(c5)oc5ccccc56)nc(-c5cc6c7ccccc7c7ccccc7c6cc5-n5c6cccc7c8ccccc8c8cccc5c8c76)nc4c3c2)cc1]

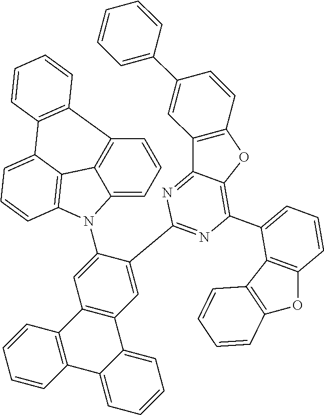 [SMILES: c1ccc(-c2ccc3oc4c(-c5cccc6oc7ccccc7c56)nc(-c5cc6c7ccccc7c7ccccc7c6cc5-n5c6cccc7c8ccccc8c8cccc5c8c76)nc4c3c2)cc1]

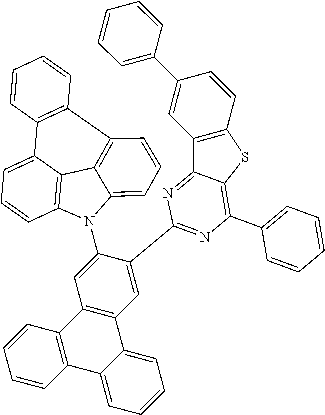 [SMILES: c1ccc(-c2ccc3sc4c(-c5ccccc5)nc(-c5cc6c7ccccc7c7ccccc7c6cc5-n5c6cccc7c8ccccc8c8cccc5c8c76)nc4c3c2)cc1]

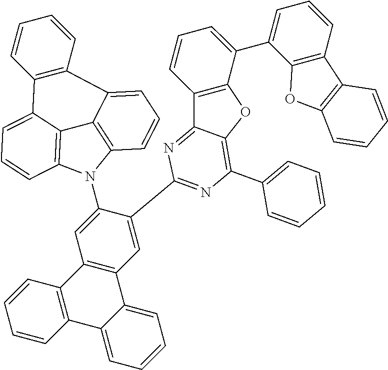 [SMILES: c1ccc(-c2nc(-c3cc4c5ccccc5c5ccccc5c4cc3-n3c4cccc5c6ccccc6c6cccc3c6c54)nc3c2oc2c(-c4cccc5c4oc4ccccc45)cccc23)cc1]

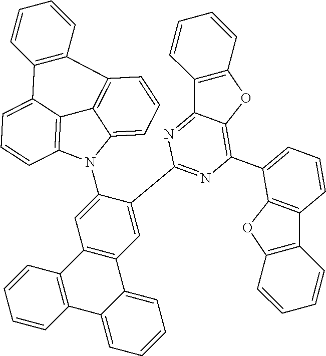 [SMILES: c1ccc2c(c1)oc1c(-c3nc(-c4cc5c6ccccc6c6ccccc6c5cc4-n4c5cccc6c7ccccc7c7cccc4c7c65)nc4c3oc3ccccc34)cccc12]

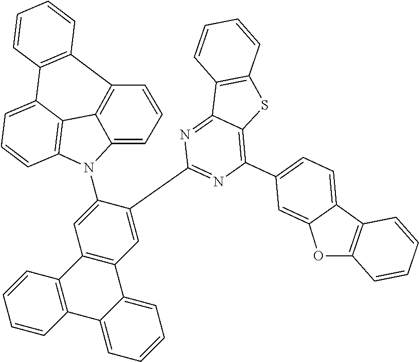 [SMILES: c1ccc2c(c1)oc1cc(-c3nc(-c4cc5c6ccccc6c6ccccc6c5cc4-n4c5cccc6c7ccccc7c7cccc4c7c65)nc4c3sc3ccccc34)ccc12]

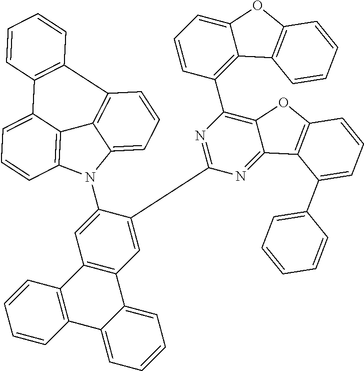 [SMILES: c1ccc(-c2cccc3oc4c(-c5cccc6oc7ccccc7c56)nc(-c5cc6c7ccccc7c7ccccc7c6cc5-n5c6cccc7c8ccccc8c8cccc5c8c76)nc4c23)cc1]

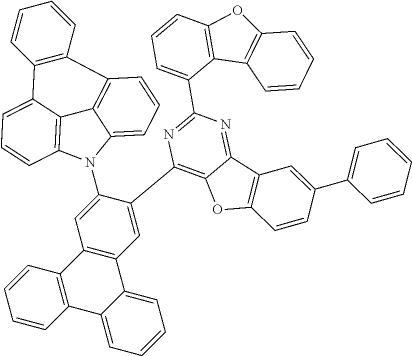 [SMILES: c1ccc(-c2ccc3oc4c(-c5cc6c7ccccc7c7ccccc7c6cc5-n5c6cccc7c8ccccc8c8cccc5c8c76)nc(-c5cccc6oc7ccccc7c56)nc4c3c2)cc1]

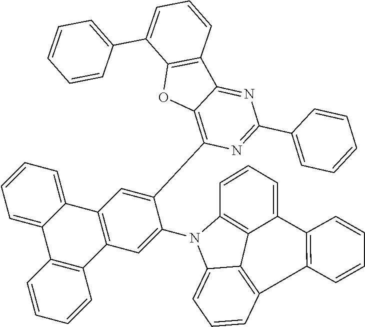 [SMILES: c1ccc(-c2nc(-c3cc4c5ccccc5c5ccccc5c4cc3-n3c4cccc5c6ccccc6c6cccc3c6c54)c3oc4c(-c5ccccc5)cccc4c3n2)cc1]

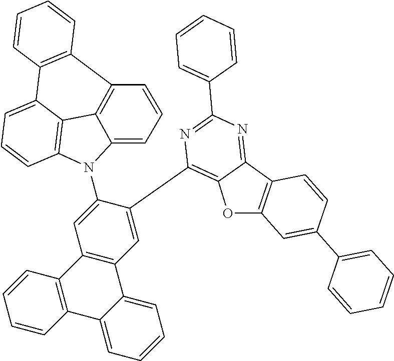 [SMILES: c1ccc(-c2ccc3c(c2)oc2c(-c4cc5c6ccccc6c6ccccc6c5cc4-n4c5cccc6c7ccccc7c7cccc4c7c65)nc(-c4ccccc4)nc23)cc1]

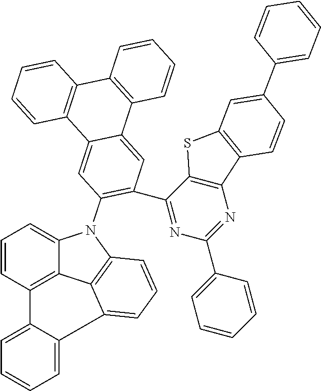 [SMILES: c1ccc(-c2ccc3c(c2)sc2c(-c4cc5c6ccccc6c6ccccc6c5cc4-n4c5cccc6c7ccccc7c7cccc4c7c65)nc(-c4ccccc4)nc23)cc1]

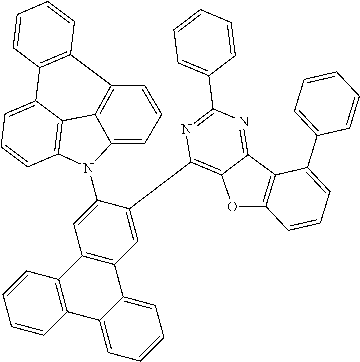 [SMILES: c1ccc(-c2nc(-c3cc4c5ccccc5c5ccccc5c4cc3-n3c4cccc5c6ccccc6c6cccc3c6c54)c3oc4cccc(-c5ccccc5)c4c3n2)cc1]

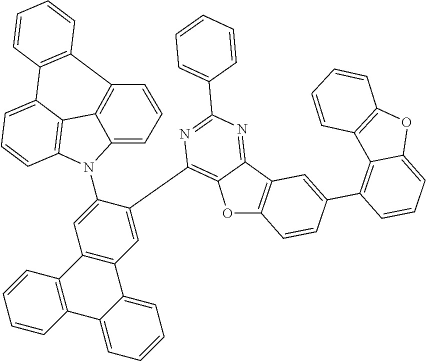 [SMILES: c1ccc(-c2nc(-c3cc4c5ccccc5c5ccccc5c4cc3-n3c4cccc5c6ccccc6c6cccc3c6c54)c3oc4ccc(-c5cccc6oc7ccccc7c56)cc4c3n2)cc1]

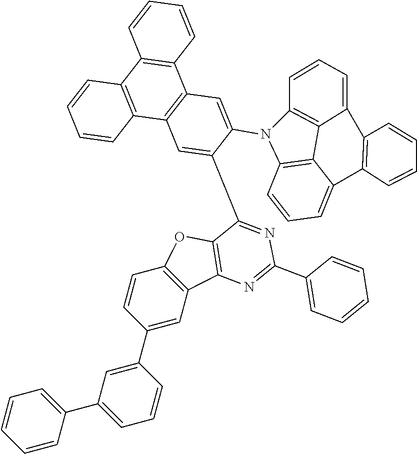 [SMILES: c1ccc(-c2cccc(-c3ccc4oc5c(-c6cc7c8ccccc8c8ccccc8c7cc6-n6c7cccc8c9ccccc9c9cccc6c9c87)nc(-c6ccccc6)nc5c4c3)c2)cc1]

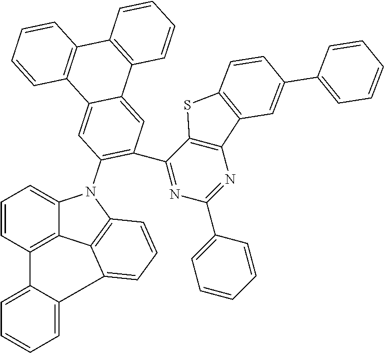 [SMILES: c1ccc(-c2ccc3sc4c(-c5cc6c7ccccc7c7ccccc7c6cc5-n5c6cccc7c8ccccc8c8cccc5c8c76)nc(-c5ccccc5)nc4c3c2)cc1]

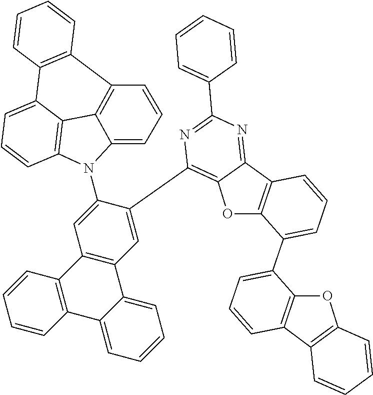 [SMILES: c1ccc(-c2nc(-c3cc4c5ccccc5c5ccccc5c4cc3-n3c4cccc5c6ccccc6c6cccc3c6c54)c3oc4c(-c5cccc6c5oc5ccccc56)cccc4c3n2)cc1]

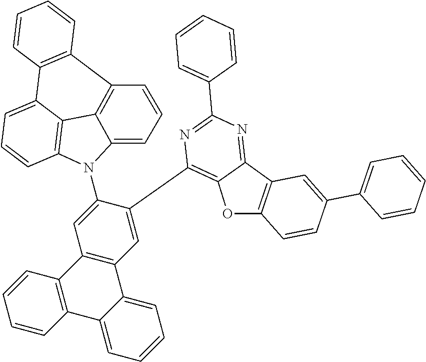 [SMILES: c1ccc(-c2ccc3oc4c(-c5cc6c7ccccc7c7ccccc7c6cc5-n5c6cccc7c8ccccc8c8cccc5c8c76)nc(-c5ccccc5)nc4c3c2)cc1]

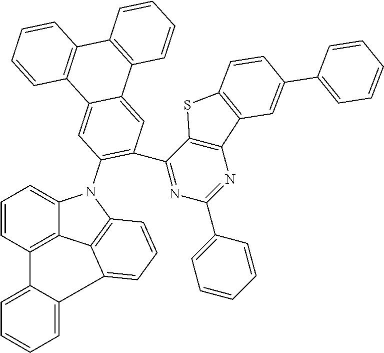 [SMILES: c1ccc(-c2ccc3sc4c(-c5cc6c7ccccc7c7ccccc7c6cc5-n5c6cccc7c8ccccc8c8cccc5c8c76)nc(-c5ccccc5)nc4c3c2)cc1]

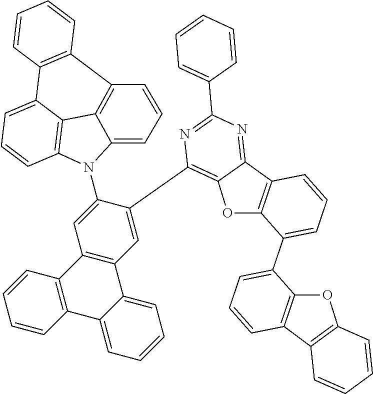 [SMILES: c1ccc(-c2nc(-c3cc4c5ccccc5c5ccccc5c4cc3-n3c4cccc5c6ccccc6c6cccc3c6c54)c3oc4c(-c5cccc6c5oc5ccccc56)cccc4c3n2)cc1]

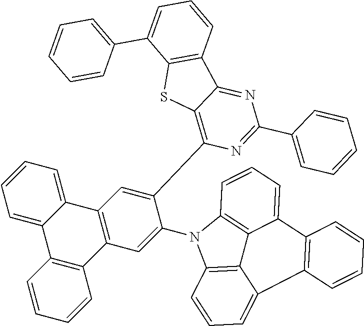 [SMILES: c1ccc(-c2nc(-c3cc4c5ccccc5c5ccccc5c4cc3-n3c4cccc5c6ccccc6c6cccc3c6c54)c3sc4c(-c5ccccc5)cccc4c3n2)cc1]

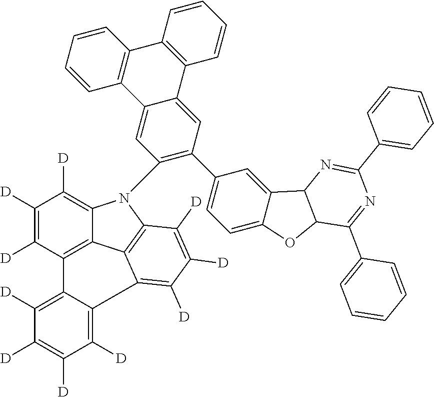 [SMILES: [2H]c1c([2H])c([2H])c2c(c1[2H])c1c([2H])c([2H])c([2H])c3c1c1c2c([2H])c([2H])c([2H])c1n3-c1cc2c3ccccc3c3ccccc3c2cc1-c1ccc2c(c1)C1N=C(c3ccccc3)N=C(c3ccccc3)C1O2]

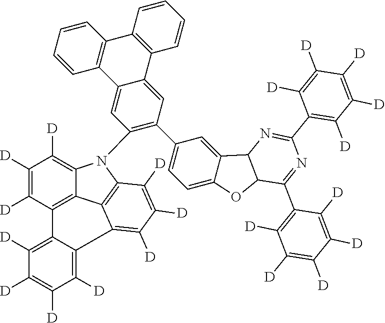 [SMILES: [2H]c1c([2H])c([2H])c(C2=NC3c4cc(-c5cc6c7ccccc7c7ccccc7c6cc5-n5c6c([2H])c([2H])c([2H])c7c8c([2H])c([2H])c([2H])c([2H])c8c8c([2H])c([2H])c([2H])c5c8c76)ccc4OC3C(c3c([2H])c([2H])c([2H])c([2H])c3[2H])=N2)c([2H])c1[2H]]

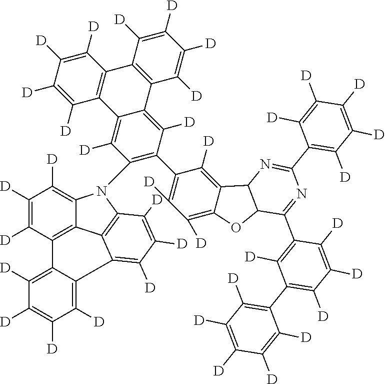 [SMILES: [2H]c1c([2H])c([2H])c(C2=NC3c4c([2H])c(-c5c(-n6c7c([2H])c([2H])c([2H])c8c9c([2H])c([2H])c([2H])c([2H])c9c9c([2H])c([2H])c([2H])c6c9c87)c([2H])c6c7c([2H])c([2H])c([2H])c([2H])c7c7c([2H])c([2H])c([2H])c([2H])c7c6c5[2H])c([2H])c([2H])c4OC3C(c3c([2H])c([2H])c([2H])c(-c4c([2H])c([2H])c([2H])c([2H])c4[2H])c3[2H])=N2)c([2H])c1[2H]]

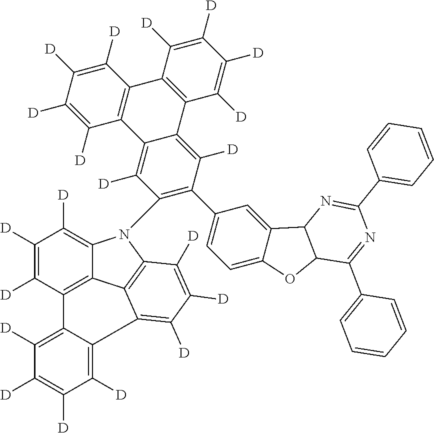 [SMILES: [2H]c1c([2H])c([2H])c2c(c1[2H])c1c([2H])c([2H])c([2H])c([2H])c1c1c([2H])c(-n3c4c([2H])c([2H])c([2H])c5c6c([2H])c([2H])c([2H])c([2H])c6c6c([2H])c([2H])c([2H])c3c6c54)c(-c3ccc4c(c3)C3N=C(c5ccccc5)N=C(c5ccccc5)C3O4)c([2H])c21]

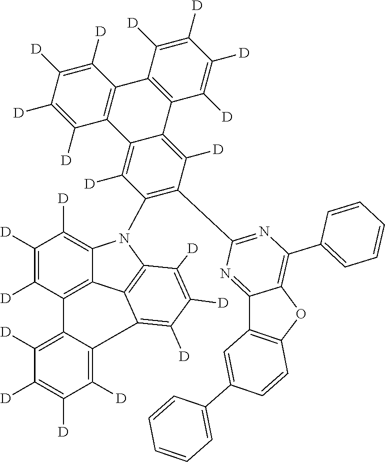 [SMILES: [2H]c1c([2H])c([2H])c2c(c1[2H])c1c([2H])c([2H])c([2H])c([2H])c1c1c([2H])c(-n3c4c([2H])c([2H])c([2H])c5c6c([2H])c([2H])c([2H])c([2H])c6c6c([2H])c([2H])c([2H])c3c6c54)c(-c3nc(-c4ccccc4)c4oc5ccc(-c6ccccc6)cc5c4n3)c([2H])c21]

Particularly suitable compounds of the formulae (1), (1a), (1b), (1c), (1d), (1e), (1f), (1g), (1h), (1i), (1j), (1k), (1l) and (1m) are the compounds E1 to E45 in table 2.

TABLE 2

| | |
|---|---|
| 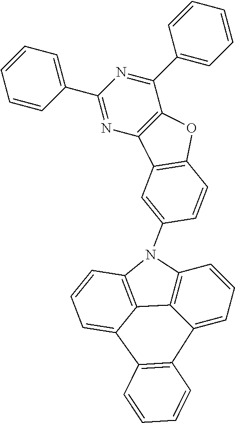 [SMILES: c1ccc(-c2nc(-c3ccccc3)c3oc4ccc(-n5c6cccc7c8ccccc8c8cccc5c8c76)cc4c3n2)cc1] | E1 |
| 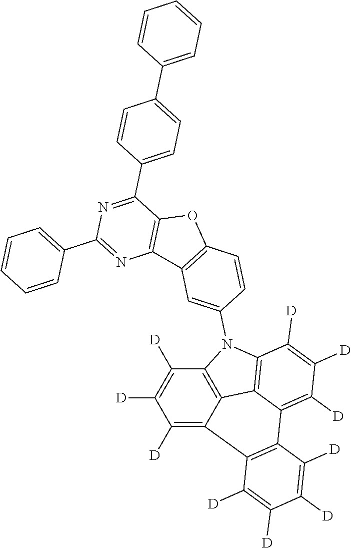 [SMILES: [2H]c1c([2H])c([2H])c2c(c1[2H])c1c([2H])c([2H])c([2H])c3c1c1c2c([2H])c([2H])c([2H])c1n3-c1ccc2oc3c(-c4ccc(-c5ccccc5)cc4)nc(-c4ccccc4)nc3c2c1] | E2 |
| 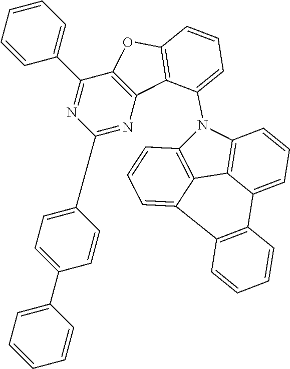 [SMILES: c1ccc(-c2ccc(-c3nc(-c4ccccc4)c4oc5cccc(-n6c7cccc8c9ccccc9c9cccc6c9c87)c5c4n3)cc2)cc1] | E3 |
| 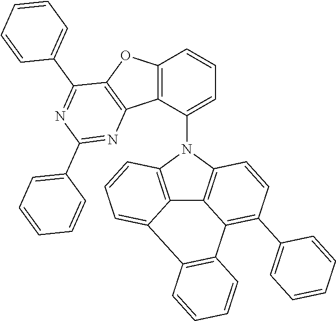 [SMILES: c1ccc(-c2nc(-c3ccccc3)c3oc4cccc(-n5c6cccc7c8ccccc8c8c(-c9ccccc9)ccc5c8c76)c4c3n2)cc1] | E4 |
| 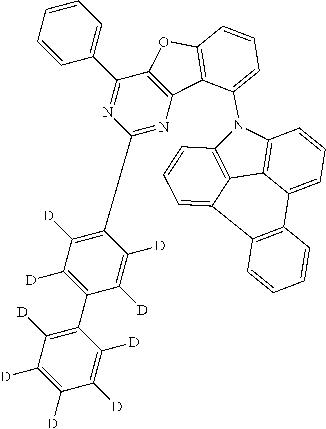 [SMILES: [2H]c1c([2H])c([2H])c(-c2c([2H])c([2H])c(-c3nc(-c4ccccc4)c4oc5cccc(-n6c7cccc8c9ccccc9c9cccc6c9c87)c5c4n3)c([2H])c2[2H])c([2H])c1[2H]] | E5 |
| 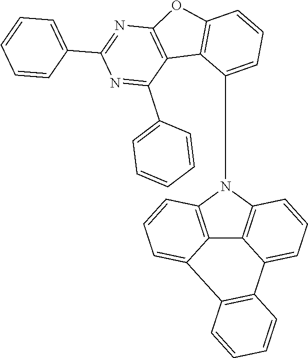 [SMILES: c1ccc(-c2nc(-c3ccccc3)c3c(n2)oc2cccc(-n4c5cccc6c7ccccc7c7cccc4c7c65)c23)cc1] | E6 |
| 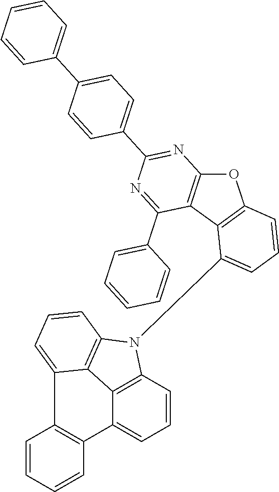 [SMILES: c1ccc(-c2ccc(-c3nc(-c4ccccc4)c4c(n3)oc3cccc(-n5c6cccc7c8ccccc8c8cccc5c8c76)c34)cc2)cc1] | E7 |
| 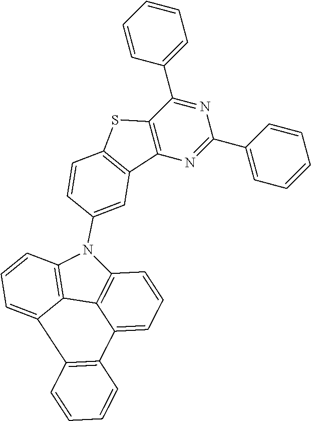 [SMILES: c1ccc(-c2nc(-c3ccccc3)c3sc4ccc(-n5c6cccc7c8ccccc8c8cccc5c8c76)cc4c3n2)cc1] | E8 |
| 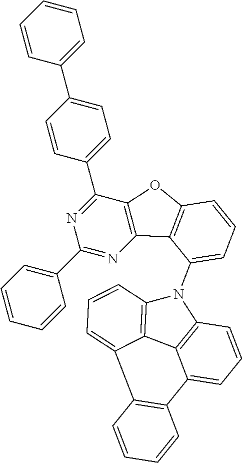 [SMILES: c1ccc(-c2ccc(-c3nc(-c4ccccc4)nc4c3oc3cccc(-n5c6cccc7c8ccccc8c8cccc5c8c76)c34)cc2)cc1] | E9 |
| 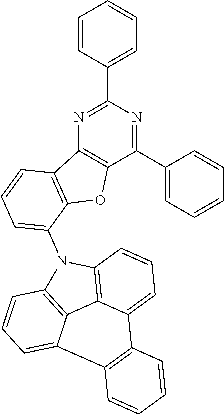 [SMILES: c1ccc(-c2nc(-c3ccccc3)c3oc4c(-n5c6cccc7c8ccccc8c8cccc5c8c76)cccc4c3n2)cc1] | E10 |
| 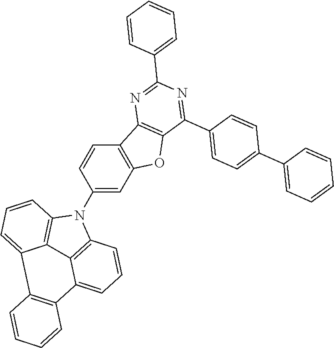 [SMILES: c1ccc(-c2ccc(-c3nc(-c4ccccc4)nc4c3oc3cc(-n5c6cccc7c8ccccc8c8cccc5c8c76)ccc34)cc2)cc1] | E11 |
| 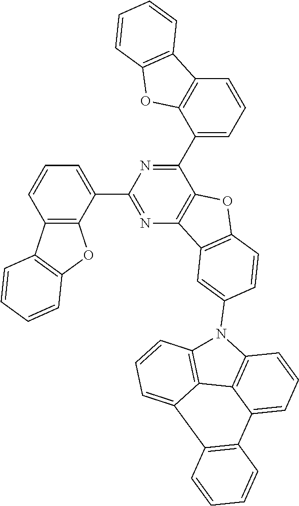 [SMILES: c1ccc2c(c1)oc1c(-c3nc(-c4cccc5c4oc4ccccc45)c4oc5ccc(-n6c7cccc8c9ccccc9c9cccc6c9c87)cc5c4n3)cccc12] | E12 |
| 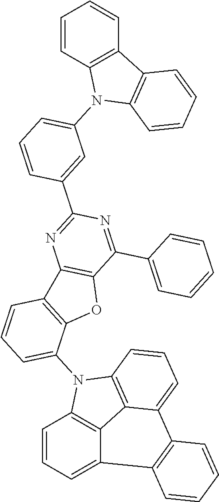 [SMILES: c1ccc(-c2nc(-c3cccc(-n4c5ccccc5c5ccccc54)c3)nc3c2oc2c(-n4c5cccc6c7ccccc7c7cccc4c7c65)cccc23)cc1] | E13 |
| 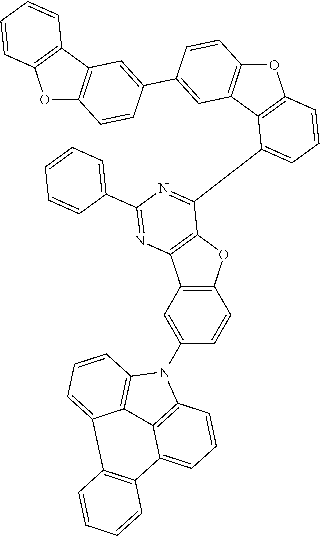 [SMILES: c1ccc(-c2nc(-c3cccc4oc5ccc(-c6ccc7oc8ccccc8c7c6)cc5c34)c3oc4ccc(-n5c6cccc7c8ccccc8c8cccc5c8c76)cc4c3n2)cc1] | E14 |
| 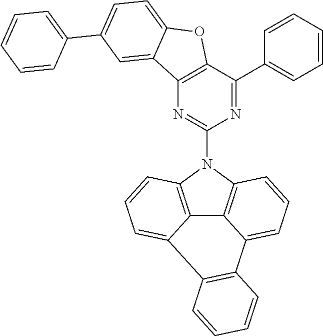 [SMILES: c1ccc(-c2ccc3oc4c(-c5ccccc5)nc(-n5c6cccc7c8ccccc8c8cccc5c8c76)nc4c3c2)cc1] | E15 |
| 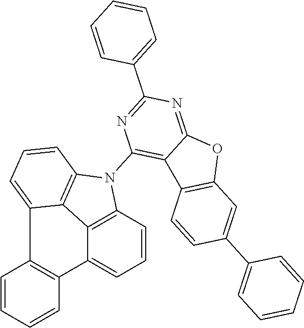 [SMILES: c1ccc(-c2ccc3c(c2)oc2nc(-c4ccccc4)nc(-n4c5cccc6c7ccccc7c7cccc4c7c65)c23)cc1] | E16 |
| 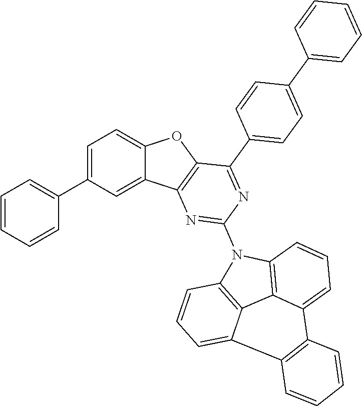 [SMILES: c1ccc(-c2ccc(-c3nc(-n4c5cccc6c7ccccc7c7cccc4c7c65)nc4c3oc3ccc(-c5ccccc5)cc34)cc2)cc1] | E17 |
| 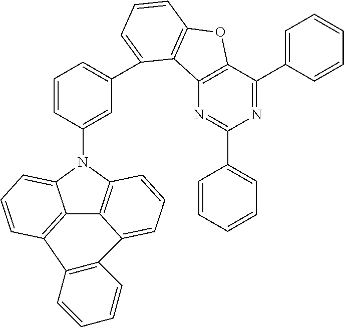 [SMILES: c1ccc(-c2nc(-c3ccccc3)c3oc4cccc(-c5cccc(-n6c7cccc8c9ccccc9c9cccc6c9c87)c5)c4c3n2)cc1] | E18 |
| 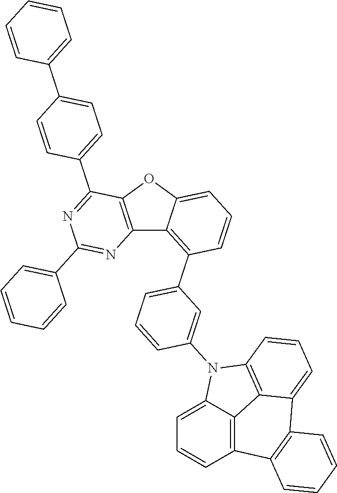 [SMILES: c1ccc(-c2ccc(-c3nc(-c4ccccc4)nc4c3oc3cccc(-c5cccc(-n6c7cccc8c9ccccc9c9cccc6c9c87)c5)c34)cc2)cc1] | E19 |
| 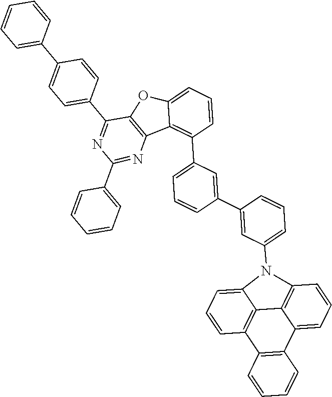 [SMILES: c1ccc(-c2ccc(-c3nc(-c4ccccc4)nc4c3oc3cccc(-c5cccc(-c6cccc(-n7c8cccc9c%10ccccc%10c%10cccc7c%10c98)c6)c5)c34)cc2)cc1] | E20 |
| 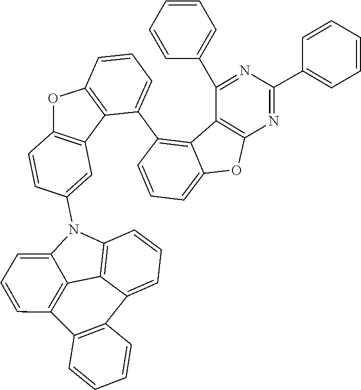 [SMILES: c1ccc(-c2nc(-c3ccccc3)c3c(n2)oc2cccc(-c4cccc5oc6ccc(-n7c8cccc9c%10ccccc%10c%10cccc7c%10c98)cc6c45)c23)cc1] | E21 |
| 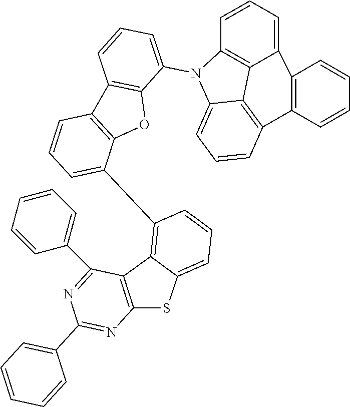 [SMILES: c1ccc(-c2nc(-c3ccccc3)c3c(n2)sc2cccc(-c4cccc5c4oc4c(-n6c7cccc8c9ccccc9c9cccc6c9c87)cccc45)c23)cc1] | E22 |
| 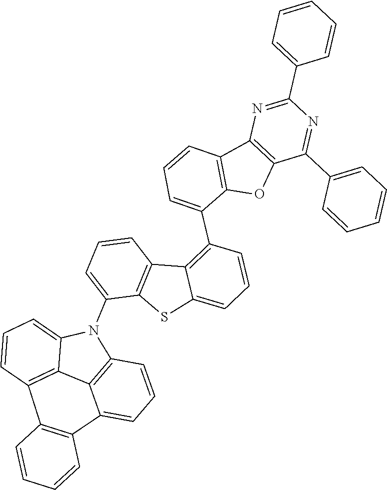 [SMILES: c1ccc(-c2nc(-c3ccccc3)c3oc4c(-c5cccc6sc7c(-n8c9cccc%10c%11ccccc%11c%11cccc8c%11c%109)cccc7c56)cccc4c3n2)cc1] | E23 |
| 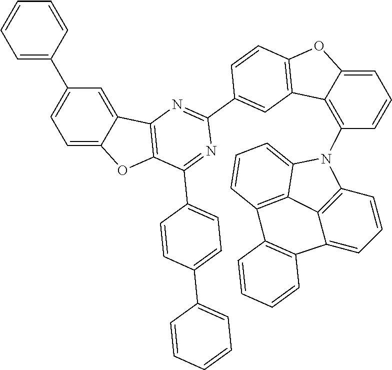 [SMILES: c1ccc(-c2ccc(-c3nc(-c4ccc5oc6cccc(-n7c8cccc9c%10ccccc%10c%10cccc7c%10c98)c6c5c4)nc4c3oc3ccc(-c5ccccc5)cc34)cc2)cc1] | E24 |
| 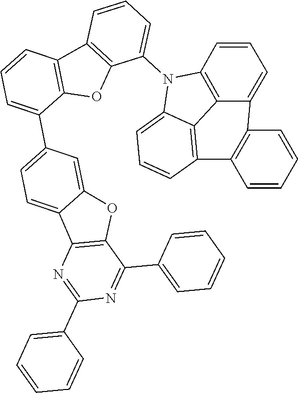 [SMILES: c1ccc(-c2nc(-c3ccccc3)c3oc4cc(-c5cccc6c5oc5c(-n7c8cccc9c%10ccccc%10c%10cccc7c%10c98)cccc56)ccc4c3n2)cc1] | E25 |
| 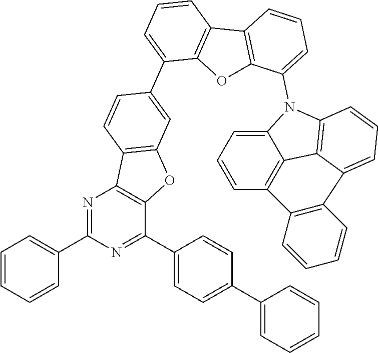 [SMILES: c1ccc(-c2ccc(-c3nc(-c4ccccc4)nc4c3oc3cc(-c5cccc6c5oc5c(-n7c8cccc9c%10ccccc%10c%10cccc7c%10c98)cccc56)ccc34)cc2)cc1] | E26 |
| 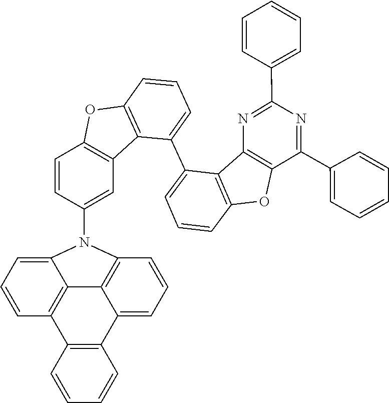 [SMILES: c1ccc(-c2nc(-c3ccccc3)c3oc4cccc(-c5cccc6oc7ccc(-n8c9cccc%10c%11ccccc%11c%11cccc8c%11c%109)cc7c56)c4c3n2)cc1] | E27 |
| 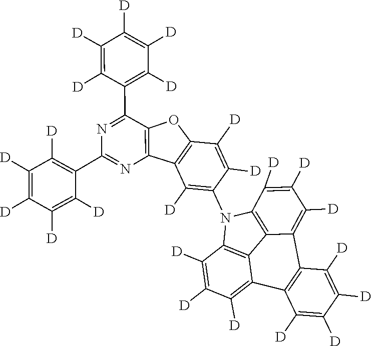 [SMILES: [2H]c1c([2H])c([2H])c(-c2nc(-c3c([2H])c([2H])c([2H])c([2H])c3[2H])c3oc4c([2H])c([2H])c(-n5c6c([2H])c([2H])c([2H])c7c8c([2H])c([2H])c([2H])c([2H])c8c8c([2H])c([2H])c([2H])c5c8c76)c([2H])c4c3n2)c([2H])c1[2H]] | E28 |
| 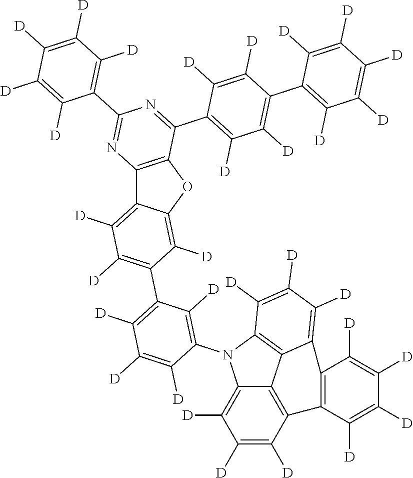 [SMILES: [2H]c1c([2H])c([2H])c(-c2nc(-c3c([2H])c([2H])c(-c4c([2H])c([2H])c([2H])c([2H])c4[2H])c([2H])c3[2H])c3oc4c([2H])c(-c5c([2H])c([2H])c([2H])c(-n6c7c([2H])c([2H])c([2H])c8c9c([2H])c([2H])c([2H])c([2H])c9c9c([2H])c([2H])c([2H])c6c9c87)c5[2H])c([2H])c([2H])c4c3n2)c([2H])c1[2H]] | E29 |
| 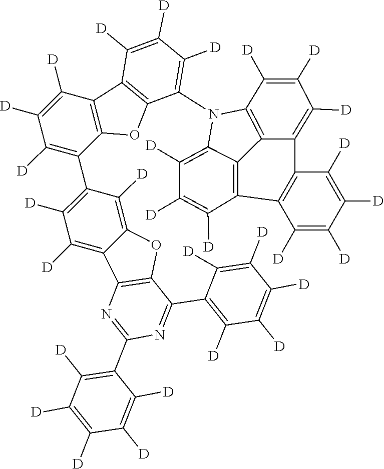 [SMILES: [2H]c1c([2H])c([2H])c(-c2nc(-c3c([2H])c([2H])c([2H])c([2H])c3[2H])c3oc4c([2H])c(-c5c([2H])c([2H])c([2H])c6c5oc5c(-n7c8c([2H])c([2H])c([2H])c9c%10c([2H])c([2H])c([2H])c([2H])c%10c%10c([2H])c([2H])c([2H])c7c%10c98)c([2H])c([2H])c([2H])c56)c([2H])c([2H])c4c3n2)c([2H])c1[2H]] | E30 |
| 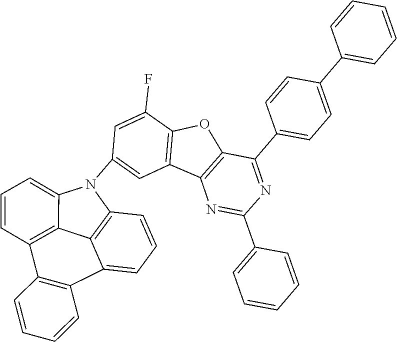 [SMILES: Fc1cc(-n2c3cccc4c5ccccc5c5cccc2c5c43)cc2c1oc1c(-c3ccc(-c4ccccc4)cc3)nc(-c3ccccc3)nc12] | E31 |
| 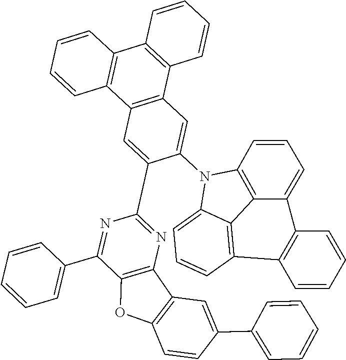 [SMILES: c1ccc(-c2ccc3oc4c(-c5ccccc5)nc(-c5cc6c7ccccc7c7ccccc7c6cc5-n5c6cccc7c8ccccc8c8cccc5c8c76)nc4c3c2)cc1] | E32 |
| 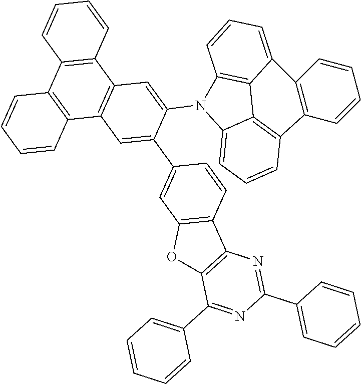 [SMILES: c1ccc(-c2nc(-c3ccccc3)c3oc4cc(-c5cc6c7ccccc7c7ccccc7c6cc5-n5c6cccc7c8ccccc8c8cccc5c8c76)ccc4c3n2)cc1] | E33 |
| 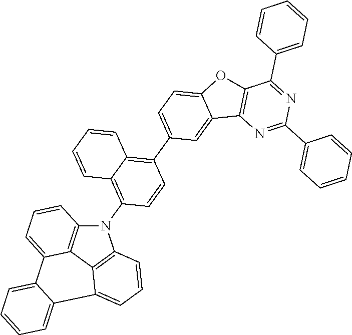 [SMILES: c1ccc(-c2nc(-c3ccccc3)c3oc4ccc(-c5ccc(-n6c7cccc8c9ccccc9c9cccc6c9c87)c6ccccc56)cc4c3n2)cc1] | E34 |
| 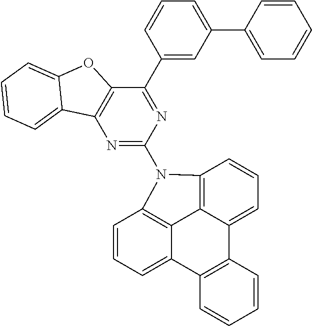 [SMILES: c1ccc(-c2cccc(-c3nc(-n4c5cccc6c7ccccc7c7cccc4c7c65)nc4c3oc3ccccc34)c2)cc1] | E35 |
| 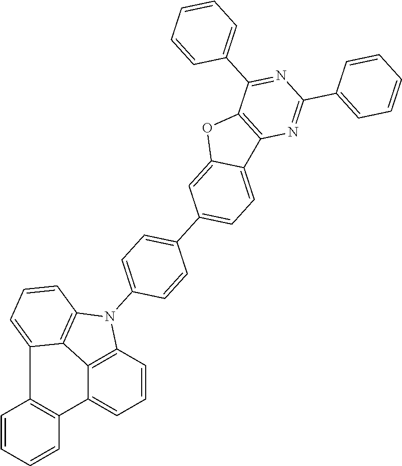 [SMILES: c1ccc(-c2nc(-c3ccccc3)c3oc4cc(-c5ccc(-n6c7cccc8c9ccccc9c9cccc6c9c87)cc5)ccc4c3n2)cc1] | E36 |
| 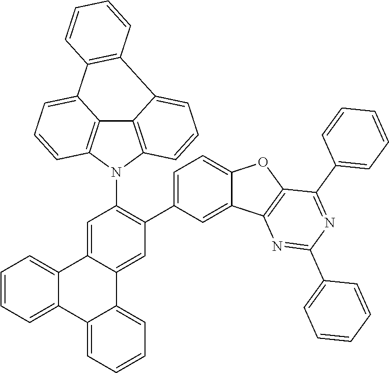 [SMILES: c1ccc(-c2nc(-c3ccccc3)c3oc4ccc(-c5cc6c7ccccc7c7ccccc7c6cc5-n5c6cccc7c8ccccc8c8cccc5c8c76)cc4c3n2)cc1] | E37 |
| 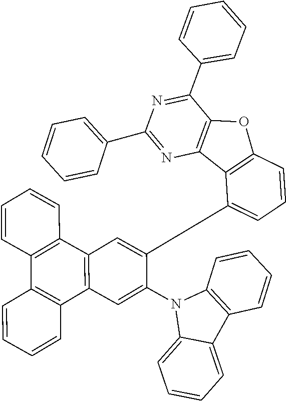 [SMILES: c1ccc(-c2nc(-c3ccccc3)c3oc4cccc(-c5cc6c7ccccc7c7ccccc7c6cc5-n5c6ccccc6c6ccccc65)c4c3n2)cc1] | E38 |
| 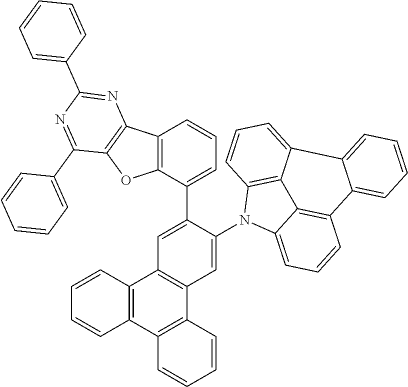 [SMILES: c1ccc(-c2nc(-c3ccccc3)c3oc4c(-c5cc6c7ccccc7c7ccccc7c6cc5-n5c6cccc7c8ccccc8c8cccc5c8c76)cccc4c3n2)cc1] | E39 |
| 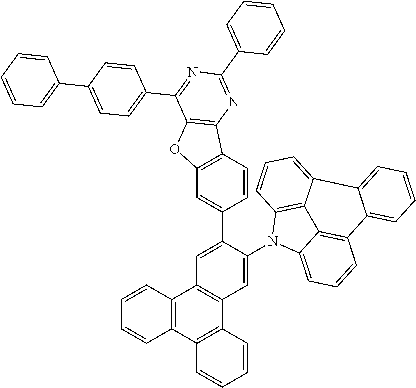 [SMILES: c1ccc(-c2ccc(-c3nc(-c4ccccc4)nc4c3oc3cc(-c5cc6c7ccccc7c7ccccc7c6cc5-n5c6cccc7c8ccccc8c8cccc5c8c76)ccc34)cc2)cc1] | E40 |
| 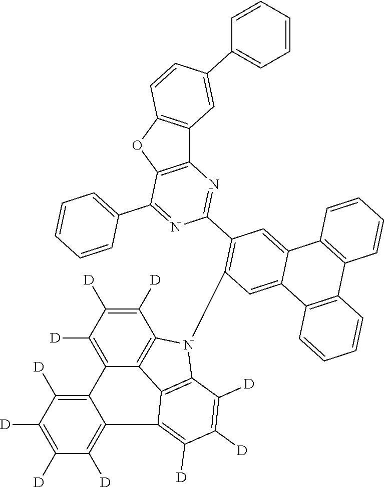 [SMILES: [2H]c1c([2H])c([2H])c2c(c1[2H])c1c([2H])c([2H])c([2H])c3c1c1c2c([2H])c([2H])c([2H])c1n3-c1cc2c3ccccc3c3ccccc3c2cc1-c1nc(-c2ccccc2)c2oc3ccc(-c4ccccc4)cc3c2n1] | E41 |
| 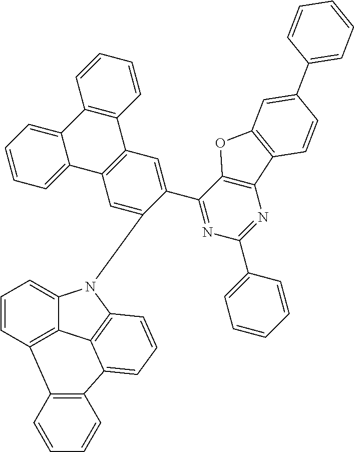 [SMILES: c1ccc(-c2ccc3c(c2)oc2c(-c4cc5c6ccccc6c6ccccc6c5cc4-n4c5cccc6c7ccccc7c7cccc4c7c65)nc(-c4ccccc4)nc23)cc1] | E42 |
| 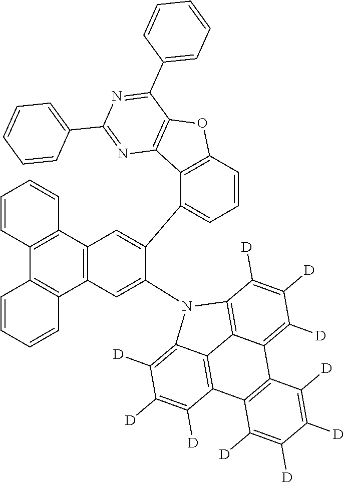 [SMILES: [2H]c1c([2H])c([2H])c2c(c1[2H])c1c([2H])c([2H])c([2H])c3c1c1c2c([2H])c([2H])c([2H])c1n3-c1cc2c3ccccc3c3ccccc3c2cc1-c1cccc2oc3c(-c4ccccc4)nc(-c4ccccc4)nc3c12] | E43 |
| 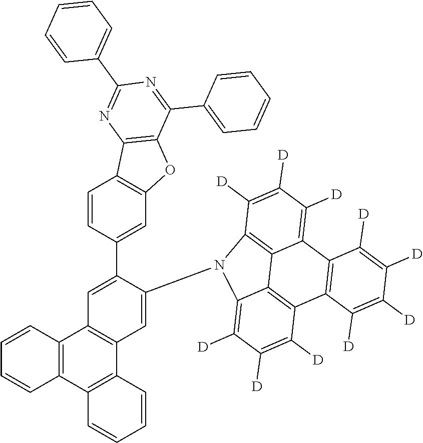 [SMILES: [2H]c1c([2H])c([2H])c2c(c1[2H])c1c([2H])c([2H])c([2H])c3c1c1c2c([2H])c([2H])c([2H])c1n3-c1cc2c3ccccc3c3ccccc3c2cc1-c1ccc2c(c1)oc1c(-c3ccccc3)nc(-c3ccccc3)nc12] | E44 |
| 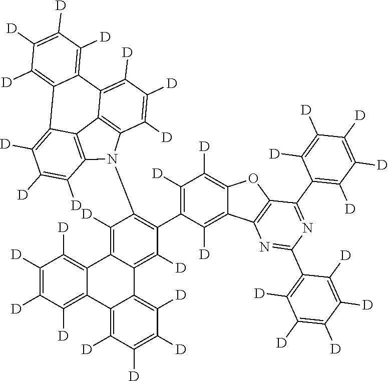 [SMILES: [2H]c1c([2H])c([2H])c(-c2nc(-c3c([2H])c([2H])c([2H])c([2H])c3[2H])c3oc4c([2H])c([2H])c(-c5c(-n6c7c([2H])c([2H])c([2H])c8c9c([2H])c([2H])c([2H])c([2H])c9c9c([2H])c([2H])c([2H])c6c9c87)c([2H])c6c7c([2H])c([2H])c([2H])c([2H])c7c7c([2H])c([2H])c([2H])c([2H])c7c6c5[2H])c([2H])c4c3n2)c([2H])c1[2H]] | E45 |

The compounds of the invention can be prepared by known synthesis steps, for example bromination, Suzuki coupling, Ullmann coupling, Hartwig-Buchwald coupling, etc.

In the synthesis schemes which follow, the compounds are shown with a small number of substituents to simplify the structures. This does not rule out the presence of any desired further substituents in the processes. The methods shown for synthesis of the compounds of the invention should be regarded as illustrative. Alternative synthesis pathways may be developed within the scope of common knowledge in the art.

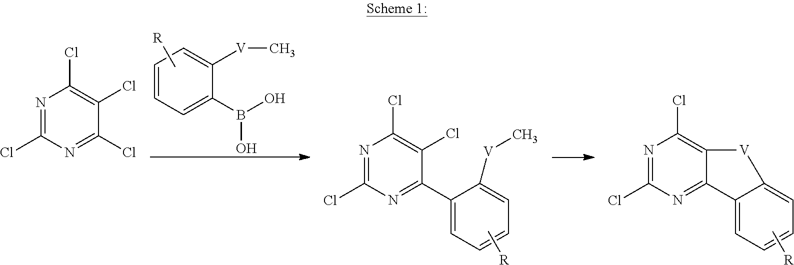

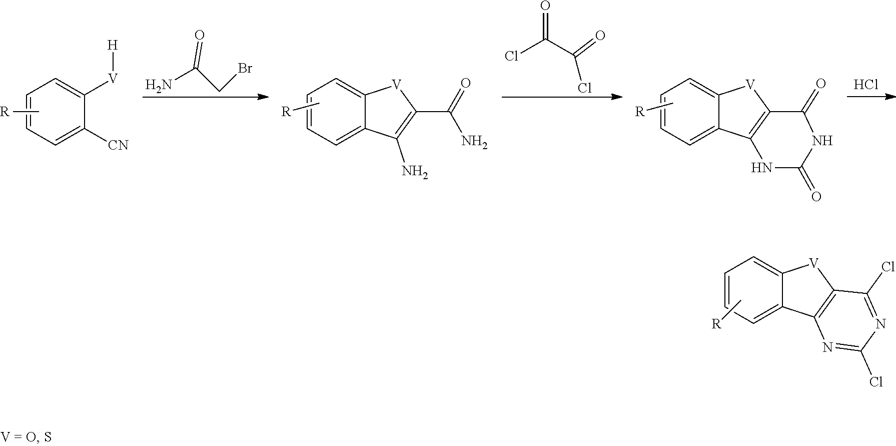

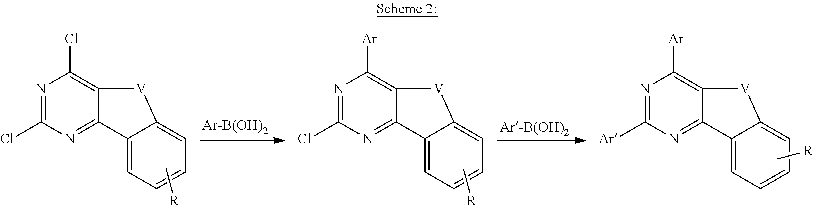

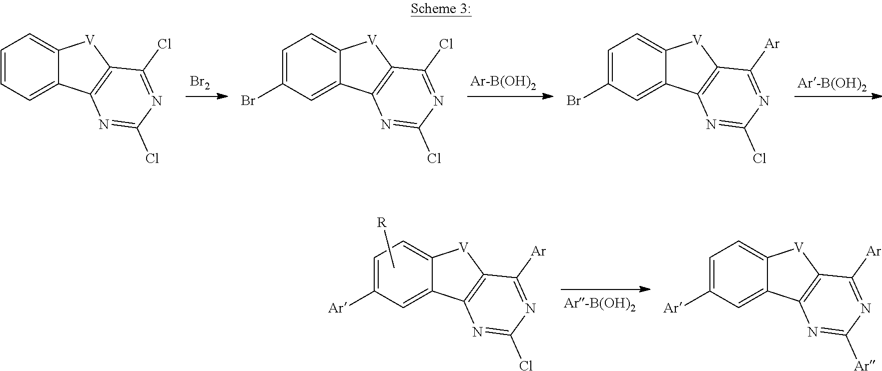

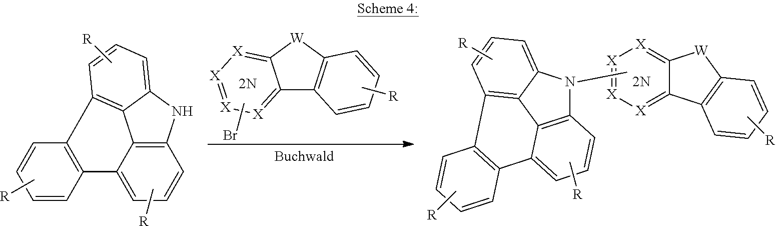

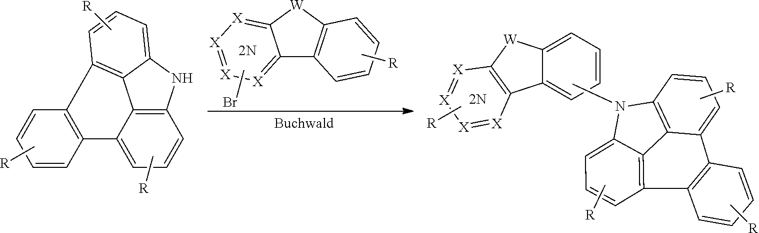

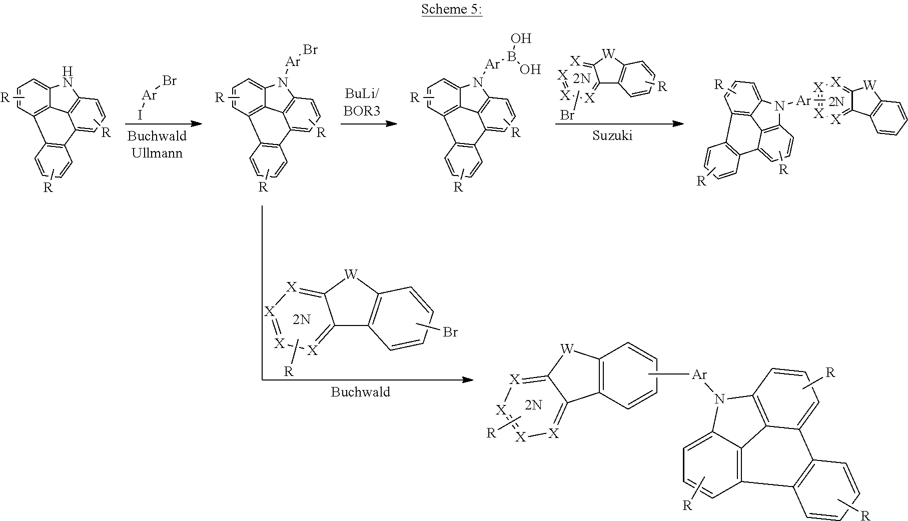

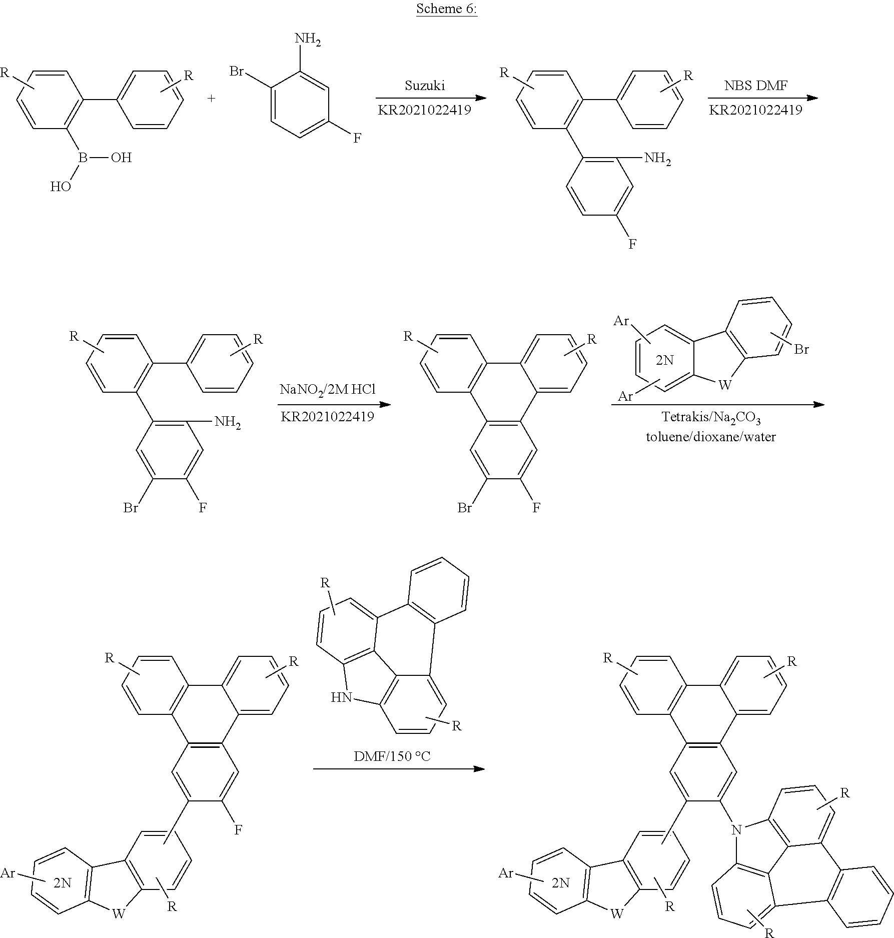

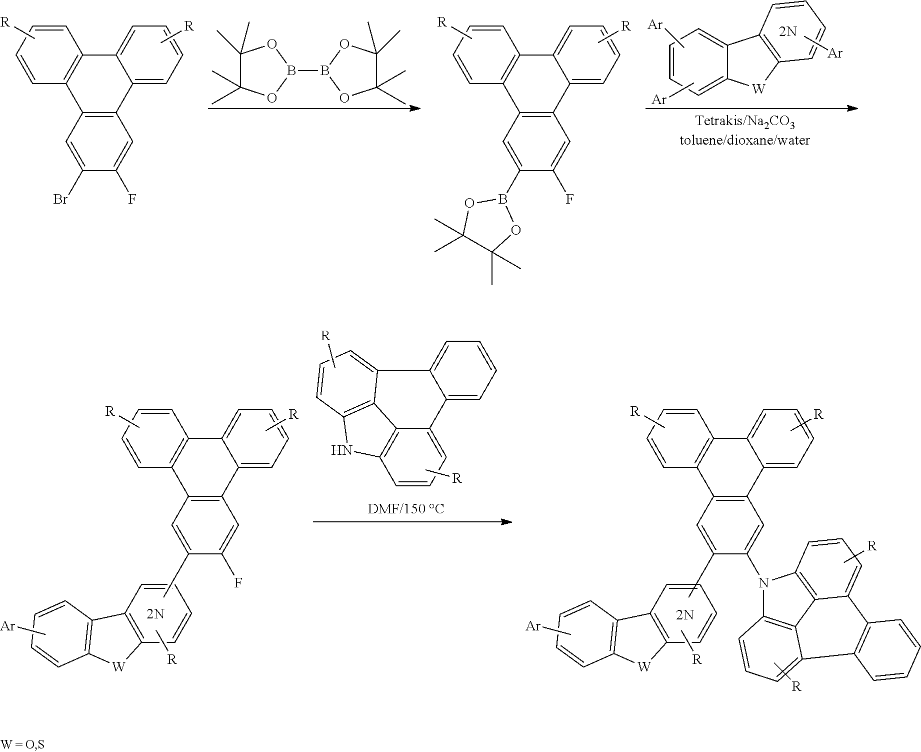

Detailed reaction conditions are known from the prior art or described in the examples section.

It is possible by these processes, if necessary followed by purification, for example recrystallization or sublimation, to obtain the compounds of the formula (1) in high purity, preferably more than 99% (determined by ¹H-NMR and/or HPLC).

For the processing of the compounds of the invention from liquid phase, for example by spin-coating or by printing methods, formulations of the compounds of the invention or of mixtures of compounds of the invention with further functional materials, such as matrix materials, fluorescent emitters, phosphorescent emitters and/or emitters that exhibit TADF, are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

A suitable formulation is a formulation comprising at least one compound of the invention, as described above, or a mixture of the invention, as described hereinafter, and at least one solvent. The solvent may be an abovementioned solvent or a mixture of these solvents.

The inventive compounds of the formulae (1), (1a), (1b), (1c), (1d), (1e), (1f), (1g), (1h), (1i), (1j), (1k), (1l) and (1m) as described above or described as preferred are suitable for use in an organic electroluminescent device, especially as matrix material.

When the compound of the invention is used as matrix material or, synonymously, host material in an emitting layer, it is preferably used in combination with a further compound.

The invention therefore further provides a mixture comprising at least one compound of the formula (1) or at least one preferred compound of one of the formulae (1), (1a), (1b), (1c), (1d), (1e), (1f), (1g), (1h), (1i), (1j), (1k), (1l) and (1m), or a compound from table 1 or one of compounds E1 to E45 and at least one further compound selected from the group of the matrix materials, of the phosphorescent emitters, of the fluorescent emitters and/or of the emitters that exhibit TADF (thermally activated delayed fluorescence). Suitable matrix materials and emitters that can be used in this mixture of the invention are described hereinafter.

The present invention further provides an organic electronic device comprising an anode, a cathode and at least one organic layer, comprising at least one compound of the formula (1), or at least one preferred compound of one of the formulae (1), (1a), (1b), (1c), (1d), (1e), (1f), (1g), (1h), (1i), (1j), (1k), (1l) and (1m), or a compound from table 1 or one of compounds E1 to E45.

The organic electronic device may be selected, for example, from organic integrated circuits (OICs), organic field-effect transistors (OFETs), organic thin-film transistors (OTFTs), organic electroluminescent devices, organic solar cells (OSCs), organic optical detectors, organic photoreceptors.

The organic electronic device is preferably an organic electroluminescent device.

The organic electroluminescent device (synonymous with organic electroluminescence device) of the invention is, for example, an organic light-emitting transistor (OLET), an organic field quench device (OFQD), an organic light-emitting electrochemical cell (OLEC, LEC, LEEC), an organic laser diode (O-laser) or an organic light-emitting diode (OLED). The organic electroluminescent device of the invention is especially an organic light-emitting diode or an organic light-emitting electrochemical cell. The device of the invention is more preferably an OLED.

The organic layer of the device of the invention preferably comprises, as well as a light-emitting layer (EML), a hole injection layer (HIL), a hole transport layer (HTL), a hole blocker layer (HBL), an electron transport layer (ETL), an electron injection layer (EIL), an exciton blocker layer, an electron blocker layer and/or charge generation layers. It is also possible for the device of the invention to include two or more layers from this group, preferably selected from EML, HIL, HTL, ETL, EIL and HBL. It is likewise possible for interlayers having an exciton-blocking function, for example, to be introduced between two emitting layers.

If a plurality of emission layers are present, these preferably have several emission maxima between 380 nm and 750 nm overall, such that the overall result is white emission; in other words, various emitting compounds which may fluoresce or phosphoresce are used in the emitting layers. It is also possible for two or more fluorescent and/or phosphorescent compounds to be present in an emitting layer. Especially preferred are systems having three emitting layers, where the three layers show blue, green and orange or red emission. As an alternative to the combination as described above, an emitting layer may also show yellow emission. Combinations of this kind are known to those skilled in the art. The organic electroluminescent device of the invention may also be a tandem electroluminescent device, especially for white-emitting OLEDs.

The device may also comprise inorganic materials or else layers formed entirely from inorganic materials.

A multitude of materials known in the prior art is suitable for use in the above-described layers of the organic electroluminescent device. The selection involves standard considerations relating to the chemical and physical properties of the materials, since the materials interact with one another in an organic electroluminescent device. This relates, for example, to the energy levels of the orbitals (HOMO, LUMO) or else the triplet and singlet energy levels, but also other material properties.

The inventive compound of the formula (1) as described above or as described as preferred can be used in different layers, according to the exact structure. Preference is given to an organic electroluminescent device comprising a compound of formula (1) or the above-recited preferred embodiments in an emitting layer as matrix material for fluorescent emitters, phosphorescent emitters or for emitters that exhibit TADF (thermally activated delayed fluorescence), especially for phosphorescent emitters. In addition, the compound of the invention can also be used in an electron transport layer and/or in a hole transport layer and/or in an exciton blocker layer and/or in a hole blocker layer. Particular preference is given to using the compound of the invention as matrix material in an emitting layer or as electron transport material or hole blocker material in an electron transport layer or hole blocker layer.

The present invention further provides an organic electronic device as described above, wherein the organic layer comprises at least one light-emitting layer comprising at least one compound of the formula (1), or the at least one preferred compound of one of the formulae (1), (1a), (1b), (1c), (1d), (1e), (1f), (1g), (1h), (1i), (1j), (1k), (1l) and (1m), or a compound from table 1 or one of compounds E1 to E45.

In one embodiment of the invention, for the device of the invention, at least one further matrix material is selected in the light-emitting layer, and this is used together with compounds of the formula (1) as described above or described as preferred or with the compounds from table 1 or the compounds E1 to E45.

The present invention accordingly further provides an organic electronic device as described above, wherein the organic layer comprises at least one light-emitting layer comprising at least one compound of the formula (1), or the at least one preferred compound of one of the formulae (1), (1a), (1b), (1c), (1d), (1e), (1f), (1g), (1h), (1i), (1j), (1k), (1l) and (1m), or a compound from table 1 or one of compounds E1 to E45, and at least one further matrix material.

The present invention accordingly further provides an organic electronic device as described above, wherein the organic layer comprises at least one light-emitting layer comprising at least one compound of the formula (1), or the at least one preferred compound of one of the formulae (1), (1a), (1b), (1c), (1d), (1e), (1f), (1g), (1h), (1i), (1j), (1k), (1l) and (1m), or a compound from table 1 or one of compounds E1 to E45, and two further matrix materials.

Suitable matrix materials that can be used in combination with the compounds of the invention are aromatic ketones, aromatic phosphine oxides or aromatic sulfoxides or sulfones, triarylamines, carbazole derivatives, biscarbazoles, indolocarbazole derivatives, indenocarbazole derivatives, azacarbazole derivatives, bipolar matrix materials, azaboroles or boronic esters, triazine derivatives, zinc complexes, diazasilole or tetraazasilole derivatives, diazaphosphole derivatives, bridged carbazole derivatives, triphenylene derivatives or dibenzofuran derivatives. It is likewise possible for a further phosphorescent emitter having shorter-wavelength emission than the actual emitter to be present as co-host in the mixture, or a compound not involved in charge transport to a significant extent, if at all, for example a wide-bandgap compound.

What is meant herein by a wide-bandgap material is a material within the scope of the disclosure of U.S. Pat. No. 7,294,849 which is characterized by a band gap of at least 3.5 eV, the band gap meaning the gap between the HOMO and LUMO energy of a material.

Particularly suitable hole-transporting matrix materials that are advantageously combined in a mixed matrix system with compounds of the formulae (1), (1a), (1b), (1c), (1d), (1e), (1f), (1g), (1h), (1i), (1j), (1k), (1l) and (1m) as described above or described as preferred may be selected from the compounds of the formulae (HH-1), (HH-2), (HH-3), (HH-4), (HH-5) or (HH-6), as described hereinafter.

The invention accordingly further provides an organic electronic device comprising an anode, a cathode and at least one organic layer comprising at least one light-emitting layer, wherein the at least one light-emitting layer comprises at least one compound of the formula (1) as matrix material 1, as described above or as described with preference, and at least one compound of the formulae (HH-1), (HH-2), (HH-3), (HH-4), (HH-5) or (HH-6) as matrix material 2,

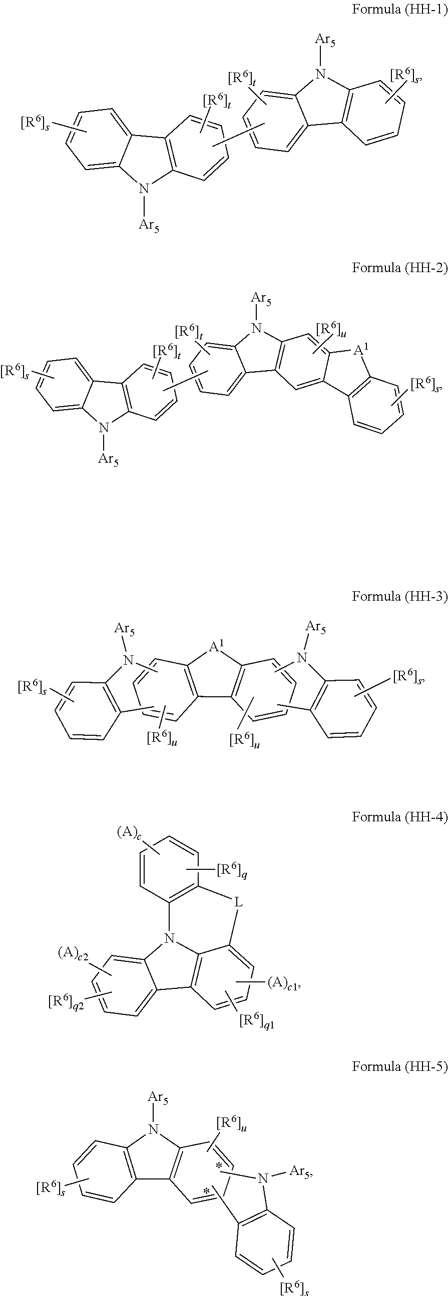

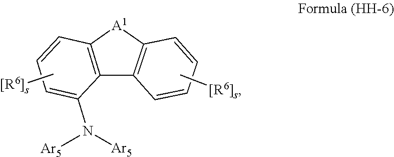

where the symbols and indices used are as follows:
A¹ is C(R⁷)₂, NR⁷, O or S;
L is a bond, O, S, C(R⁷)₂ or NR⁷;
A at each instance is independently a group of the formula (HH-4-1) or (HH-4-2),

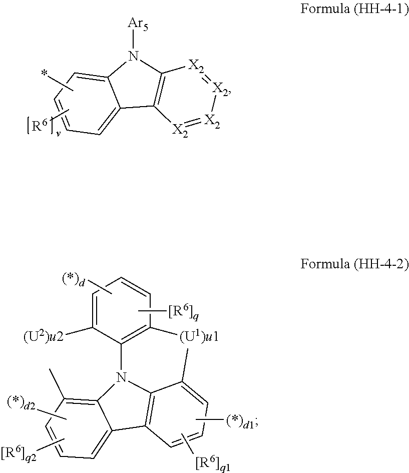

X₂ is the same or different at each instance and is CH, CR⁶ or N, where not more than 2 symbols X₂ can be N;
* indicates the binding site to the formula (HH-4);
U¹, U² where they occur are a bond, O, S, C(R⁷)₂ or NR⁷;
R⁶ at each instance is the same or different and is D, F, CN, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals and where one or more nonadjacent CH₂ groups may be replaced by Si(R⁷)₂, C═O, NR⁷, O, S or CONR⁷, or an aromatic or heteroaromatic ring system which has 5 to 60 ring atoms and may be substituted in each case by one or more R⁷ radicals; it is also possible here for two R⁶ radicals together to form an aromatic, heteroaromatic, aliphatic or heteroaliphatic ring system;
Ar₅ is the same or different at each instance and is independently an aromatic or heteroaromatic ring system which has 5 to 40 ring atoms and may be substituted by one or more R⁷ radicals;
R⁷ is the same or different at each instance and is D, F, C, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals, where one or more nonadjacent CH₂ groups may be replaced by Si(R⁸)₂, C═O, NR⁸, O, S or CONR⁸, or an aromatic or heteroaromatic ring system which has 5 to 40 ring atoms and may be substituted in each case by one or more R⁸ radicals, where R⁸ is not H; at the same time, two or more R⁷ radicals together may form an aromatic, heteroaromatic, aliphatic or heteroaliphatic ring system; preferably, the R⁷ radicals do not form any such ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
c, c1, c2 at each instance are each independently 0 or 1, where the sum total of the indices at each instance c+c1+c2=1;
d, d1, d2 at each instance are each independently 0 or 1, where the sum total of the indices at each instance d+d1+d2=1;
q, q1, q2 at each instance are each independently 0, 1, 2, 3 or 4;
s is the same or different at each instance and is 0, 1, 2, 3 or 4;
t is the same or different at each instance and is 0, 1, 2 or 3;
u is the same or different at each instance and is 0, 1 or 2;
u1, u2 at each instance are each independently 0 or 1, where the sum total u1+u2=1; and
v is 0, 1, 2 or 3.

Preferred compounds of the formula (HH-5) are compounds of the formulae (HH-5-A) to (HH-5-E):

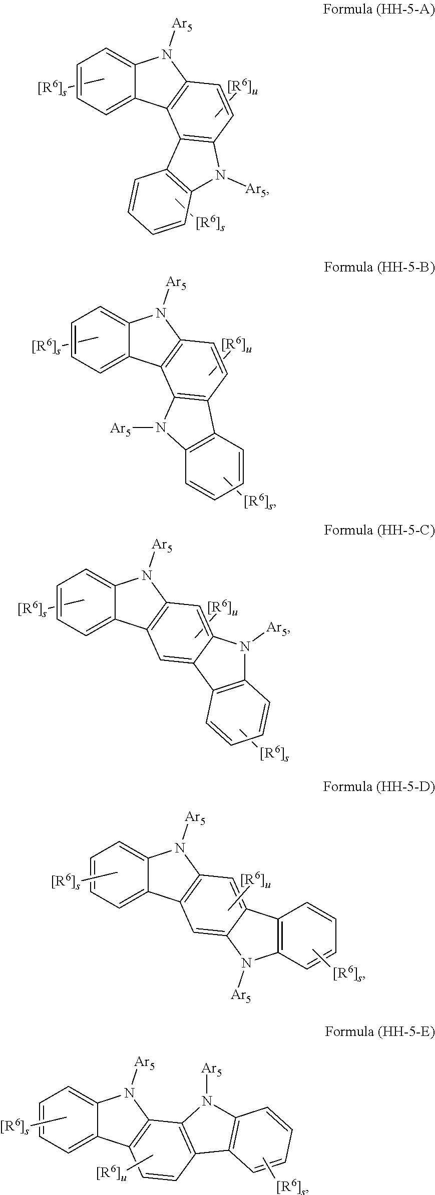

where Ar₅, R⁶, s and u have a definition given above or given with preference.

In compounds of the formulae (HH-1), (HH-2), (HH-3), (HH-5), (HH-5-A), (HH-5-B), (HH-5-C), (HH-5-D), (HH-5-E) or (HH-6), s is preferably 0 or 1 when the R⁶ radical is not D, or more preferably 0.

In compounds of the formulae (HH-1), (HH-2) or (HH-3), t is preferably 0 or 1 when the R⁶ radical is not D, or more preferably 0.

In compounds of the formulae (HH-1), (HH-2), (HH-3), (HH-5), (HH-5-A), (HH-5-B), (HH-5-C), (HH-5-D) or (HH-5-E), u is preferably 0 or 1 when the R⁶ radical is not D, or more preferably 0.

The sum total of the indices s, t and u in compounds of the formulae (HH-1), (HH-2), (HH-3), (HH-5), (HH-5-A), (HH-5-B), (HH-5-C), (HH-5-D), (HH-5-E) or (HH-6) is preferably not more than 6, especially preferably not more than 4 and more preferably not more than 2. This is preferably the case when R⁶ is not D.

In compounds of the formula (HH-4), c, c1, c2 at each instance are each independently 0 or 1, where the sum total of the indices at each instance c+c1+c2 is 1. c2 is preferably defined as 1.

In compounds of the formula (HH-4), L is preferably a single bond or C(R⁷)₂ where R⁷ has a definition given above; more preferably, L is a single bond.

In formula (HH-4-1), v is preferably 0 or 1 when the R⁶ radical is not D.

In formula (HH-4-2), U¹ or U² where they occur are preferably a single bond or C(R⁷)₂ where R⁷ as a definition given above; more preferably, U¹ or U² where they occur are a single bond.

In formula (HH-4-2), q, q1, q2 are preferably 0 or 1 when the R⁶ radical is not D.

In a preferred embodiment of the compounds of the formulae (HH-1), (HH-2), (HH-3), (HH-4), (HH-5), (HH-5-A), (HH-5-B), (HH-5-C), (HH-5-D), (HH-5-E) or (HH-6) that can be combined in accordance with the invention with compounds of the formula (1) or preferred compounds of the formula (1), as described above, R⁶ is the same or different at each instance and is selected from the group consisting of D, F, CN, a straight-chain alkyl group having 1 to 20 carbon atoms or a branched or cyclic alkyl group having 3 to 20 carbon atoms, where the alkyl group may in each case be substituted by one or more R⁷ radicals, or an aromatic or heteroaromatic ring system which has 5 to 60 ring atoms, preferably has 5 to 40 ring atoms, and may be substituted in each case by one or more R⁷ radicals.

In a preferred embodiment of the compounds of the formulae (HH-1), (HH-2), (HH-3), (HH-4), (HH-5), (HH-5-A), (HH-5-B), (HH-5-C), (HH-5-D), (HH-5-E) or (HH-6) that can be combined in accordance with the invention with compounds of the formula (1) or preferred compounds of the formula (1), as described above, R⁶ is the same or different at each instance and is selected from the group consisting of D or an aromatic or heteroaromatic ring system which has 6 to 30 ring atoms and may be substituted by one or more R⁷ radicals.

Preferably, Ar₅ in compounds of the formulae (HH-1), (HH-2), (HH-3), (HH-5), (HH-5-A), (HH-5-B), (HH-5-C), (HH-5-D), (HH-5-E) or (HH-6) is selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorenyl which may be joined via the 1, 2, 3 or 4 position, spirobifluorenyl which may be joined via the 1, 2, 3 or 4 position, naphthyl, especially 1- or 2-bonded naphthyl, or radicals derived from indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals. Ar₅ is preferably unsubstituted.

When A¹ in formula (HH-2) or (HH-3) or (HH-6) is NR⁷, the substituent R⁷ bonded to the nitrogen atom is preferably an aromatic or heteroaromatic ring system which has 5 to 24 ring atoms and may also be substituted by one or more R⁸ radicals. In a particularly preferred embodiment, this substituent R⁷ is the same or different at each instance and is an aromatic or heteroaromatic ring system having 6 to 24 ring atoms, especially having 6 to 18 ring atoms. Preferred embodiments of R⁷ are phenyl, biphenyl, terphenyl and quaterphenyl, which are preferably unsubstituted, and radicals derived from triazine, pyrimidine and quinazoline, which may be substituted by one or more R⁸ radicals, where R⁸ is not H.

When A¹ in formula (HH-2) or (HH-3) or (HH-6) is C(R⁷)₂, the substituents R⁷ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 ring atoms, which may also be substituted by one or more R⁸ radicals, where R⁸ is not H. Most preferably, R⁷ is a methyl group or a phenyl group. In this case, the R⁷ radicals together may also form a ring system, which leads to a spiro system.

In a preferred embodiment of the compounds of the formulae (HH-1), (HH-2), (HH-3), (HH-4), (HH-5), (HH-5-A), (HH-5-B), (HH-5-C), (HH-5-D), (HH-5-E) and (HH-6), these compounds are partly or fully deuterated, more preferably fully deuterated.

The preparation of the compounds of the formulae (HH-1), (HH-2), (HH-3), (HH-4), (HH-5), (HH-5-A), (HH-5-B), (HH-5-C), (HH-5-D), (HH-5-E) and (HH-6) is generally known, and some of the compounds are commercially available.

Compounds of the formula (HH-4) are disclosed, for example, in WO2021/180614, pages 110 to 119, especially as examples on pages 120 to 127. The preparation thereof is disclosed in WO2021/180614 A1 on page 128, and in the synthesis examples on pages 214 to 218.

The preparation of the triarylamines of the formula (HH-6) is known to the person skilled in the art, and some of the compounds are commercially available.

If the at least one further matrix material is a deuterated compound, it is possible that this at least one matrix material is a mixture of deuterated compounds of the same chemical base structure that differ merely by the level of deuteration and/or the deuteration pattern. The remarks relating to deuterated mixtures and to the production of deuterated materials as described above for compounds of the formula (1) are applicable mutatis mutandis here.

In a preferred embodiment of the at least one further matrix material, this is a mixture of deuterated compounds of the formulae (HH-1), (HH-2), (HH-3), (HH-4), (HH-5), (HH-5-A), (HH-5-B), (HH-5-C), (HH-5-D), (HH-5-E) or (HH-6), as described above, where the average deuteration level of these compounds is at least 50 mol % to 90 mol %, preferably 70 mol % to 100 mol %.

Examples of suitable further matrix materials for a combination with compounds of the formula (1), as described above or described as preferred, are the compounds described in WO2019/229011 A1, table 3, pages 137 to 203, which may also be partly or fully deuterated.

Examples of suitable further matrix materials for a combination with compounds of the formula (1) or preferred compounds of the formula (1), as described above or described as preferred, are the compounds described in WO2021/180625 A1, table 3, pages 131 to 137, and in table 4, pages 137 to 139, which may also be partly or fully deuterated.

Examples of suitable further matrix materials for a combination with compounds of the formula (1) or preferred compounds of the formula (1), as described above or described as preferred, are the compounds described in KR20230034896 A, on pages 42 to 47, compounds [2-1] to [2-110], or on pages 49 to 51, compounds [3-1] to [3-26].

Examples of suitable further matrix materials for a combination with compounds of the formula (1) or preferred compounds of the formula (1), as described above or described as preferred, are the compounds described in KR20230154750 A, on pages 39 to 49, compounds [B-1] to [B-243], or on pages 49 to 53, compounds [C-1] to [C-102], or on pages 54 to 57, compounds [D-1] to [D-120].

For a combination with compounds of the formulae (1), (1a), (1b), (1c), (1d), (1e), (1f), (1g), (1h), (1i), (1j), (1k), (1l) and (1m), as described above or described as preferred, especially suitable compounds are those of the formula (HH-1) and/or of the formula (HH-4) and/or of the formula (HH-5), as described above or described as preferred.

For a combination with compounds of the formulae ((1), (1a), (1b), (1c), (1d), (1e), (1f), (1g), (1h), (1i), (1j), (1k), (1l) and (1m), as described above or described as preferred, especially suitable compounds are those of the formula (HH-1) in which at least one Ar₅ group is a heteroaromatic ring system which has 5 to 40 ring atoms and may be substituted by one or more R⁷ radicals and/or compounds of the formula (HH-4) and/or compounds of the formula (HH-5).

In the subgroup of compounds of the formula (HH-5) selected from compounds of the formulae (HH-5-A), (HH-5-B), (HH-5-C), (HH-5-D), (HH-5-E), compounds of the formulae (HH-5A, (HH-5-B) and (HH-5-D) are preferred, with particular preference for compounds of the formula (HH-5-A).

For a combination with a compound of the formulae (1), (1a), (1b), (1c), (1d), (1e), (1f), (1g), (1h), (1i), (1j), (1k), (1l) and (1m), as described above or described as preferred, very particular preferably suitable compounds are those of the formula (HH-4) or (HH-5) or (HH-5-A).

Further examples of suitable host materials of the formulae (HH-1), (HH-2), (HH-3), (HH-4), (HH-5), (HH-5-A), (HH-5-B), (HH-5-C), (HH-5-D), (HH-5-E) and (HH-6) for a combination with compounds of the formula (1) or preferred compounds of the formula (1), as described above or described as preferred, are the structures in table 3 and table 4 that are given below.

TABLE 3

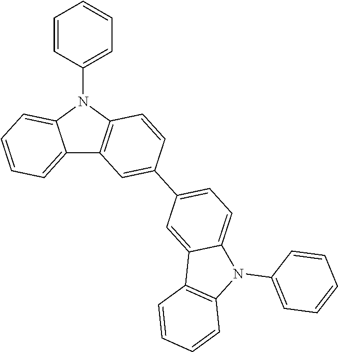 [SMILES: c1ccc(-n2c3ccccc3c3cc(-c4ccc5c(c4)c4ccccc4n5-c4ccccc4)ccc32)cc1]

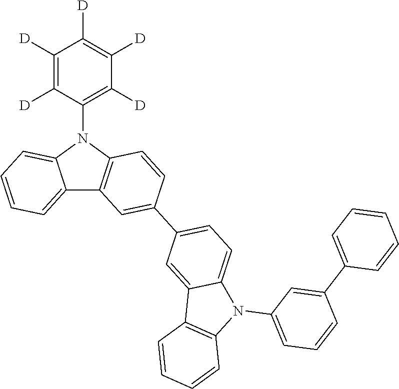 [SMILES: [2H]c1c([2H])c([2H])c(-n2c3ccccc3c3cc(-c4ccc5c(c4)c4ccccc4n5-c4cccc(-c5ccccc5)c4)ccc32)c([2H])c1[2H]]

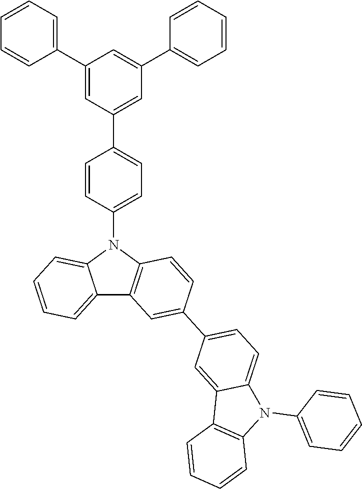 [SMILES: c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1]

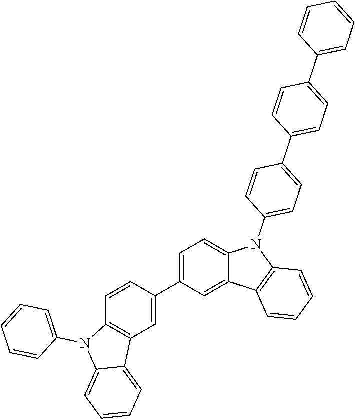 [SMILES: c1ccc(-c2ccc(-c3ccc(-n4c5ccccc5c5cc(-c6ccc7c(c6)c6ccccc6n7-c6ccccc6)ccc54)cc3)cc2)cc1]

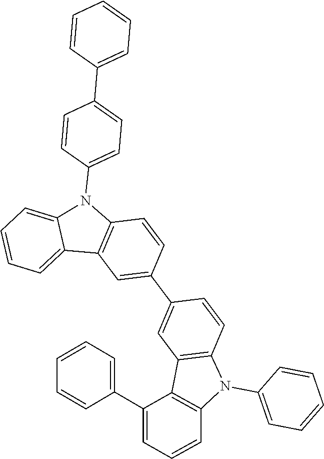 [SMILES: c1ccc(-c2ccc(-n3c4ccccc4c4cc(-c5ccc6c(c5)c5c(-c7ccccc7)cccc5n6-c5ccccc5)ccc43)cc2)cc1]

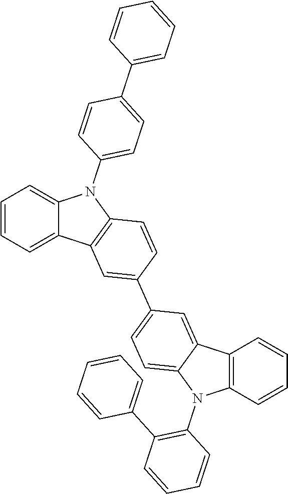 [SMILES: c1ccc(-c2ccc(-n3c4ccccc4c4cc(-c5ccc6c(c5)c5ccccc5n6-c5ccccc5-c5ccccc5)ccc43)cc2)cc1]

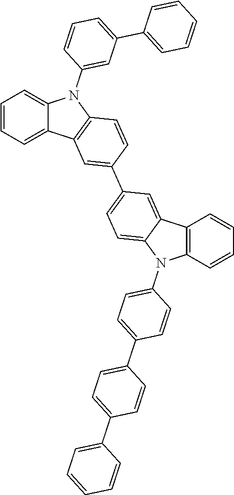 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

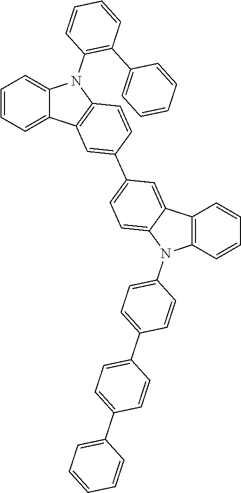 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1]

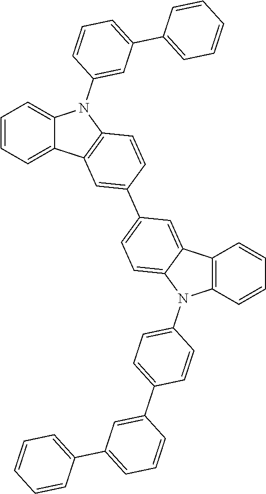 [SMILES: c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1]

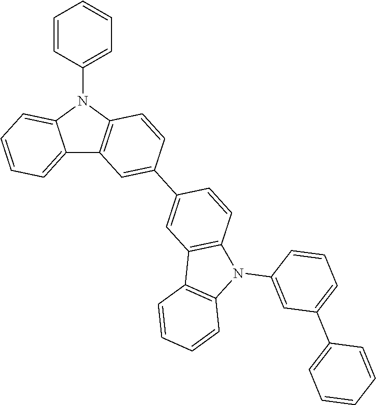 [SMILES: c1ccc(-c2cccc(-n3c4ccccc4c4cc(-c5ccc6c(c5)c5ccccc5n6-c5ccccc5)ccc43)c2)cc1]

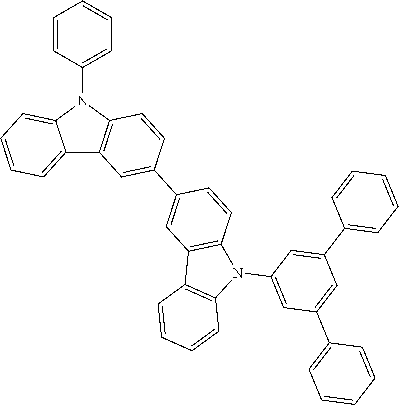 [SMILES: c1ccc(-c2cc(-c3ccccc3)cc(-n3c4ccccc4c4cc(-c5ccc6c(c5)c5ccccc5n6-c5ccccc5)ccc43)c2)cc1]

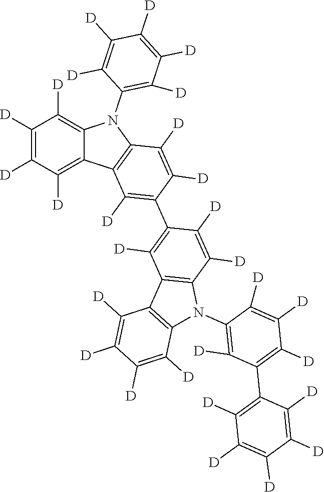 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]]

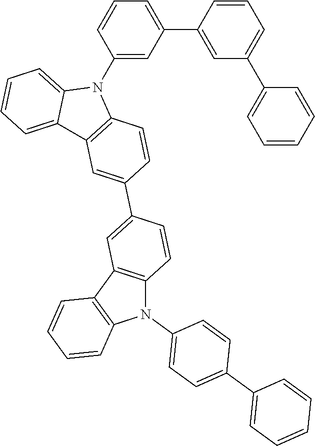 [SMILES: c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1]

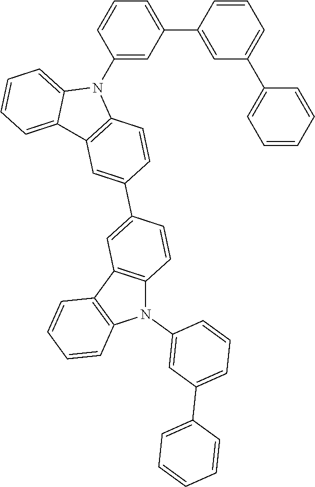 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

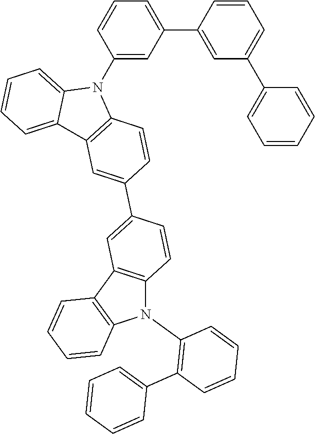 [SMILES: c1ccc(-c2cccc(-c3cccc(-n4c5ccccc5c5cc(-c6ccc7c(c6)c6ccccc6n7-c6ccccc6-c6ccccc6)ccc54)c3)c2)cc1]

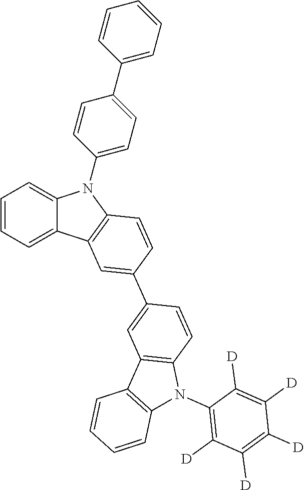 [SMILES: [2H]c1c([2H])c([2H])c(-n2c3ccccc3c3cc(-c4ccc5c(c4)c4ccccc4n5-c4ccc(-c5ccccc5)cc4)ccc32)c([2H])c1[2H]]

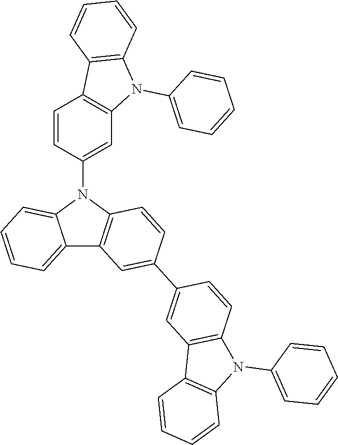 [SMILES: c1ccc(-n2c3ccccc3c3cc(-c4ccc5c(c4)c4ccccc4n5-c4ccc5c6ccccc6n(-c6ccccc6)c5c4)ccc32)cc1]

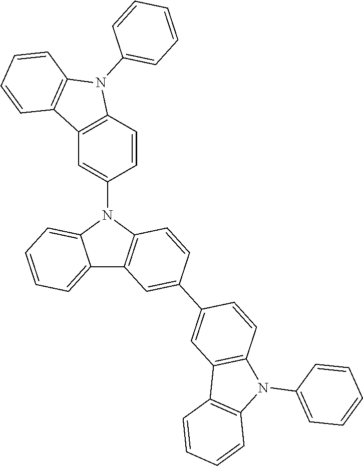 [SMILES: c1ccc(-n2c3ccccc3c3cc(-c4ccc5c(c4)c4ccccc4n5-c4ccc5c(c4)c4ccccc4n5-c4ccccc4)ccc32)cc1]

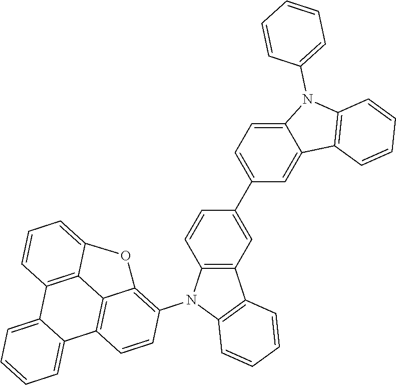 [SMILES: c1ccc(-n2c3ccccc3c3cc(-c4ccc5c(c4)c4ccccc4n5-c4ccc5c6ccccc6c6cccc7oc4c5c76)ccc32)cc1]

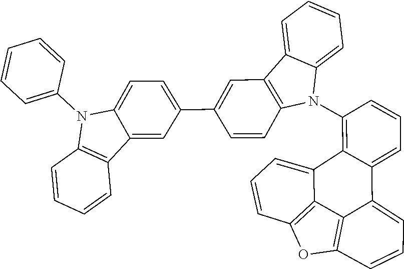 [SMILES: c1ccc(-n2c3ccccc3c3cc(-c4ccc5c(c4)c4ccccc4n5-c4cccc5c6cccc7oc8cccc(c45)c8c76)ccc32)cc1]

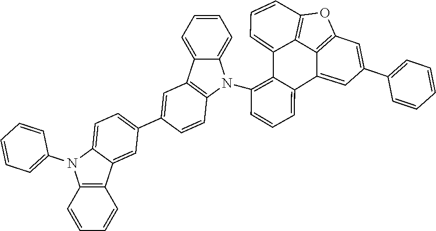 [SMILES: c1ccc(-c2cc3oc4cccc5c6c(-n7c8ccccc8c8cc(-c9ccc%10c(c9)c9ccccc9n%10-c9ccccc9)ccc87)cccc6c(c2)c3c45)cc1]

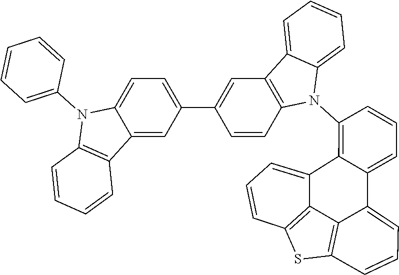 [SMILES: c1ccc(-n2c3ccccc3c3cc(-c4ccc5c(c4)c4ccccc4n5-c4cccc5c6cccc7sc8cccc(c45)c8c76)ccc32)cc1]

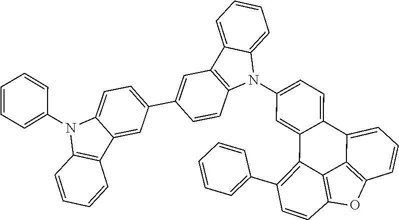 [SMILES: c1ccc(-c2ccc3oc4cccc5c6ccc(-n7c8ccccc8c8cc(-c9ccc%10c(c9)c9ccccc9n%10-c9ccccc9)ccc87)cc6c2c3c45)cc1]

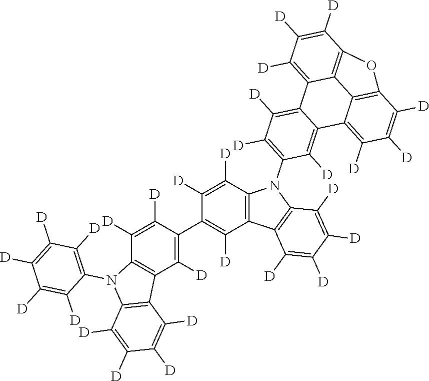 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(c4[2H])c4c([2H])c([2H])c([2H])c6oc7c([2H])c([2H])c([2H])c5c7c64)c([2H])c([2H])c32)c([2H])c1[2H]]

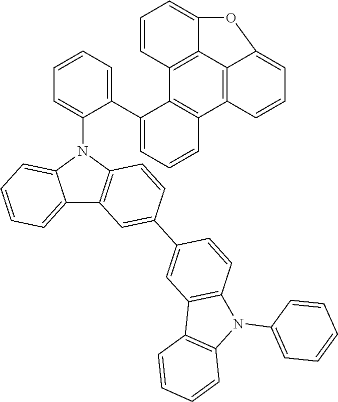 [SMILES: c1ccc(-n2c3ccccc3c3cc(-c4ccc5c(c4)c4ccccc4n5-c4ccccc4-c4cccc5c6cccc7oc8cccc(c45)c8c76)ccc32)cc1]

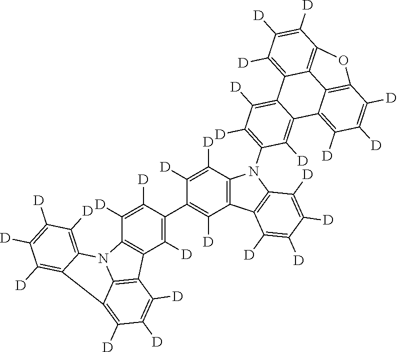 [SMILES: [2H]c1c(-n2c3c([2H])c([2H])c([2H])c([2H])c3c3c([2H])c(-c4c([2H])c([2H])c5c(c4[2H])c4c([2H])c([2H])c([2H])c6c7c([2H])c([2H])c([2H])c([2H])c7n5c64)c([2H])c([2H])c32)c([2H])c2c(c1[2H])c1c([2H])c([2H])c([2H])c3oc4c([2H])c([2H])c([2H])c2c4c31]

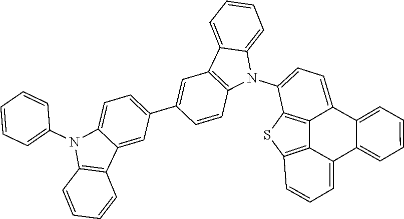 [SMILES: c1ccc(-n2c3ccccc3c3cc(-c4ccc5c(c4)c4ccccc4n5-c4ccc5c6ccccc6c6cccc7sc4c5c76)ccc32)cc1]

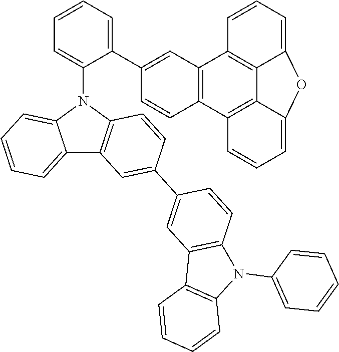 [SMILES: c1ccc(-n2c3ccccc3c3cc(-c4ccc5c(c4)c4ccccc4n5-c4ccccc4-c4ccc5c(c4)c4cccc6oc7cccc5c7c64)ccc32)cc1]

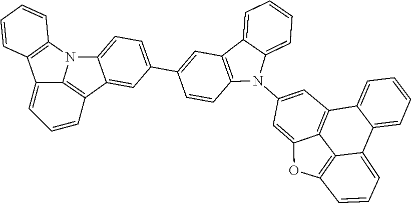 [SMILES: c1ccc2c(c1)c1cccc3oc4cc(-n5c6ccccc6c6cc(-c7ccc8c(c7)c7cccc9c%10ccccc%10n8c97)ccc65)cc2c4c31]

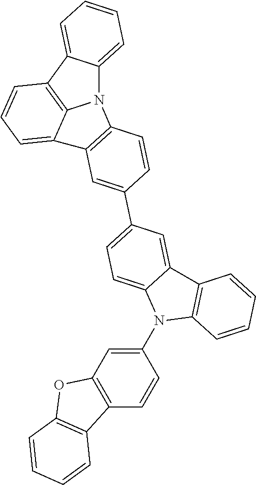 [SMILES: c1ccc2c(c1)oc1cc(-n3c4ccccc4c4cc(-c5ccc6c(c5)c5cccc7c8ccccc8n6c75)ccc43)ccc12]

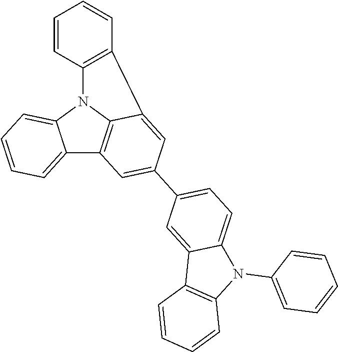 [SMILES: c1ccc(-n2c3ccccc3c3cc(-c4cc5c6ccccc6n6c7ccccc7c(c4)c56)ccc32)cc1]

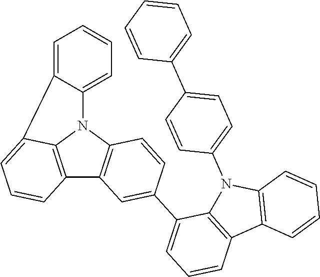 [SMILES: c1ccc(-c2ccc(-n3c4ccccc4c4cccc(-c5ccc6c(c5)c5cccc7c8ccccc8n6c75)c43)cc2)cc1]

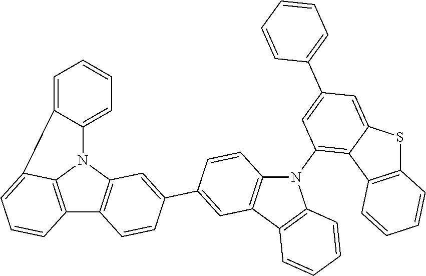 [SMILES: c1ccc(-c2cc(-n3c4ccccc4c4cc(-c5ccc6c7cccc8c9ccccc9n(c6c5)c87)ccc43)c3c(c2)sc2ccccc23)cc1]

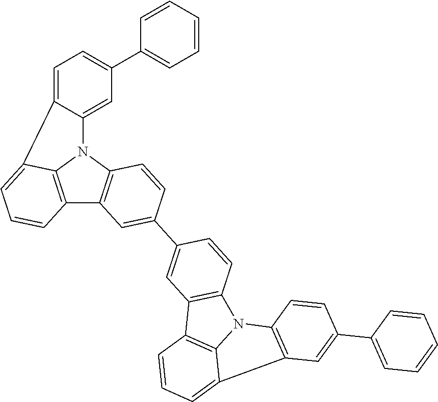 [SMILES: c1ccc(-c2ccc3c(c2)c2cccc4c5cc(-c6ccc7c(c6)c6cccc8c9ccc(-c%10ccccc%10)cc9n7c68)ccc5n3c24)cc1]

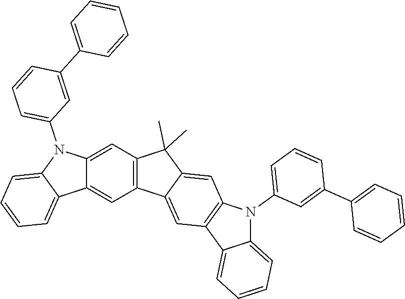 [SMILES: CC1(C)c2cc3c(cc2-c2cc4c5ccccc5n(-c5cccc(-c6ccccc6)c5)c4cc21)c1ccccc1n3-c1cccc(-c2ccccc2)c1]

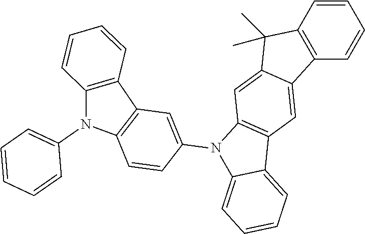 [SMILES: CC1(C)c2ccccc2-c2cc3c4ccccc4n(-c4ccc5c(c4)c4ccccc4n5-c4ccccc4)c3cc21]

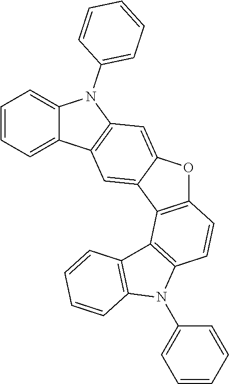 [SMILES: c1ccc(-n2c3ccccc3c3cc4c(cc32)oc2ccc3c(c5ccccc5n3-c3ccccc3)c24)cc1]

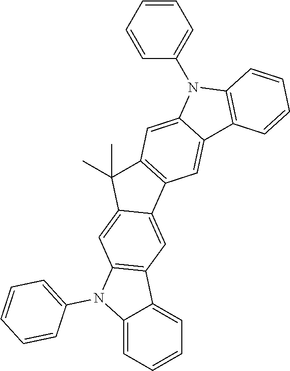 [SMILES: CC1(C)c2cc3c(cc2-c2cc4c5ccccc5n(-c5ccccc5)c4cc21)c1ccccc1n3-c1ccccc1]

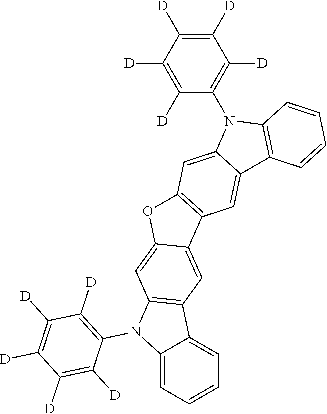 [SMILES: [2H]c1c([2H])c([2H])c(-n2c3ccccc3c3cc4c(cc32)oc2cc3c(cc24)c2ccccc2n3-c2c([2H])c([2H])c([2H])c([2H])c2[2H])c([2H])c1[2H]]

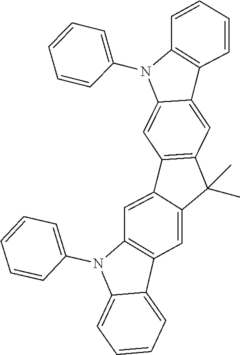 [SMILES: CC1(C)c2cc3c4ccccc4n(-c4ccccc4)c3cc2-c2cc3c(cc21)c1ccccc1n3-c1ccccc1]

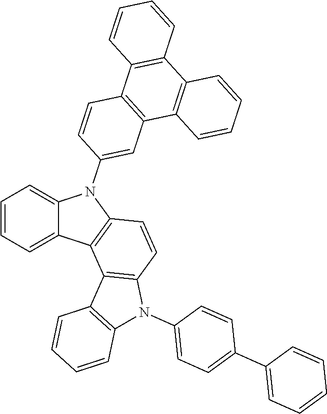 [SMILES: c1ccc(-c2ccc(-n3c4ccccc4c4c5c6ccccc6n(-c6ccc7c8ccccc8c8ccccc8c7c6)c5ccc43)cc2)cc1]

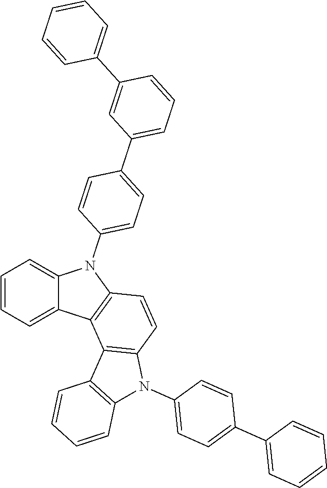 [SMILES: c1ccc(-c2ccc(-n3c4ccccc4c4c5c6ccccc6n(-c6ccc(-c7cccc(-c8ccccc8)c7)cc6)c5ccc43)cc2)cc1]

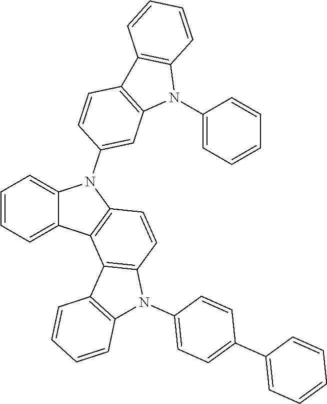 [SMILES: c1ccc(-c2ccc(-n3c4ccccc4c4c5c6ccccc6n(-c6ccc7c8ccccc8n(-c8ccccc8)c7c6)c5ccc43)cc2)cc1]

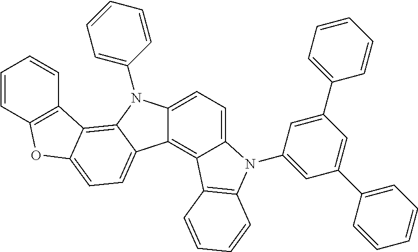 [SMILES: c1ccc(-c2cc(-c3ccccc3)cc(-n3c4ccccc4c4c5c6ccc7oc8ccccc8c7c6n(-c6ccccc6)c5ccc43)c2)cc1]

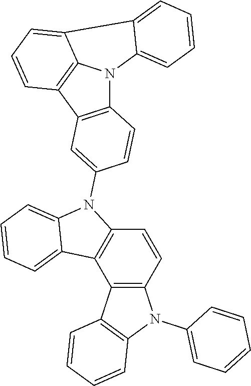 [SMILES: c1ccc(-n2c3ccccc3c3c4c5ccccc5n(-c5ccc6c(c5)c5cccc7c8ccccc8n6c75)c4ccc32)cc1]

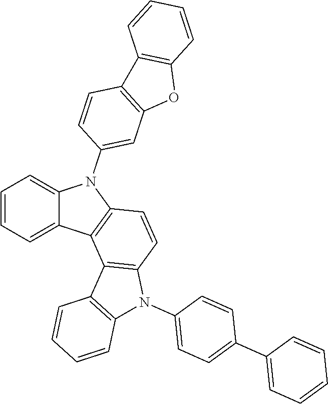 [SMILES: c1ccc(-c2ccc(-n3c4ccccc4c4c5c6ccccc6n(-c6ccc7c(c6)oc6ccccc67)c5ccc43)cc2)cc1]

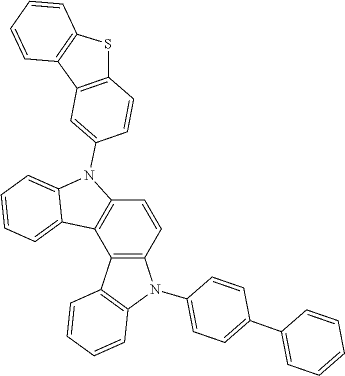 [SMILES: c1ccc(-c2ccc(-n3c4ccccc4c4c5c6ccccc6n(-c6ccc7sc8ccccc8c7c6)c5ccc43)cc2)cc1]

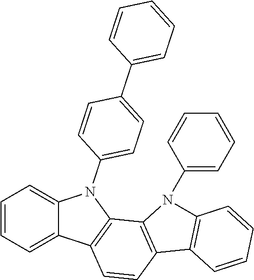 [SMILES: c1ccc(-c2ccc(-n3c4ccccc4c4ccc5c6ccccc6n(-c6ccccc6)c5c43)cc2)cc1]

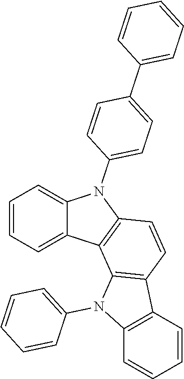 [SMILES: c1ccc(-c2ccc(-n3c4ccccc4c4c3ccc3c5ccccc5n(-c5ccccc5)c34)cc2)cc1]

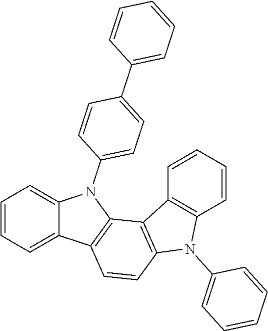 [SMILES: c1ccc(-c2ccc(-n3c4ccccc4c4ccc5c(c6ccccc6n5-c5ccccc5)c43)cc2)cc1]

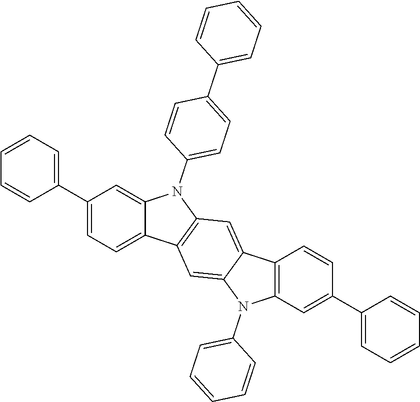 [SMILES: c1ccc(-c2ccc(-n3c4cc(-c5ccccc5)ccc4c4cc5c(cc43)c3ccc(-c4ccccc4)cc3n5-c3ccccc3)cc2)cc1]

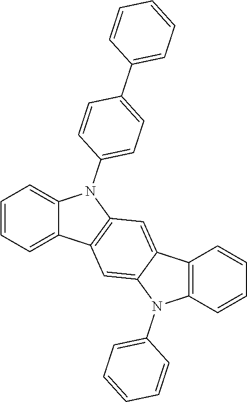 [SMILES: c1ccc(-c2ccc(-n3c4ccccc4c4cc5c(cc43)c3ccccc3n5-c3ccccc3)cc2)cc1]

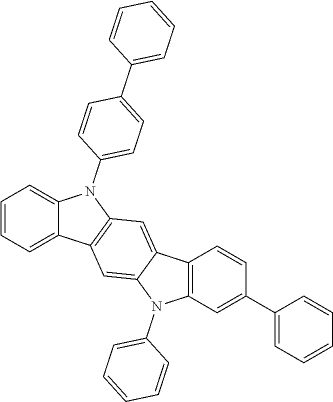 [SMILES: c1ccc(-c2ccc(-n3c4ccccc4c4cc5c(cc43)c3ccc(-c4ccccc4)cc3n5-c3ccccc3)cc2)cc1]

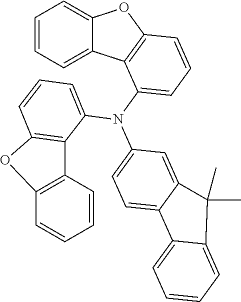 [SMILES: CC1(C)c2ccccc2-c2ccc(N(c3cccc4oc5ccccc5c34)c3cccc4oc5ccccc5c34)cc21]

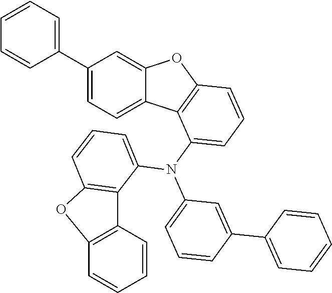 [SMILES: c1ccc(-c2cccc(N(c3cccc4oc5ccccc5c34)c3cccc4oc5cc(-c6ccccc6)ccc5c34)c2)cc1]

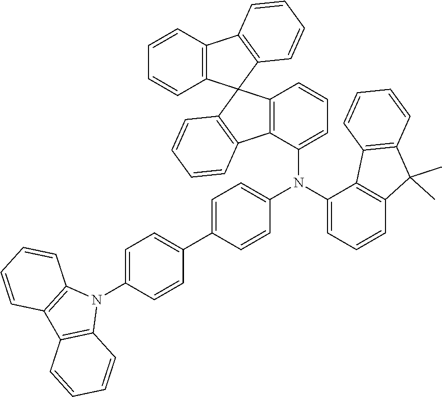 [SMILES: CC1(C)c2ccccc2-c2c(N(c3ccc(-c4ccc(-n5c6ccccc6c6ccccc65)cc4)cc3)c3cccc4c3-c3ccccc3C43c4ccccc4-c4ccccc43)cccc21]

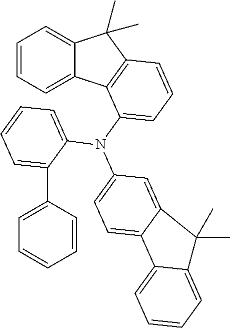 [SMILES: CC1(C)c2ccccc2-c2ccc(N(c3ccccc3-c3ccccc3)c3cccc4c3-c3ccccc3C4(C)C)cc21]

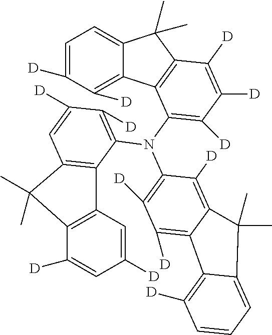

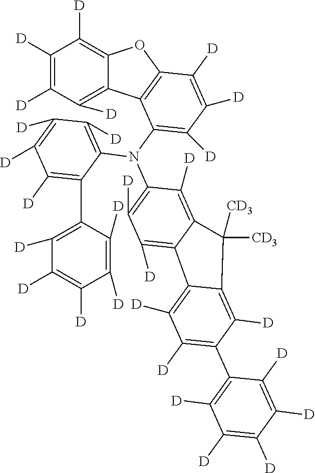 [SMILES: [2H]c1c([2H])c([2H])c(-c2c([2H])c([2H])c3c(c2[2H])C(C([2H])([2H])[2H])(C([2H])([2H])[2H])c2c([2H])c(N(c4c([2H])c([2H])c([2H])c([2H])c4-c4c([2H])c([2H])c([2H])c([2H])c4[2H])c4c([2H])c([2H])c([2H])c5oc6c([2H])c([2H])c([2H])c([2H])c6c45)c([2H])c([2H])c2-3)c([2H])c1[2H]]

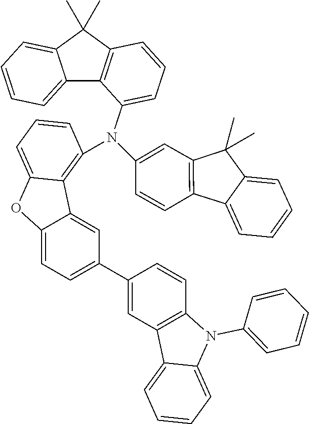 [SMILES: CC1(C)c2ccccc2-c2ccc(N(c3cccc4c3-c3ccccc3C4(C)C)c3cccc4oc5ccc(-c6ccc7c(c6)c6ccccc6n7-c6ccccc6)cc5c34)cc21]

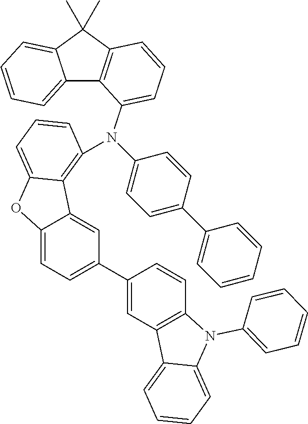 [SMILES: CC1(C)c2ccccc2-c2c(N(c3ccc(-c4ccccc4)cc3)c3cccc4oc5ccc(-c6ccc7c(c6)c6ccccc6n7-c6ccccc6)cc5c34)cccc21]

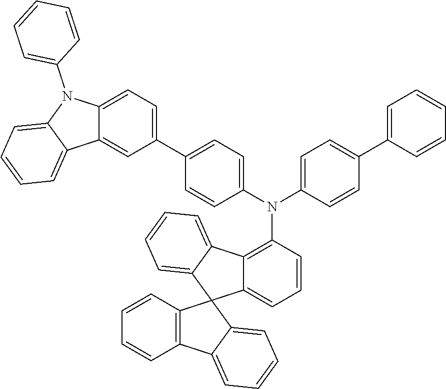 [SMILES: c1ccc(-c2ccc(N(c3ccc(-c4ccc5c(c4)c4ccccc4n5-c4ccccc4)cc3)c3cccc4c3-c3ccccc3C43c4ccccc4-c4ccccc43)cc2)cc1]

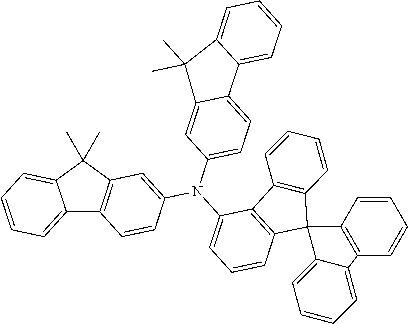 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21]

Particularly suitable compounds of the formulae (HH-1), (HH-2), (HH-3), (HH-4), (HH-5), (HH-5-A), (HH-5-B), (HH-5-C), (HH-5-D), (HH-5-E) or (HH-6) that are selected in accordance with the invention and are preferably used in combination with at least one compound of the formula (1) in the electroluminescent device of the invention are the compounds in table 4.

TABLE 4

| | |
|---|---|
| 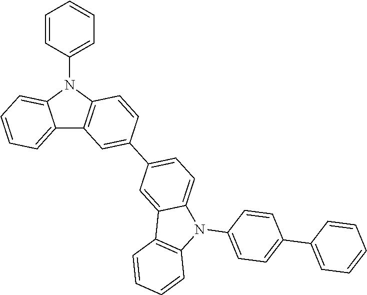 [SMILES: c1ccc(-c2ccc(-n3c4ccccc4c4cc(-c5ccc6c(c5)c5ccccc5n6-c5ccccc5)ccc43)cc2)cc1] | H1 |
| 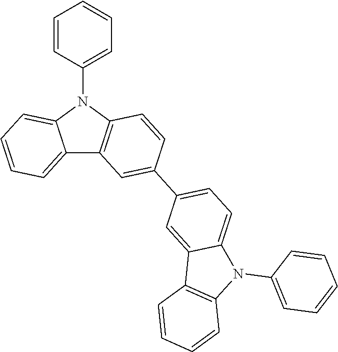 [SMILES: c1ccc(-n2c3ccccc3c3cc(-c4ccc5c(c4)c4ccccc4n5-c4ccccc4)ccc32)cc1] | H2 |
| 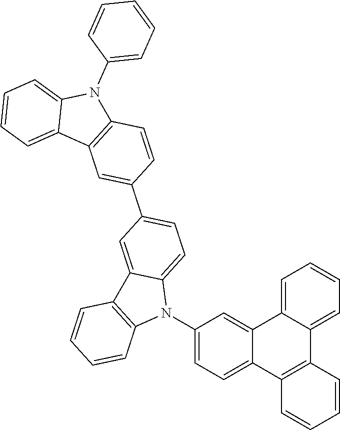 [SMILES: c1ccc(-n2c3ccccc3c3cc(-c4ccc5c(c4)c4ccccc4n5-c4ccc5c6ccccc6c6ccccc6c5c4)ccc32)cc1] | H3 |
| 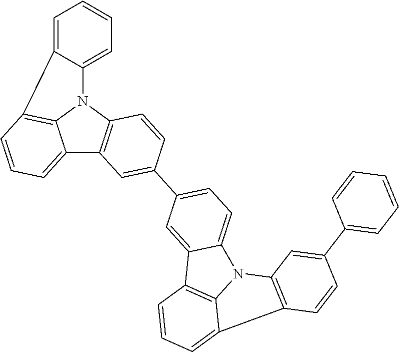 [SMILES: c1ccc(-c2ccc3c4cccc5c6cc(-c7ccc8c(c7)c7cccc9c%10ccccc%10n8c97)ccc6n(c3c2)c54)cc1] | H4 |
| 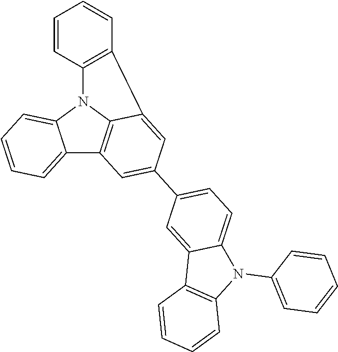 [SMILES: c1ccc(-n2c3ccccc3c3cc(-c4cc5c6ccccc6n6c7ccccc7c(c4)c56)ccc32)cc1] | H5 |
| 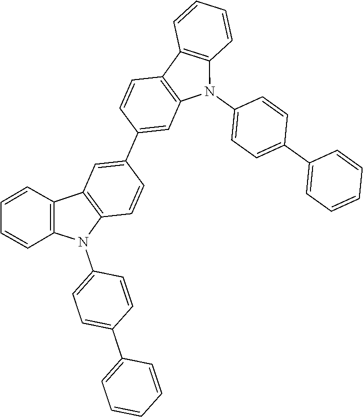 [SMILES: c1ccc(-c2ccc(-n3c4ccccc4c4cc(-c5ccc6c7ccccc7n(-c7ccc(-c8ccccc8)cc7)c6c5)ccc43)cc2)cc1] | H6 |
| 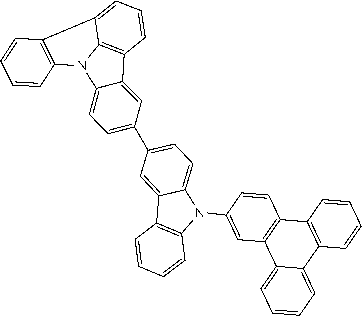 [SMILES: c1ccc2c(c1)c1ccccc1c1cc(-n3c4ccccc4c4cc(-c5ccc6c(c5)c5cccc7c8ccccc8n6c75)ccc43)ccc21] | H7 |
| 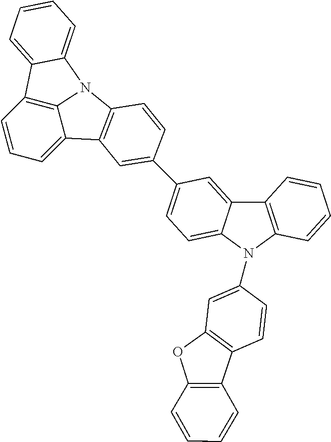 [SMILES: c1ccc2c(c1)oc1cc(-n3c4ccccc4c4cc(-c5ccc6c(c5)c5cccc7c8ccccc8n6c75)ccc43)ccc12] | H8 |
| 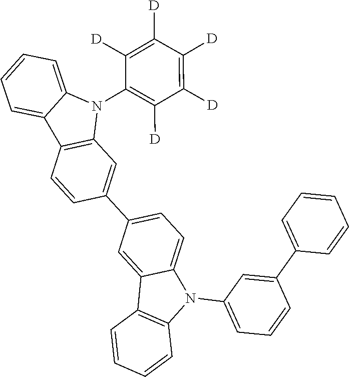 [SMILES: [2H]c1c([2H])c([2H])c(-n2c3ccccc3c3ccc(-c4ccc5c(c4)c4ccccc4n5-c4cccc(-c5ccccc5)c4)cc32)c([2H])c1[2H]] | H9 |
| 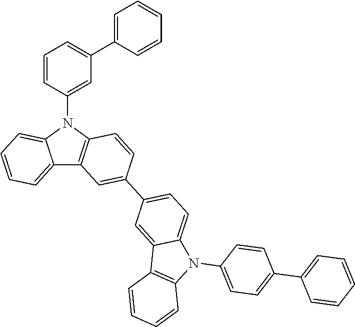 [SMILES: c1ccc(-c2ccc(-n3c4ccccc4c4cc(-c5ccc6c(c5)c5ccccc5n6-c5cccc(-c6ccccc6)c5)ccc43)cc2)cc1] | H10 |
| 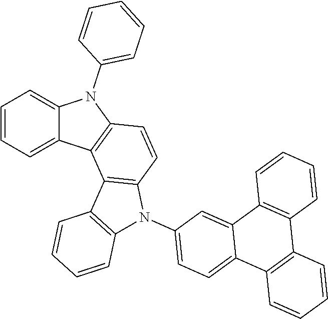 [SMILES: c1ccc(-n2c3ccccc3c3c4c5ccccc5n(-c5ccc6c7ccccc7c7ccccc7c6c5)c4ccc32)cc1] | H11 |
| 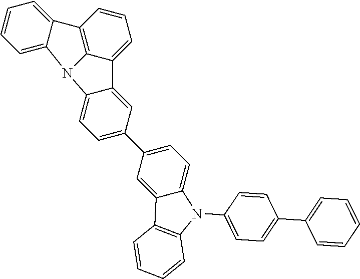 [SMILES: c1ccc(-c2ccc(-n3c4ccccc4c4cc(-c5ccc6c(c5)c5cccc7c8ccccc8n6c75)ccc43)cc2)cc1] | H12 |
| 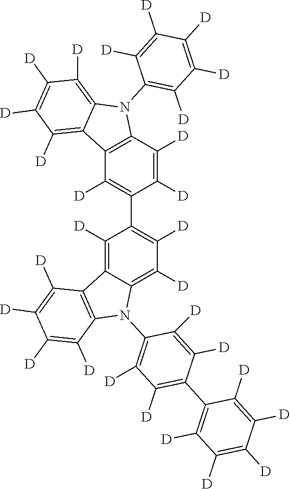 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]] | H13 |
| 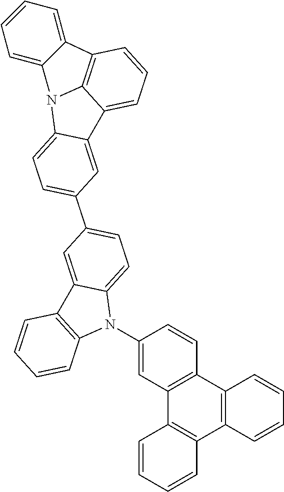 [SMILES: c1ccc2c(c1)c1ccccc1c1cc(-n3c4ccccc4c4cc(-c5ccc6c(c5)c5cccc7c8ccccc8n6c75)ccc43)ccc21] | H14 |
| 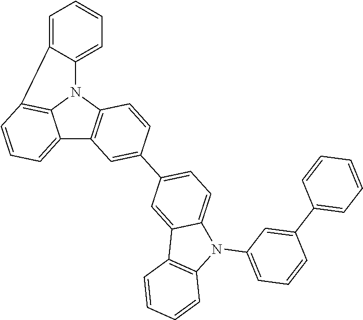 [SMILES: c1ccc(-c2cccc(-n3c4ccccc4c4cc(-c5ccc6c(c5)c5cccc7c8ccccc8n6c75)ccc43)c2)cc1] | H15 |
| 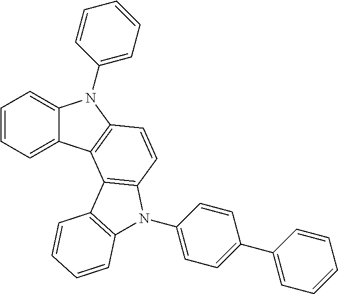 [SMILES: c1ccc(-c2ccc(-n3c4ccccc4c4c5c6ccccc6n(-c6ccccc6)c5ccc43)cc2)cc1] | H16 |
| 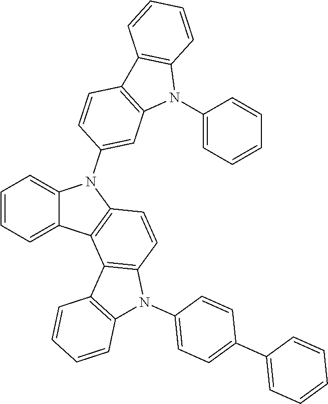 [SMILES: c1ccc(-c2ccc(-n3c4ccccc4c4c5c6ccccc6n(-c6ccc7c8ccccc8n(-c8ccccc8)c7c6)c5ccc43)cc2)cc1] | H17 |
| 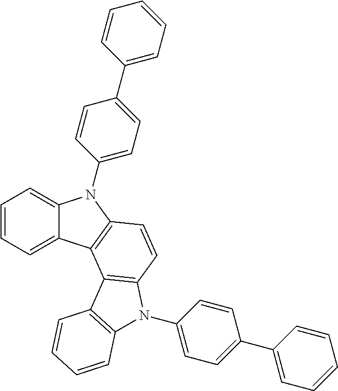 [SMILES: c1ccc(-c2ccc(-n3c4ccccc4c4c5c6ccccc6n(-c6ccc(-c7ccccc7)cc6)c5ccc43)cc2)cc1] | H18 |
| 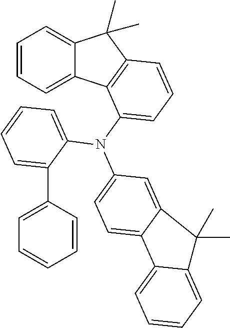 [SMILES: CC1(C)c2ccccc2-c2ccc(N(c3ccccc3-c3ccccc3)c3cccc4c3-c3ccccc3C4(C)C)cc21] | H19 |
| 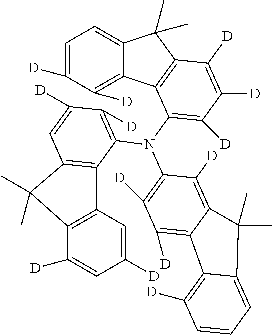 | H20 |
| 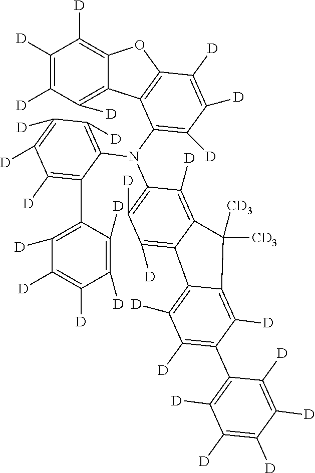 [SMILES: [2H]c1c([2H])c([2H])c(-c2c([2H])c([2H])c3c(c2[2H])C(C([2H])([2H])[2H])(C([2H])([2H])[2H])c2c([2H])c(N(c4c([2H])c([2H])c([2H])c([2H])c4-c4c([2H])c([2H])c([2H])c([2H])c4[2H])c4c([2H])c([2H])c([2H])c5oc6c([2H])c([2H])c([2H])c([2H])c6c45)c([2H])c([2H])c2-3)c([2H])c1[2H]] | H21 |
| 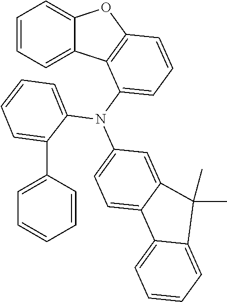 [SMILES: CC1(C)c2ccccc2-c2ccc(N(c3ccccc3-c3ccccc3)c3cccc4oc5ccccc5c34)cc21] | H22 |
| 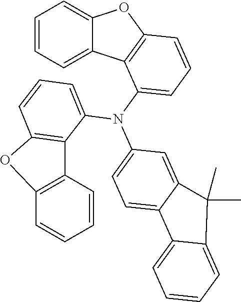 [SMILES: CC1(C)c2ccccc2-c2ccc(N(c3cccc4oc5ccccc5c34)c3cccc4oc5ccccc5c34)cc21] | H23 |
| 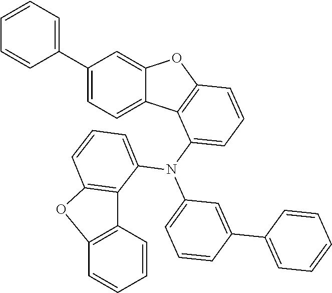 [SMILES: c1ccc(-c2cccc(N(c3cccc4oc5ccccc5c34)c3cccc4oc5cc(-c6ccccc6)ccc5c34)c2)cc1] | H24 |
| 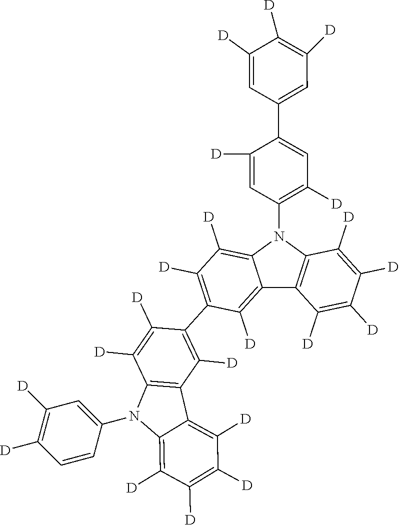 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc([2H])c(-c5cc([2H])c([2H])c([2H])c5)cc4[2H])c([2H])c([2H])c32)cc1[2H]] | H25 |
| 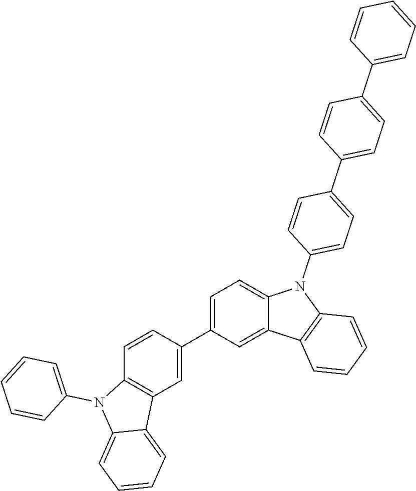 [SMILES: c1ccc(-c2ccc(-c3ccc(-n4c5ccccc5c5cc(-c6ccc7c(c6)c6ccccc6n7-c6ccccc6)ccc54)cc3)cc2)cc1] | H26 |
| 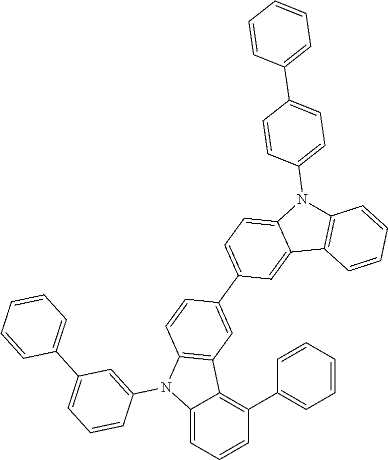 [SMILES: c1ccc(-c2ccc(-n3c4ccccc4c4cc(-c5ccc6c(c5)c5c(-c7ccccc7)cccc5n6-c5cccc(-c6ccccc6)c5)ccc43)cc2)cc1] | H27 |
| 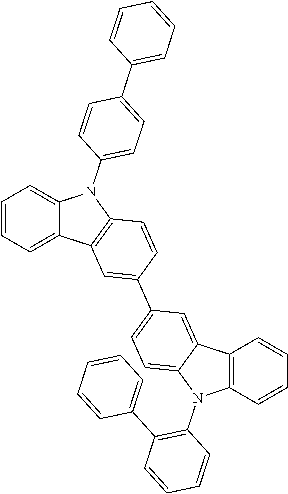 [SMILES: c1ccc(-c2ccc(-n3c4ccccc4c4cc(-c5ccc6c(c5)c5ccccc5n6-c5ccccc5-c5ccccc5)ccc43)cc2)cc1] | H28 |
| 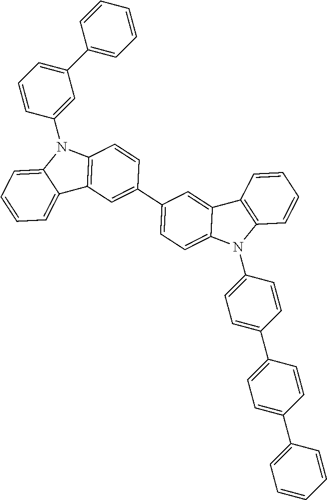 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] | H29 |
| 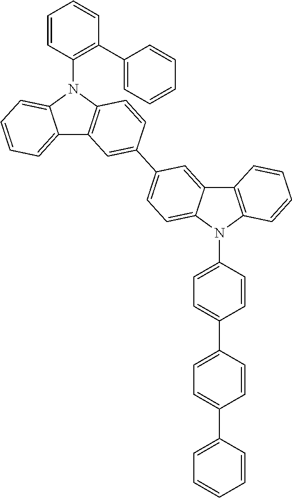 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1] | H30 |
| 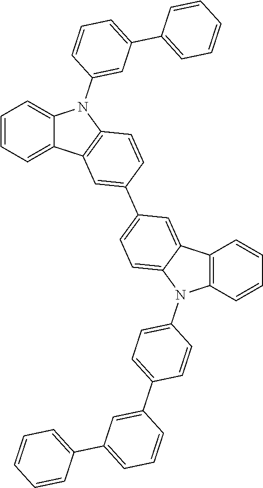 [SMILES: c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1] | H31 |
| 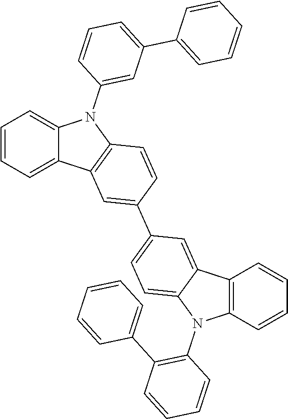 [SMILES: c1ccc(-c2cccc(-n3c4ccccc4c4cc(-c5ccc6c(c5)c5ccccc5n6-c5ccccc5-c5ccccc5)ccc43)c2)cc1] | H32 |
| 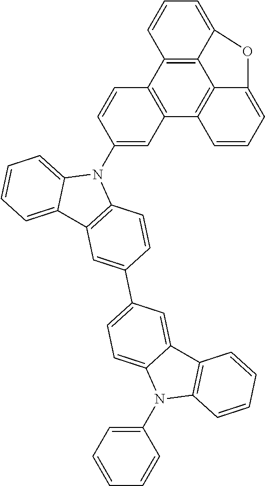 [SMILES: c1ccc(-n2c3ccccc3c3cc(-c4ccc5c(c4)c4ccccc4n5-c4ccc5c(c4)c4cccc6oc7cccc5c7c64)ccc32)cc1] | H33 |

The aforementioned host materials of the formula (1) and the embodiments thereof that are described as preferred or the compounds from table 1 and compounds E1 to E45 can be combined as desired in the device of the invention with the aforementioned matrix materials/host materials, the matrix materials/host materials of the formulae (HH-1), (HH-2), (HH-3), (HH-4), (HH-5), (HH-5-A), (HH-5-B), (HH-5-C), (HH-5-D), (HH-5-E) or (HH-6) and their embodiments in table 3 that are described as preferred, or compounds H1 to H33.

Very particularly preferred mixtures of the compounds of the formula (1) with the host materials of the formulae (HH-1), (HH-2), (HH-3), (HH-4), (HH-5), (HH-5-A), (HH-5-B), (HH-5-C), (HH-5-D), (HH-5-E) or (HH-6) for the device of the invention are obtained by combination of the compounds E1 to E45 with the compounds H1 to H33 as shown hereinafter in table 5. The first mixture M1, for example, is a combination of compound E1 with H1.

TABLE 5

| | | | | | | | | |
|---|---|---|---|---|---|---|---|---|
| M1 | E1 | H1 | M2 | E2 | H1 | M3 | E3 | H1 |
| M4 | E4 | H1 | M5 | E5 | H1 | M6 | E6 | H1 |
| M7 | E7 | H1 | M8 | E8 | H1 | M9 | E9 | H1 |
| M10 | E10 | H1 | M11 | E11 | H1 | M12 | E12 | H1 |
| M13 | E13 | H1 | M14 | E14 | H1 | M15 | E15 | H1 |
| M16 | E16 | H1 | M17 | E17 | H1 | M18 | E18 | H1 |
| M19 | E19 | H1 | M20 | E20 | H1 | M21 | E21 | H1 |
| M22 | E22 | H1 | M23 | E23 | H1 | M24 | E24 | H1 |
| M25 | E25 | H1 | M26 | E26 | H1 | M27 | E27 | H1 |
| M28 | E28 | H1 | M29 | E29 | H1 | M30 | E30 | H1 |
| M31 | E31 | H1 | M32 | E32 | H1 | M33 | E33 | H1 |
| M34 | E34 | H1 | M35 | E35 | H1 | M36 | E36 | H1 |
| M37 | E1 | H2 | M38 | E2 | H2 | M39 | E3 | H2 |
| M40 | E4 | H2 | M41 | E5 | H2 | M42 | E6 | H2 |
| M43 | E7 | H2 | M44 | E8 | H2 | M45 | E9 | H2 |
| M46 | E10 | H2 | M47 | E11 | H2 | M48 | E12 | H2 |
| M49 | E13 | H2 | M50 | E14 | H2 | M51 | E15 | H2 |
| M52 | E16 | H2 | M53 | E17 | H2 | M54 | E18 | H2 |
| M55 | E19 | H2 | M56 | E20 | H2 | M57 | E21 | H2 |
| M58 | E22 | H2 | M59 | E23 | H2 | M60 | E24 | H2 |
| M61 | E25 | H2 | M62 | E26 | H2 | M63 | E27 | H2 |
| M64 | E28 | H2 | M65 | E29 | H2 | M66 | E30 | H2 |
| M67 | E31 | H2 | M68 | E32 | H2 | M69 | E33 | H2 |
| M70 | E34 | H2 | M71 | E35 | H2 | M72 | E36 | H2 |
| M73 | E1 | H3 | M74 | E2 | H3 | M75 | E3 | H3 |
| M76 | E4 | H3 | M77 | E5 | H3 | M78 | E6 | H3 |
| M79 | E7 | H3 | M80 | E8 | H3 | M81 | E9 | H3 |
| M82 | E10 | H3 | M83 | E11 | H3 | M84 | E12 | H3 |
| M85 | E13 | H3 | M86 | E14 | H3 | M87 | E15 | H3 |
| M88 | E16 | H3 | M89 | E17 | H3 | M90 | E18 | H3 |
| M91 | E19 | H3 | M92 | E20 | H3 | M93 | E21 | H3 |
| M94 | E22 | H3 | M95 | E23 | H3 | M96 | E24 | H3 |
| M97 | E25 | H3 | M98 | E26 | H3 | M99 | E27 | H3 |
| M100 | E28 | H3 | M101 | E29 | H3 | M102 | E30 | H3 |
| M103 | E31 | H3 | M104 | E32 | H3 | M105 | E33 | H3 |
| M106 | E34 | H3 | M107 | E35 | H3 | M108 | E36 | H3 |
| M109 | E1 | H4 | M110 | E2 | H4 | M111 | E3 | H4 |
| M112 | E4 | H4 | M113 | E5 | H4 | M114 | E6 | H4 |
| M115 | E7 | H4 | M116 | E8 | H4 | M117 | E9 | H4 |
| M118 | E10 | H4 | M119 | E11 | H4 | M120 | E12 | H4 |
| M121 | E13 | H4 | M122 | E14 | H4 | M123 | E15 | H4 |
| M124 | E16 | H4 | M125 | E17 | H4 | M126 | E18 | H4 |
| M127 | E19 | H4 | M128 | E20 | H4 | M129 | E21 | H4 |
| M130 | E22 | H4 | M131 | E23 | H4 | M132 | E24 | H4 |
| M133 | E25 | H4 | M134 | E26 | H4 | M135 | E27 | H4 |
| M136 | E28 | H4 | M137 | E29 | H4 | M138 | E30 | H4 |
| M139 | E31 | H4 | M140 | E32 | H4 | M141 | E33 | H4 |
| M142 | E34 | H4 | M143 | E35 | H4 | M144 | E36 | H4 |
| M145 | E1 | H5 | M146 | E2 | H5 | M147 | E3 | H5 |
| M148 | E4 | H5 | M149 | E5 | H5 | M150 | E6 | H5 |
| M151 | E7 | H5 | M152 | E8 | H5 | M153 | E9 | H5 |
| M154 | E10 | H5 | M155 | E11 | H5 | M156 | E12 | H5 |
| M157 | E13 | H5 | M158 | E14 | H5 | M159 | E15 | H5 |
| M160 | E16 | H5 | M161 | E17 | H5 | M162 | E18 | H5 |
| M163 | E19 | H5 | M164 | E20 | H5 | M165 | E21 | H5 |
| M166 | E22 | H5 | M167 | E23 | H5 | M168 | E24 | H5 |
| M169 | E25 | H5 | M170 | E26 | H5 | M171 | E27 | H5 |
| M172 | E28 | H5 | M173 | E29 | H5 | M174 | E30 | H5 |
| M175 | E31 | H5 | M176 | E32 | H5 | M177 | E33 | H5 |
| M178 | E34 | H5 | M179 | E35 | H5 | M180 | E36 | H5 |
| M181 | E1 | H6 | M182 | E2 | H6 | M183 | E3 | H6 |
| M184 | E4 | H6 | M185 | E5 | H6 | M186 | E6 | H6 |
| M187 | E7 | H6 | M188 | E8 | H6 | M189 | E9 | H6 |
| M190 | E10 | H6 | M191 | E11 | H6 | M192 | E12 | H6 |
| M193 | E13 | H6 | M194 | E14 | H6 | M195 | E15 | H6 |
| M196 | E16 | H6 | M197 | E17 | H6 | M198 | E18 | H6 |
| M199 | E19 | H6 | M200 | E20 | H6 | M201 | E21 | H6 |
| M202 | E22 | H6 | M203 | E23 | H6 | M204 | E24 | H6 |
| M205 | E25 | H6 | M206 | E26 | H6 | M207 | E27 | H6 |
| M208 | E28 | H6 | M209 | E29 | H6 | M210 | E30 | H6 |
| M211 | E31 | H6 | M212 | E32 | H6 | M213 | E33 | H6 |
| M214 | E34 | H6 | M215 | E35 | H6 | M216 | E36 | H6 |
| M217 | E1 | H7 | M218 | E2 | H7 | M219 | E3 | H7 |
| M220 | E4 | H7 | M221 | E5 | H7 | M222 | E6 | H7 |
| M223 | E7 | H7 | M224 | E8 | H7 | M225 | E9 | H7 |
| M226 | E10 | H7 | M227 | E11 | H7 | M228 | E12 | H7 |
| M229 | E13 | H7 | M230 | E14 | H7 | M231 | E15 | H7 |
| M232 | E16 | H7 | M233 | E17 | H7 | M234 | E18 | H7 |
| M235 | E19 | H7 | M236 | E20 | H7 | M237 | E21 | H7 |
| M238 | E22 | H7 | M239 | E23 | H7 | M240 | E24 | H7 |
| M241 | E25 | H7 | M242 | E26 | H7 | M243 | E27 | H7 |
| M244 | E28 | H7 | M245 | E29 | H7 | M246 | E30 | H7 |
| M247 | E31 | H7 | M248 | E32 | H7 | M249 | E33 | H7 |
| M250 | E34 | H7 | M251 | E35 | H7 | M252 | E36 | H7 |
| M253 | E1 | H8 | M254 | E2 | H8 | M255 | E3 | H8 |
| M256 | E4 | H8 | M257 | E5 | H8 | M258 | E6 | H8 |
| M259 | E7 | H8 | M260 | E8 | H8 | M261 | E9 | H8 |
| M262 | E10 | H8 | M263 | E11 | H8 | M264 | E12 | H8 |
| M265 | E13 | H8 | M266 | E14 | H8 | M267 | E15 | H8 |
| M268 | E16 | H8 | M269 | E17 | H8 | M270 | E18 | H8 |
| M271 | E19 | H8 | M272 | E20 | H8 | M273 | E21 | H8 |
| M274 | E22 | H8 | M275 | E23 | H8 | M276 | E24 | H8 |
| M277 | E25 | H8 | M278 | E26 | H8 | M279 | E27 | H8 |
| M280 | E28 | H8 | M281 | E29 | H8 | M282 | E30 | H8 |
| M283 | E31 | H8 | M284 | E32 | H8 | M285 | E33 | H8 |
| M286 | E34 | H8 | M287 | E35 | H8 | M288 | E36 | H8 |
| M289 | E1 | H9 | M290 | E2 | H9 | M291 | E3 | H9 |
| M292 | E4 | H9 | M293 | E5 | H9 | M294 | E6 | H9 |
| M295 | E7 | H9 | M296 | E8 | H9 | M297 | E9 | H9 |
| M298 | E10 | H9 | M299 | E11 | H9 | M300 | E12 | H9 |
| M301 | E13 | H9 | M302 | E14 | H9 | M303 | E15 | H9 |
| M304 | E16 | H9 | M305 | E17 | H9 | M306 | E18 | H9 |
| M307 | E19 | H9 | M308 | E20 | H9 | M309 | E21 | H9 |
| M310 | E22 | H9 | M311 | E23 | H9 | M312 | E24 | H9 |
| M313 | E25 | H9 | M314 | E26 | H9 | M315 | E27 | H9 |
| M316 | E28 | H9 | M317 | E29 | H9 | M318 | E30 | H9 |
| M319 | E31 | H9 | M320 | E32 | H9 | M321 | E33 | H9 |
| M322 | E34 | H9 | M323 | E35 | H9 | M324 | E36 | H9 |
| M325 | E1 | H10 | M326 | E2 | H10 | M327 | E3 | H10 |
| M328 | E4 | H10 | M329 | E5 | H10 | M330 | E6 | H10 |
| M331 | E7 | H10 | M332 | E8 | H10 | M333 | E9 | H10 |
| M334 | E10 | H10 | M335 | E11 | H10 | M336 | E12 | H10 |
| M337 | E13 | H10 | M338 | E14 | H10 | M339 | E15 | H10 |
| M340 | E16 | H10 | M341 | E17 | H10 | M342 | E18 | H10 |
| M343 | E19 | H10 | M344 | E20 | H10 | M345 | E21 | H10 |
| M346 | E22 | H10 | M347 | E23 | H10 | M348 | E24 | H10 |
| M349 | E25 | H10 | M350 | E26 | H10 | M351 | E27 | H10 |
| M352 | E28 | H10 | M353 | E29 | H10 | M354 | E30 | H10 |
| M355 | E31 | H10 | M356 | E32 | H10 | M357 | E33 | H10 |
| M358 | E34 | H10 | M359 | E35 | H10 | M360 | E36 | H10 |
| M361 | E1 | H11 | M362 | E2 | H11 | M363 | E3 | H11 |
| M364 | E4 | H11 | M365 | E5 | H11 | M366 | E6 | H11 |
| M367 | E7 | H11 | M368 | E8 | H11 | M369 | E9 | H11 |
| M370 | E10 | H11 | M371 | E11 | H11 | M372 | E12 | H11 |
| M373 | E13 | H11 | M374 | E14 | H11 | M375 | E15 | H11 |
| M376 | E16 | H11 | M377 | E17 | H11 | M378 | E18 | H11 |
| M379 | E19 | H11 | M380 | E20 | H11 | M381 | E21 | H11 |
| M382 | E22 | H11 | M383 | E23 | H11 | M384 | E24 | H11 |
| M385 | E25 | H11 | M386 | E26 | H11 | M387 | E27 | H11 |
| M388 | E28 | H11 | M389 | E29 | H11 | M390 | E30 | H11 |
| M391 | E31 | H11 | M392 | E32 | H11 | M393 | E33 | H11 |
| M394 | E34 | H11 | M395 | E35 | H11 | M396 | E36 | H11 |
| M397 | E1 | H12 | M398 | E2 | H12 | M399 | E3 | H12 |
| M400 | E4 | H12 | M401 | E5 | H12 | M402 | E6 | H12 |
| M403 | E7 | H12 | M404 | E8 | H12 | M405 | E9 | H12 |
| M406 | E10 | H12 | M407 | E11 | H12 | M408 | E12 | H12 |
| M409 | E13 | H12 | M410 | E14 | H12 | M411 | E15 | H12 |
| M412 | E16 | H12 | M413 | E17 | H12 | M414 | E18 | H12 |
| M415 | E19 | H12 | M416 | E20 | H12 | M417 | E21 | H12 |
| M418 | E22 | H12 | M419 | E23 | H12 | M420 | E24 | H12 |
| M421 | E25 | H12 | M422 | E26 | H12 | M423 | E27 | H12 |
| M424 | E28 | H12 | M425 | E29 | H12 | M426 | E30 | H12 |
| M427 | E31 | H12 | M428 | E32 | H12 | M429 | E33 | H12 |
| M430 | E34 | H12 | M431 | E35 | H12 | M432 | E36 | H12 |
| M433 | E1 | H13 | M434 | E2 | H13 | M435 | E3 | H13 |
| M436 | E4 | H13 | M437 | E5 | H13 | M438 | E6 | H13 |
| M439 | E7 | H13 | M440 | E8 | H13 | M441 | E9 | H13 |
| M442 | E10 | H13 | M443 | E11 | H13 | M444 | E12 | H13 |
| M445 | E13 | H13 | M446 | E14 | H13 | M447 | E15 | H13 |
| M448 | E16 | H13 | M449 | E17 | H13 | M450 | E18 | H13 |
| M451 | E19 | H13 | M452 | E20 | H13 | M453 | E21 | H13 |
| M454 | E22 | H13 | M455 | E23 | H13 | M456 | E24 | H13 |
| M457 | E25 | H13 | M458 | E26 | H13 | M459 | E27 | H13 |
| M460 | E28 | H13 | M461 | E29 | H13 | M462 | E30 | H13 |
| M463 | E31 | H13 | M464 | E32 | H13 | M465 | E33 | H13 |
| M466 | E34 | H13 | M467 | E35 | H13 | M468 | E36 | H13 |
| M469 | E1 | H14 | M470 | E2 | H14 | M471 | E3 | H14 |
| M472 | E4 | H14 | M473 | E5 | H14 | M474 | E6 | H14 |
| M475 | E7 | H14 | M476 | E8 | H14 | M477 | E9 | H14 |
| M478 | E10 | H14 | M479 | E11 | H14 | M480 | E12 | H14 |
| M481 | E13 | H14 | M482 | E14 | H14 | M483 | E15 | H14 |
| M484 | E16 | H14 | M485 | E17 | H14 | M486 | E18 | H14 |
| M487 | E19 | H14 | M488 | E20 | H14 | M489 | E21 | H14 |
| M490 | E22 | H14 | M491 | E23 | H14 | M492 | E24 | H14 |
| M493 | E25 | H14 | M494 | E26 | H14 | M495 | E27 | H14 |
| M496 | E28 | H14 | M497 | E29 | H14 | M498 | E30 | H14 |
| M499 | E31 | H14 | M500 | E32 | H14 | M501 | E33 | H14 |
| M502 | E34 | H14 | M503 | E35 | H14 | M504 | E36 | H14 |
| M505 | E1 | H15 | M506 | E2 | H15 | M507 | E3 | H15 |
| M508 | E4 | H15 | M509 | E5 | H15 | M510 | E6 | H15 |
| M511 | E7 | H15 | M512 | E8 | H15 | M513 | E9 | H15 |
| M514 | E10 | H15 | M515 | E11 | H15 | M516 | E12 | H15 |
| M517 | E13 | H15 | M518 | E14 | H15 | M519 | E15 | H15 |
| M520 | E16 | H15 | M521 | E17 | H15 | M522 | E18 | H15 |
| M523 | E19 | H15 | M524 | E20 | H15 | M525 | E21 | H15 |
| M526 | E22 | H15 | M527 | E23 | H15 | M528 | E24 | H15 |
| M529 | E25 | H15 | M530 | E26 | H15 | M531 | E27 | H15 |
| M532 | E28 | H15 | M533 | E29 | H15 | M534 | E30 | H15 |
| M535 | E31 | H15 | M536 | E32 | H15 | M537 | E33 | H15 |
| M538 | E34 | H15 | M539 | E35 | H15 | M540 | E36 | H15 |
| M541 | E1 | H16 | M542 | E2 | H16 | M543 | E3 | H16 |
| M544 | E4 | H16 | M545 | E5 | H16 | M546 | E6 | H16 |
| M547 | E7 | H16 | M548 | E8 | H16 | M549 | E9 | H16 |
| M550 | E10 | H16 | M551 | E11 | H16 | M552 | E12 | H16 |
| M553 | E13 | H16 | M554 | E14 | H16 | M555 | E15 | H16 |
| M556 | E16 | H16 | M557 | E17 | H16 | M558 | E18 | H16 |
| M559 | E19 | H16 | M560 | E20 | H16 | M561 | E21 | H16 |
| M562 | E22 | H16 | M563 | E23 | H16 | M564 | E24 | H16 |
| M565 | E25 | H16 | M566 | E26 | H16 | M567 | E27 | H16 |
| M568 | E28 | H16 | M569 | E29 | H16 | M570 | E30 | H16 |
| M571 | E31 | H16 | M572 | E32 | H16 | M573 | E33 | H16 |
| M574 | E34 | H16 | M575 | E35 | H16 | M576 | E36 | H16 |
| M577 | E1 | H17 | M578 | E2 | H17 | M579 | E3 | H17 |
| M580 | E4 | H17 | M581 | E5 | H17 | M582 | E6 | H17 |
| M583 | E7 | H17 | M584 | E8 | H17 | M585 | E9 | H17 |
| M586 | E10 | H17 | M587 | E11 | H17 | M588 | E12 | H17 |
| M589 | E13 | H17 | M590 | E14 | H17 | M591 | E15 | H17 |
| M592 | E16 | H17 | M593 | E17 | H17 | M594 | E18 | H17 |
| M595 | E19 | H17 | M596 | E20 | H17 | M597 | E21 | H17 |
| M598 | E22 | H17 | M599 | E23 | H17 | M600 | E24 | H17 |
| M601 | E25 | H17 | M602 | E26 | H17 | M603 | E27 | H17 |
| M604 | E28 | H17 | M605 | E29 | H17 | M606 | E30 | H17 |
| M607 | E31 | H17 | M608 | E32 | H17 | M609 | E33 | H17 |
| M610 | E34 | H17 | M611 | E35 | H17 | M612 | E36 | H17 |
| M613 | E1 | H18 | M614 | E2 | H18 | M615 | E3 | H18 |
| M616 | E4 | H18 | M617 | E5 | H18 | M618 | E6 | H18 |
| M619 | E7 | H18 | M620 | E8 | H18 | M621 | E9 | H18 |
| M622 | E10 | H18 | M623 | E11 | H18 | M624 | E12 | H18 |
| M625 | E13 | H18 | M626 | E14 | H18 | M627 | E15 | H18 |
| M628 | E16 | H18 | M629 | E17 | H18 | M630 | E18 | H18 |
| M631 | E19 | H18 | M632 | E20 | H18 | M633 | E21 | H18 |
| M634 | E22 | H18 | M635 | E23 | H18 | M636 | E24 | H18 |
| M637 | E25 | H18 | M638 | E26 | H18 | M639 | E27 | H18 |
| M640 | E28 | H18 | M641 | E29 | H18 | M642 | E30 | H18 |
| M643 | E31 | H18 | M644 | E32 | H18 | M645 | E33 | H18 |
| M646 | E34 | H18 | M647 | E35 | H18 | M648 | E36 | H18 |
| M649 | E1 | H19 | M650 | E2 | H19 | M651 | E3 | H19 |
| M652 | E4 | H19 | M653 | E5 | H19 | M654 | E6 | H19 |
| M655 | E7 | H19 | M656 | E8 | H19 | M657 | E9 | H19 |
| M658 | E10 | H19 | M659 | E11 | H19 | M660 | E12 | H19 |
| M661 | E13 | H19 | M662 | E14 | H19 | M663 | E15 | H19 |
| M664 | E16 | H19 | M665 | E17 | H19 | M666 | E18 | H19 |
| M667 | E19 | H19 | M668 | E20 | H19 | M669 | E21 | H19 |
| M670 | E22 | H19 | M671 | E23 | H19 | M672 | E24 | H19 |
| M673 | E25 | H19 | M674 | E26 | H19 | M675 | E27 | H19 |
| M676 | E28 | H19 | M677 | E29 | H19 | M678 | E30 | H19 |
| M679 | E31 | H19 | M680 | E32 | H19 | M681 | E33 | H19 |
| M682 | E34 | H19 | M683 | E35 | H19 | M684 | E36 | H19 |
| M685 | E1 | H20 | M686 | E2 | H20 | M687 | E3 | H20 |
| M688 | E4 | H20 | M689 | E5 | H20 | M690 | E6 | H20 |
| M691 | E7 | H20 | M692 | E8 | H20 | M693 | E9 | H20 |
| M694 | E10 | H20 | M695 | E11 | H20 | M696 | E12 | H20 |
| M697 | E13 | H20 | M698 | E14 | H20 | M699 | E15 | H20 |
| M700 | E16 | H20 | M701 | E17 | H20 | M702 | E18 | H20 |
| M703 | E19 | H20 | M704 | E20 | H20 | M705 | E21 | H20 |
| M706 | E22 | H20 | M707 | E23 | H20 | M708 | E24 | H20 |
| M709 | E25 | H20 | M710 | E26 | H20 | M711 | E27 | H20 |
| M712 | E28 | H20 | M713 | E29 | H20 | M714 | E30 | H20 |
| M715 | E31 | H20 | M716 | E32 | H20 | M717 | E33 | H20 |
| M718 | E34 | H20 | M719 | E35 | H20 | M720 | E36 | H20 |
| M721 | E1 | H21 | M722 | E2 | H21 | M723 | E3 | H21 |
| M724 | E4 | H21 | M725 | E5 | H21 | M726 | E6 | H21 |
| M727 | E7 | H21 | M728 | E8 | H21 | M729 | E9 | H21 |
| M730 | E10 | H21 | M731 | E11 | H21 | M732 | E12 | H21 |
| M733 | E13 | H21 | M734 | E14 | H21 | M735 | E15 | H21 |
| M736 | E16 | H21 | M737 | E17 | H21 | M738 | E18 | H21 |
| M739 | E19 | H21 | M740 | E20 | H21 | M741 | E21 | H21 |
| M742 | E22 | H21 | M743 | E23 | H21 | M744 | E24 | H21 |
| M745 | E25 | H21 | M746 | E26 | H21 | M747 | E27 | H21 |
| M748 | E28 | H21 | M749 | E29 | H21 | M750 | E30 | H21 |
| M751 | E31 | H21 | M752 | E32 | H21 | M753 | E33 | H21 |
| M754 | E34 | H21 | M755 | E35 | H21 | M756 | E36 | H21 |
| M757 | E1 | H22 | M758 | E2 | H22 | M759 | E3 | H22 |
| M760 | E4 | H22 | M761 | E5 | H22 | M762 | E6 | H22 |
| M763 | E7 | H22 | M764 | E8 | H22 | M765 | E9 | H22 |
| M766 | E10 | H22 | M767 | E11 | H22 | M768 | E12 | H22 |
| M769 | E13 | H22 | M770 | E14 | H22 | M771 | E15 | H22 |
| M772 | E16 | H22 | M773 | E17 | H22 | M774 | E18 | H22 |
| M775 | E19 | H22 | M776 | E20 | H22 | M777 | E21 | H22 |
| M778 | E22 | H22 | M779 | E23 | H22 | M780 | E24 | H22 |
| M781 | E25 | H22 | M782 | E26 | H22 | M783 | E27 | H22 |
| M784 | E28 | H22 | M785 | E29 | H22 | M786 | E30 | H22 |
| M787 | E31 | H22 | M788 | E32 | H22 | M789 | E33 | H22 |
| M790 | E34 | H22 | M791 | E35 | H22 | M792 | E36 | H22 |
| M793 | E1 | H23 | M794 | E2 | H23 | M795 | E3 | H23 |
| M796 | E4 | H23 | M797 | E5 | H23 | M798 | E6 | H23 |
| M799 | E7 | H23 | M800 | E8 | H23 | M801 | E9 | H23 |
| M802 | E10 | H23 | M803 | E11 | H23 | M804 | E12 | H23 |
| M805 | E13 | H23 | M806 | E14 | H23 | M807 | E15 | H23 |
| M808 | E16 | H23 | M809 | E17 | H23 | M810 | E18 | H23 |
| M811 | E19 | H23 | M812 | E20 | H23 | M813 | E21 | H23 |
| M814 | E22 | H23 | M815 | E23 | H23 | M816 | E24 | H23 |
| M817 | E25 | H23 | M818 | E26 | H23 | M819 | E27 | H23 |
| M820 | E28 | H23 | M821 | E29 | H23 | M822 | E30 | H23 |
| M823 | E31 | H23 | M824 | E32 | H23 | M825 | E33 | H23 |
| M826 | E34 | H23 | M827 | E35 | H23 | M828 | E36 | H23 |
| M829 | E1 | H24 | M830 | E2 | H24 | M831 | E3 | H24 |
| M832 | E4 | H24 | M833 | E5 | H24 | M834 | E6 | H24 |
| M835 | E7 | H24 | M836 | E8 | H24 | M837 | E9 | H24 |
| M838 | E10 | H24 | M839 | E11 | H24 | M840 | E12 | H24 |
| M841 | E13 | H24 | M842 | E14 | H24 | M843 | E15 | H24 |
| M844 | E16 | H24 | M845 | E17 | H24 | M846 | E18 | H24 |
| M847 | E19 | H24 | M848 | E20 | H24 | M849 | E21 | H24 |
| M850 | E22 | H24 | M851 | E23 | H24 | M852 | E24 | H24 |
| M853 | E25 | H24 | M854 | E26 | H24 | M855 | E27 | H24 |
| M856 | E28 | H24 | M857 | E29 | H24 | M858 | E30 | H24 |
| M859 | E31 | H24 | M860 | E32 | H24 | M861 | E33 | H24 |
| M862 | E34 | H24 | M863 | E35 | H24 | M864 | E36 | H24 |
| M865 | E1 | H25 | M866 | E2 | H25 | M867 | E3 | H25 |
| M868 | E4 | H25 | M869 | E5 | H25 | M870 | E6 | H25 |
| M871 | E7 | H25 | M872 | E8 | H25 | M873 | E9 | H25 |
| M874 | E10 | H25 | M875 | E11 | H25 | M876 | E12 | H25 |
| M877 | E13 | H25 | M878 | E14 | H25 | M879 | E15 | H25 |
| M880 | E16 | H25 | M881 | E17 | H25 | M882 | E18 | H25 |
| M883 | E19 | H25 | M884 | E20 | H25 | M885 | E21 | H25 |
| M886 | E22 | H25 | M887 | E23 | H25 | M888 | E24 | H25 |
| M889 | E25 | H25 | M890 | E26 | H25 | M891 | E27 | H25 |
| M892 | E28 | H25 | M893 | E29 | H25 | M894 | E30 | H25 |
| M895 | E31 | H25 | M896 | E32 | H25 | M897 | E33 | H25 |
| M898 | E34 | H25 | M899 | E35 | H25 | M900 | E36 | H25 |
| M901 | E1 | H26 | M902 | E2 | H26 | M903 | E3 | H26 |
| M904 | E4 | H26 | M905 | E5 | H26 | M906 | E6 | H26 |
| M907 | E7 | H26 | M908 | E8 | H26 | M909 | E9 | H26 |
| M910 | E10 | H26 | M911 | E11 | H26 | M912 | E12 | H26 |
| M913 | E13 | H26 | M914 | E14 | H26 | M915 | E15 | H26 |
| M916 | E16 | H26 | M917 | E17 | H26 | M918 | E18 | H26 |
| M919 | E19 | H26 | M920 | E20 | H26 | M921 | E21 | H26 |
| M922 | E22 | H26 | M923 | E23 | H26 | M924 | E24 | H26 |
| M925 | E25 | H26 | M926 | E26 | H26 | M927 | E27 | H26 |
| M928 | E28 | H26 | M929 | E29 | H26 | M930 | E30 | H26 |
| M931 | E31 | H26 | M932 | E32 | H26 | M933 | E33 | H26 |
| M934 | E34 | H26 | M935 | E35 | H26 | M936 | E36 | H26 |
| M937 | E1 | H27 | M938 | E2 | H27 | M939 | E3 | H27 |
| M940 | E4 | H27 | M941 | E5 | H27 | M942 | E6 | H27 |
| M943 | E7 | H27 | M944 | E8 | H27 | M945 | E9 | H27 |
| M946 | E10 | H27 | M947 | E11 | H27 | M948 | E12 | H27 |
| M949 | E13 | H27 | M950 | E14 | H27 | M951 | E15 | H27 |
| M952 | E16 | H27 | M953 | E17 | H27 | M954 | E18 | H27 |
| M955 | E19 | H27 | M956 | E20 | H27 | M957 | E21 | H27 |
| M958 | E22 | H27 | M959 | E23 | H27 | M960 | E24 | H27 |
| M961 | E25 | H27 | M962 | E26 | H27 | M963 | E27 | H27 |
| M964 | E28 | H27 | M965 | E29 | H27 | M966 | E30 | H27 |
| M967 | E31 | H27 | M968 | E32 | H27 | M969 | E33 | H27 |
| M970 | E34 | H27 | M971 | E35 | H27 | M972 | E36 | H27 |
| M973 | E1 | H28 | M974 | E2 | H28 | M975 | E3 | H28 |
| M976 | E4 | H28 | M977 | E5 | H28 | M978 | E6 | H28 |
| M979 | E7 | H28 | M980 | E8 | H28 | M981 | E9 | H28 |
| M982 | E10 | H28 | M983 | E11 | H28 | M984 | E12 | H28 |
| M985 | E13 | H28 | M986 | E14 | H28 | M987 | E15 | H28 |
| M988 | E16 | H28 | M989 | E17 | H28 | M990 | E18 | H28 |
| M991 | E19 | H28 | M992 | E20 | H28 | M993 | E21 | H28 |
| M994 | E22 | H28 | M995 | E23 | H28 | M996 | E24 | H28 |
| M997 | E25 | H28 | M998 | E26 | H28 | M999 | E27 | H28 |
| M1000 | E28 | H28 | M1001 | E29 | H28 | M1002 | E30 | H28 |
| M1003 | E31 | H28 | M1004 | E32 | H28 | M1005 | E33 | H28 |
| M1006 | E34 | H28 | M1007 | E35 | H28 | M1008 | E36 | H28 |
| M1009 | E1 | H29 | M1010 | E2 | H29 | M1011 | E3 | H29 |
| M1012 | E4 | H29 | M1013 | E5 | H29 | M1014 | E6 | H29 |
| M1015 | E7 | H29 | M1016 | E8 | H29 | M1017 | E9 | H29 |
| M1018 | E10 | H29 | M1019 | E11 | H29 | M1020 | E12 | H29 |
| M1021 | E13 | H29 | M1022 | E14 | H29 | M1023 | E15 | H29 |
| M1024 | E16 | H29 | M1025 | E17 | H29 | M1026 | E18 | H29 |
| M1027 | E19 | H29 | M1028 | E20 | H29 | M1029 | E21 | H29 |
| M1030 | E22 | H29 | M1031 | E23 | H29 | M1032 | E24 | H29 |
| M1033 | E25 | H29 | M1034 | E26 | H29 | M1035 | E27 | H29 |
| M1036 | E28 | H29 | M1037 | E29 | H29 | M1038 | E30 | H29 |
| M1039 | E31 | H29 | M1040 | E32 | H29 | M1041 | E33 | H29 |
| M1042 | E34 | H29 | M1043 | E35 | H29 | M1044 | E36 | H29 |
| M1045 | E1 | H30 | M1046 | E2 | H30 | M1047 | E3 | H30 |
| M1048 | E4 | H30 | M1049 | E5 | H30 | M1050 | E6 | H30 |
| M1051 | E7 | H30 | M1052 | E8 | H30 | M1053 | E9 | H30 |
| M1054 | E10 | H30 | M1055 | E11 | H30 | M1056 | E12 | H30 |
| M1057 | E13 | H30 | M1058 | E14 | H30 | M1059 | E15 | H30 |
| M1060 | E16 | H30 | M1061 | E17 | H30 | M1062 | E18 | H30 |
| M1063 | E19 | H30 | M1064 | E20 | H30 | M1065 | E21 | H30 |
| M1066 | E22 | H30 | M1067 | E23 | H30 | M1068 | E24 | H30 |
| M1069 | E25 | H30 | M1070 | E26 | H30 | M1071 | E27 | H30 |
| M1072 | E28 | H30 | M1073 | E29 | H30 | M1074 | E30 | H30 |
| M1075 | E31 | H30 | M1076 | E32 | H30 | M1077 | E33 | H30 |
| M1078 | E34 | H30 | M1079 | E35 | H30 | M1080 | E36 | H30 |
| M1081 | E1 | H31 | M1082 | E2 | H31 | M1083 | E3 | H31 |
| M1084 | E4 | H31 | M1085 | E5 | H31 | M1086 | E6 | H31 |
| M1087 | E7 | H31 | M1088 | E8 | H31 | M1089 | E9 | H31 |
| M1090 | E10 | H31 | M1091 | E11 | H31 | M1092 | E12 | H31 |
| M1093 | E13 | H31 | M1094 | E14 | H31 | M1095 | E15 | H31 |
| M1096 | E16 | H31 | M1097 | E17 | H31 | M1098 | E18 | H31 |
| M1099 | E19 | H31 | M1100 | E20 | H31 | M1101 | E21 | H31 |
| M1102 | E22 | H31 | M1103 | E23 | H31 | M1104 | E24 | H31 |
| M1105 | E25 | H31 | M1106 | E26 | H31 | M1107 | E27 | H31 |
| M1108 | E28 | H31 | M1109 | E29 | H31 | M1110 | E30 | H31 |
| M1111 | E31 | H31 | M1112 | E32 | H31 | M1113 | E33 | H31 |
| M1114 | E34 | H31 | M1115 | E35 | H31 | M1116 | E36 | H31 |
| M1117 | E1 | H32 | M1118 | E2 | H32 | M1119 | E3 | H32 |
| M1120 | E4 | H32 | M1121 | E5 | H32 | M1122 | E6 | H32 |
| M1123 | E7 | H32 | M1124 | E8 | H32 | M1125 | E9 | H32 |
| M1126 | E10 | H32 | M1127 | E11 | H32 | M1128 | E12 | H32 |
| M1129 | E13 | H32 | M1130 | E14 | H32 | M1131 | E15 | H32 |
| M1132 | E16 | H32 | M1133 | E17 | H32 | M1134 | E18 | H32 |
| M1135 | E19 | H32 | M1136 | E20 | H32 | M1137 | E21 | H32 |
| M1138 | E22 | H32 | M1139 | E23 | H32 | M1140 | E24 | H32 |
| M1141 | E25 | H32 | M1142 | E26 | H32 | M1143 | E27 | H32 |
| M1144 | E28 | H32 | M1145 | E29 | H32 | M1146 | E30 | H32 |
| M1147 | E31 | H32 | M1148 | E32 | H32 | M1149 | E33 | H32 |
| M1150 | E34 | H32 | M1151 | E35 | H32 | M1152 | E36 | H32 |
| M1153 | E1 | H33 | M1154 | E2 | H33 | M1155 | E3 | H33 |
| M1156 | E4 | H33 | M1157 | E5 | H33 | M1158 | E6 | H33 |
| M1159 | E7 | H33 | M1160 | E8 | H33 | M1161 | E9 | H33 |
| M1162 | E10 | H33 | M1163 | E11 | H33 | M1164 | E12 | H33 |
| M1165 | E13 | H33 | M1166 | E14 | H33 | M1167 | E15 | H33 |
| M1168 | E16 | H33 | M1169 | E17 | H33 | M1170 | E18 | H33 |
| M1171 | E19 | H33 | M1172 | E20 | H33 | M1173 | E21 | H33 |
| M1174 | E22 | H33 | M1175 | E23 | H33 | M1176 | E24 | H33 |
| M1177 | E25 | H33 | M1178 | E26 | H33 | M1179 | E27 | H33 |
| M1180 | E28 | H33 | M1181 | E29 | H33 | M1182 | E30 | H33 |
| M1183 | E31 | H33 | M1184 | E32 | H33 | M1185 | E33 | H33 |
| M1186 | E34 | H33 | M1187 | E35 | H33 | M1188 | E36 | H33 |
| M1189 | E37 | H1 | M1190 | E38 | H1 | M1191 | E39 | H1 |
| M1192 | E40 | H1 | M1193 | E41 | H1 | M1194 | E42 | H1 |
| M1195 | E43 | H1 | M1196 | E44 | H1 | M1197 | E45 | H1 |
| M1198 | E37 | H2 | M1199 | E38 | H2 | M1200 | E39 | H2 |
| M1201 | E40 | H2 | M1202 | E41 | H2 | M1203 | E42 | H2 |
| M1204 | E43 | H2 | M1205 | E44 | H2 | M1206 | E45 | H2 |
| M1207 | E37 | H3 | M1208 | E38 | H3 | M1209 | E39 | H3 |
| M1210 | E40 | H3 | M1211 | E41 | H3 | M1212 | E42 | H3 |
| M1213 | E43 | H3 | M1214 | E44 | H3 | M1215 | E45 | H3 |
| M1216 | E37 | H4 | M1217 | E38 | H4 | M1218 | E39 | H4 |
| M1219 | E40 | H4 | M1220 | E41 | H4 | M1221 | E42 | H4 |
| M1222 | E43 | H4 | M1223 | E44 | H4 | M1224 | E45 | H4 |
| M1225 | E37 | H5 | M1226 | E38 | H5 | M1227 | E39 | H5 |
| M1228 | E40 | H5 | M1229 | E41 | H5 | M1230 | E42 | H5 |
| M1231 | E43 | H5 | M1232 | E44 | H5 | M1233 | E45 | H5 |
| M1234 | E37 | H6 | M1235 | E38 | H6 | M1236 | E39 | H6 |
| M1237 | E40 | H6 | M1238 | E41 | H6 | M1239 | E42 | H6 |
| M1240 | E43 | H6 | M1241 | E44 | H6 | M1242 | E45 | H6 |
| M1243 | E37 | H7 | M1244 | E38 | H7 | M1245 | E39 | H7 |
| M1246 | E40 | H7 | M1247 | E41 | H7 | M1248 | E42 | H7 |
| M1249 | E43 | H7 | M1250 | E44 | H7 | M1251 | E45 | H7 |
| M1252 | E37 | H8 | M1253 | E38 | H8 | M1254 | E39 | H8 |
| M1255 | E40 | H8 | M1256 | E41 | H8 | M1257 | E42 | H8 |
| M1258 | E43 | H8 | M1259 | E44 | H8 | M1260 | E45 | H8 |
| M1261 | E37 | H9 | M1262 | E38 | H9 | M1263 | E39 | H9 |
| M1264 | E40 | H9 | M1265 | E41 | H9 | M1266 | E42 | H9 |
| M1267 | E43 | H9 | M1268 | E44 | H9 | M1269 | E45 | H9 |
| M1270 | E37 | H10 | M1271 | E38 | H10 | M1272 | E39 | H10 |
| M1273 | E40 | H10 | M1274 | E41 | H10 | M1275 | E42 | H10 |
| M1276 | E43 | H10 | M1277 | E44 | H10 | M1278 | E45 | H10 |
| M1279 | E37 | H11 | M1280 | E38 | H11 | M1281 | E39 | H11 |
| M1282 | E40 | H11 | M1283 | E41 | H11 | M1284 | E42 | H11 |
| M1285 | E43 | H11 | M1286 | E44 | H11 | M1287 | E45 | H11 |
| M1288 | E37 | H12 | M1289 | E38 | H12 | M1290 | E39 | H12 |
| M1291 | E40 | H12 | M1292 | E41 | H12 | M1293 | E42 | H12 |
| M1294 | E43 | H12 | M1295 | E44 | H12 | M1296 | E45 | H12 |
| M1297 | E37 | H13 | M1298 | E38 | H13 | M1299 | E39 | H13 |
| M1300 | E40 | H13 | M1301 | E41 | H13 | M1302 | E42 | H13 |
| M1303 | E43 | H13 | M1304 | E44 | H13 | M1305 | E45 | H13 |
| M1306 | E37 | H14 | M1307 | E38 | H14 | M1308 | E39 | H14 |
| M1309 | E40 | H14 | M1310 | E41 | H14 | M1311 | E42 | H14 |
| M1312 | E43 | H14 | M1313 | E44 | H14 | M1314 | E45 | H14 |
| M1315 | E37 | H15 | M1316 | E38 | H15 | M1317 | E39 | H15 |
| M1318 | E40 | H15 | M1319 | E41 | H15 | M1320 | E42 | H15 |
| M1321 | E43 | H15 | M1322 | E44 | H15 | M1323 | E45 | H15 |
| M1324 | E37 | H16 | M1325 | E38 | H16 | M1326 | E39 | H16 |
| M1327 | E40 | H16 | M1328 | E41 | H16 | M1329 | E42 | H16 |
| M1330 | E43 | H16 | M1331 | E44 | H16 | M1332 | E45 | H16 |
| M1333 | E37 | H17 | M1334 | E38 | H17 | M1335 | E39 | H17 |
| M1336 | E40 | H17 | M1337 | E41 | H17 | M1338 | E42 | H17 |
| M1339 | E43 | H17 | M1340 | E44 | H17 | M1341 | E45 | H17 |
| M1342 | E37 | H18 | M1343 | E38 | H18 | M1344 | E39 | H18 |
| M1345 | E40 | H18 | M1346 | E41 | H18 | M1347 | E42 | H18 |
| M1348 | E43 | H18 | M1349 | E44 | H18 | M1350 | E45 | H18 |
| M1351 | E37 | H19 | M1352 | E38 | H19 | M1353 | E39 | H19 |
| M1354 | E40 | H19 | M1355 | E41 | H19 | M1356 | E42 | H19 |
| M1357 | E43 | H19 | M1358 | E44 | H19 | M1359 | E45 | H19 |
| M1360 | E37 | H20 | M1361 | E38 | H20 | M1362 | E39 | H20 |
| M1363 | E40 | H20 | M1364 | E41 | H20 | M1365 | E42 | H20 |
| M1366 | E43 | H20 | M1367 | E44 | H20 | M1368 | E45 | H20 |
| M1369 | E37 | H21 | M1370 | E38 | H21 | M1371 | E39 | H21 |
| M1372 | E40 | H21 | M1373 | E41 | H21 | M1374 | E42 | H21 |
| M1375 | E43 | H21 | M1376 | E44 | H21 | M1377 | E45 | H21 |
| M1378 | E37 | H22 | M1379 | E38 | H22 | M1380 | E39 | H22 |
| M1381 | E40 | H22 | M1382 | E41 | H22 | M1383 | E42 | H22 |
| M1384 | E43 | H22 | M1385 | E44 | H22 | M1386 | E45 | H22 |
| M1387 | E37 | H23 | M1388 | E38 | H23 | M1389 | E39 | H23 |
| M1390 | E40 | H23 | M1391 | E41 | H23 | M1392 | E42 | H23 |
| M1393 | E43 | H23 | M1394 | E44 | H23 | M1395 | E45 | H23 |
| M1396 | E37 | H24 | M1397 | E38 | H24 | M1398 | E39 | H24 |
| M1399 | E40 | H24 | M1400 | E41 | H24 | M1401 | E42 | H24 |
| M1402 | E43 | H24 | M1403 | E44 | H24 | M1404 | E45 | H24 |
| M1405 | E37 | H25 | M1406 | E38 | H25 | M1407 | E39 | H25 |
| M1408 | E40 | H25 | M1409 | E41 | H25 | M1410 | E42 | H25 |
| M1411 | E43 | H25 | M1412 | E44 | H25 | M1413 | E45 | H25 |
| M1414 | E37 | H26 | M1415 | E38 | H26 | M1416 | E39 | H26 |
| M1417 | E40 | H26 | M1418 | E41 | H26 | M1419 | E42 | H26 |
| M1420 | E43 | H26 | M1421 | E44 | H26 | M1422 | E45 | H26 |
| M1423 | E37 | H27 | M1424 | E38 | H27 | M1425 | E39 | H27 |
| M1426 | E40 | H27 | M1427 | E41 | H27 | M1428 | E42 | H27 |
| M1429 | E43 | H27 | M1430 | E44 | H27 | M1431 | E45 | H27 |
| M1432 | E37 | H28 | M1433 | E38 | H28 | M1434 | E39 | H28 |
| M1435 | E40 | H28 | M1436 | E41 | H28 | M1437 | E42 | H28 |
| M1438 | E43 | H28 | M1439 | E44 | H28 | M1440 | E45 | H28 |
| M1441 | E37 | H29 | M1442 | E38 | H29 | M1443 | E39 | H29 |
| M1444 | E40 | H29 | M1445 | E41 | H29 | M1446 | E42 | H29 |
| M1447 | E43 | H29 | M1448 | E44 | H29 | M1449 | E45 | H29 |
| M1450 | E37 | H30 | M1451 | E38 | H30 | M1452 | E39 | H30 |
| M1453 | E40 | H30 | M1454 | E41 | H30 | M1455 | E42 | H30 |
| M1456 | E43 | H30 | M1457 | E44 | H30 | M1458 | E45 | H30 |
| M1459 | E37 | H31 | M1460 | E38 | H31 | M1461 | E39 | H31 |
| M1462 | E40 | H31 | M1463 | E41 | H31 | M1464 | E42 | H31 |
| M1465 | E43 | H31 | M1466 | E44 | H31 | M1467 | E45 | H31 |
| M1468 | E37 | H32 | M1469 | E38 | H32 | M1470 | E39 | H32 |
| M1471 | E40 | H32 | M1472 | E41 | H32 | M1473 | E42 | H32 |
| M1474 | E43 | H32 | M1475 | E44 | H32 | M1476 | E45 | H32 |
| M1477 | E37 | H33 | M1478 | E38 | H33 | M1479 | E39 | H33 |
| M1480 | E40 | H33 | M1481 | E41 | H33 | M1482 | E42 | H33 |
| M1483 | E43 | H33 | M1484 | E44 | H33 | M1485 | E45 | H33 |

The concentration of the host material of the formula (1) as described above or described as preferred in the mixture of the invention or in the light-emitting layer of the device of the invention is typically in the range from 5% by weight to 90% by weight, preferably in the range from 10% by weight to 85% by weight, more preferably in the range from 20% by weight to 85% by weight, even more preferably in the range from 30% by weight to 80% by weight, very especially preferably in the range from 20% by weight to 60% by weight and most preferably in the range from 30% by weight to 50% by weight, based on the overall mixture or based on the overall composition of the light-emitting layer.

The concentration of the sum total of all host materials of the formulae (HH-1), (HH-2), (HH-3), (HH-4), (HH-5), (HH-5-A), (HH-5-B), (HH-5-C), (HH-5-D), (HH-5-E) and (HH-6) as described above or described as preferred in the mixture of the invention or in the light-emitting layer of the device of the invention is typically in the range from 10% by weight to 95% by weight, preferably in the range from 15% by weight to 90% by weight, more preferably in the range from 15% by weight to 80% by weight, even more preferably in the range from 20% by weight to 70% by weight, very especially preferably in the range from 40% by weight to 80% by weight and most preferably in the range from 50% by weight to 70% by weight, based on the overall mixture or based on the overall composition of the light-emitting layer.

The present invention also relates to a mixture which, as well as the aforementioned host materials of the formula (1), called host material 1 hereinafter, and the host material of at least one of the formulae (HH-1), (HH-2), (HH-3), (HH-4), (HH-5), (HH-5-A), (HH-5-B), (HH-5-C), (HH-5-D), (HH-5-E) and (HH-6), called host material 2 hereinafter, as described above or described as preferred, also comprises at least one phosphorescent emitter.

The present invention also relates to a mixture selected from M1 to M1485 that also comprises at least one phosphorescent emitter.

The present invention also relates to an organic electroluminescent device as described above or described as preferred, wherein the light-emitting layer, as well as the aforementioned host materials of the formulae (1) and at least one of the formulae (HH-1), (HH-2), (HH-3), (HH-4), (HH-5), (HH-5-A), (HH-5-B), (HH-5-C), (HH-5-D), (HH-5-E) and (HH-6), as described above or described as preferred, especially the material combinations M1 to M1485, also comprises at least one phosphorescent emitter.

The term “phosphorescent emitters” typically encompasses compounds where the light is emitted through a spin-forbidden transition from an excited state having higher spin multiplicity, i.e. a spin state >1, for example through a transition from a triplet state or a state having an even higher spin quantum number, for example a quintet state. This preferably means a transition from a triplet state.

Suitable phosphorescent emitters (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent emitters.

In general, all phosphorescent complexes as used for phosphorescent OLEDs according to the prior art and as known to those skilled in the art in the field of organic electroluminescent devices are suitable.

Preferred phosphorescent emitters according to the present invention conform to the formula (IIIa)

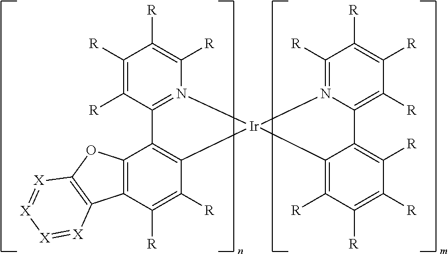

where the symbols and indices for this formula (IIIa) are defined as follows:
n+m is 3, n is 1 or 2, m is 2 or 1,
X is the same or different at each instance and is N or CR,
R is the same or different at each instance and is H, D, F, CN or a branched or linear alkyl group having 1 to 10 carbon atoms or a partly or fully deuterated, branched or linear alkyl group having 1 to 10 carbon atoms or a cycloalkyl group having 4 to 7 carbon atoms, which may be partly or fully substituted by deuterium, or an aromatic heteroaromatic ring system which has 5 to 60 ring atoms and may be partly or fully substituted by deuterium.

The invention accordingly further provides an organic electroluminescent device as described above or described as preferred, characterized in that the light-emitting layer, as well as the host materials 1 and 2, comprises at least one phosphorescent emitter conforming to the formula (IIIa) as described above.

In emitters of the formula (IIIa), n is preferably 1 and m is preferably 2.

In emitters of the formula (IIIa), preferably, one X is selected from N and the other X are CR, or all X are the same or different at each instance and are CR.

In emitters of the formula (IIIa), at least one R is preferably different than H. In emitters of the formula (IIIa), preferably two R are different than H and have one of the other definitions given above for the emitters of the formula (IIIa).

Preferred phosphorescent emitters according to the present invention conform to the formulae (I), (II), (III), (IV) or (V)

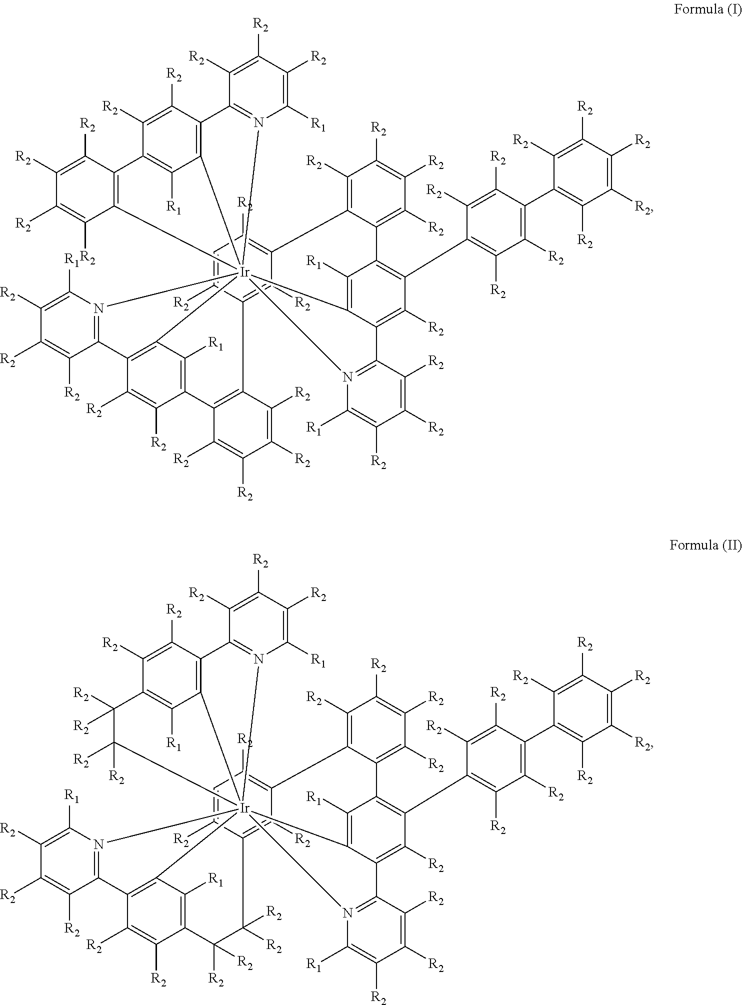

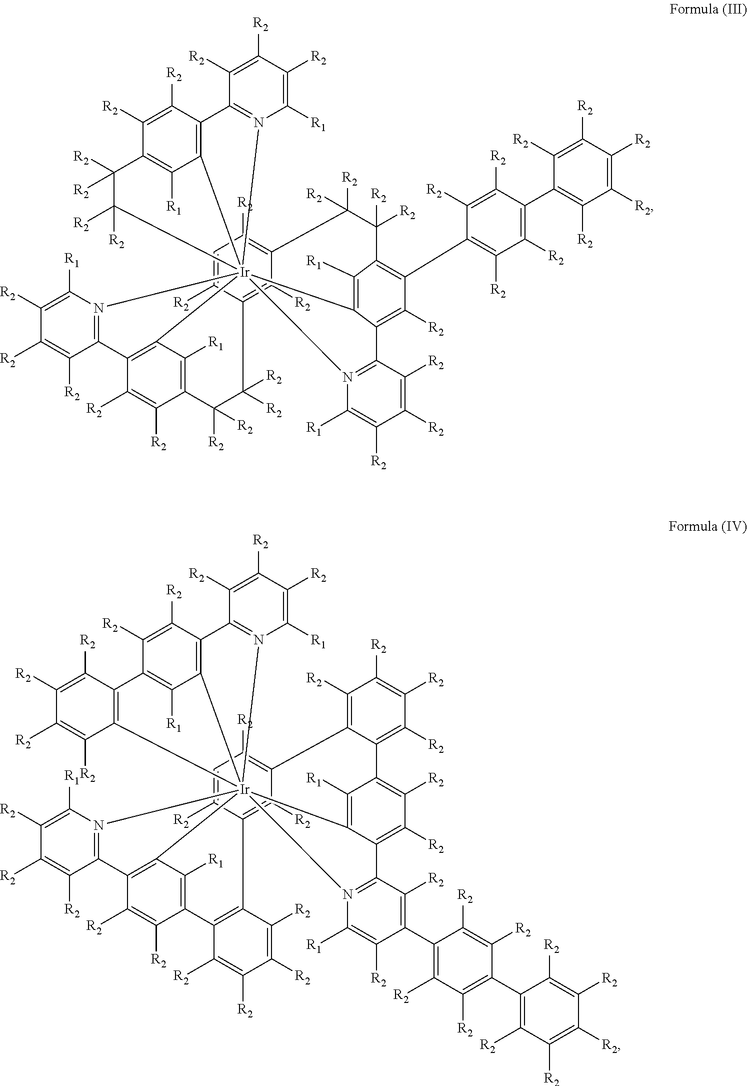

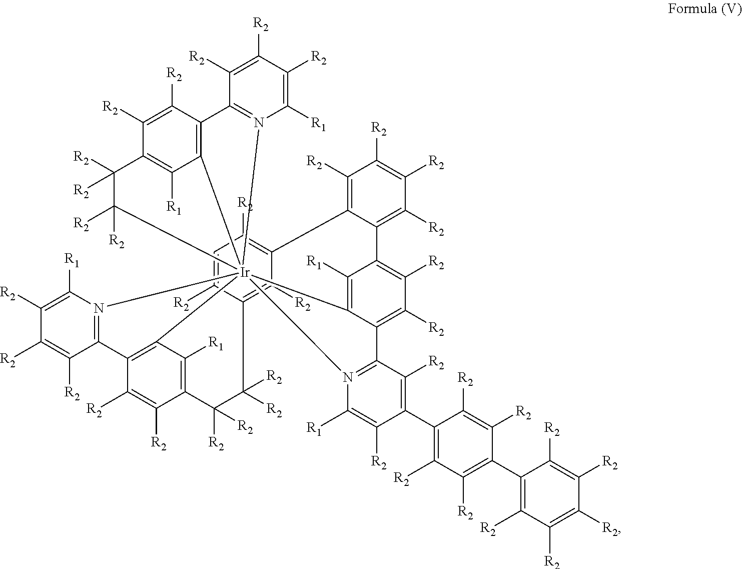

where the symbols and indices for these formulae (I), (II), (III), (IV) and (V) are defined as follows:
R₁ is H or D, R₂ is H, D, F, CN or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred phosphorescent emitters according to the present invention conform to the formulae (VI), (VII) or (VIII)

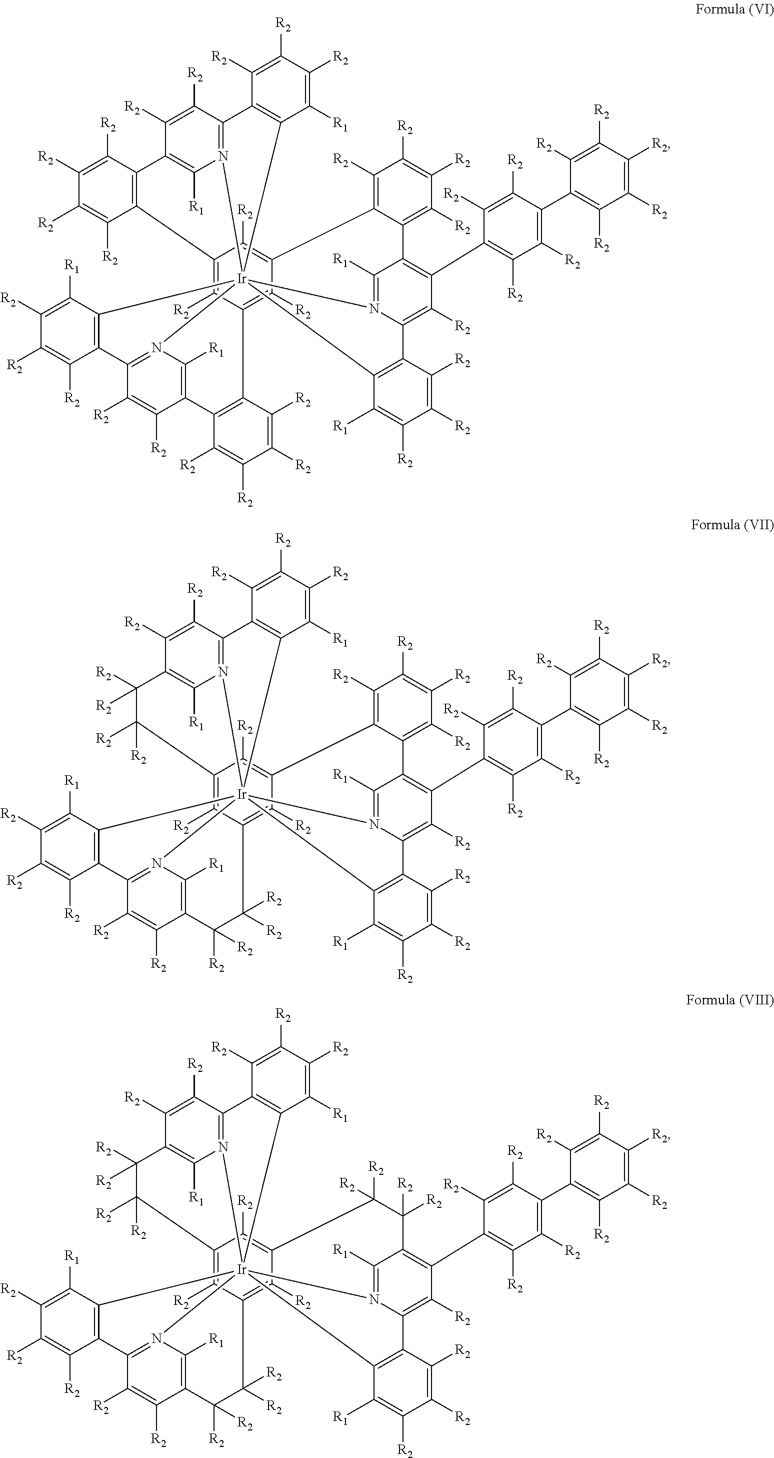

where the symbols and indices for these formulae (VI), (VII) and (VIII) are defined as follows:
R₁ is H or D, R₂ is H, D, F, CN or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred examples of phosphorescent emitters are described in WO2019/007867 on pages 120 to 126 in table 5, and on pages 127 to 129 in table 6. The emitters are incorporated into description by this reference.

Particularly preferred examples of phosphorescent emitters are listed in table 6 below.

TABLE 6

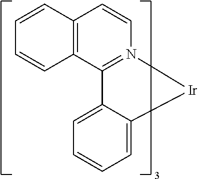

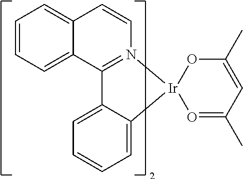

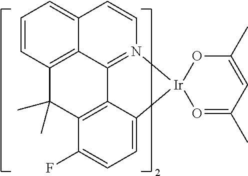

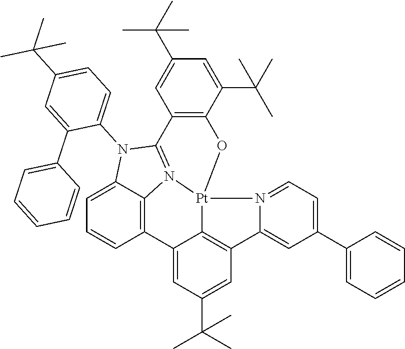

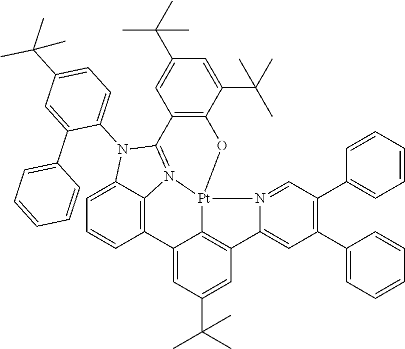

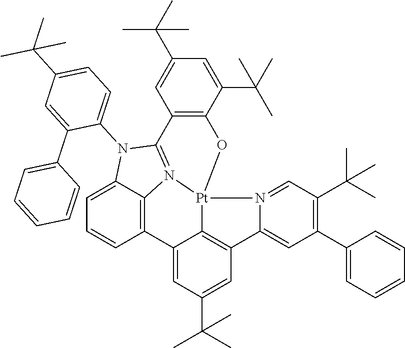

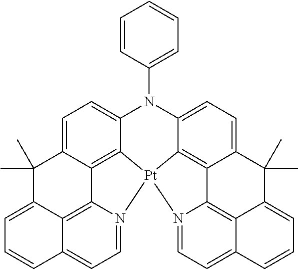

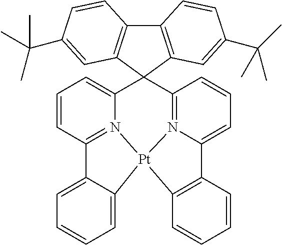

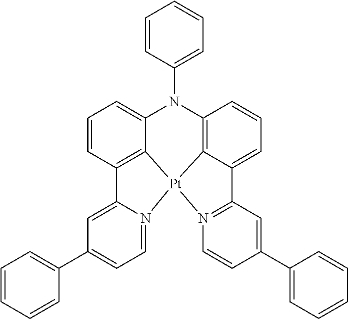

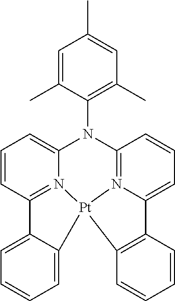

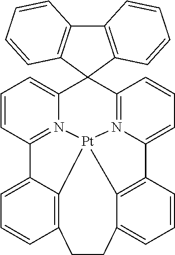

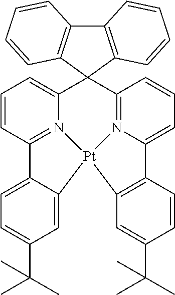

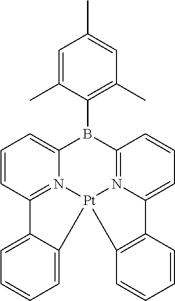

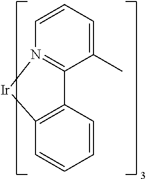

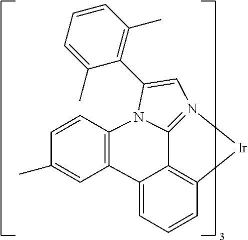

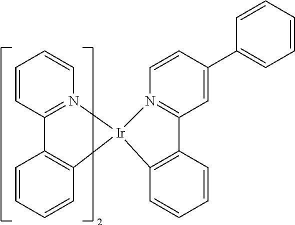

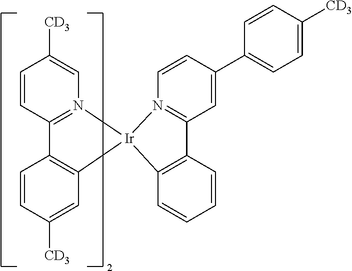

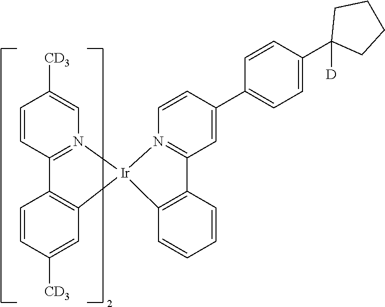

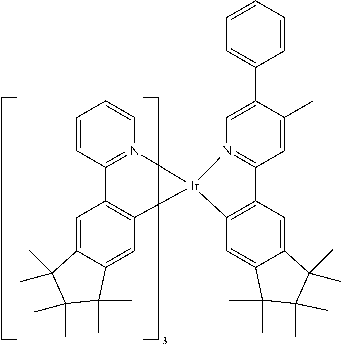

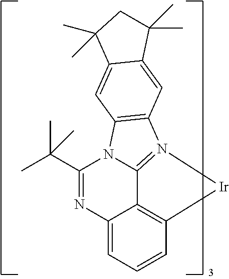

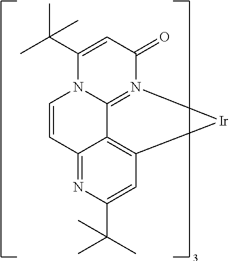

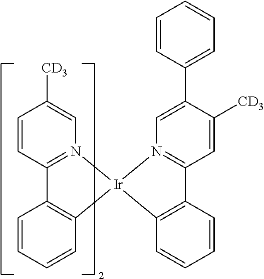

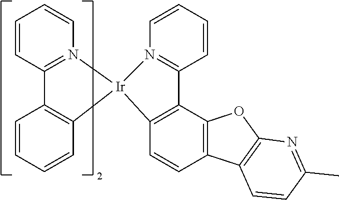

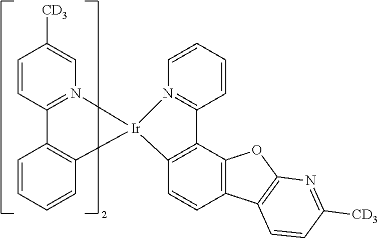

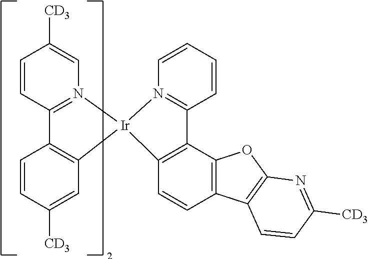

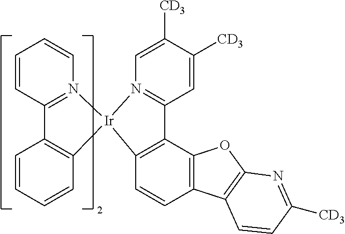

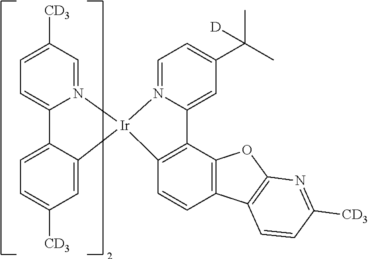

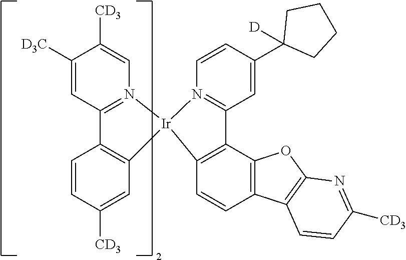

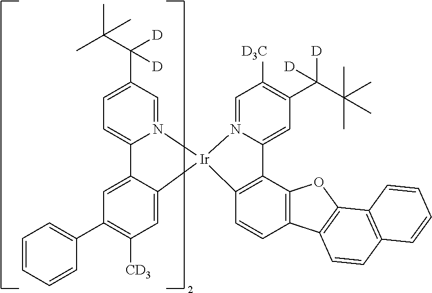

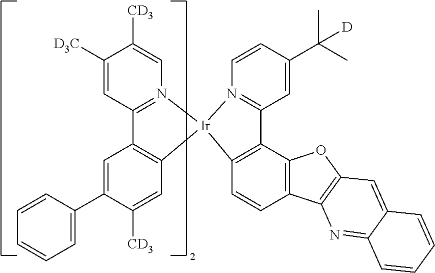

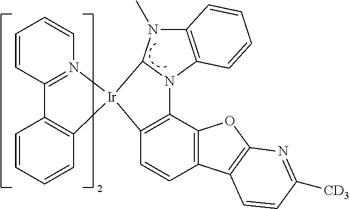

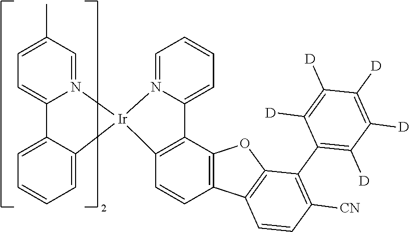

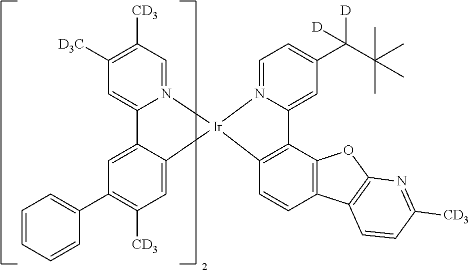

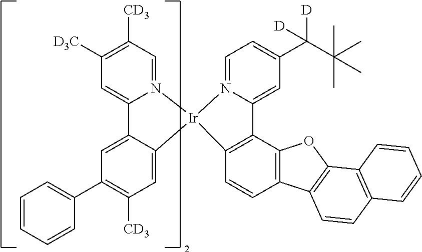

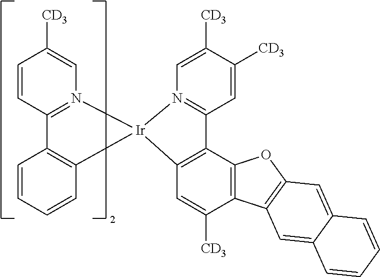

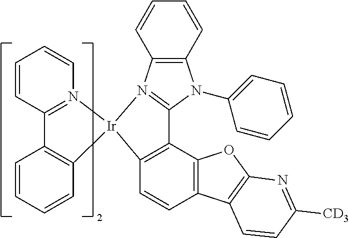

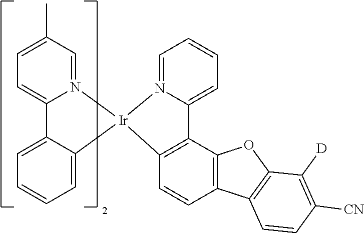

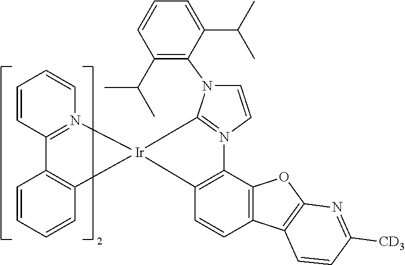

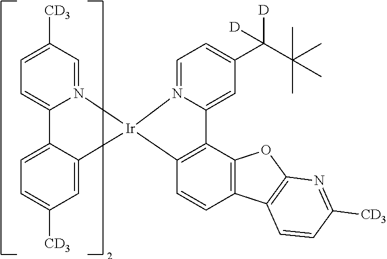

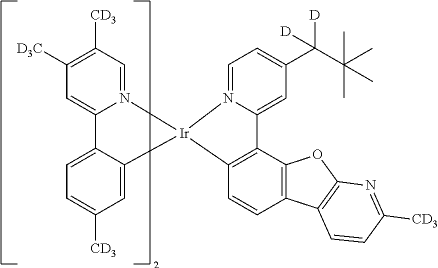

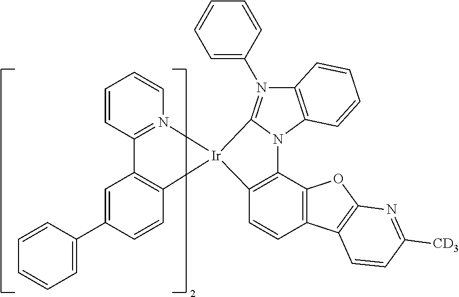

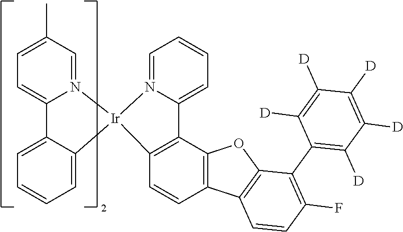

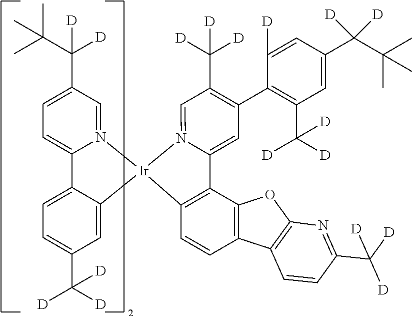

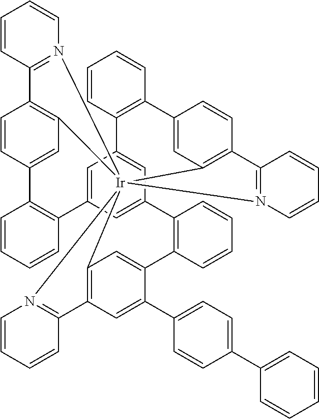

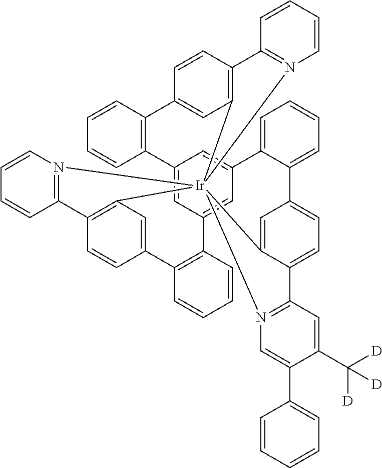

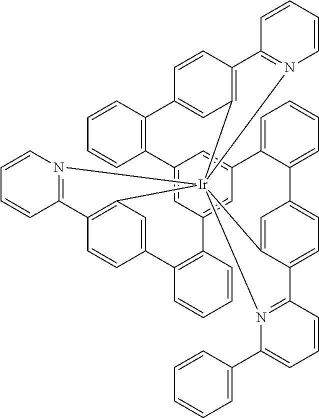

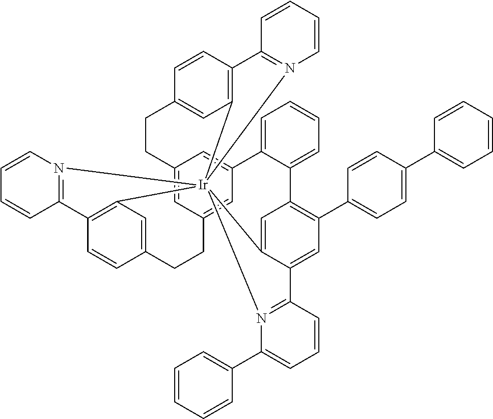

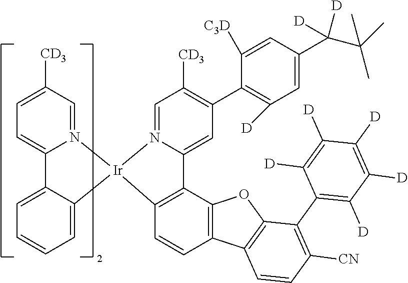

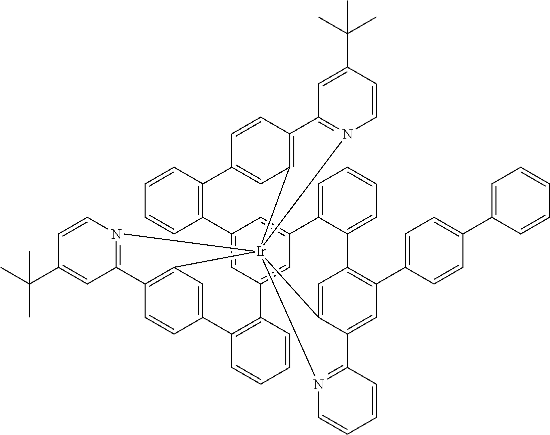

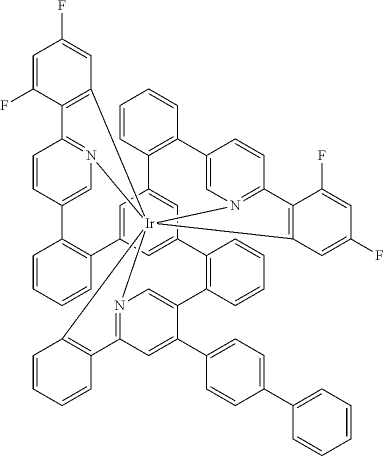

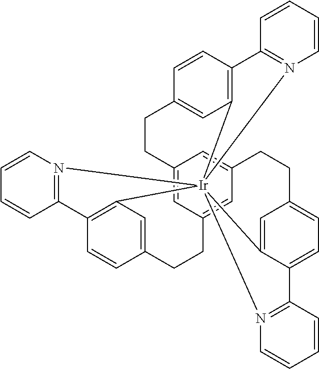

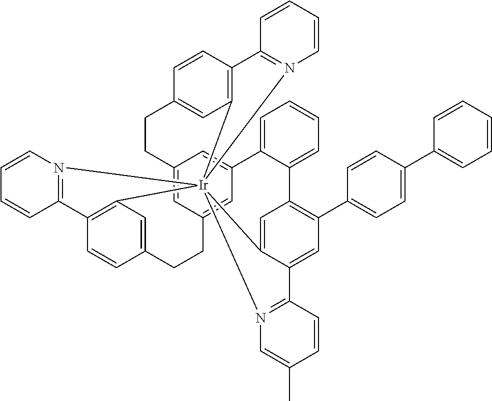

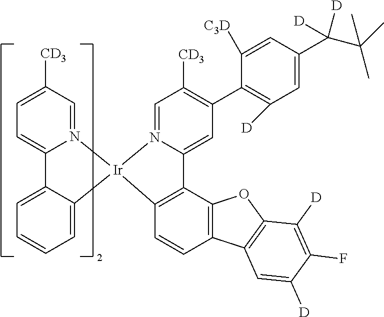

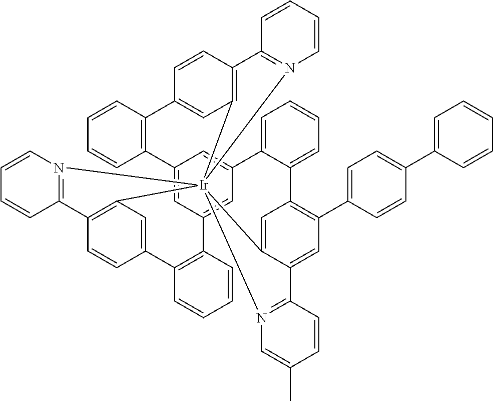

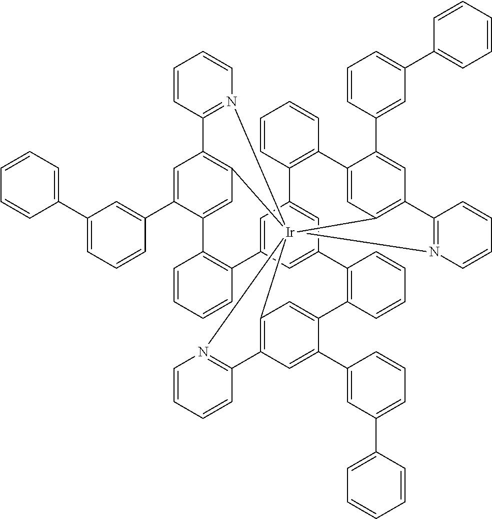

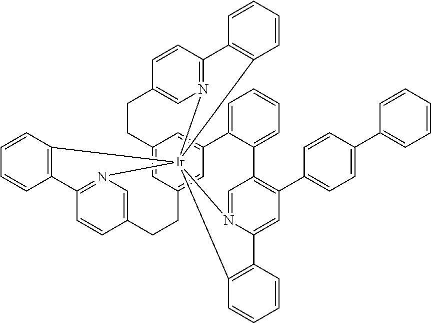

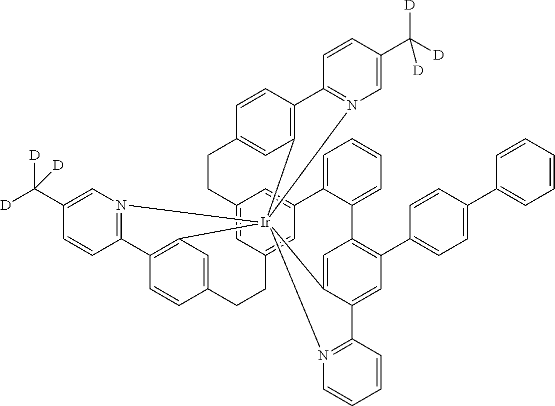

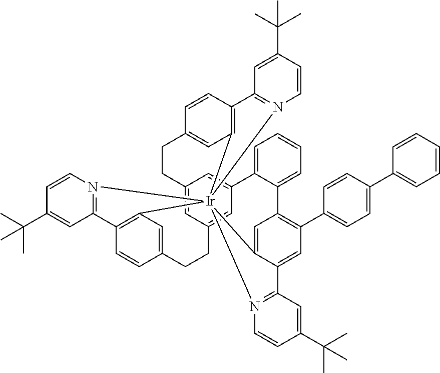

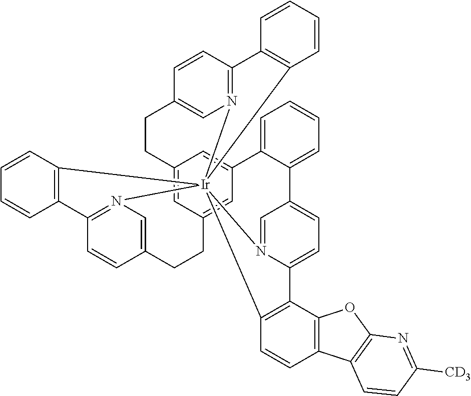

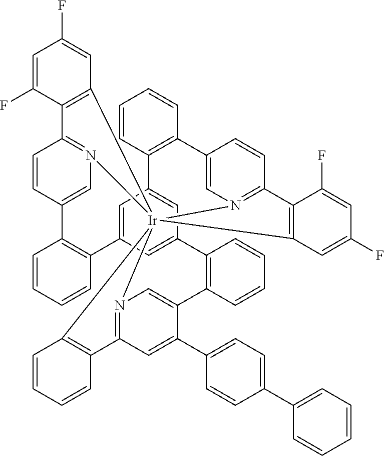

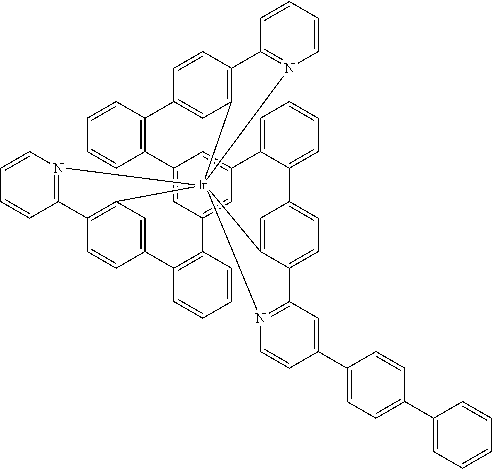

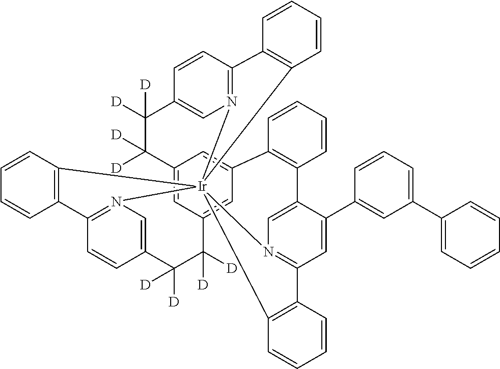

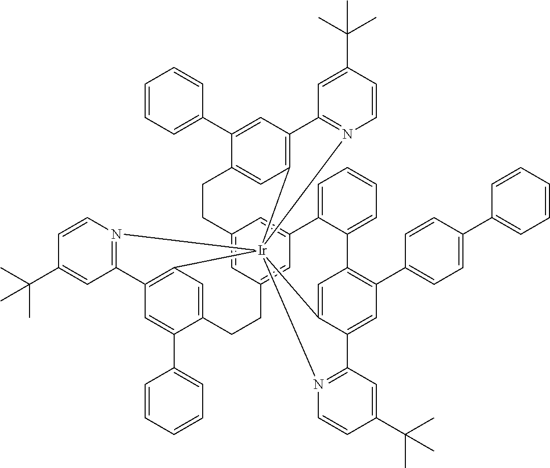

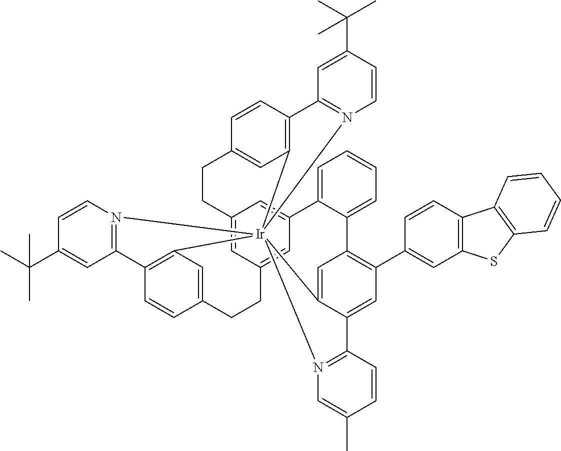

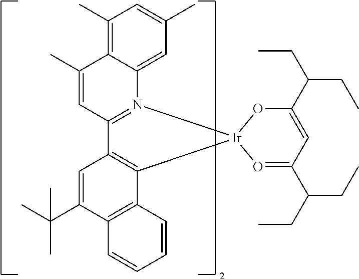

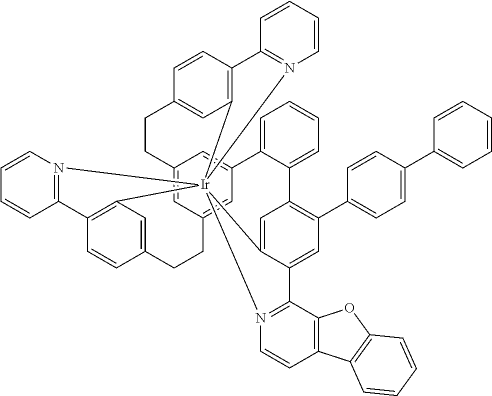

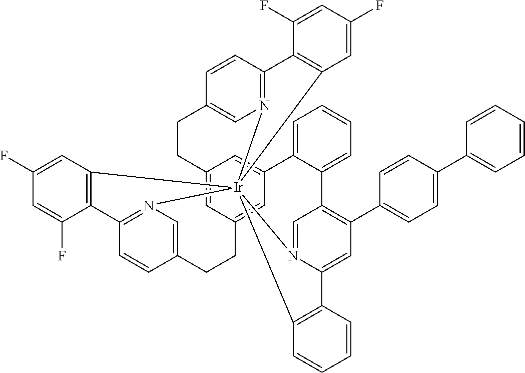

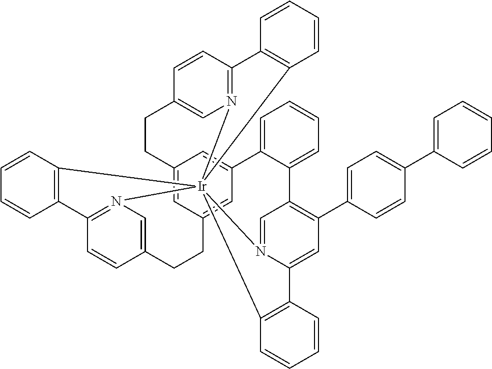

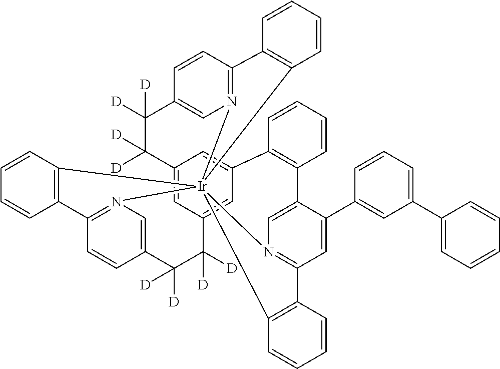

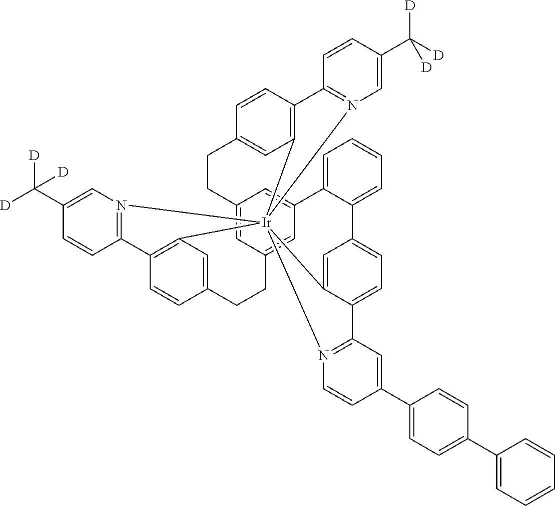

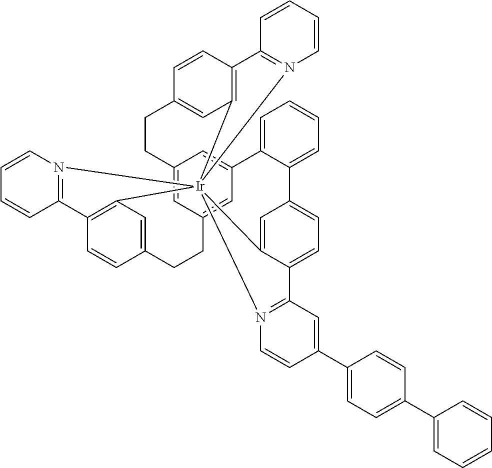

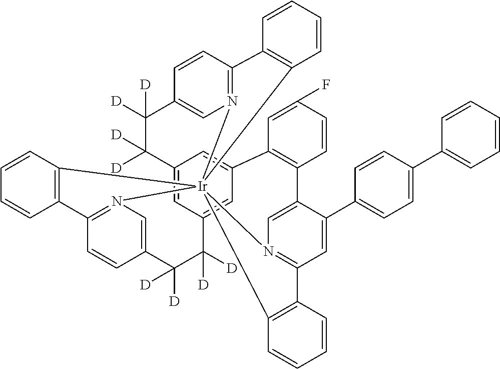

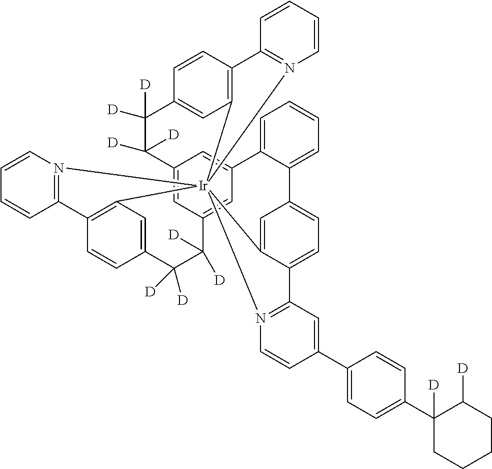

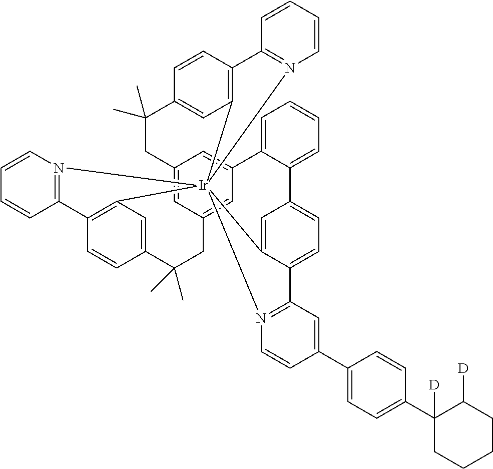

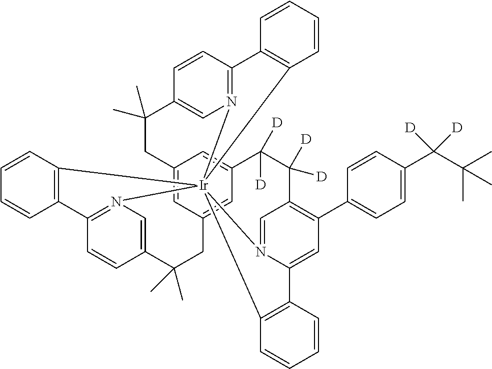

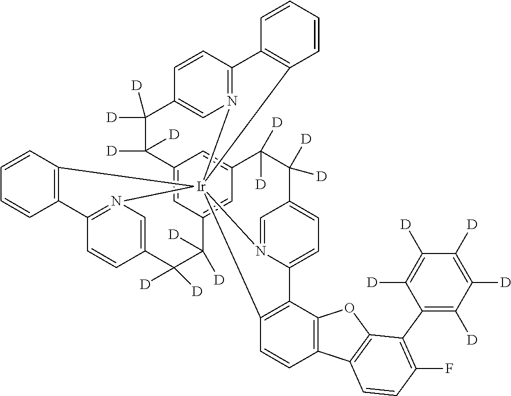

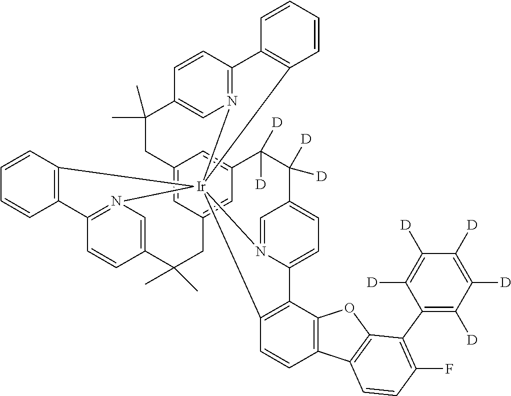

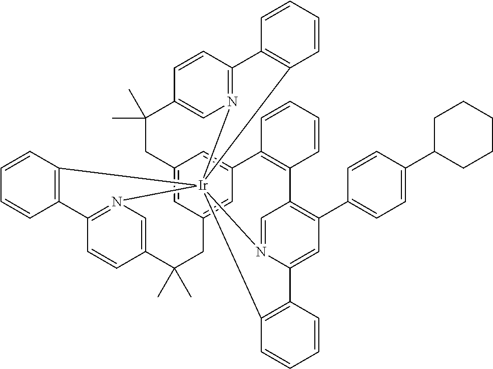

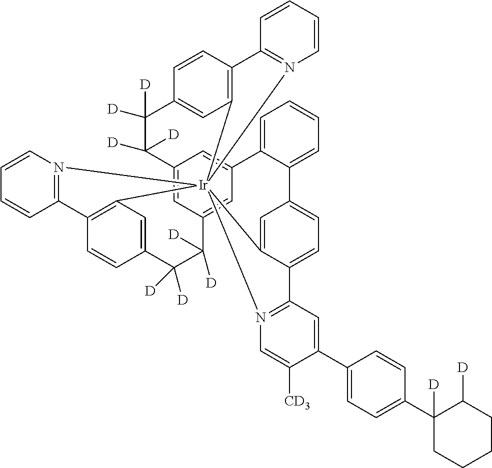

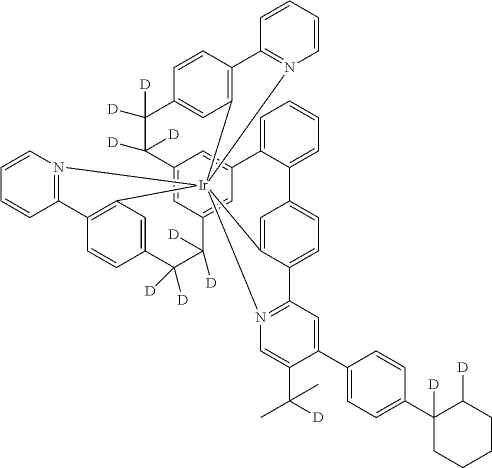

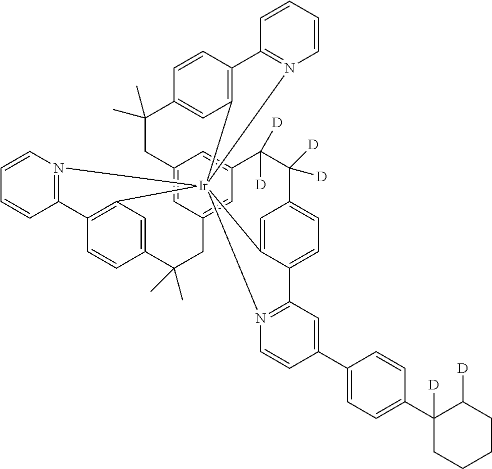

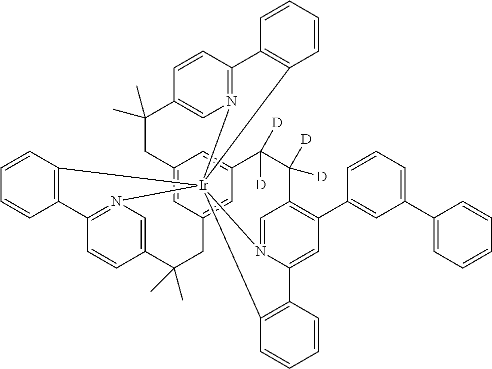

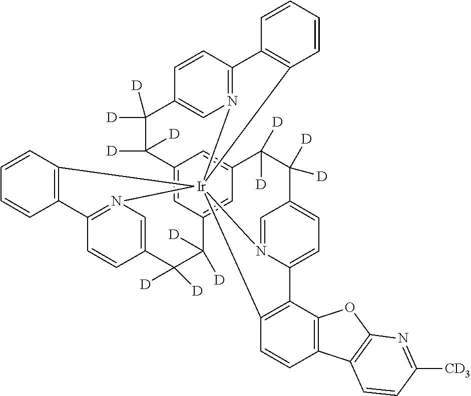

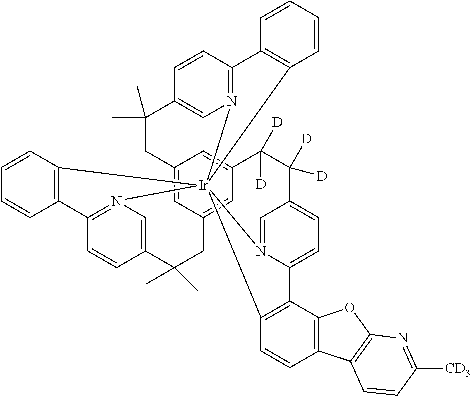

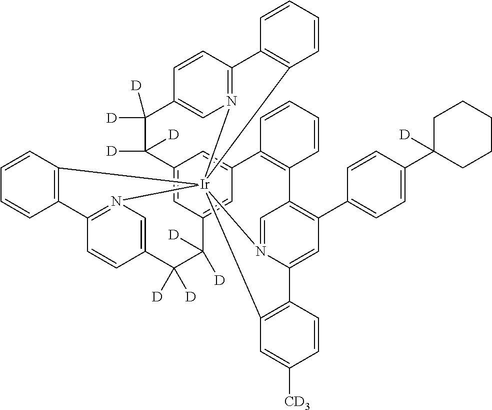

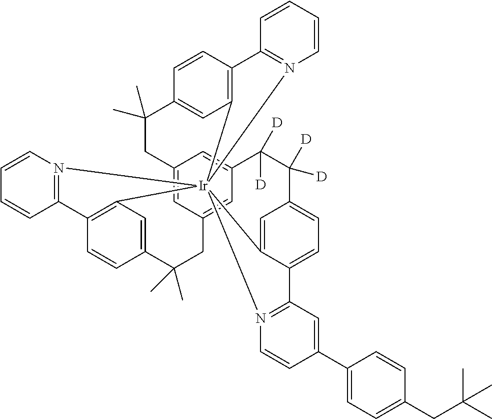

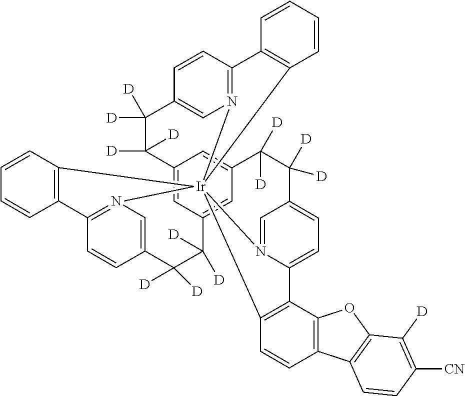

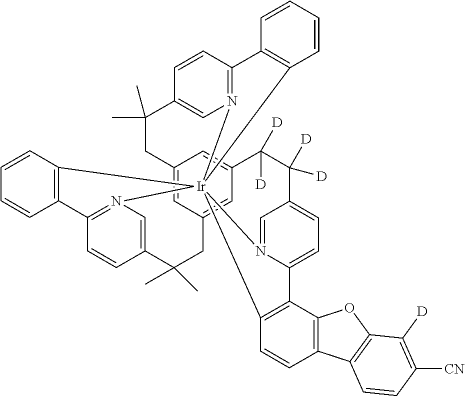

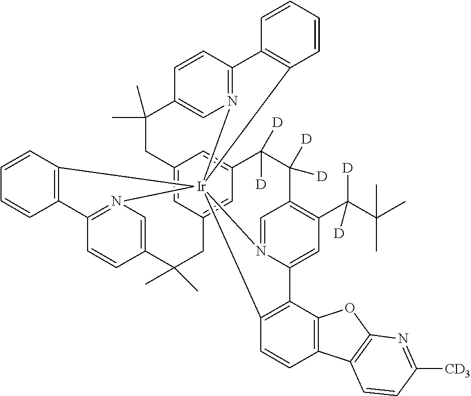

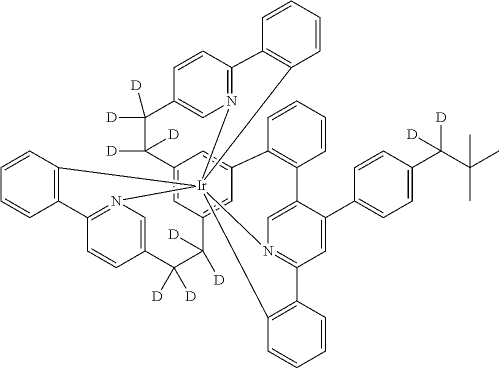

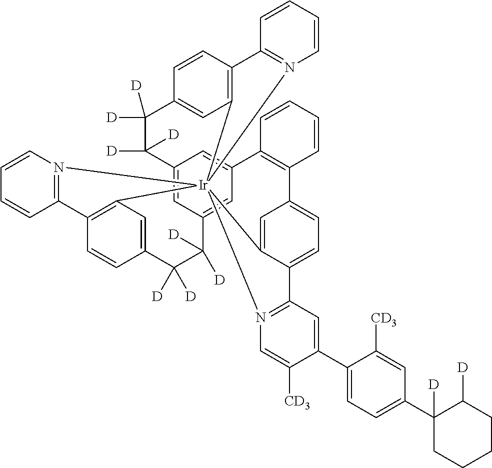

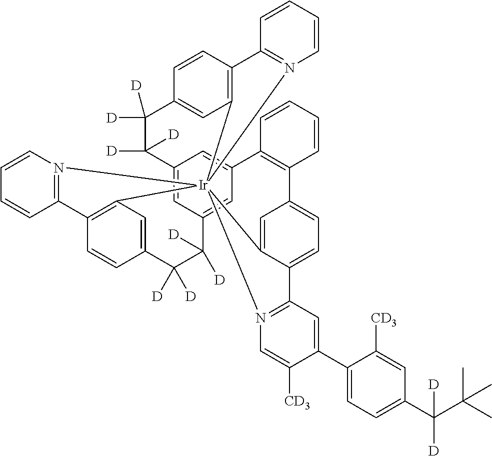

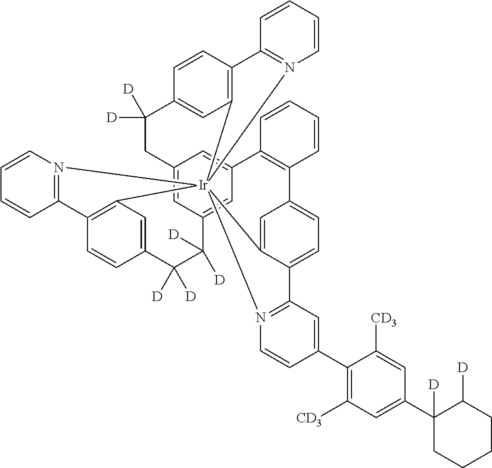

In the mixtures of the invention or in the light-emitting layer of the device of the invention, any mixture selected from the sum of the mixtures M1 to M1485 is preferably combined with a compound of the formula (IIIa) or a compound of the formulae (I) to (VIII) or a compound from table 6.

The light-emitting layer in the organic electroluminescent device of the invention, comprising at least one phosphorescent emitter, is preferably an infrared-emitting or yellow-, orange-, red-, green-, blue- or ultraviolet-emitting layer, more preferably a yellow- or green-emitting layer and most preferably a green-emitting layer.

What is meant here by a yellow-emitting layer is a layer having a photoluminescence maximum within the range from 540 to 570 nm. What is meant by an orange-emitting layer is a layer having a photoluminescence maximum within the range from 570 to 600 nm. What is meant by a red-emitting layer is a layer having a photoluminescence maximum within the range from 600 to 750 nm. What is meant by a green-emitting layer is a layer having a photoluminescence maximum within the range from 490 to 540 nm. What is meant by a blue-emitting layer is a layer having a photoluminescence maximum within the range from 440 to 490 nm. The photoluminescence maximum of the layer is determined here by measuring the photoluminescence spectrum of the layer having a layer thickness of 50 nm at room temperature, the layer having the inventive combination of the host material 1 of the formulae (1), (1a), (1b), (1c), (1d), (1e), (1f), (1g), (1h), (1i), (1j), (1k), (1l) or (1m) and the host material 2 consisting of at least one of the formulae (HH-1), (HH-2), (HH-3), (HH-4), (HH-5), (HH-5-A), (HH-5-B), (HH-5-C), (HH-5-D), (HH-5-E) and (HH-6), and the appropriate emitter.

The photoluminescence spectrum of the layer is recorded, for example, with a commercial photoluminescence spectrometer.

The photoluminescence spectrum of the emitter chosen is generally measured in oxygen-free solution, 10⁻⁵ molar, at room temperature, a suitable solvent being any in which the chosen emitter dissolves in the concentration mentioned. Particularly suitable solvents are typically toluene or 2-methyl-THF, but also dichloromethane. Measurement is effected with a commercial photoluminescence spectrometer. The triplet energy T₁ in eV is determined from the photoluminescence spectra of the emitters. First the peak maximum Plmax. (in nm) of the photoluminescence spectrum is determined. The peak maximum Plmax. (in nm) is then converted to eV by: E(T₁ in eV)=1240/E(T₁ in nm)=1240/PLmax. (in nm).

Preferred phosphorescent emitters are accordingly yellow emitters, preferably of the formula (IIIa), of the formulae (I) to (VIII) or from table 6, the triplet energy T₁ of which is preferably ~2.3 eV to ~2.1 eV.

Preferred phosphorescent emitters are accordingly green emitters, preferably of the formula (IIIa), of the formulae (I) to (VIII) or from table 6, the triplet energy T₁ of which is preferably ~2.5 eV to ~2.3 eV.

Particularly preferred phosphorescent emitters are accordingly green emitters, preferably of the formula (IIIa), of the formulae (I) to (VIII) or from table 6 as described above, the triplet energy T₁ of which is preferably ~2.5 eV to ~2.3 eV.

Most preferably, green emitters, preferably of the formula (IIIa), of the formulae (I) to (VIII) or from table 6, as described above, are selected for the mixture of the invention or emitting layer of the invention.

It is also possible for fluorescent emitters to be present in the light-emitting layer of the device of the invention or in the mixture of the invention.

Preferred fluorescent emitting compounds are selected from the class of the arylamines, where preferably at least one of the aromatic or heteroaromatic ring systems of the arylamine is a fused ring system, more preferably having at least 14 ring atoms. Preferred examples of these are aromatic anthraceneamines, aromatic anthracenediamines, aromatic pyreneamines, aromatic pyrenediamines, aromatic chryseneamines or aromatic chrysenediamines. What is meant by an aromatic anthraceneamine is a compound in which a diarylamino group is bonded directly to an anthracene group, preferably in the 9 position. What is meant by an aromatic anthracenediamine is a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units. The light-emitting device or the mixture of the invention may additionally also comprise materials that exhibit TADF (thermally activated delayed fluorescence).

In a further preferred embodiment of the invention, the at least one light-emitting layer of the organic electroluminescent device may have three or four different matrix materials, preferably three different matrix materials. These corresponding mixed matrix systems may consist of the matrix materials described for the host material 1 and the host material 2, but they may also comprise, as a third or fourth matrix material, for example alongside a host material 1 or host material 2, wide-band-gap materials, bipolar host materials, electron transport materials (ETM) or hole transport materials (HTM).

Preferably, the mixed matrix system is optimized for an emitter of the formula (IIIa), of the formulae (I) to (VIII), or for an emitter from table 6.

In one embodiment of the present invention, the mixture, aside from the constituents of the host material 1 and of the host material 2 as described above or described as preferred, does not comprise any further constituents, i.e. functional materials. These are material mixtures that are used as such for production of the light-emitting layer. These mixtures are also referred to as premix systems that are used as the sole material source in the vapor deposition of the host materials for the light-emitting layer and have a constant mixing ratio in the vapor deposition. In this way, it is possible in a simple and rapid manner to achieve the vapor deposition of a layer with homogeneous distribution of the components without the need for precise actuation of a multitude of material sources.

In an alternative embodiment of the present invention, the mixture, aside from the constituents of the host material of the formula (1) and the host material 2, as described above or described with preference, also comprises a phosphorescent emitter, as described above. In the case of a suitable mixing ratio in the vapor deposition, this mixture may also be used as the sole material source.

Preference is given to premix systems consisting of two matrix materials, namely one compound of the formulae (1), (1a), (1b), (1c), (1d), (1e), (1f), (1g), (1h), (1i), (1j), (1k), (1l) or (1m) and one compound of one of the formulae (HH-1), (HH-2), (HH-3), (HH-4), (HH-5), (HH-5-A), (HH-5-B), (HH-5-C), (HH-5-D), (HH-5-E) or (HH-6).

Preference is given to premix systems consisting of three matrix materials, namely one compound of the formulae (1), (1a), (1b), (1c), (1d), (1e), (1f), (1g), (1h), (1i), (1j), (1k), (1l) or (1m) and two compounds of one of the formulae (HH-1), (HH-2), (HH-3), (HH-4), (HH-5), (HH-5-A), (HH-5-B), (HH-5-C), (HH-5-D), (HH-5-E) or (HH-6).

The components or constituents of the light-emitting layer of the device of the invention may thus be processed by vapor deposition or from solution. The material combination of host materials 1 and 2 as described above or described as preferred, optionally with the phosphorescent emitter as described above or described as preferred, are provided for that purpose in a formulation containing at least one solvent. Suitable formulations have been described above.

The light-emitting layer in the device of the invention, according to the preferred embodiments and the emitting compound, contains preferably between 99.9% and 1% by volume, further preferably between 99% and 10% by volume, especially preferably between 98% and 60% by volume, very especially preferably between 97% and 80% by volume, of matrix material composed of at least one compound of the formulae (1), (1a), (1b), (1c), (1d), (1e), (1f), (1g), (1h), (1i), (1j), (1k), (1l) or (1m) and at least one compound of one of the formulae (HH-1), (HH-2), (HH-3), (HH-4), (HH-5), (HH-5-A), (HH-5-B), (HH-5-C), (HH-5-D), (HH-5-E) or (HH-6) according to the preferred embodiments, based on the overall composition of emitter and matrix material. Correspondingly, the light-emitting layer in the device of the invention preferably contains between 0.1% and 99% by volume, further preferably between 1% and 90% by volume, more preferably between 2% and 40% by volume, most preferably between 3% and 20% by volume, of the emitter based on the overall composition of the light-emitting layer composed of emitter and matrix material. If the compounds are processed from solution, preference is given to using the corresponding amounts in % by weight rather than the above-specified amounts in % by volume.

The present invention also relates to an organic electroluminescent device as described above or described as preferred, wherein the organic layer comprises a hole injection layer (HIL) and/or a hole transport layer (HTL), the hole-injecting material and hole-transporting material of which belongs to the class of the arylamines.

The sequence of layers in the organic electroluminescent device of the invention is preferably as follows:
anode/hole injection layer/hole transport layer/emitting layer/hole blocker layer/electron transport layer/electron injection layer/cathode.

This sequence of the layers is a preferred sequence.

At the same time, it should be pointed out again that not all the layers mentioned need be present and/or that further layers may additionally be present.

Materials used for the electron transport layer may be any materials as used according to the prior art as electron transport materials in the electron transport layer. Especially suitable are aluminum complexes, for example Alq₃, zirconium complexes, for example Zrq₄, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

The present invention also relates to an organic electroluminescent device as described above or described as preferred, wherein the organic layer comprises an electron injection layer (EIL) and/or an electron transport layer (ETL) and/or a hole blocker layer, the electron-injecting material and electron-transporting material of which is selected from the compounds of the formulae (1), (1a), (1b), (1c), (1d), (1e), (1f), (1g), (1h), (1i), (1j), (1k), (1l) or (1m) as described above or described as preferred.

Suitable cathodes of the device of the invention are metals having a low work function, metal alloys or multilayer structures composed of various metals, for example alkaline earth metals, alkali metals, main group metals or lanthanoids (e.g. Ca, Ba, Mg, Al, In, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the outcoupling of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The organic electroluminescent device of the invention, in the course of production, is appropriately (according to the application) structured, contact-connected and finally sealed, since the lifetime of the devices of the invention is shortened in the presence of water and/or air.

The production of the device of the invention is not restricted here. It is possible that one or more organic layers, including the light-emitting layer, are coated by a sublimation method. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

The organic electroluminescent device of the invention is preferably characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

The organic electroluminescent device of the invention is further preferably characterized in that one or more organic layers comprising the composition of the invention are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble host materials 1 and 2 and phosphorescent emitters are needed. Processing from solution has the advantage that, for example, the light-emitting layer can be applied in a very simple and inexpensive manner. This technique is especially suitable for the mass production of organic electroluminescent devices.

In addition, hybrid methods are possible, in which, for example, one or more layers are applied from solution and one or more further layers are applied by vapor deposition.

These methods are known in general terms to those skilled in the art and can be applied to organic electroluminescent devices.

The invention therefore further provides a process for producing the organic electroluminescent device of the invention as described above or described as preferred, characterized in that the organic layer, preferably the light-emitting layer, the hole injection layer and/or hole transport layer, is applied by gas phase deposition, especially by a sublimation method and/or by an OVPD (organic vapor phase deposition) method and/or with the aid of a carrier gas sublimation, or from solution, especially by spin-coating or by a printing method.

In the case of production by means of gas phase deposition, there are in principle two ways in which the organic layer, preferably the light-emitting layer, of the invention can be applied or vapor-deposited onto any substrate or the prior layer. Firstly, initial charges of each of the materials used in a material source can ultimately be evaporated from the different material sources (“co-evaporation”). Secondly, the various materials can be premixed (premix systems) and an initial charge of the mixture can ultimately be evaporated from a single material source (“premix evaporation”). In this way, it is possible in a simple and rapid manner to achieve the vapor deposition of the light-emitting layer with homogeneous distribution of the components without the need for precise actuation of a multitude of material sources.

The following methods are possible:

A process for producing the organic electroluminescent device of the invention as described above or described as preferred, characterized in that the organic layer, preferably the light-emitting layer, the electron transport layer and/or hole blocker layer, is applied by gas phase deposition, especially by a sublimation method and/or by an OVPD (organic vapor phase deposition) method and/or with the aid of a carrier gas sublimation, or from solution, especially by spin-coating or by a printing method.

A process for producing the organic electroluminescent device of the invention, as described above or described as preferred, characterized in that the light-emitting layer of the organic layer is applied by gas phase deposition, wherein the at least one compound of the formulae (1), (1a), (1b), (1c), (1d), (1e), (1f), (1g), (1h), (1i), (1j), (1k), (1l) or (1m) is deposited from the gas phase together with the further materials that form the light-emitting layer, successively or simultaneously from at least two material sources.

A process for producing the device of the invention, characterized in that the light-emitting layer of the organic layer is applied by gas phase deposition, wherein the at least one compound of the formulae (1), (1a), (1b), (1c), (1d), (1e), (1f), (1g), (1h), (1i), (1j), (1k), (1l) or (1m) is deposited from the gas phase together with at least one further matrix material as premix, successively or simultaneously with the light-emitting materials selected from the group of the phosphorescent emitters, of the fluorescent emitters and/or of the emitters that exhibit TADF (thermally activated delayed fluorescence).

The electronic devices of the invention, especially organic electroluminescent devices, are notable for one or more of the following surprising advantages over the prior art:
1. Electronic devices, especially organic electroluminescent devices, comprising compounds of formula (1) or the preferred embodiments recited above and hereinafter, especially as matrix material or as electron-conducting materials, have a very good lifetime. In this context, these compounds especially bring about low roll-off, i.e. a small drop in power efficiency of the device at high luminances.
2. Electronic devices, especially organic electroluminescent devices, comprising compounds of formula (1) or the preferred embodiments recited above and hereinafter, as electron-conducting materials and/or matrix materials, have excellent efficiency. In this context, compounds of the invention having structures of formula (1) or the preferred embodiments recited above and hereinafter bring about a low operating voltage when used in electronic devices.
3. The inventive compounds of formula (1) or the preferred embodiments recited above and hereinafter exhibit very high stability and lifetime.
4. With compounds of formula (1) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. The use of compounds of formula (1) or the preferred embodiments recited above and hereinafter in layers of electronic devices, especially organic electroluminescent devices, leads to high mobility of the electron conductor structures.
6. Compounds of formula (1) or the preferred embodiments recited above and hereinafter have excellent glass film formation.
7. Compounds of formula (1) or the preferred embodiments recited above and hereinafter form very good films from solutions.
8. The compounds of formula (1) or the preferred embodiments recited above and hereinafter have a low triplet level T₁ which may be in the range of 2.50 eV-2.90 eV.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Any feature disclosed in the present invention, unless stated otherwise, should therefore be considered as an example from a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

The technical teaching disclosed with the present invention may be abstracted and combined with other examples.

The invention is illustrated in detail by the examples which follow, without any intention of restricting it thereby.

### EXAMPLES

### General Methods:

In all quantum-chemical calculations, the Gaussian16 (Rev. B.01) software package is used. The neutral singlet ground state is optimized at the B3LYP/6-31G(d) level. HOMO and LUMO values are determined at the B3LYP/6-31G(d) level for the B3LYP/6-31G(d)-optimized ground state energy. Then TD-DFT singlet and triplet excitations (vertical excitations) are calculated by the same method (B3LYP/6-31G(d)) and with the optimized ground state geometry. The standard settings for SCF and gradient convergence are used.

From the energy calculation, the HOMO is obtained as the last orbital occupied by two electrons (alpha occ. eigenvalues) and LUMO as the first unoccupied orbital (alpha virt. eigenvalues) in Hartree units, where HEh and LEh represent the HOMO energy in Hartree units and the LUMO energy in Hartree units respectively. This is used to determine the HOMO and LUMO value in electron volts, calibrated by cyclic voltammetry measurements, as follows:

HOMOcorr=0.90603*HOMO−0.84836

LUMOcorr=0.99687*LUMO−0.72445

The triplet level T1 of a material is defined as the relative excitation energy (in eV) of the triplet state having the lowest energy which is found by the quantum-chemical energy calculation.

The singlet level S1 of a material is defined as the relative excitation energy (in eV) of the singlet state having the second-lowest energy which is found by the quantum-chemical energy calculation.

The energetically lowest singlet state is referred to as S0.

The method described herein is independent of the software package used and always gives the same results. Examples of frequently utilized programs for this purpose are “Gaussian09” (Gaussian Inc.) and Q-Chem 4.1 (Q-Chem, Inc.). In the present case, the energies are calculated using the software package “Gaussian16 (Rev. B.01)”.

### Synthesis Examples

The syntheses which follow, unless stated otherwise, are conducted under a protective gas atmosphere in dried solvents. The compounds of the invention can be prepared by means of synthesis methods known to those skilled in the art.

### a) (3-Amino-4-chloro-2-benzofuranyl)phenylmethanone

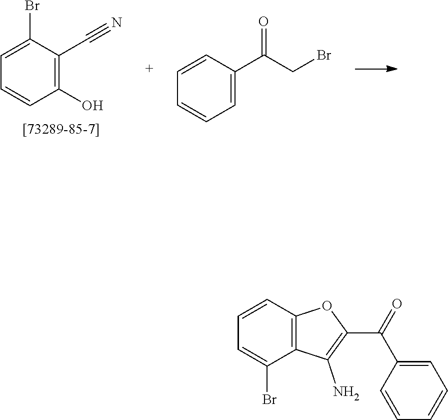 [SMILES: N#Cc1c(O)cccc1Br ; O=C(CBr)c1ccccc1 ; Nc1c(C(=O)c2ccccc2)oc2cccc(Br)c12]

To a solution of 99 g (489 mmol) of 2-bromo-6-hydroxybenzonitrile and 99.5 g (489 mmol) of bromoacetophenone in 790 ml of acetone is added in portions, at room temperature under argon, 319 g (979 mmol) of cesium carbonate.

The reaction mixture is heated to 60° for 2 hours. Subsequently, the mixture is cooled to room temperature, then filtered and then concentrated to dryness under reduced pressure, and recrystallized from heptane.

The yield is 107 g (316 mmol), corresponding to 69% of theory.

In an analogous manner, the following brominated compounds are prepared:

| | Reactant 1 | Reactant 2 | Product | Yield |
|---|---|---|---|---|
| 1a | 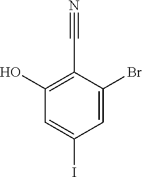 [SMILES: N#Cc1c(O)cc(I)cc1Br] [2091585-53-2] | 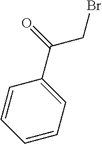 | 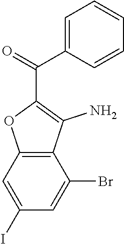 [SMILES: Nc1c(C(=O)c2ccccc2)oc2cc(I)cc(Br)c12] | 66% |
| 2a | 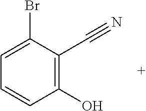 [73289-85-7] | 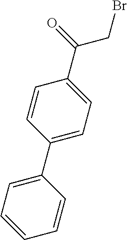 [SMILES: O=C(CBr)c1ccc(-c2ccccc2)cc1] | 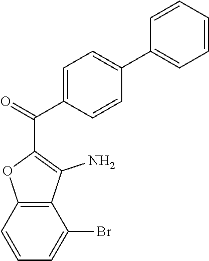 [SMILES: Nc1c(C(=O)c2ccc(-c3ccccc3)cc2)oc2cccc(Br)c12] | 54% |
| 3a | 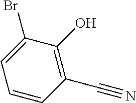 [13073-28-4] | 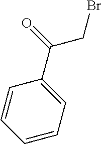 | 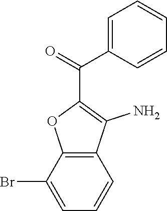 [SMILES: Nc1c(C(=O)c2ccccc2)oc2c(Br)cccc12] | 57% |
| 4a | 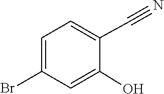 [288067-35-6] | 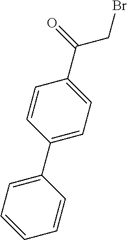 [SMILES: O=C(CBr)c1ccc(-c2ccccc2)cc1] | 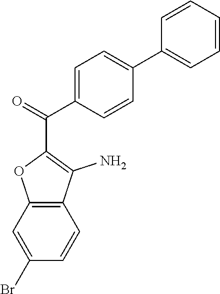 [SMILES: Nc1c(C(=O)c2ccc(-c3ccccc3)cc2)oc2cc(Br)ccc12] | 52% |
| 5a | 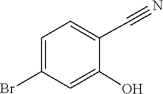 [288067-35-6] | 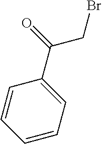 | 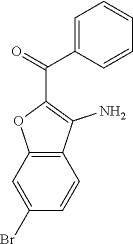 [SMILES: Nc1c(C(=O)c2ccccc2)oc2cc(Br)ccc12] | 57% |

### a) 6-Bromo-2-cyanophenyl Benzoate

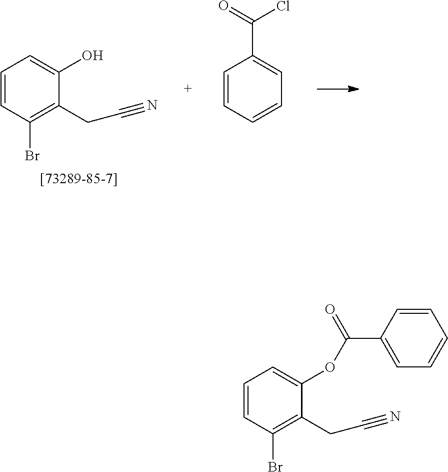 [SMILES: N#CCc1c(O)cccc1Br ; O=C(Cl)c1ccccc1 ; N#CCc1c(Br)cccc1OC(=O)c1ccccc1]

An initially charged solution of 10 g (50 mmol) of 2-bromo-6-hydroxybenzonitrile, 10.4 ml (75 mmol) of triethylamine and 61 mg (0.5 mmol) of 4-N,N-dimethylaminopyridine in 200 ml of CH₂Cl₂ is cooled to 0 °C, and then 10.5 g (75 mmol) of benzoyl chloride is added. The mixture is stirred at room temperature for 3 h. The reaction mixture is poured into 20 ml of sodium chloride solution and extracted three times with Et₂O. The combined organic phase is dried over MgSO₄. The organic solvent is removed under reduced pressure, and the residue is subjected to flash column chromatography on silica gel (hexane/AcOEt=20/1-7/1).

Yield: 10.4 g (33 mmol), 70% of theory.

### b) S-(2-Cyano-3-bromophenyl) benzenecarbothioate

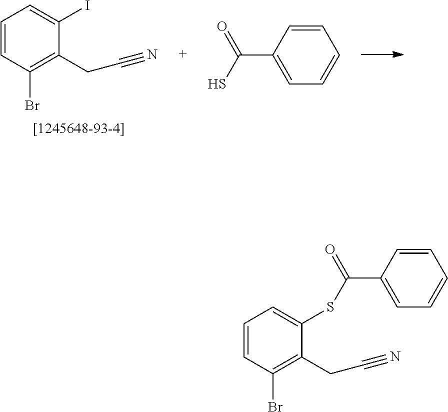 [SMILES: N#CCc1c(Br)cccc1I ; O=C(S)c1ccccc1 ; N#CCc1c(Br)cccc1SC(=O)c1ccccc1]

In a baked-out flask, under argon, 8 g (25 mmol) of 2-bromo-6-iodobenzonitrile, 0.47 g (10 mol %, 2.5 mmol) of CuI, 0.9 (20 mol %, 5 mmol) of 1,10-phenanthroline and 5.1 g (37.5 mmol) of thiobenzoic acid are added under nitrogen to 20 ml of degassed toluene, and the mixture is stirred at 100 °C for 24 h. The reaction mixture is cooled to room temperature. Diethyl ether (1000 ml) and saturated sodium chloride solution (1000 ml) are added, and the mixture is stirred. The organic phase is separated, and the aqueous phase is extracted with diethyl ether (2×1000 ml). The combined organic phases are dried over Na₂SO₄, and the product is isolated by column chromatography.

Yield: 5.3 g (16.2 mmol), 65% of theory

### c) (2-Amino-4-Bromo-3-benzofuranyl)phenylmethanone

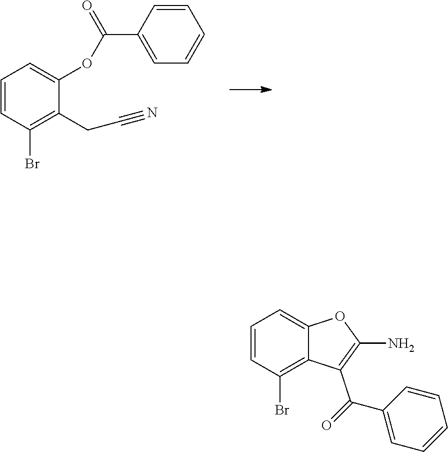 [SMILES: N#CCc1c(Br)cccc1OC(=O)c1ccccc1 ; Nc1oc2cccc(Br)c2c1C(=O)c1ccccc1]

A baked-out flask under argon is initially charged with 0.67 g (30 mmol) of Pd(OAc)₂, 1.69 g (6 mmol) of PCy₃ (tricyclohexylphosphine), 1.96 g (30 mmol) of zinc powder and 3 g of 4 Å molecular sieve (MS4A) in 1200 ml of DMF. After stirring at room temperature for 20 min, 9.4 g (30 mmol) of bromo-2-cyanophenyl benzoate is added and the mixture is then stirred at 100 °C overnight. Then the mixture is admixed with saturated NaCl solution and the aqueous phase is extracted with Et₂O (100 ml×3). The combined organic phases are dried over MgSO₄ and filtered. The organic solvent is removed under reduced pressure, and the residue is purified by flash column chromatography on silica gel (hexane/AcOEt=7/1-2/1).

Yield: 6.2 g (20 mmol), 67% of theory.

In an analogous manner, the following brominated compounds are prepared:

| | Reactant 1 | Product | Yield |
|---|---|---|---|
| 1c | 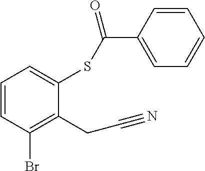 [SMILES: N#CCc1c(Br)cccc1SC(=O)c1ccccc1] | 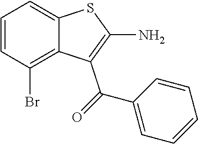 [SMILES: Nc1sc2cccc(Br)c2c1C(=O)c1ccccc1] | 41% |
| 2c | 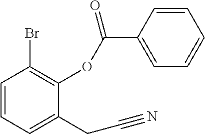 [SMILES: N#CCc1cccc(Br)c1OC(=O)c1ccccc1] | 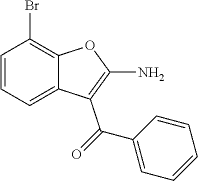 [SMILES: Nc1oc2c(Br)cccc2c1C(=O)c1ccccc1] | 52% |

### d) 9-Bromo-2,4-diphenylbenzofuro[3,2-d]pyrimidine

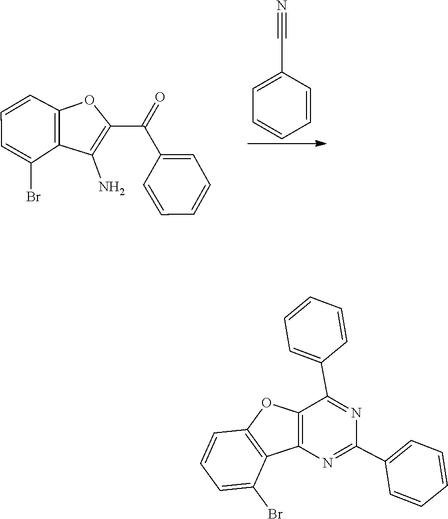 [SMILES: Nc1c(C(=O)c2ccccc2)oc2cccc(Br)c12 ; N#Cc1ccccc1 ; Brc1cccc2oc3c(-c4ccccc4)nc(-c4ccccc4)nc3c12]

To an initial charge under argon of 107 g (316 mmol) of (3-amino-4-chloro-2-benzofuranyl)phenylmethanone and 104 g (1015 mmol) of benzonitrile in 1000 ml of o-xylene is added 56 g (677 mmol) of sodium 2-methylprop-2-oxide. The mixture is stirred at 140 °C for 5 hours. 30 ml is discharged from the water separator, and then a little acetone is added and the mixture is stirred for a further hour. After cooling, the mixture is quenched with one liter of water. The organic phase is separated off, washed three times with 300 ml of water, dried over MgSO₄ and filtered, and the solvent is removed under reduced pressure. The residue is purified by column chromatography.

The yield is 64 g (160 mmol), corresponding to 48% of theory.

The following compounds are prepared in an analogous manner:

| | Reactant 1 | Reactant 2 | Product | Yield |
|---|---|---|---|---|
| 1d | 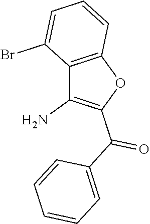 [SMILES: Nc1c(C(=O)c2ccccc2)oc2cccc(Br)c12] | 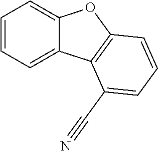 [SMILES: N#Cc1cccc2oc3ccccc3c12] [141104-58-7] | 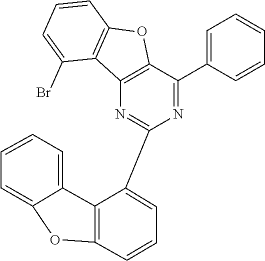 [SMILES: Brc1cccc2oc3c(-c4ccccc4)nc(-c4cccc5oc6ccccc6c45)nc3c12] | 49% |
| 2d | 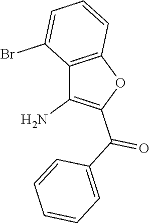 [SMILES: Nc1c(C(=O)c2ccccc2)oc2cccc(Br)c12] | 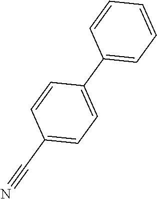 [SMILES: N#Cc1ccc(-c2ccccc2)cc1] [2920-38-9] | 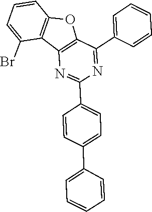 [SMILES: Brc1cccc2oc3c(-c4ccccc4)nc(-c4ccc(-c5ccccc5)cc4)nc3c12] | 42% |
| 3d | 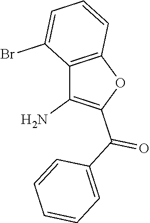 [SMILES: Nc1c(C(=O)c2ccccc2)oc2cccc(Br)c12] | 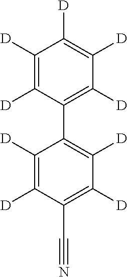 [SMILES: [2H]c1c([2H])c([2H])c(-c2c([2H])c([2H])c(C#N)c([2H])c2[2H])c([2H])c1[2H]] [142475-01-2] | 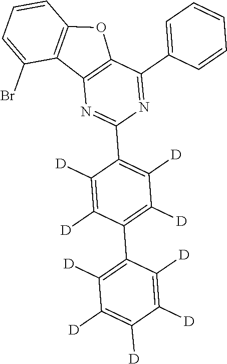 [SMILES: [2H]c1c([2H])c([2H])c(-c2c([2H])c([2H])c(-c3nc(-c4ccccc4)c4oc5cccc(Br)c5c4n3)c([2H])c2[2H])c([2H])c1[2H]] | 41% |
| 4d | 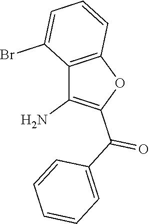 [SMILES: Nc1c(C(=O)c2ccccc2)oc2cccc(Br)c12] | 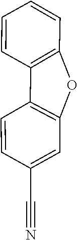 [SMILES: N#Cc1ccc2c(c1)oc1ccccc12] [29021-90-7] | 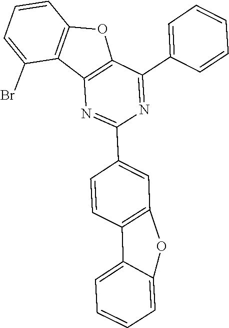 [SMILES: Brc1cccc2oc3c(-c4ccccc4)nc(-c4ccc5c(c4)oc4ccccc45)nc3c12] | 45% |
| 5d | 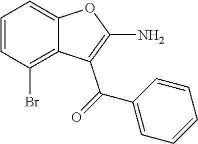 [SMILES: Nc1oc2cccc(Br)c2c1C(=O)c1ccccc1] | 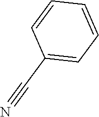 | 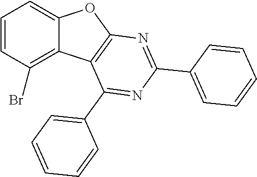 [SMILES: Brc1cccc2oc3nc(-c4ccccc4)nc(-c4ccccc4)c3c12] | 52% |
| 6d | 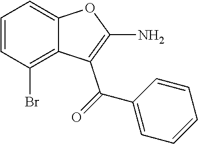 [SMILES: Nc1oc2cccc(Br)c2c1C(=O)c1ccccc1] | 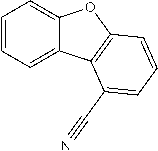 [SMILES: N#Cc1cccc2oc3ccccc3c12] [141104-58-7] | 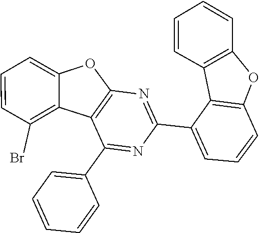 [SMILES: Brc1cccc2oc3nc(-c4cccc5oc6ccccc6c45)nc(-c4ccccc4)c3c12] | 46% |
| 7d | 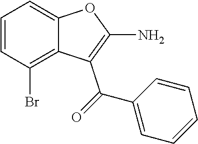 [SMILES: Nc1oc2cccc(Br)c2c1C(=O)c1ccccc1] | 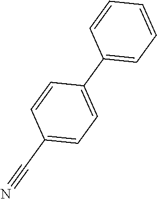 [SMILES: N#Cc1ccc(-c2ccccc2)cc1] [2920-38-9] | 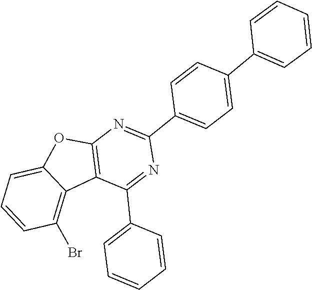 [SMILES: Brc1cccc2oc3nc(-c4ccc(-c5ccccc5)cc4)nc(-c4ccccc4)c3c12] | 40% |
| 8d | 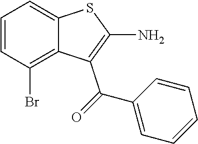 [SMILES: Nc1sc2cccc(Br)c2c1C(=O)c1ccccc1] | 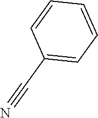 | 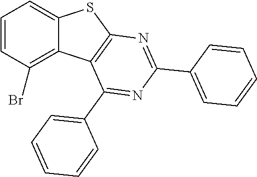 [SMILES: Brc1cccc2sc3nc(-c4ccccc4)nc(-c4ccccc4)c3c12] | 46% |
| 9d | 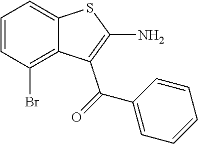 [SMILES: Nc1sc2cccc(Br)c2c1C(=O)c1ccccc1] | 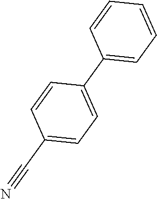 [SMILES: N#Cc1ccc(-c2ccccc2)cc1] [2920-38-9] | 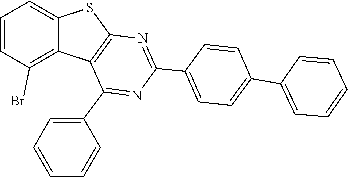 [SMILES: Brc1cccc2sc3nc(-c4ccc(-c5ccccc5)cc4)nc(-c4ccccc4)c3c12] | 47% |
| 10d | 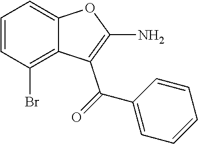 [SMILES: Nc1oc2cccc(Br)c2c1C(=O)c1ccccc1] | 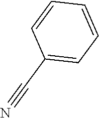 | 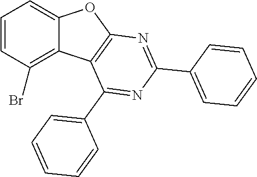 [SMILES: Brc1cccc2oc3nc(-c4ccccc4)nc(-c4ccccc4)c3c12] | 42% |
| 11d | 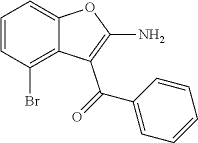 [SMILES: Nc1oc2cccc(Br)c2c1C(=O)c1ccccc1] | 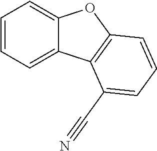 [SMILES: N#Cc1cccc2oc3ccccc3c12] [141104-58-7] | 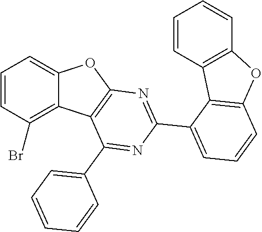 [SMILES: Brc1cccc2oc3nc(-c4cccc5oc6ccccc6c45)nc(-c4ccccc4)c3c12] | 47% |
| 12d | 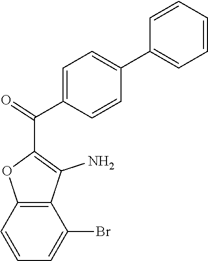 [SMILES: Nc1c(C(=O)c2ccc(-c3ccccc3)cc2)oc2cccc(Br)c12] | 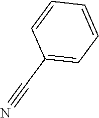 | 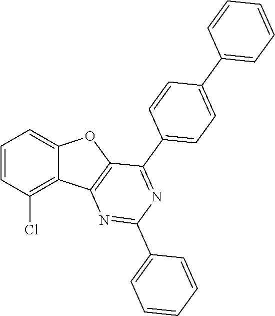 [SMILES: Clc1cccc2oc3c(-c4ccc(-c5ccccc5)cc4)nc(-c4ccccc4)nc3c12] | 43% |
| 13d | 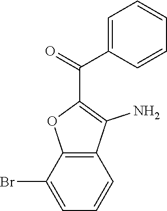 [SMILES: Nc1c(C(=O)c2ccccc2)oc2c(Br)cccc12] | 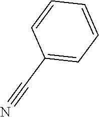 | 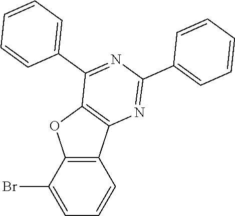 [SMILES: Brc1cccc2c1oc1c(-c3ccccc3)nc(-c3ccccc3)nc12] | 49% |
| 14d | 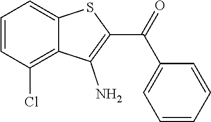 [SMILES: Nc1c(C(=O)c2ccccc2)sc2cccc(Cl)c12] [52673-90-2] | 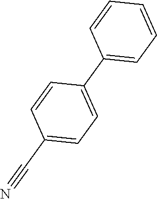 [SMILES: N#Cc1ccc(-c2ccccc2)cc1] [2920-38-9] | 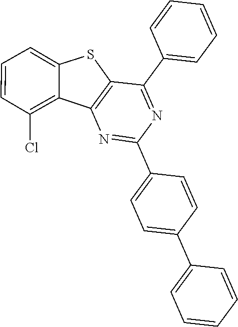 [SMILES: Clc1cccc2sc3c(-c4ccccc4)nc(-c4ccc(-c5ccccc5)cc4)nc3c12] | 43% |
| 15d | 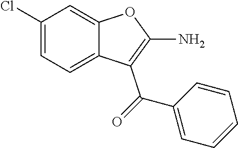 [SMILES: Nc1oc2cc(Cl)ccc2c1C(=O)c1ccccc1] [1777814-10-4] | 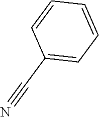 | 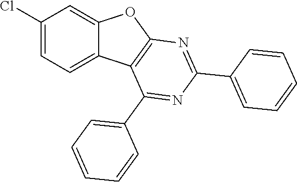 [SMILES: Clc1ccc2c(c1)oc1nc(-c3ccccc3)nc(-c3ccccc3)c12] | 42% |
| 16d | 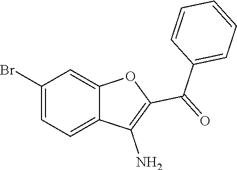 [SMILES: Nc1c(C(=O)c2ccccc2)oc2cc(Br)ccc12] [1251582-17-8] | 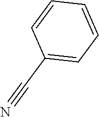 | 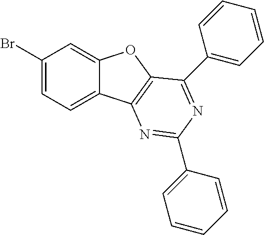 [SMILES: Brc1ccc2c(c1)oc1c(-c3ccccc3)nc(-c3ccccc3)nc12] | 46% |
| 17d | 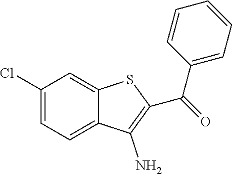 [SMILES: Nc1c(C(=O)c2ccccc2)sc2cc(Cl)ccc12] [2007154-59-6] | 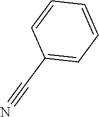 | 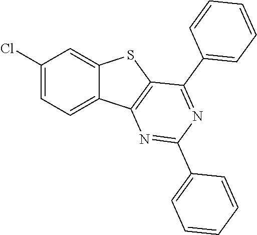 [SMILES: Clc1ccc2c(c1)sc1c(-c3ccccc3)nc(-c3ccccc3)nc12] | 41% |
| 18d | 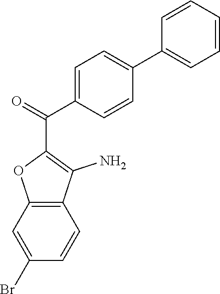 [SMILES: Nc1c(C(=O)c2ccc(-c3ccccc3)cc2)oc2cc(Br)ccc12] | 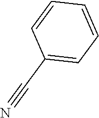 | 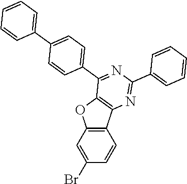 [SMILES: Brc1ccc2c(c1)oc1c(-c3ccc(-c4ccccc4)cc3)nc(-c3ccccc3)nc12] | 45% |
| 19d | 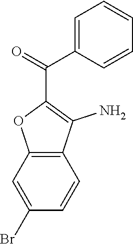 [SMILES: Nc1c(C(=O)c2ccccc2)oc2cc(Br)ccc12] | 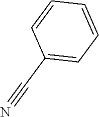 | 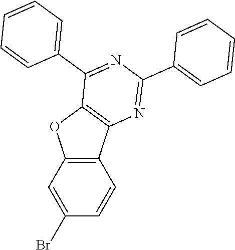 [SMILES: Brc1ccc2c(c1)oc1c(-c3ccccc3)nc(-c3ccccc3)nc12] | 47% |
| 20d | 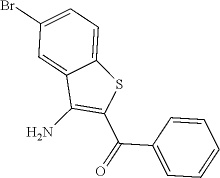 [SMILES: Nc1c(C(=O)c2ccccc2)sc2ccc(Br)cc12] [1678536-27-0] | 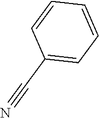 | 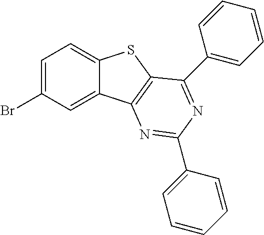 [SMILES: Brc1ccc2sc3c(-c4ccccc4)nc(-c4ccccc4)nc3c2c1] | 51% |

### e) 2-Chloro-4,8-diphenylbenzofuro[3,2-d]pyrimidine

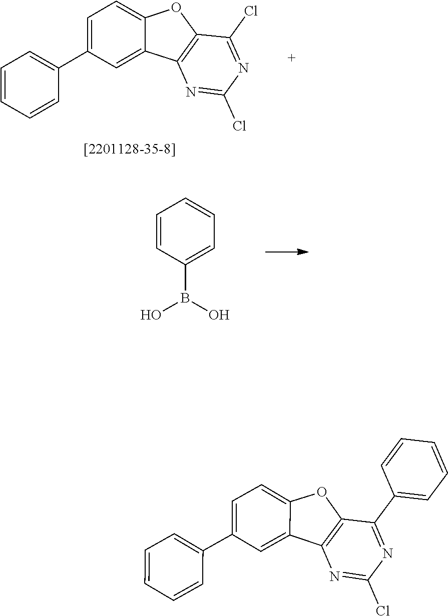 [SMILES: Clc1nc(Cl)c2oc3ccc(-c4ccccc4)cc3c2n1 ; OB(O)c1ccccc1 ; Clc1nc(-c2ccccc2)c2oc3ccc(-c4ccccc4)cc3c2n1]

31.4 (100 mmol) of 2,4-dichloro-8-phenylbenzofuro[3,2-d]pyrimidine, 12.2 g (100 mmol) of phenylboronic acid and 11.8 g (111 mmol) of sodium carbonate are dissolved in 800 ml of 1,4-dioxane, 800 ml of water and 250 ml of toluene, and stirred under an argon atmosphere. 1.2 g (1 mmol) of tetrakis(triphenylphosphine) palladium is added to the flask. The reaction mixture is stirred under reflux overnight. After cooling, the mixture is quenched. The organic phase is separated, washed three times with 300 ml of water, dried over MgSO₄ and filtered, and the solvent is removed under reduced pressure. The residue is purified by column chromatography using silica gel (eluent: DCM/heptane (1:10)). The yield is 28 g (80 mmol), corresponding to 80% of theory.

The following compounds are prepared in an analogous manner:

| No. | Reactant 1 | Reactant 1 | Product | Yield |
|---|---|---|---|---|
| 1e | 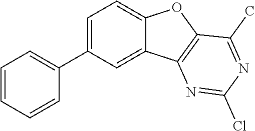 [SMILES: Cc1nc(Cl)nc2c1oc1ccc(-c3ccccc3)cc12] [1835207-37-8] | 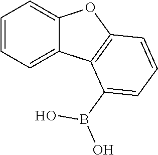 [SMILES: OB(O)c1cccc2oc3ccccc3c12] [162607-19-4] | 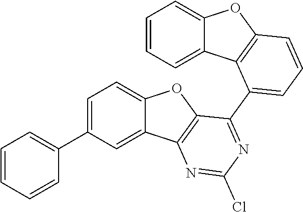 [SMILES: Clc1nc(-c2cccc3oc4ccccc4c23)c2oc3ccc(-c4ccccc4)cc3c2n1] | 75% |
| 2e | 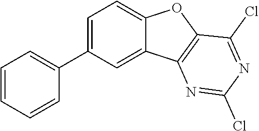 [SMILES: Clc1nc(Cl)c2oc3ccc(-c4ccccc4)cc3c2n1] [1835207-37-8] | 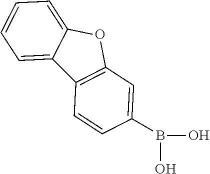 [SMILES: OB(O)c1ccc2c(c1)oc1ccccc12] [395087-89-5] | 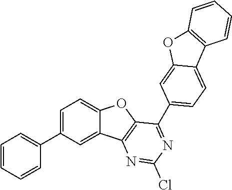 [SMILES: Clc1nc(-c2ccc3c(c2)oc2ccccc23)c2oc3ccc(-c4ccccc4)cc3c2n1] | 73% |
| 3e | 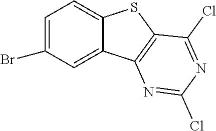 [SMILES: Clc1nc(Cl)c2sc3ccc(Br)cc3c2n1] [2201128-35-8] | 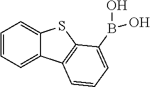 [SMILES: OB(O)c1cccc2c1sc1ccccc12] [108847-20-7] | 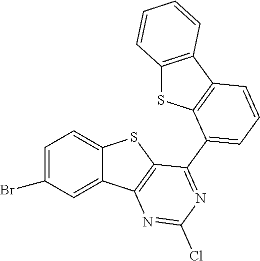 [SMILES: Clc1nc(-c2cccc3c2sc2ccccc23)c2sc3ccc(Br)cc3c2n1] | 67% |
| 4e | 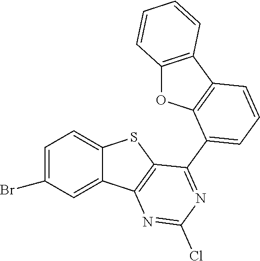 [SMILES: Clc1nc(-c2cccc3c2oc2ccccc23)c2sc3ccc(Br)cc3c2n1] | 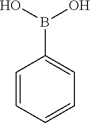 | 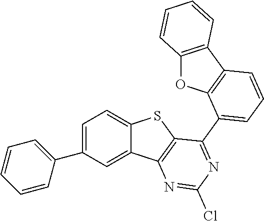 [SMILES: Clc1nc(-c2cccc3c2oc2ccccc23)c2sc3ccc(-c4ccccc4)cc3c2n1] | 80% |
| 5e | 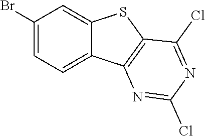 [SMILES: Clc1nc(Cl)c2sc3cc(Br)ccc3c2n1] [2201128-36-9] | 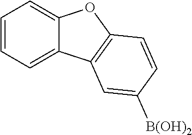 [SMILES: OB(O)c1ccc2oc3ccccc3c2c1] [402936-15-6] | 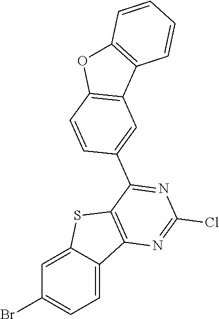 [SMILES: Clc1nc(-c2ccc3oc4ccccc4c3c2)c2sc3cc(Br)ccc3c2n1] | 73% |
| 6e | 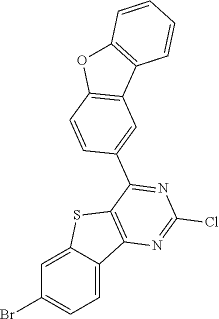 [SMILES: Clc1nc(-c2ccc3oc4ccccc4c3c2)c2sc3cc(Br)ccc3c2n1] | 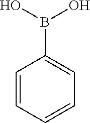 | 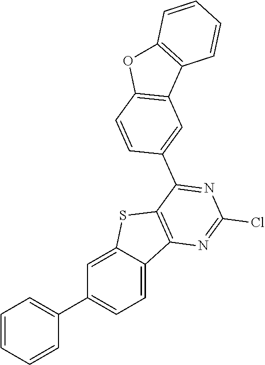 [SMILES: Clc1nc(-c2ccc3oc4ccccc4c3c2)c2sc3cc(-c4ccccc4)ccc3c2n1] | 86% |
| 7e | 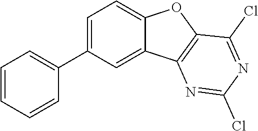 [SMILES: Clc1nc(Cl)c2oc3ccc(-c4ccccc4)cc3c2n1] [1835207-37-8] | 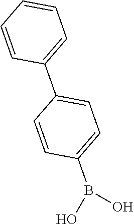 [SMILES: OB(O)c1ccc(-c2ccccc2)cc1] [5122-94-1] | 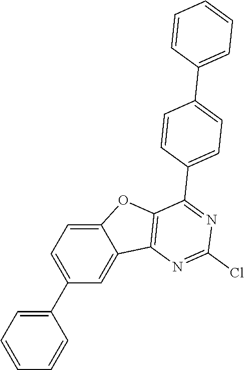 [SMILES: Clc1nc(-c2ccc(-c3ccccc3)cc2)c2oc3ccc(-c4ccccc4)cc3c2n1] | 84% |
| 8e | 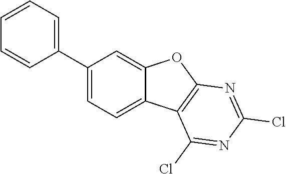 [SMILES: Clc1nc(Cl)c2c(n1)oc1cc(-c3ccccc3)ccc12] [2201128-28-9] | 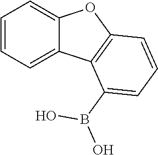 [SMILES: OB(O)c1cccc2oc3ccccc3c12] [162607-19-4] | 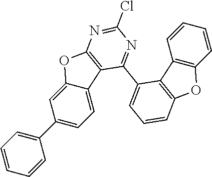 [SMILES: Clc1nc(-c2cccc3oc4ccccc4c23)c2c(n1)oc1cc(-c3ccccc3)ccc12] | 76% |
| 9e | 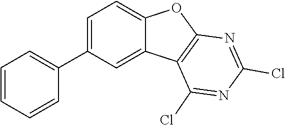 [SMILES: Clc1nc(Cl)c2c(n1)oc1ccc(-c3ccccc3)cc12] [2201128-33-6] | 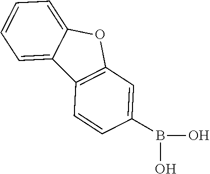 [SMILES: OB(O)c1ccc2c(c1)oc1ccccc12] [395087-89-5] | 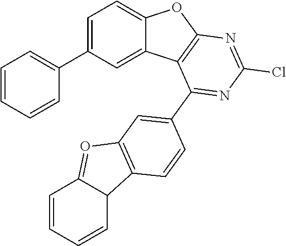 | 73% |
| 10e | 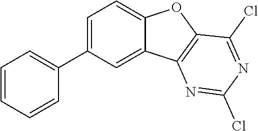 [SMILES: Clc1nc(Cl)c2oc3ccc(-c4ccccc4)cc3c2n1] [1835207-37-8] | 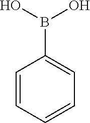 | 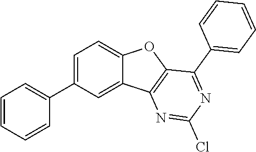 [SMILES: Clc1nc(-c2ccccc2)c2oc3ccc(-c4ccccc4)cc3c2n1] | 82% |
| 11e | 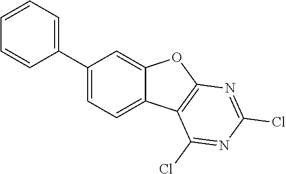 [SMILES: Clc1nc(Cl)c2c(n1)oc1cc(-c3ccccc3)ccc12] [2201128-28-9] | 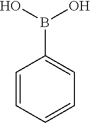 | 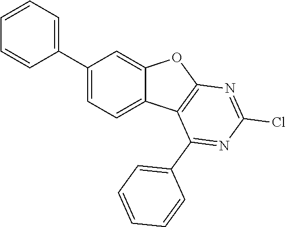 [SMILES: Clc1nc(-c2ccccc2)c2c(n1)oc1cc(-c3ccccc3)ccc12] | 79% |
| 12e | 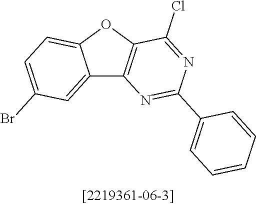 [SMILES: Clc1nc(-c2ccccc2)nc2c1oc1ccc(Br)cc12] [2219361-06-3] | 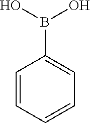 | 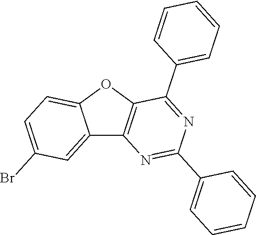 [SMILES: Brc1ccc2oc3c(-c4ccccc4)nc(-c4ccccc4)nc3c2c1] | 86% |
| 13e | 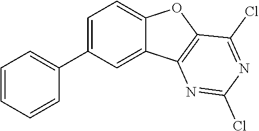 [SMILES: Clc1nc(Cl)c2oc3ccc(-c4ccccc4)cc3c2n1] [1835207-37-8] | 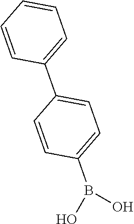 [SMILES: OB(O)c1ccc(-c2ccccc2)cc1] [5122-94-1] | 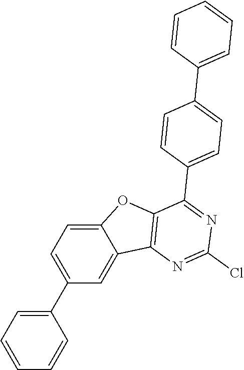 [SMILES: Clc1nc(-c2ccc(-c3ccccc3)cc2)c2oc3ccc(-c4ccccc4)cc3c2n1] | 84% |
| 14e | 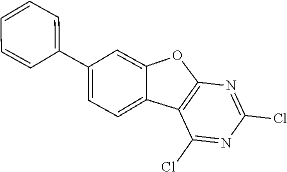 [SMILES: Clc1nc(Cl)c2c(n1)oc1cc(-c3ccccc3)ccc12] [2201128-28-9] | 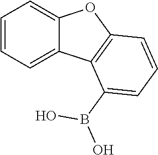 [SMILES: OB(O)c1cccc2oc3ccccc3c12] [162607-19-4] | 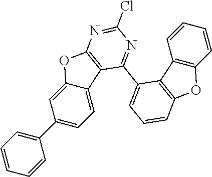 [SMILES: Clc1nc(-c2cccc3oc4ccccc4c23)c2c(n1)oc1cc(-c3ccccc3)ccc12] | 76% |
| 15e | 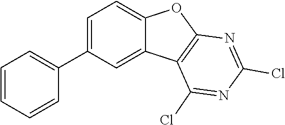 [SMILES: Clc1nc(Cl)c2c(n1)oc1ccc(-c3ccccc3)cc12] [2201128-33-6] | 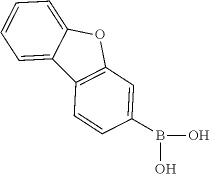 [SMILES: OB(O)c1ccc2c(c1)oc1ccccc12] [395087-89-5] | 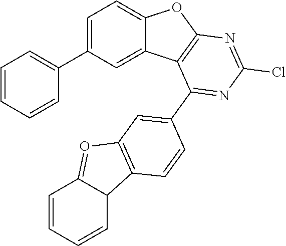 | 73% |
| 16e | 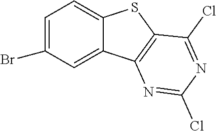 [SMILES: Clc1nc(Cl)c2sc3ccc(Br)cc3c2n1] [2201128-35-8] | 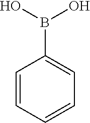 | 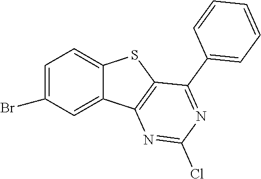 [SMILES: Clc1nc(-c2ccccc2)c2sc3ccc(Br)cc3c2n1] | 77% |
| 17e | 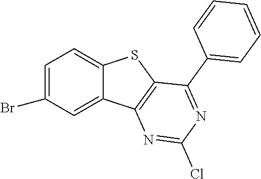 [SMILES: Clc1nc(-c2ccccc2)c2sc3ccc(Br)cc3c2n1] | 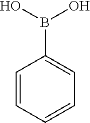 | 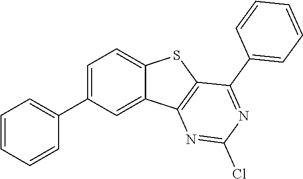 [SMILES: Clc1nc(-c2ccccc2)c2sc3ccc(-c4ccccc4)cc3c2n1] | 81% |
| 18e | 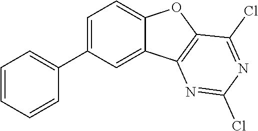 [SMILES: Clc1nc(Cl)c2oc3ccc(-c4ccccc4)cc3c2n1] [1835207-37-8] | 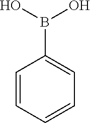 | 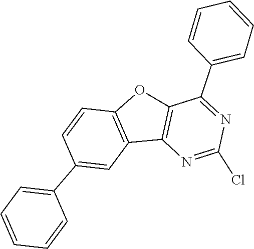 [SMILES: Clc1nc(-c2ccccc2)c2oc3ccc(-c4ccccc4)cc3c2n1] | 65% |
| 19e | 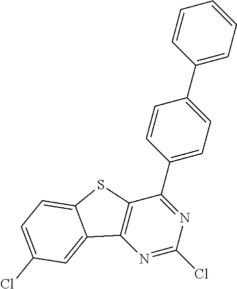 [SMILES: Clc1ccc2sc3c(-c4ccc(-c5ccccc5)cc4)nc(Cl)nc3c2c1] [2376887-11-3] | 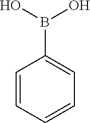 | 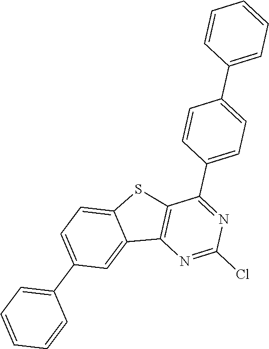 [SMILES: Clc1nc(-c2ccc(-c3ccccc3)cc2)c2sc3ccc(-c4ccccc4)cc3c2n1] | 41% |
| 20e | 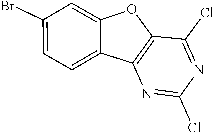 [SMILES: Clc1nc(Cl)c2oc3cc(Br)ccc3c2n1] 2201128-36-9 | 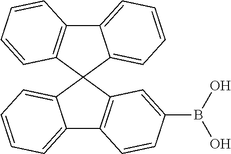 [SMILES: OB(O)c1ccc2c(c1)C1(c3ccccc3-c3ccccc31)c1ccccc1-2] [236389-21-2] | 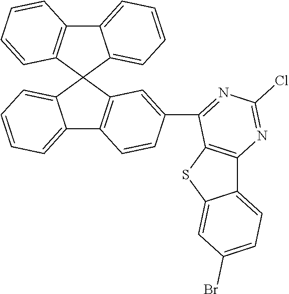 [SMILES: Clc1nc(-c2ccc3c(c2)C2(c4ccccc4-c4ccccc42)c2ccccc2-3)c2sc3cc(Br)ccc3c2n1] | 68% |
| 21e | 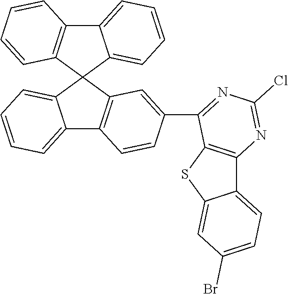 [SMILES: Clc1nc(-c2ccc3c(c2)C2(c4ccccc4-c4ccccc42)c2ccccc2-3)c2sc3cc(Br)ccc3c2n1] | 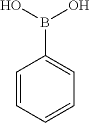 | 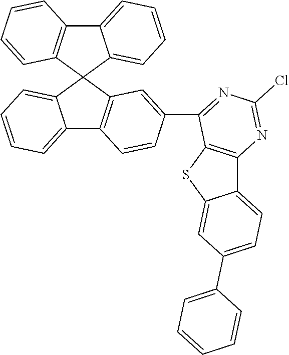 [SMILES: Clc1nc(-c2ccc3c(c2)C2(c4ccccc4-c4ccccc42)c2ccccc2-3)c2sc3cc(-c4ccccc4)ccc3c2n1] | 79% |
| 22e | 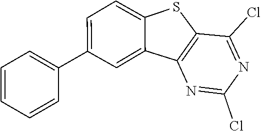 [SMILES: Clc1nc(Cl)c2sc3ccc(-c4ccccc4)cc3c2n1] 2201127-92-4 | 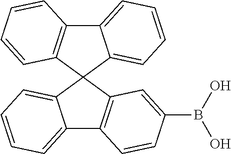 [SMILES: OB(O)c1ccc2c(c1)C1(c3ccccc3-c3ccccc31)c1ccccc1-2] [236389-21-2] | 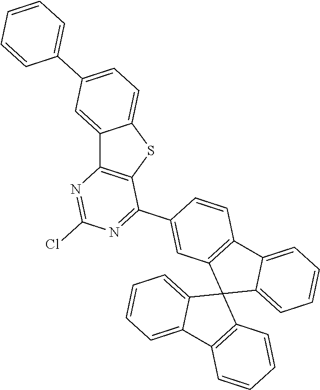 [SMILES: Clc1nc(-c2ccc3c(c2)C2(c4ccccc4-c4ccccc42)c2ccccc2-3)c2sc3ccc(-c4ccccc4)cc3c2n1] | 85% |
| 23e | 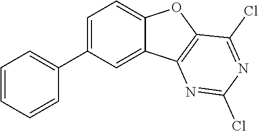 [SMILES: Clc1nc(Cl)c2oc3ccc(-c4ccccc4)cc3c2n1] [1835207-37-8] | 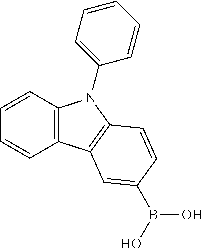 [SMILES: OB(O)c1ccc2c(c1)c1ccccc1n2-c1ccccc1] | 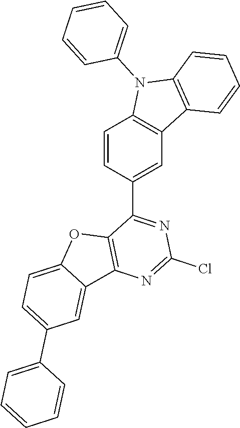 [SMILES: Clc1nc(-c2ccc3c(c2)c2ccccc2n3-c2ccccc2)c2oc3ccc(-c4ccccc4)cc3c2n1] | 57% |
| 24e | 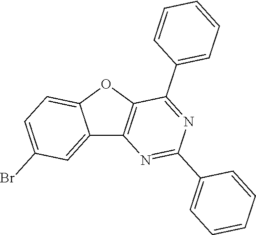 [SMILES: Brc1ccc2oc3c(-c4ccccc4)nc(-c4ccccc4)nc3c2c1] | 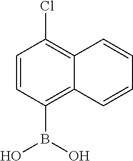 [SMILES: OB(O)c1ccc(Cl)c2ccccc12] | 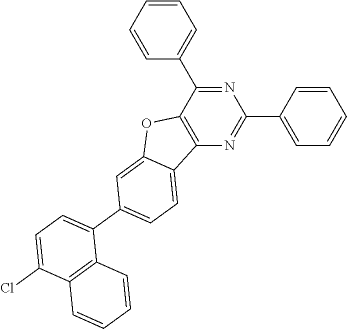 [SMILES: Clc1ccc(-c2ccc3c(c2)oc2c(-c4ccccc4)nc(-c4ccccc4)nc23)c2ccccc12] | 46% |
| 25e | 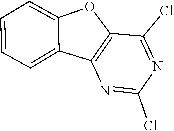 [SMILES: Clc1nc(Cl)c2oc3ccccc3c2n1] [160199-95-1] | 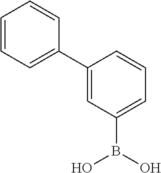 [SMILES: OB(O)c1cccc(-c2ccccc2)c1] | 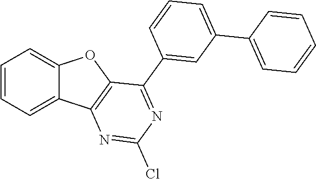 [SMILES: Clc1nc(-c2cccc(-c3ccccc3)c2)c2oc3ccccc3c2n1] | 56% |

### f) 2-Phenyl-4H-naphtho[1,2,3,4-def]carbazole

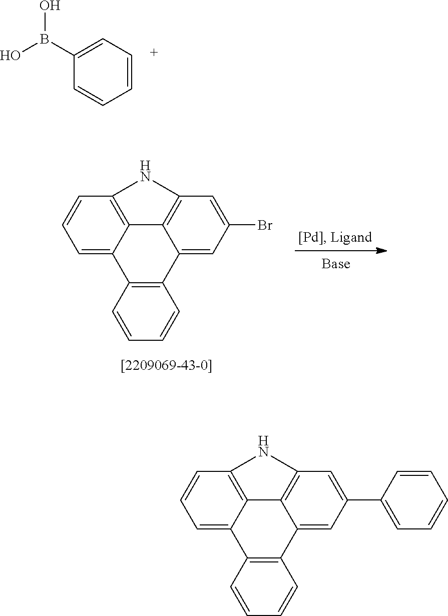 [SMILES: OB(O)c1ccccc1 ; Brc1cc2[nH]c3cccc4c5ccccc5c(c1)c2c34 ; c1ccc(-c2cc3[nH]c4cccc5c6ccccc6c(c2)c3c45)cc1]

25.6 g (210 mmol; 1.00 eq.) of phenylboronic acid, 81 g (255 mmol; 1.21 eq.) of 2-phenyl-4H-naphtho[1,2,3,4-def]carbazole and 44.5 g (420 mmol, 2.00 eq.) of sodium carbonate [CAS 497-19-8] are suspended in a mixture of 1000 ml of dioxane [CAS 123-91-1], 1000 ml of toluene [CAS 108-88-3] and 400 ml of water. To this suspension is added 4.85 g (4.20 mmol; 0.02 eq.) of tetrakis(triphenylphosphine)palladium(0) [CAS 14221-01-3], and the reaction mixture is heated under reflux for 16 h. After cooling, the organic phase is removed, filtered through silica gel, washed three times with 200 ml of water and then concentrated to dryness. The yield is 38 g (121 mmol; 79% of theory).

The following compounds can be obtained analogously:

| No. | Reactant 1 | Product | Yield |
|---|---|---|---|
| 1f | 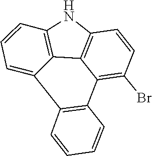 [SMILES: Brc1ccc2[nH]c3cccc4c5ccccc5c1c2c34] [2637505-49-6] | 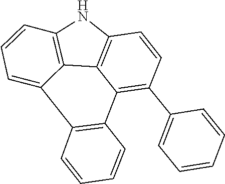 [SMILES: c1ccc(-c2ccc3[nH]c4cccc5c6ccccc6c2c3c45)cc1] | 81% |
| 2f | 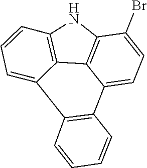 [SMILES: Brc1ccc2c3ccccc3c3cccc4[nH]c1c2c43] [2209068-40-4] | 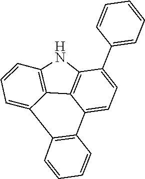 [SMILES: c1ccc(-c2ccc3c4ccccc4c4cccc5[nH]c2c3c54)cc1] | 63% |
| 3f | 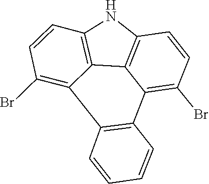 [SMILES: Brc1ccc2[nH]c3ccc(Br)c4c5ccccc5c1c2c34] | 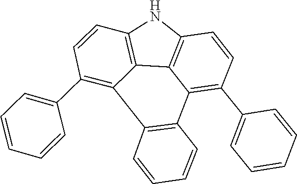 [SMILES: c1ccc(-c2ccc3[nH]c4ccc(-c5ccccc5)c5c6ccccc6c2c3c45)cc1] | 57% |

### g) 4-(3-Bromophenyl)-4H-naphtho[1,2,3,4-def]carbazole

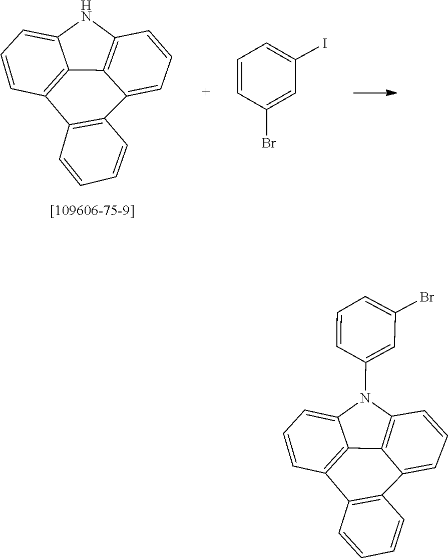 [SMILES: c1ccc2c(c1)c1cccc3[nH]c4cccc2c4c31 ; Brc1cccc(I)c1 ; Brc1cccc(-n2c3cccc4c5ccccc5c5cccc2c5c43)c1]

19.5 g (81 mmol) of 4H-naphtho[1,2,3,4-def]carbazole, 108 g (380 mmol) of 1-bromo-3-iodobenzene, 22.4 g (162 mmol) of potassium carbonate, 1.84 g (8.1 mmol) of 1,3-di(2-pyridyl)propane-1,3-dione, 1.55 g (8.1 mmol) of copper iodide and 1000 ml of DMF are heated under reflux for 30 h. This is followed by concentration to dryness on a rotary evaporator. The residue is dissolved in THF and filtered through a short silica gel bed. The solvent is then removed by means of reduced pressure. Subsequently, the solids are recrystallized from heptane/THF and then subjected to hot extraction with heptane/toluene over alumina. The solid material that precipitates out in the course of cooling is filtered and dried.

Yield: 28 g (71 mmol), 88%.

The following compounds can be prepared analogously:

| No. | Reactant 1 | Reactant 2 | Product | Yield |
|---|---|---|---|---|
| 1g | 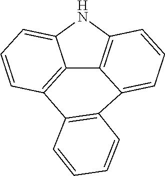 [SMILES: c1ccc2c(c1)c1cccc3[nH]c4cccc2c4c31] [109606-75-9] | 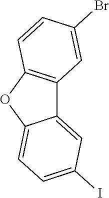 [SMILES: Brc1ccc2oc3ccc(I)cc3c2c1] [916435-41-1] | 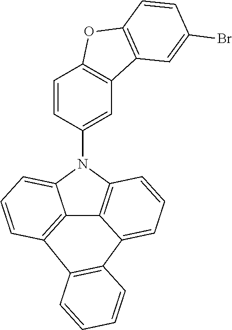 [SMILES: Brc1ccc2oc3ccc(-n4c5cccc6c7ccccc7c7cccc4c7c65)cc3c2c1] | 82% |
| 2g | 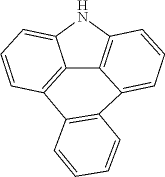 [SMILES: c1ccc2c(c1)c1cccc3[nH]c4cccc2c4c31] [109606-75-9] | 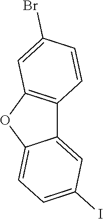 [SMILES: Brc1ccc2c(c1)oc1ccc(I)cc12] [1627589-28-9] | 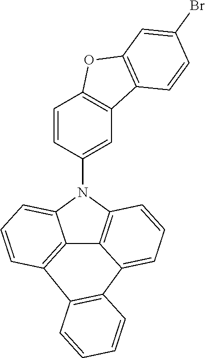 [SMILES: Brc1ccc2c(c1)oc1ccc(-n3c4cccc5c6ccccc6c6cccc3c6c54)cc12] | 80% |
| 3g | 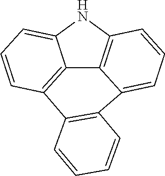 [SMILES: c1ccc2c(c1)c1cccc3[nH]c4cccc2c4c31] [109606-75-9] | 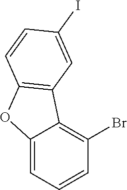 [SMILES: Brc1cccc2oc3ccc(I)cc3c12] [1822311-11-4] | 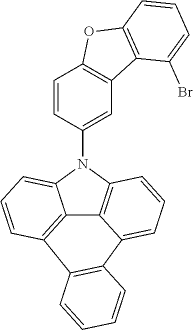 [SMILES: Brc1cccc2oc3ccc(-n4c5cccc6c7ccccc7c7cccc4c7c65)cc3c12] | 84% |
| 4g | 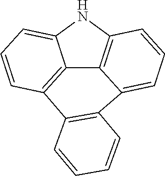 [SMILES: c1ccc2c(c1)c1cccc3[nH]c4cccc2c4c31] [109606-75-9] | 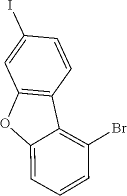 [SMILES: Brc1cccc2oc3cc(I)ccc3c12] [2244710-46-9] | 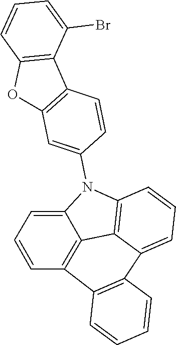 [SMILES: Brc1cccc2oc3cc(-n4c5cccc6c7ccccc7c7cccc4c7c65)ccc3c12] | 86% |
| 5g | 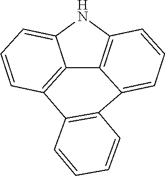 [SMILES: c1ccc2c(c1)c1cccc3[nH]c4cccc2c4c31] [109606-75-9] | 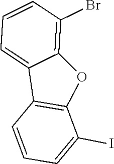 [SMILES: Brc1cccc2c1oc1c(I)cccc12] [1110690-60-2] | 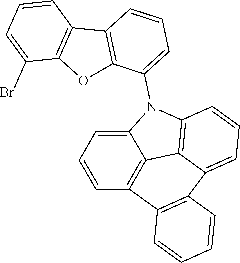 [SMILES: Brc1cccc2c1oc1c(-n3c4cccc5c6ccccc6c6cccc3c6c54)cccc12] | 79% |
| 6g | 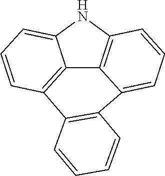 [SMILES: c1ccc2c(c1)c1cccc3[nH]c4cccc2c4c31] [109606-75-9] | 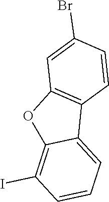 [SMILES: Brc1ccc2c(c1)oc1c(I)cccc12] [2361171-48-2] | 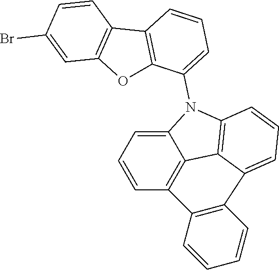 [SMILES: Brc1ccc2c(c1)oc1c(-n3c4cccc5c6ccccc6c6cccc3c6c54)cccc12] | 81% |
| 7g | 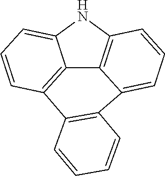 [SMILES: c1ccc2c(c1)c1cccc3[nH]c4cccc2c4c31] [109606-75-9] | 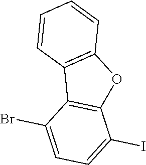 [SMILES: Brc1ccc(I)c2oc3ccccc3c12] [1923736-35-9] | 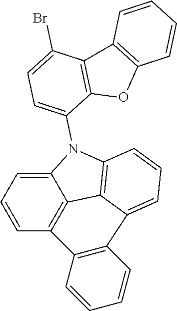 [SMILES: Brc1ccc(-n2c3cccc4c5ccccc5c5cccc2c5c43)c2oc3ccccc3c12] | 77% |
| 8g | 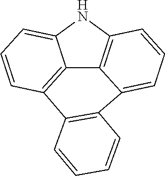 [SMILES: c1ccc2c(c1)c1cccc3[nH]c4cccc2c4c31] [109606-75-9] | 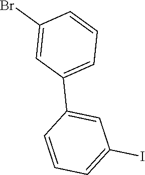 [SMILES: Brc1cccc(-c2cccc(I)c2)c1] [187275-76-9] | 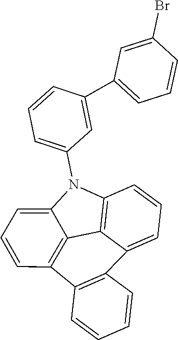 [SMILES: Brc1cccc(-c2cccc(-n3c4cccc5c6ccccc6c6cccc3c6c54)c2)c1] | 84% |
| 9g | 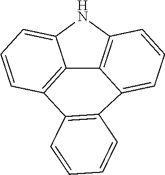 [SMILES: c1ccc2c(c1)c1cccc3[nH]c4cccc2c4c31] [109606-75-9] | 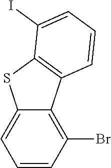 [SMILES: Brc1cccc2sc3c(I)cccc3c12] [2361168-70-7] | 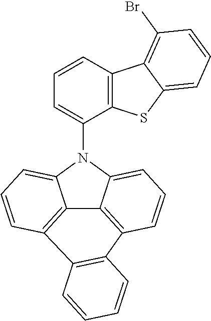 [SMILES: Brc1cccc2sc3c(-n4c5cccc6c7ccccc7c7cccc4c7c65)cccc3c12] [109606-75-9] | 76% |
| 10g | 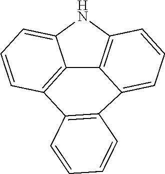 [SMILES: c1ccc2c(c1)c1cccc3[nH]c4cccc2c4c31] [109606-75-9] | 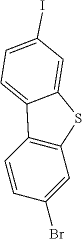 [SMILES: Brc1ccc2c(c1)sc1cc(I)ccc12] [1448787-84-5] | 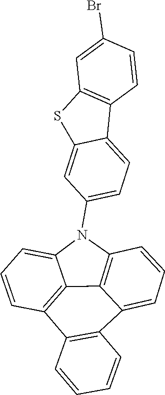 [SMILES: Brc1ccc2c(c1)sc1cc(-n3c4cccc5c6ccccc6c6cccc3c6c54)ccc12] | 79% |
| 11g | 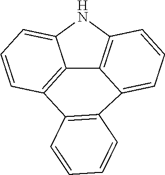 [SMILES: c1ccc2c(c1)c1cccc3[nH]c4cccc2c4c31] [109606-75-9] | 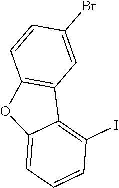 [SMILES: Brc1ccc2oc3cccc(I)c3c2c1] [2098866-56-7] | 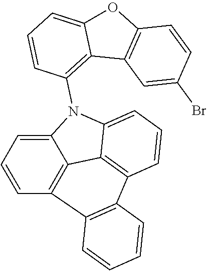 [SMILES: Brc1ccc2oc3cccc(-n4c5cccc6c7ccccc7c7cccc4c7c65)c3c2c1] | 75% |
| 12g | 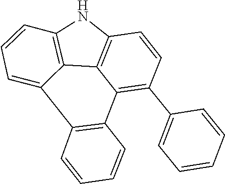 [SMILES: c1ccc(-c2ccc3[nH]c4cccc5c6ccccc6c2c3c45)cc1] | 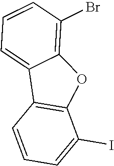 [SMILES: Brc1cccc2c1oc1c(I)cccc12] [1110690-60-2] | 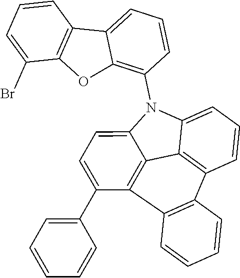 [SMILES: Brc1cccc2c1oc1c(-n3c4cccc5c6ccccc6c6c(-c7ccccc7)ccc3c6c54)cccc12] | 73% |

### h) 4H-Naphtho[1,2,3,4-def]carbazole-3-phenylboronic Acid

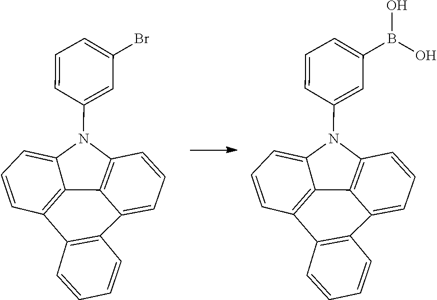 [SMILES: Brc1cccc(-n2c3cccc4c5ccccc5c5cccc2c5c43)c1 ; OB(O)c1cccc(-n2c3cccc4c5ccccc5c5cccc2c5c43)c1]

To a solution, cooled to −78 °C, of 51.3 g (130 mmol) of 4-(3-bromophenyl)-4H-naphtho[1,2,3,4-def]carbazole in 1500 ml of THF is added dropwise 55 ml (138 mmol) of n-butyllithium (2.5 M in hexane). The reaction mixture is stirred at −78 °C for 30 min. The mixture is allowed to come to room temperature and cooled again to −78 °C, and then a mixture of 20 ml (176 mmol) of trimethyl borate in 50 ml of THF is added rapidly. After warming to −10 °C, hydrolysis is effected with 135 ml of 2 N hydrochloric acid. The organic phase is removed, washed with water, dried over sodium sulphate and concentrated to dryness. The residue is taken up in 300 ml of n-heptane, and the colourless solid is filtered off with suction, washed with n-heptane and dried under reduced pressure. Yield: 45 g (126 mmol); 97% of theory; purity: 99% by HPLC.

The following compounds can be prepared analogously:

| No. | Reactant 1 | Product | Yield |
|---|---|---|---|
| 1h | 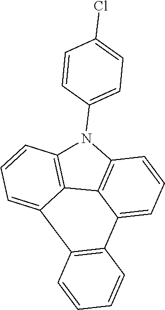 [SMILES: Clc1ccc(-n2c3cccc4c5ccccc5c5cccc2c5c43)cc1] [2871765-18-1] | 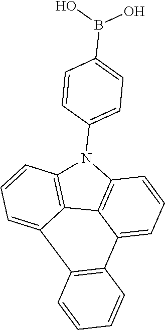 [SMILES: OB(O)c1ccc(-n2c3cccc4c5ccccc5c5cccc2c5c43)cc1] | 80% |
| 2h | 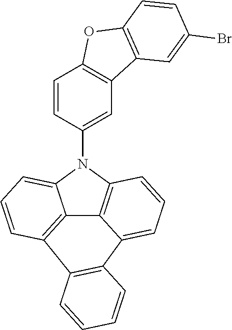 [SMILES: Brc1ccc2oc3ccc(-n4c5cccc6c7ccccc7c7cccc4c7c65)cc3c2c1] | 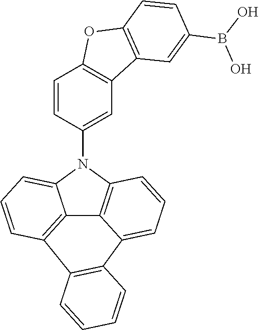 [SMILES: OB(O)c1ccc2oc3ccc(-n4c5cccc6c7ccccc7c7cccc4c7c65)cc3c2c1] | 76% |
| 3h | 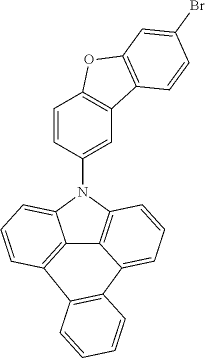 [SMILES: Brc1ccc2c(c1)oc1ccc(-n3c4cccc5c6ccccc6c6cccc3c6c54)cc12] | 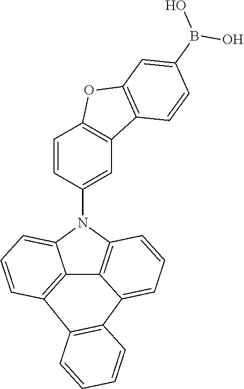 [SMILES: OB(O)c1ccc2c(c1)oc1ccc(-n3c4cccc5c6ccccc6c6cccc3c6c54)cc12] | 80% |
| 4h | 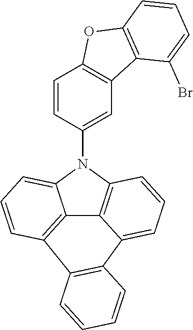 [SMILES: Brc1cccc2oc3ccc(-n4c5cccc6c7ccccc7c7cccc4c7c65)cc3c12] | 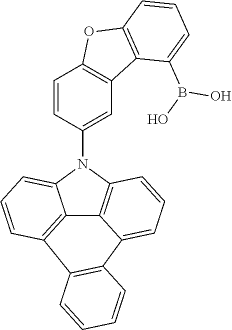 [SMILES: OB(O)c1cccc2oc3ccc(-n4c5cccc6c7ccccc7c7cccc4c7c65)cc3c12] | 79% |
| 5h | 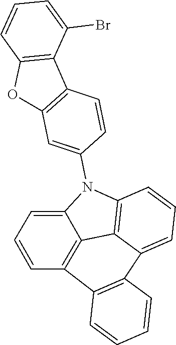 [SMILES: Brc1cccc2oc3cc(-n4c5cccc6c7ccccc7c7cccc4c7c65)ccc3c12] | 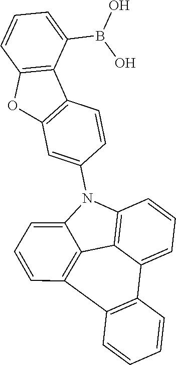 [SMILES: OB(O)c1cccc2oc3cc(-n4c5cccc6c7ccccc7c7cccc4c7c65)ccc3c12] | 77% |
| 6h | 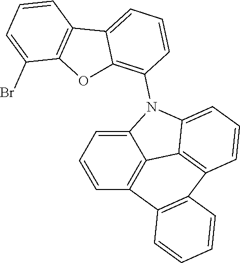 [SMILES: Brc1cccc2c1oc1c(-n3c4cccc5c6ccccc6c6cccc3c6c54)cccc12] | 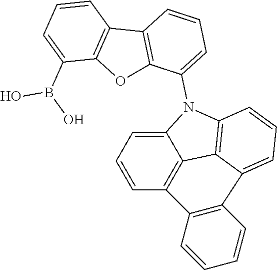 [SMILES: OB(O)c1cccc2c1oc1c(-n3c4cccc5c6ccccc6c6cccc3c6c54)cccc12] | 74% |
| 7h | 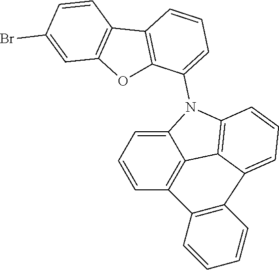 [SMILES: Brc1ccc2c(c1)oc1c(-n3c4cccc5c6ccccc6c6cccc3c6c54)cccc12] | 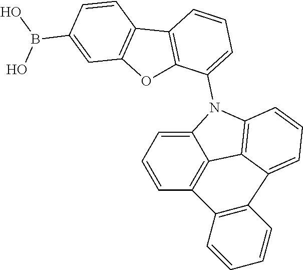 [SMILES: OB(O)c1ccc2c(c1)oc1c(-n3c4cccc5c6ccccc6c6cccc3c6c54)cccc12] | 81% |
| 8h | 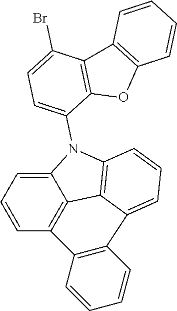 [SMILES: Brc1ccc(-n2c3cccc4c5ccccc5c5cccc2c5c43)c2oc3ccccc3c12] | 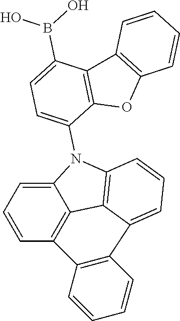 [SMILES: OB(O)c1ccc(-n2c3cccc4c5ccccc5c5cccc2c5c43)c2oc3ccccc3c12] | 72% |
| 9h | 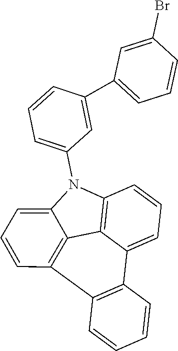 [SMILES: Brc1cccc(-c2cccc(-n3c4cccc5c6ccccc6c6cccc3c6c54)c2)c1] | 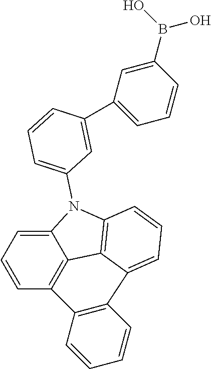 [SMILES: OB(O)c1cccc(-c2cccc(-n3c4cccc5c6ccccc6c6cccc3c6c54)c2)c1] | 84% |
| 10h | 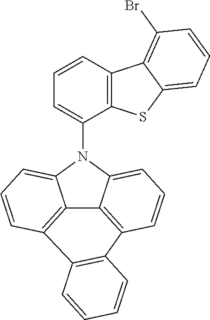 [SMILES: Brc1cccc2sc3c(-n4c5cccc6c7ccccc7c7cccc4c7c65)cccc3c12] [109606-75-9] | 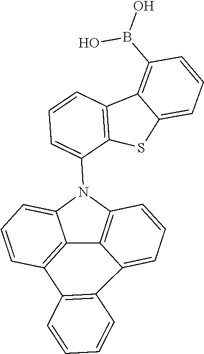 [SMILES: OB(O)c1cccc2sc3c(-n4c5cccc6c7ccccc7c7cccc4c7c65)cccc3c12] [109606-75-9] | 70% |
| 12h | 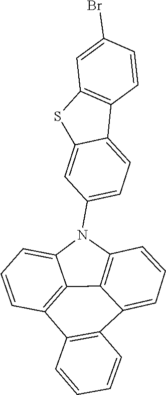 [SMILES: Brc1ccc2c(c1)sc1cc(-n3c4cccc5c6ccccc6c6cccc3c6c54)ccc12] | 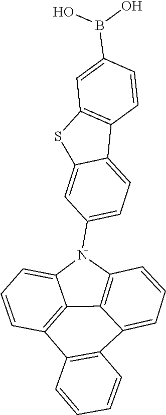 [SMILES: OB(O)c1ccc2c(c1)sc1cc(-n3c4cccc5c6ccccc6c6cccc3c6c54)ccc12] | 78% |
| 13h | 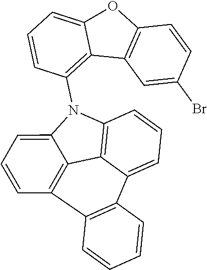 [SMILES: Brc1ccc2oc3cccc(-n4c5cccc6c7ccccc7c7cccc4c7c65)c3c2c1] | 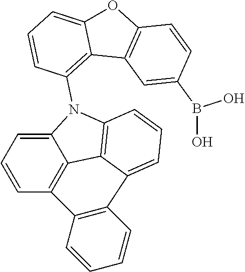 [SMILES: OB(O)c1ccc2oc3cccc(-n4c5cccc6c7ccccc7c7cccc4c7c65)c3c2c1] | 80% |
| 14h | 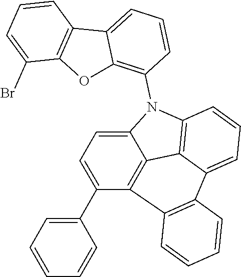 [SMILES: Brc1cccc2c1oc1c(-n3c4cccc5c6ccccc6c6c(-c7ccccc7)ccc3c6c54)cccc12] | 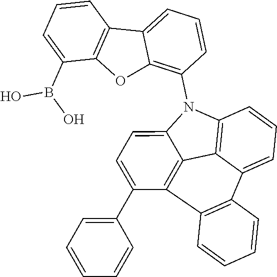 [SMILES: OB(O)c1cccc2c1oc1c(-n3c4cccc5c6ccccc6c6c(-c7ccccc7)ccc3c6c54)cccc12] | 81% |

### i) 4-(2,4-Diphenylbenzofuro[3,2-d]pyrimidin-8-yl)4H-naphtho[1,2,3,4-def]carbazole

### Route A for Bromides:

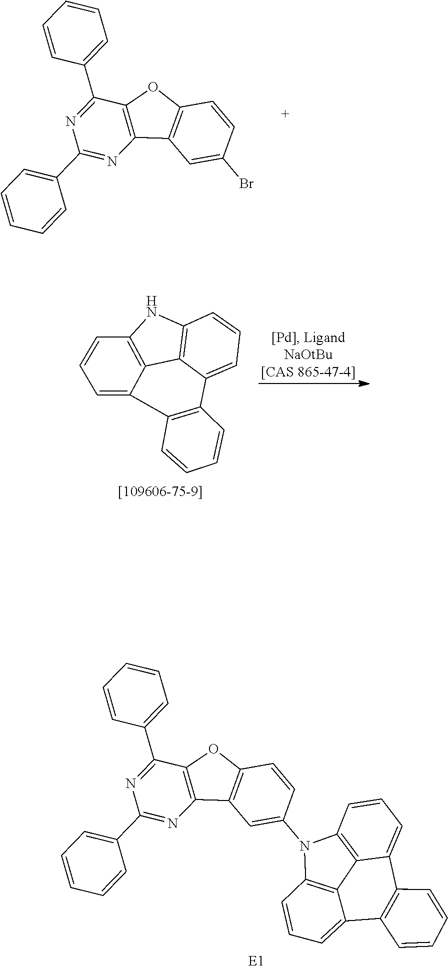 [SMILES: Brc1ccc2oc3c(-c4ccccc4)nc(-c4ccccc4)nc3c2c1 ; c1ccc2c(c1)c1cccc3[nH]c4cccc2c4c31 ; c1ccc(-c2nc(-c3ccccc3)c3oc4ccc(-n5c6cccc7c8ccccc8c8cccc5c8c76)cc4c3n2)cc1]

17 g (43 mmol; 1.00 eq.) of 8-bromo-2,4-diphenylbenzofuro[3,2-d]pyrimidine, 9.7 g (40.7 mmol; 1.10 eq.) of 4H-naphtho[1,2,3,4-def]carbazole and 7.82 g (81.4 mmol; 2.00 eq.) of sodium tert-butoxide [CAS 865-47-4] are suspended in 500 ml of ortho-xylene [CAS 95-47-6]. To this suspension are added 1.50 g (3.66 mmol; 9 mol %) of dicyclohexyl(2′,6′-dimethoxybiphenyl-2-yl)phosphine (SPhos) [CAS 657408-07-6] and 1.12 g (1.22 mmol; 3 mol %) of tris(dibenzylideneacetone)dipalladium [CAS 51364-51-3], and the reaction mixture is heated under reflux for 16 h. The reaction mixture is cooled down to room temperature and the solvent is removed under reduced pressure. The solids obtained are washed with 300 ml of ethanol and the recrystallized repeatedly for a mixture of heptane and xylene. After a hot filtration through Alox followed by sublimation under high vacuum, the purified product is obtained as a colorless solid, 16 g (28 mmol; 70%).

### Route B for Chlorides:

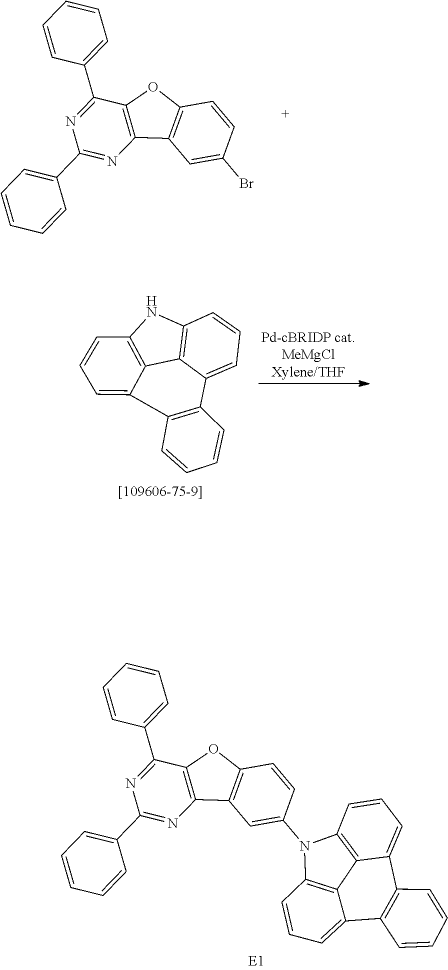 [SMILES: Brc1ccc2oc3c(-c4ccccc4)nc(-c4ccccc4)nc3c2c1 ; c1ccc2c(c1)c1cccc3[nH]c4cccc2c4c31 ; c1ccc(-c2nc(-c3ccccc3)c3oc4ccc(-n5c6cccc7c8ccccc8c8cccc5c8c76)cc4c3n2)cc1]

Under nitrogen, 16.0 g (66.0 mmol) of 4H-naphtho[1,2,3,4-def]carbazole is admixed with 60 ml of xylene and 6 ml of THF, and cooled to 5 °C. In a dropping funnel, a THF solution of MeMgCl (3.22 mol/l, 20.0 ml, 64.4 mmol) and 18 ml of THF is slowly added dropwise within 10 min to the carbazole solution such that the temperature does not exceed 25 °C. Subsequently added to that solution is a solution of 25 g (63.0 mmol) of 8-bromo-2,4-diphenylbenzofuro[3,2-d]pyrimidine and 14 ml of Pd-cBRIDP catalyst solution (prepared from PdCl(allyl)]2 (5.8 mg, 0.025 mol %) and cBRIDP (22.2 mg, 0.1 mol %) in 3 ml of THF and 11 ml of xylene). The mixture is heated to reflux under nitrogen for 3 hours, then the reaction mixture is allowed to cool down to room temperature, and the mixture is admixed with 25 ml of water and 1.7 g of NH₄Cl (31.8 mmol) and stirred at room temperature for 5 min. The organic phase is separated off, and the solution is concentrated and purified by chromatography (n-hexane, toluene 3/1). After hot extraction three times over Alox, followed by sublimation under high vacuum, the purified product is obtained as a colorless solid, 22.7 g (40 mmol; 65%).

It is possible to obtain the compounds that follow in an analogous manner via route A or route B. Workup and purification can also be accomplished using other standard solvents and purification methods.

| No. | Reactant 1 | Reactant 2 | Product | Yield |
|---|---|---|---|---|
| 1i | 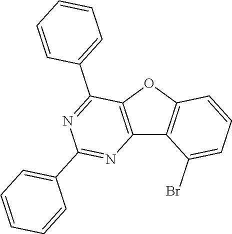 [SMILES: Brc1cccc2oc3c(-c4ccccc4)nc(-c4ccccc4)nc3c12] [2765540-98-3] | 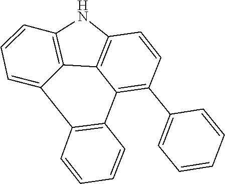 [SMILES: c1ccc(-c2ccc3[nH]c4cccc5c6ccccc6c2c3c45)cc1] | 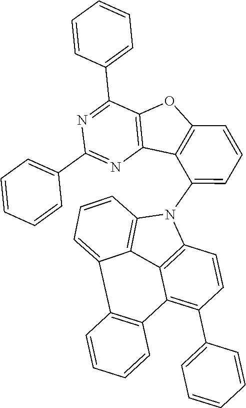 E4 | 67% |
| 2i | 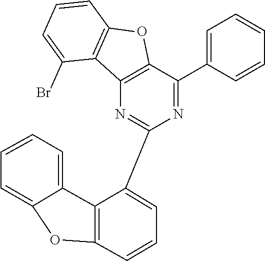 [SMILES: Brc1cccc2oc3c(-c4ccccc4)nc(-c4cccc5oc6ccccc6c45)nc3c12] | 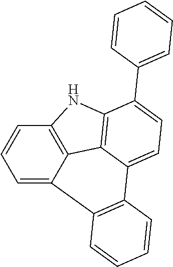 [SMILES: c1ccc(-c2ccc3c4ccccc4c4cccc5[nH]c2c3c54)cc1] | 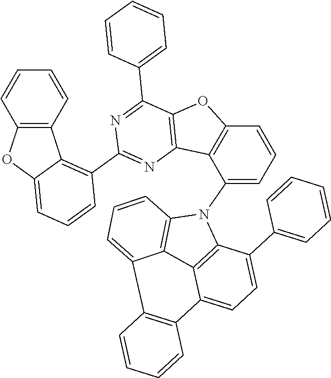 | 67% |
| 3i | 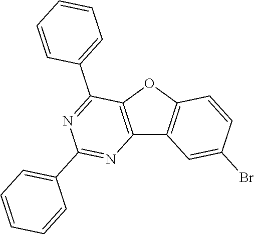 [SMILES: Brc1ccc2oc3c(-c4ccccc4)nc(-c4ccccc4)nc3c2c1] [2144459-61-8] | 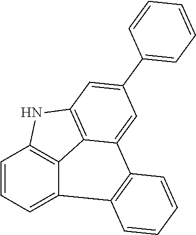 [SMILES: c1ccc(-c2cc3[nH]c4cccc5c6ccccc6c(c2)c3c45)cc1] | 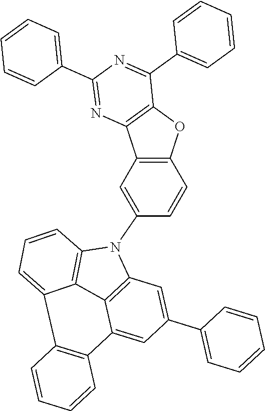 | 74% |
| 4i | 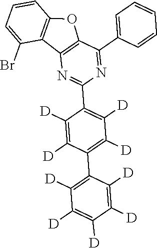 [SMILES: [2H]c1c([2H])c([2H])c(-c2c([2H])c([2H])c(-c3nc(-c4ccccc4)c4oc5cccc(Br)c5c4n3)c([2H])c2[2H])c([2H])c1[2H]] | 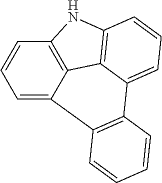 [SMILES: c1ccc2c(c1)c1cccc3[nH]c4cccc2c4c31] [109606-75-9] | 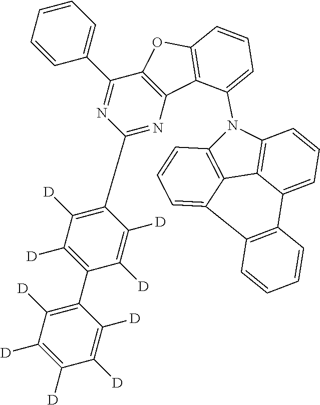 E5 | 63% |
| 5i | 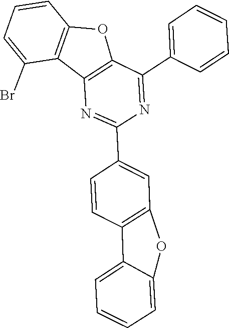 [SMILES: Brc1cccc2oc3c(-c4ccccc4)nc(-c4ccc5c(c4)oc4ccccc45)nc3c12] | 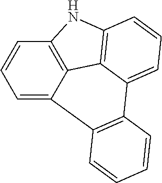 [SMILES: c1ccc2c(c1)c1cccc3[nH]c4cccc2c4c31] [109606-75-9] | 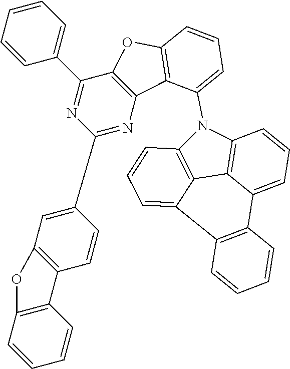 | 65% |
| 6i | 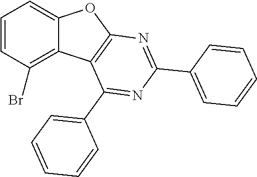 [SMILES: Brc1cccc2oc3nc(-c4ccccc4)nc(-c4ccccc4)c3c12] | 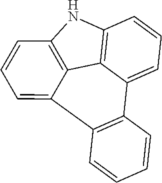 [SMILES: c1ccc2c(c1)c1cccc3[nH]c4cccc2c4c31] [109606-75-9] | 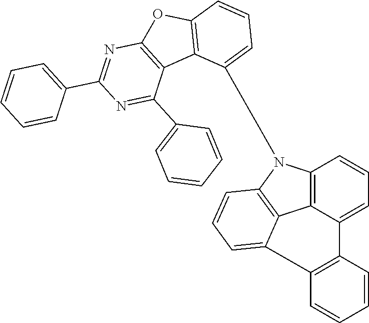 E6 | 61% |
| 7i | 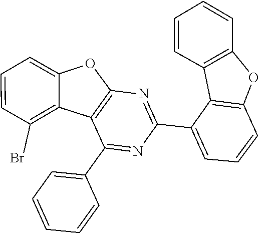 [SMILES: Brc1cccc2oc3nc(-c4cccc5oc6ccccc6c45)nc(-c4ccccc4)c3c12] | 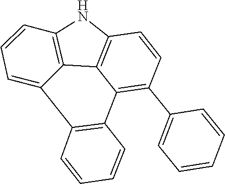 [SMILES: c1ccc(-c2ccc3[nH]c4cccc5c6ccccc6c2c3c45)cc1] | 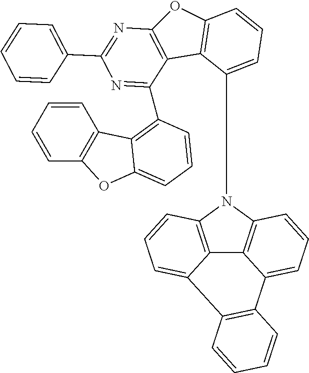 | 61% |
| 8i | 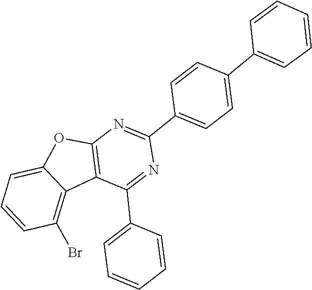 [SMILES: Brc1cccc2oc3nc(-c4ccc(-c5ccccc5)cc4)nc(-c4ccccc4)c3c12] | 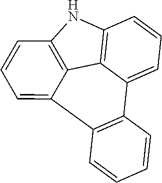 [SMILES: c1ccc2c(c1)c1cccc3[nH]c4cccc2c4c31] [109606-75-9] | 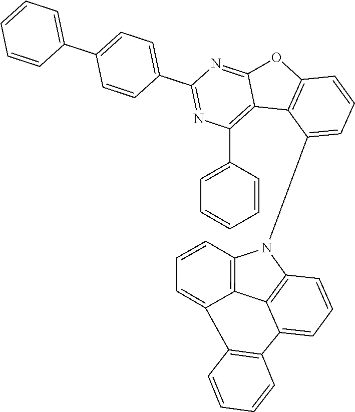 E7 | 68% |
| 9i | 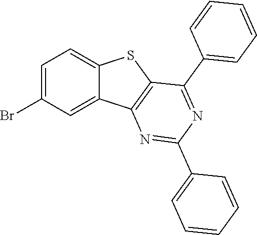 [SMILES: Brc1ccc2sc3c(-c4ccccc4)nc(-c4ccccc4)nc3c2c1] | 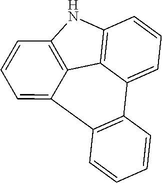 [SMILES: c1ccc2c(c1)c1cccc3[nH]c4cccc2c4c31] [109606-75-9] | 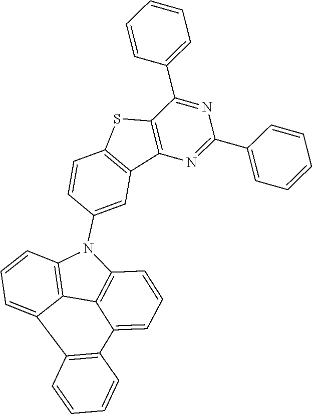 E8 | 64% |
| 10i | 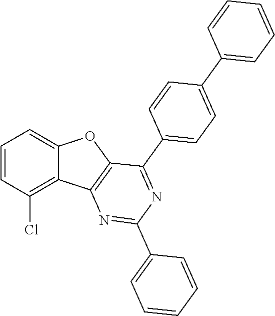 [SMILES: Clc1cccc2oc3c(-c4ccc(-c5ccccc5)cc4)nc(-c4ccccc4)nc3c12] | 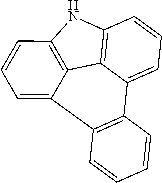 [SMILES: c1ccc2c(c1)c1cccc3[nH]c4cccc2c4c31] [109606-75-9] | 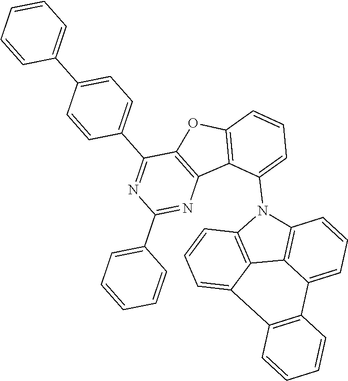 E9 | 62% |
| 11i | 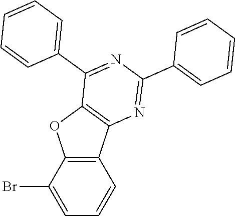 [SMILES: Brc1cccc2c1oc1c(-c3ccccc3)nc(-c3ccccc3)nc12] | 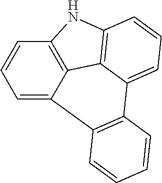 [SMILES: c1ccc2c(c1)c1cccc3[nH]c4cccc2c4c31] [109606-75-9] | 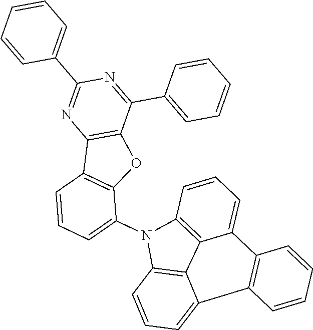 E10 | 72% |
| 12i | 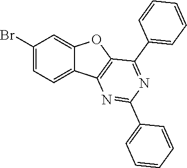 [SMILES: Brc1ccc2c(c1)oc1c(-c3ccccc3)nc(-c3ccccc3)nc12] | 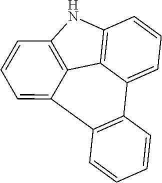 [SMILES: c1ccc2c(c1)c1cccc3[nH]c4cccc2c4c31] [109606-75-9] | 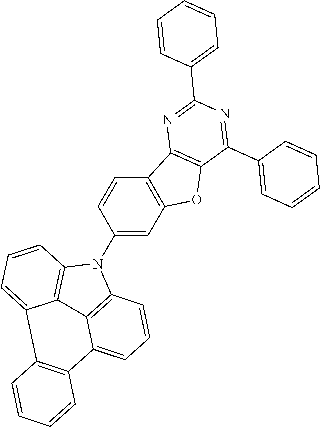 | 68% |
| 13i | 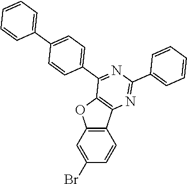 [SMILES: Brc1ccc2c(c1)oc1c(-c3ccc(-c4ccccc4)cc3)nc(-c3ccccc3)nc12] | 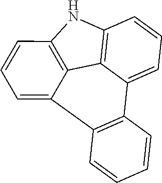 [SMILES: c1ccc2c(c1)c1cccc3[nH]c4cccc2c4c31] [109606-75-9] | 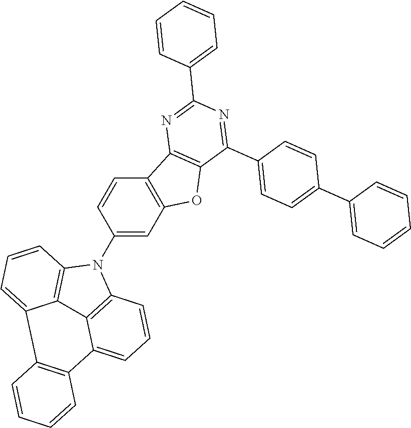 E11 | 65% |
| 14i | 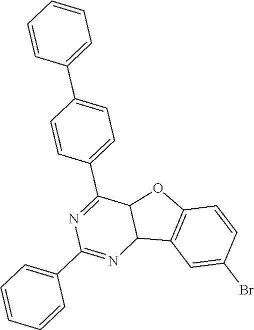 [SMILES: Brc1ccc2c(c1)C1N=C(c3ccccc3)N=C(c3ccc(-c4ccccc4)cc3)C1O2] [2765540-35-8] | 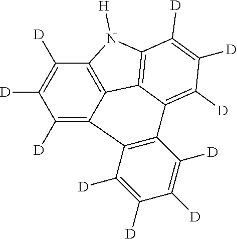 [SMILES: [2H]c1c([2H])c([2H])c2c(c1[2H])c1c([2H])c([2H])c([2H])c3[nH]c4c([2H])c([2H])c([2H])c2c4c31] | 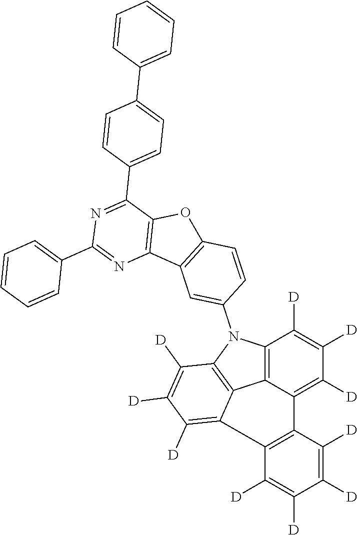 E2 | 61% |
| 15i | 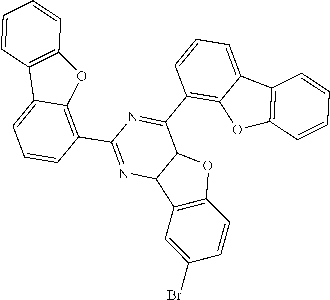 [SMILES: Brc1ccc2c(c1)C1N=C(c3cccc4c3oc3ccccc34)N=C(c3cccc4c3oc3ccccc34)C1O2] [2844364-95-8] | 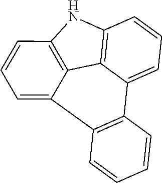 [SMILES: c1ccc2c(c1)c1cccc3[nH]c4cccc2c4c31] [109606-75-9] | 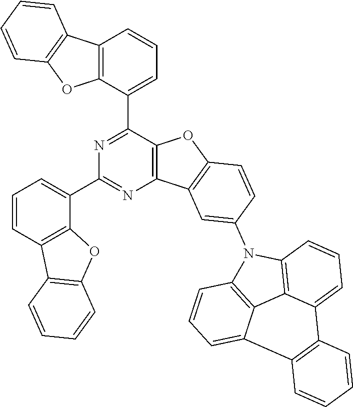 E12 | 63% |
| 16i | 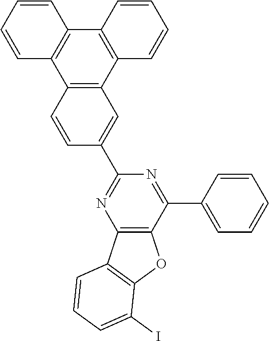 [SMILES: Ic1cccc2c1oc1c(-c3ccccc3)nc(-c3ccc4c5ccccc5c5ccccc5c4c3)nc12] [2162151-93-9] | 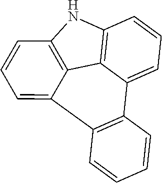 [SMILES: c1ccc2c(c1)c1cccc3[nH]c4cccc2c4c31] [109606-75-9] | 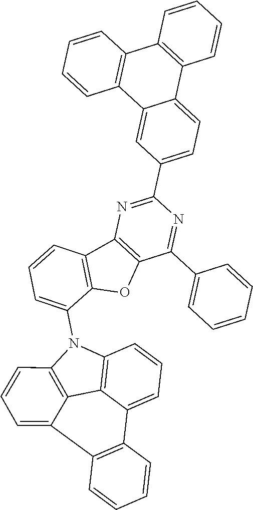 | 65% |
| 17i | 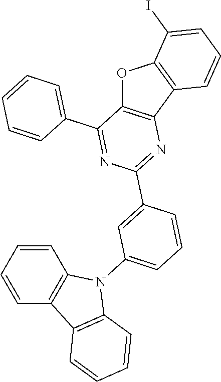 [SMILES: Ic1cccc2c1oc1c(-c3ccccc3)nc(-c3cccc(-n4c5ccccc5c5ccccc54)c3)nc12] [2162151-81-5] | 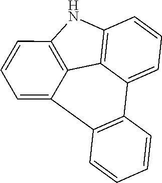 [SMILES: c1ccc2c(c1)c1cccc3[nH]c4cccc2c4c31] [109606-75-9] | 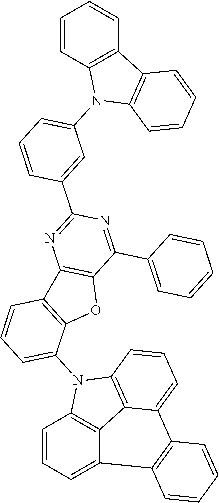 E13 | 66% |
| 18i | 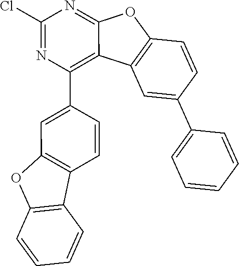 [SMILES: Clc1nc(-c2ccc3c(c2)oc2ccccc23)c2c(n1)oc1ccc(-c3ccccc3)cc12] [2768983-31-9]] | 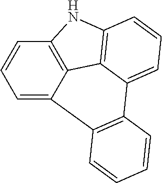 [SMILES: c1ccc2c(c1)c1cccc3[nH]c4cccc2c4c31] [109606-75-9] | 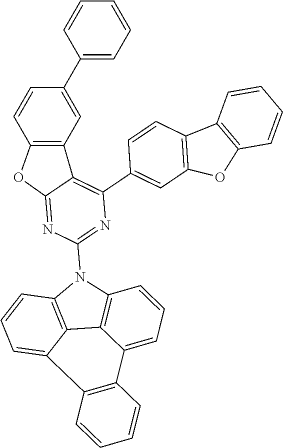 | 61% |
| 19i | 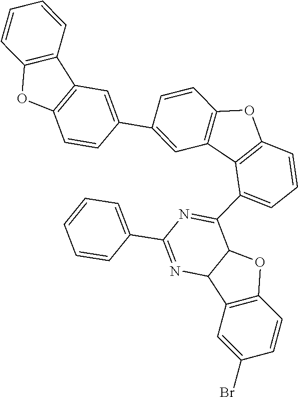 [SMILES: Brc1ccc2c(c1)C1N=C(c3ccccc3)N=C(c3cccc4oc5ccc(-c6ccc7oc8ccccc8c7c6)cc5c34)C1O2] [2768983-80-6] | 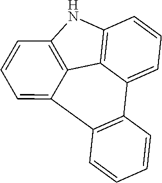 [SMILES: c1ccc2c(c1)c1cccc3[nH]c4cccc2c4c31] [109606-75-9] | 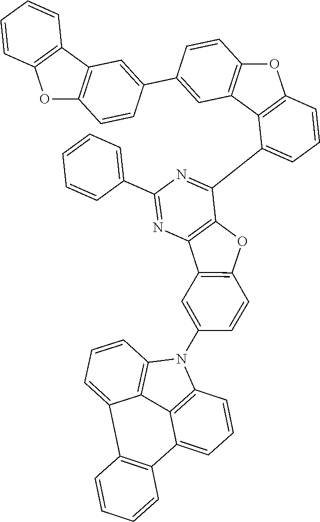 E14 | 64% |
| 20i | 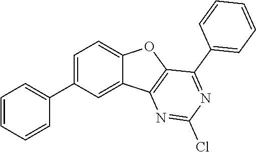 [SMILES: Clc1nc(-c2ccccc2)c2oc3ccc(-c4ccccc4)cc3c2n1] | 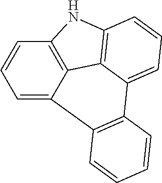 [SMILES: c1ccc2c(c1)c1cccc3[nH]c4cccc2c4c31] [109606-75-9] | 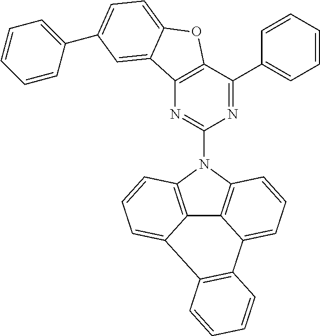 E15 | 57% |
| 21i | 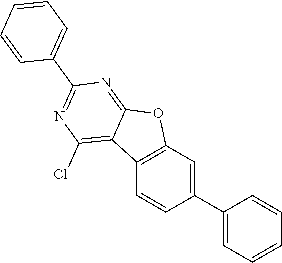 [SMILES: Clc1nc(-c2ccccc2)nc2oc3cc(-c4ccccc4)ccc3c12] [2165540-45-0] | 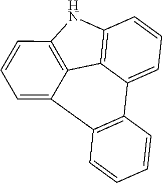 [SMILES: c1ccc2c(c1)c1cccc3[nH]c4cccc2c4c31] [109606-75-9] | 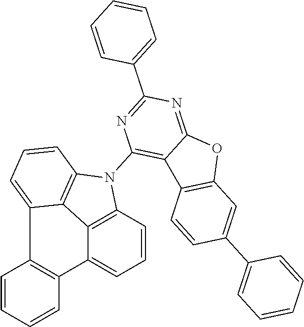 E16 | 69% |
| 22i | 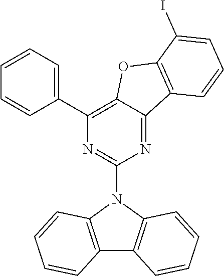 [SMILES: Ic1cccc2c1oc1c(-c3ccccc3)nc(-n3c4ccccc4c4ccccc43)nc12] [2162151-58-6] | 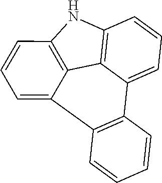 [SMILES: c1ccc2c(c1)c1cccc3[nH]c4cccc2c4c31] [109606-75-9] | 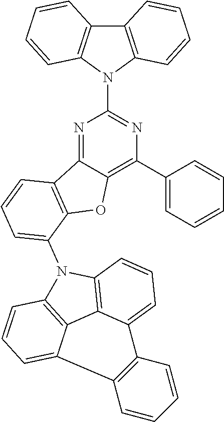 | 63% |
| 23i | 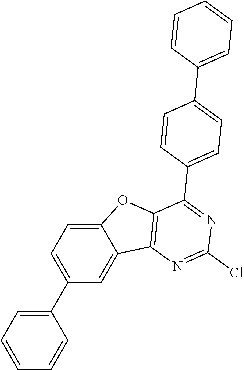 [SMILES: Clc1nc(-c2ccc(-c3ccccc3)cc2)c2oc3ccc(-c4ccccc4)cc3c2n1] | 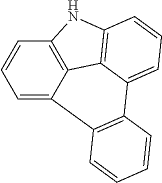 [SMILES: c1ccc2c(c1)c1cccc3[nH]c4cccc2c4c31] [109606-75-9] | 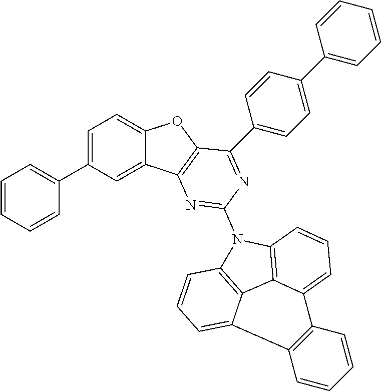 E17 | 54% |
| 24i | 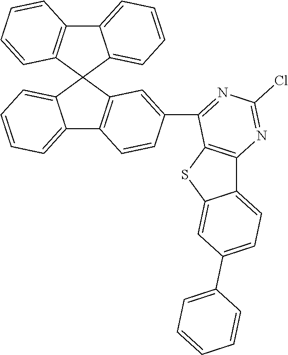 [SMILES: Clc1nc(-c2ccc3c(c2)C2(c4ccccc4-c4ccccc42)c2ccccc2-3)c2sc3cc(-c4ccccc4)ccc3c2n1] | 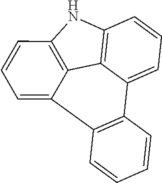 [SMILES: c1ccc2c(c1)c1cccc3[nH]c4cccc2c4c31] [109606-75-9] | 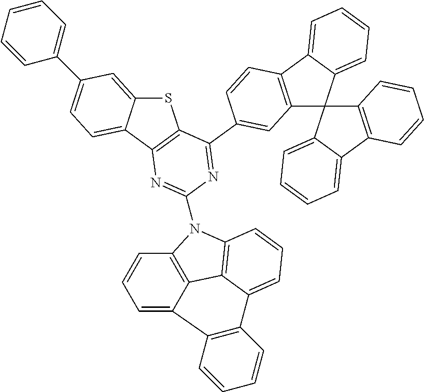 | 57% |
| 25i | 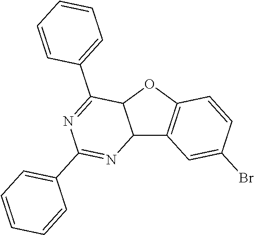 [SMILES: Brc1ccc2c(c1)C1N=C(c3ccccc3)N=C(c3ccccc3)C1O2] [2144459-61-8] | 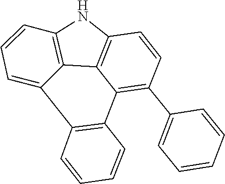 [SMILES: c1ccc(-c2ccc3[nH]c4cccc5c6ccccc6c2c3c45)cc1] | 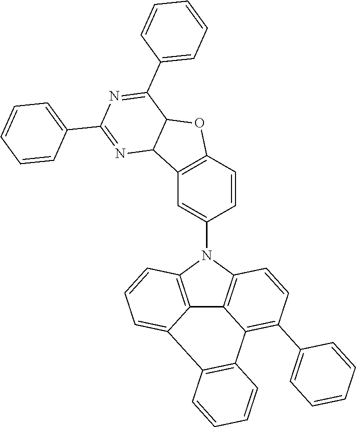 | 58% |
| 26i | 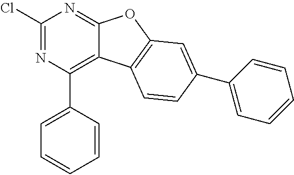 [SMILES: Clc1nc(-c2ccccc2)c2c(n1)oc1cc(-c3ccccc3)ccc12] [2346543-21-1] | 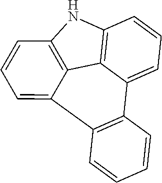 [SMILES: c1ccc2c(c1)c1cccc3[nH]c4cccc2c4c31] [109606-75-9] | 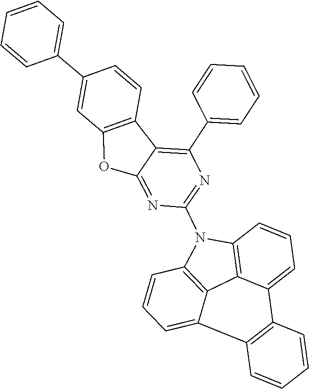 | 59% |
| 27i | 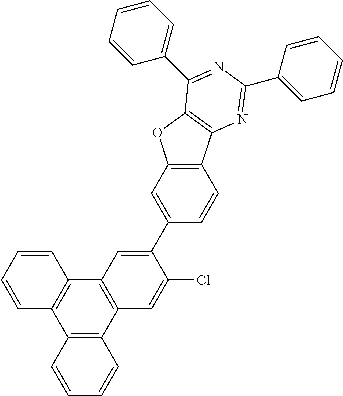 [SMILES: Clc1cc2c3ccccc3c3ccccc3c2cc1-c1ccc2c(c1)oc1c(-c3ccccc3)nc(-c3ccccc3)nc12] | 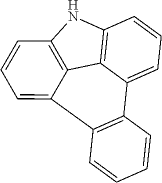 [SMILES: c1ccc2c(c1)c1cccc3[nH]c4cccc2c4c31] [109606-75-9] | 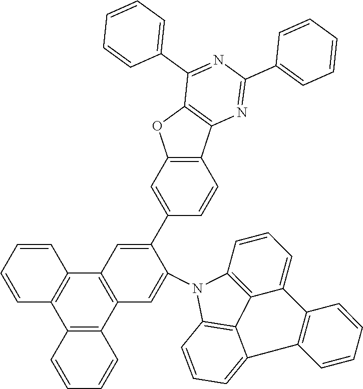 E33 | 52% |
| 28i | 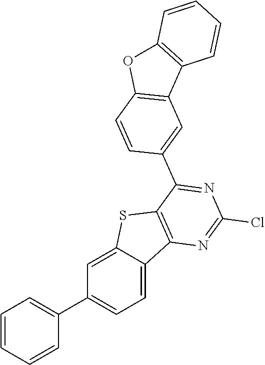 [SMILES: Clc1nc(-c2ccc3oc4ccccc4c3c2)c2sc3cc(-c4ccccc4)ccc3c2n1] | 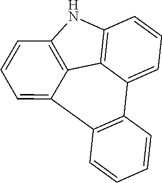 [SMILES: c1ccc2c(c1)c1cccc3[nH]c4cccc2c4c31] [109606-75-9] | 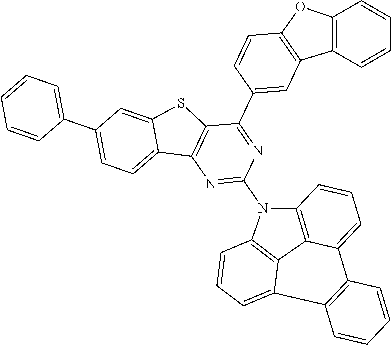 | 57% |
| 29i | 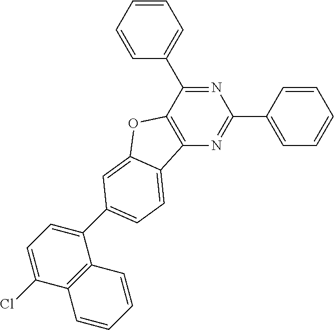 [SMILES: Clc1ccc(-c2ccc3c(c2)oc2c(-c4ccccc4)nc(-c4ccccc4)nc23)c2ccccc12] | 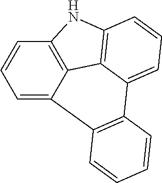 [SMILES: c1ccc2c(c1)c1cccc3[nH]c4cccc2c4c31] [109606-75-9] | 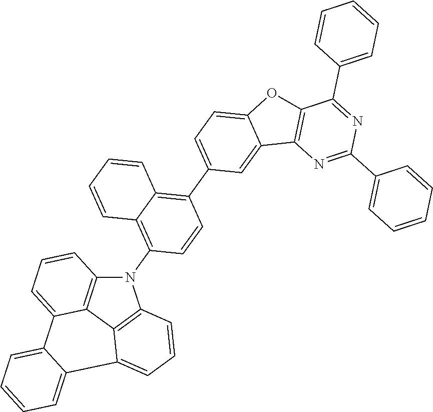 E34 | 51% |
| 30i | 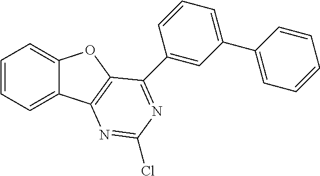 [SMILES: Clc1nc(-c2cccc(-c3ccccc3)c2)c2oc3ccccc3c2n1] | 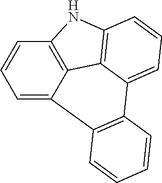 [SMILES: c1ccc2c(c1)c1cccc3[nH]c4cccc2c4c31] [109606-75-9] | 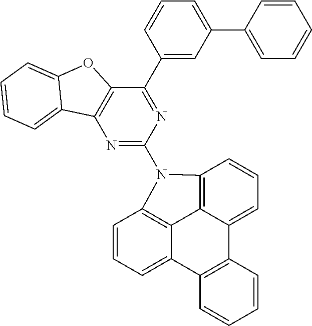 E35 | 47% |

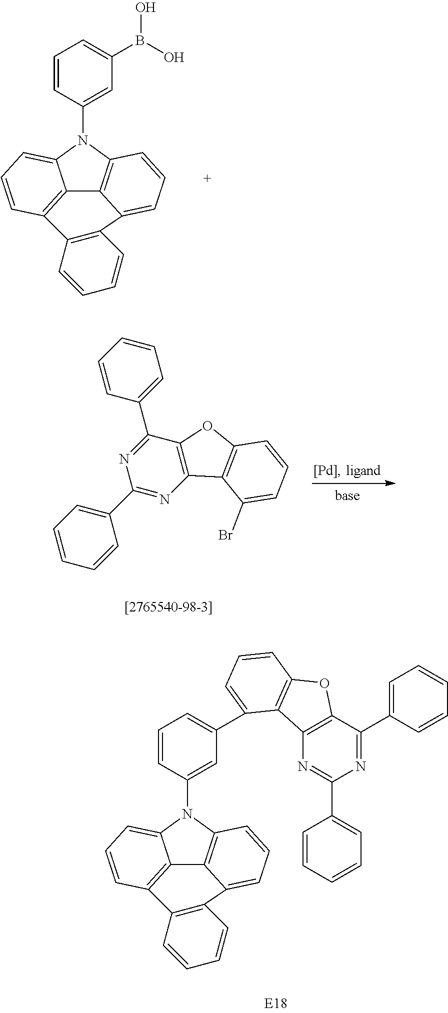

76.1 g (211 mmol; 1.00 eq.) of 3-(4H-naphtho[1,2,3,4-def]carbazol-4-yl)phenylboronic acid, 101 g (253 mmol; 1.20 eq.) of 9-bromo-2,4-diphenylbenzofuro[3,2-d]pyrimidine and 44.5 g (420 mmol, 2.00 eq.) of sodium carbonate are suspended in a mixture of 1000 ml of dioxane, 1000 ml of toluene and 400 ml of water. To that suspension is added 4.85 g (4.20 mmol; 0.02 eq.) of tetrakis(triphenylphosphine)palladium(0), and the reaction mixture is heated under reflux for 16 h. After cooling, the organic phase is removed, filtered through silica gel, washed three times with 200 ml of water and then concentrated to dryness. The yield is 93 g (147 mmol; 70% of theory).

The following compounds can be obtained analogously:

| No. | Reactant 1 | Reactant 2 | Product | Yield |
|---|---|---|---|---|
| 1j | 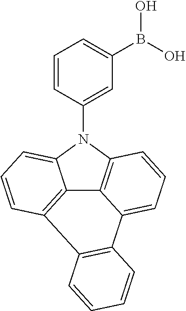 [SMILES: OB(O)c1cccc(-n2c3cccc4c5ccccc5c5cccc2c5c43)c1] | 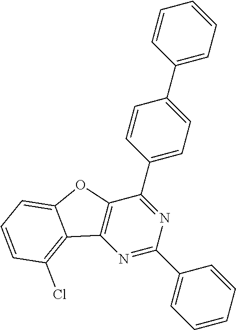 [SMILES: Clc1cccc2oc3c(-c4ccc(-c5ccccc5)cc4)nc(-c4ccccc4)nc3c12] | 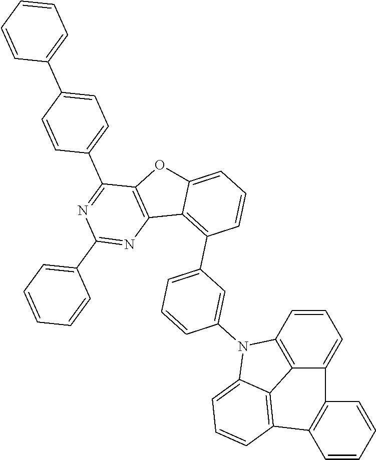 E19 | 60% |
| 2j | 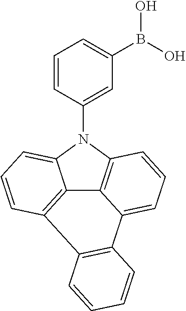 [SMILES: OB(O)c1cccc(-n2c3cccc4c5ccccc5c5cccc2c5c43)c1] | 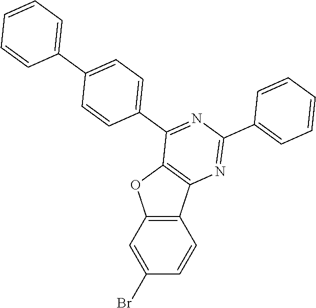 [SMILES: Brc1ccc2c(c1)oc1c(-c3ccc(-c4ccccc4)cc3)nc(-c3ccccc3)nc12] | 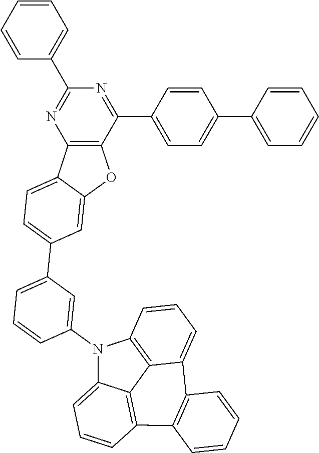 | 72% |
| 3j | 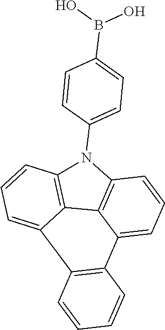 [SMILES: OB(O)c1ccc(-n2c3cccc4c5ccccc5c5cccc2c5c43)cc1] | 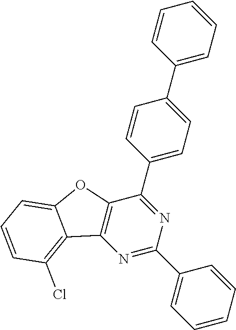 [SMILES: Clc1cccc2oc3c(-c4ccc(-c5ccccc5)cc4)nc(-c4ccccc4)nc3c12] | 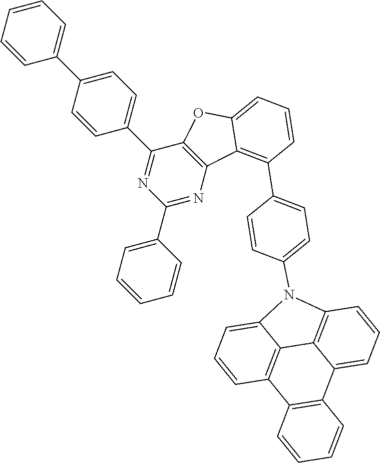 | 67% |
| 4j | 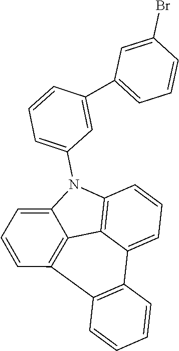 [SMILES: Brc1cccc(-c2cccc(-n3c4cccc5c6ccccc6c6cccc3c6c54)c2)c1] | 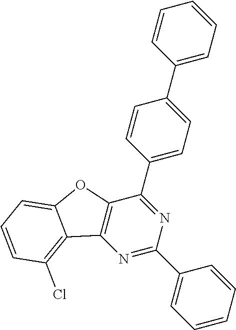 [SMILES: Clc1cccc2oc3c(-c4ccc(-c5ccccc5)cc4)nc(-c4ccccc4)nc3c12] | 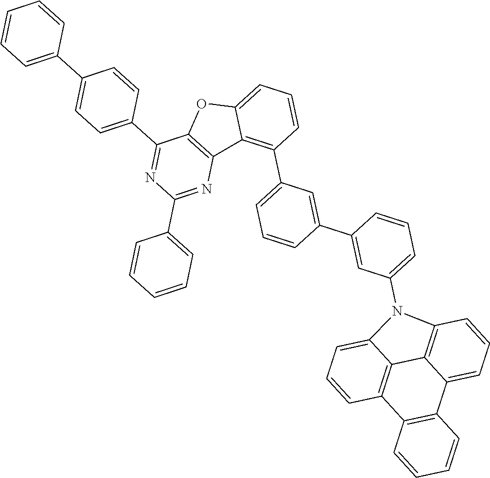 E20 | 62% |
| 5j | 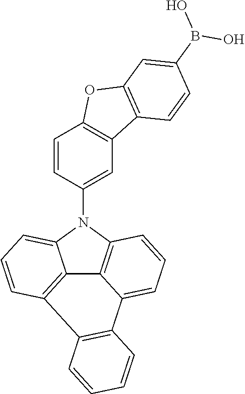 [SMILES: OB(O)c1ccc2c(c1)oc1ccc(-n3c4cccc5c6ccccc6c6cccc3c6c54)cc12] | 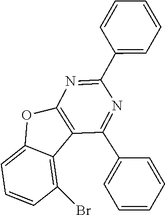 [SMILES: Brc1cccc2oc3nc(-c4ccccc4)nc(-c4ccccc4)c3c12] | 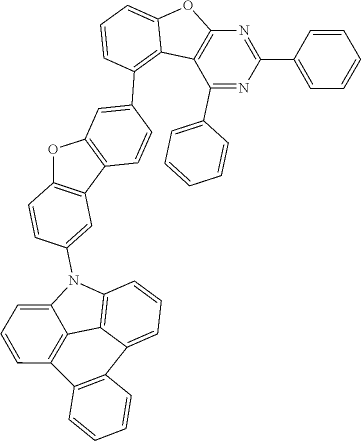 | 75% |
| 6j | 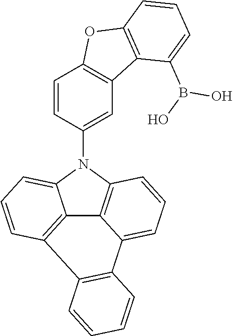 [SMILES: OB(O)c1cccc2oc3ccc(-n4c5cccc6c7ccccc7c7cccc4c7c65)cc3c12] | 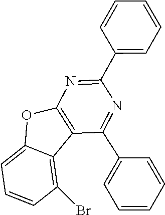 [SMILES: Brc1cccc2oc3nc(-c4ccccc4)nc(-c4ccccc4)c3c12] | 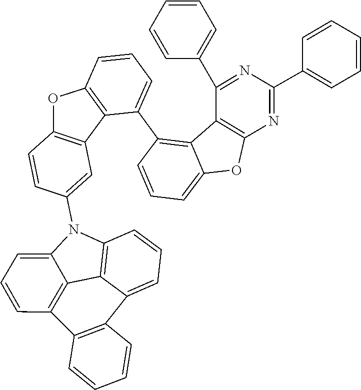 E21 | 70% |
| 7j | 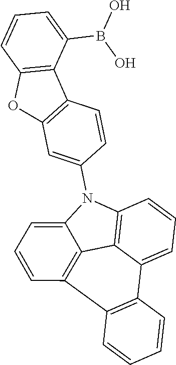 [SMILES: OB(O)c1cccc2oc3cc(-n4c5cccc6c7ccccc7c7cccc4c7c65)ccc3c12] | 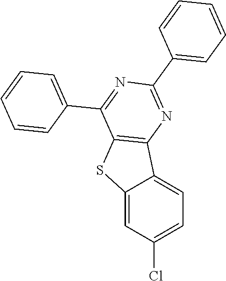 [SMILES: Clc1ccc2c(c1)sc1c(-c3ccccc3)nc(-c3ccccc3)nc12] | 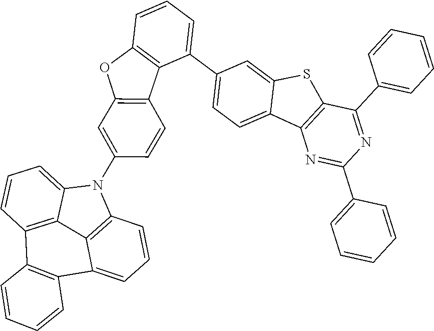 | 63% |
| 8j | 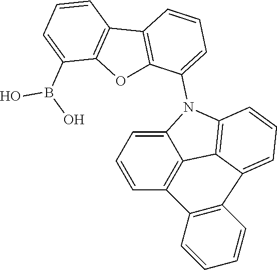 [SMILES: OB(O)c1cccc2c1oc1c(-n3c4cccc5c6ccccc6c6cccc3c6c54)cccc12] | 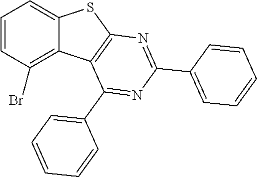 [SMILES: Brc1cccc2sc3nc(-c4ccccc4)nc(-c4ccccc4)c3c12] | 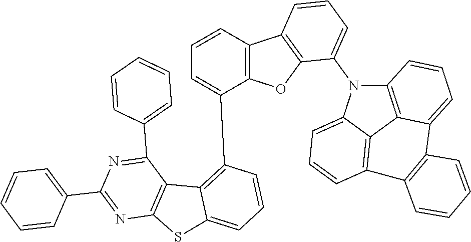 E22 | 69% |
| 9j | 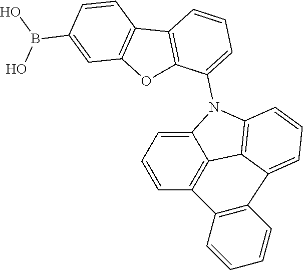 [SMILES: OB(O)c1ccc2c(c1)oc1c(-n3c4cccc5c6ccccc6c6cccc3c6c54)cccc12] | 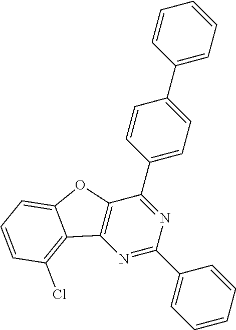 [SMILES: Clc1cccc2oc3c(-c4ccc(-c5ccccc5)cc4)nc(-c4ccccc4)nc3c12] | 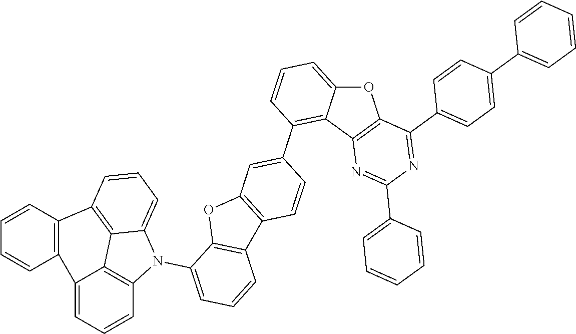 | 60% |
| 10j | 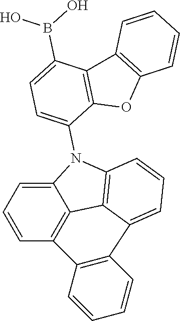 [SMILES: OB(O)c1ccc(-n2c3cccc4c5ccccc5c5cccc2c5c43)c2oc3ccccc3c12] | 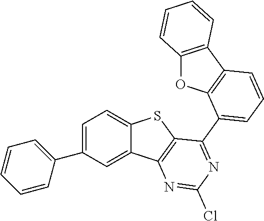 [SMILES: Clc1nc(-c2cccc3c2oc2ccccc23)c2sc3ccc(-c4ccccc4)cc3c2n1] | 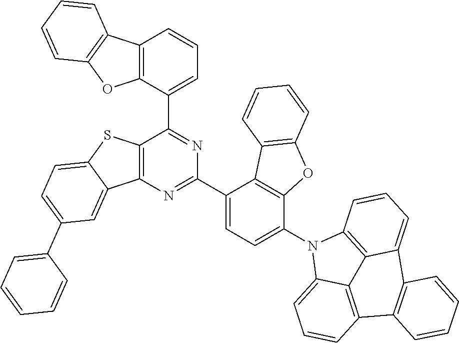 | 63% |
| 11j | 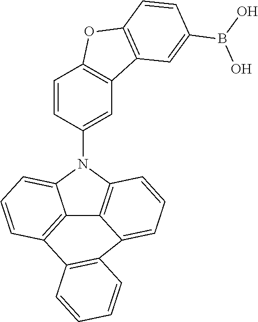 [SMILES: OB(O)c1ccc2oc3ccc(-n4c5cccc6c7ccccc7c7cccc4c7c65)cc3c2c1] | 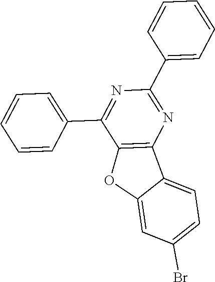 [SMILES: Brc1ccc2c(c1)oc1c(-c3ccccc3)nc(-c3ccccc3)nc12] | 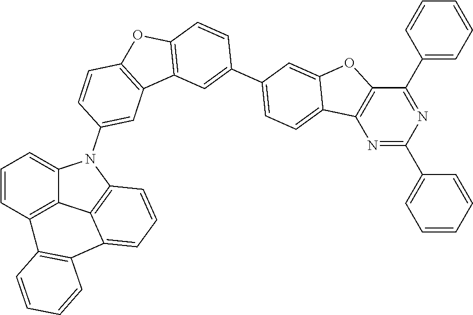 | 74% |
| 12j | 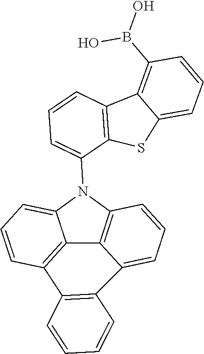 [SMILES: OB(O)c1cccc2sc3c(-n4c5cccc6c7ccccc7c7cccc4c7c65)cccc3c12] [109606-75-9] | 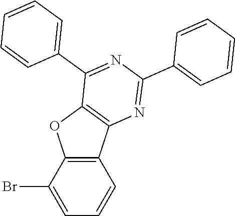 [SMILES: Brc1cccc2c1oc1c(-c3ccccc3)nc(-c3ccccc3)nc12] | 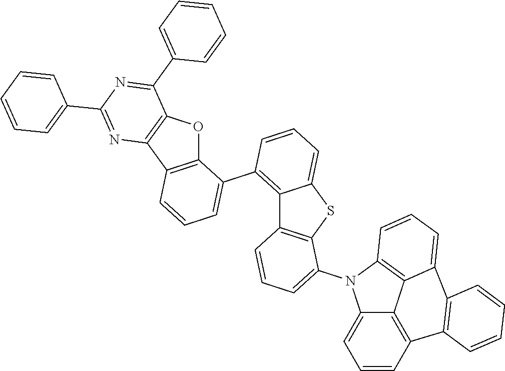 E23 | 67% |
| 13j | 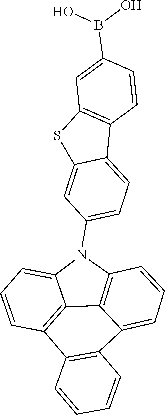 [SMILES: OB(O)c1ccc2c(c1)sc1cc(-n3c4cccc5c6ccccc6c6cccc3c6c54)ccc12] | 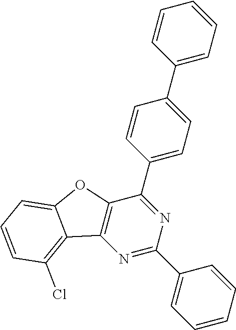 [SMILES: Clc1cccc2oc3c(-c4ccc(-c5ccccc5)cc4)nc(-c4ccccc4)nc3c12] | 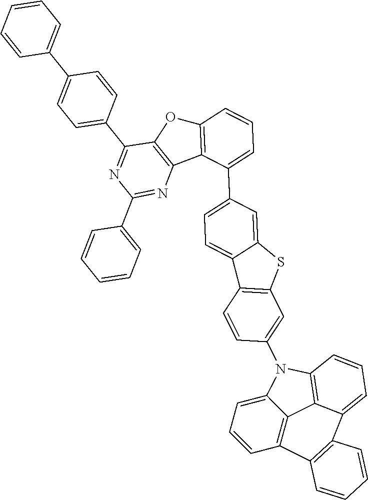 | 63% |
| 14j | 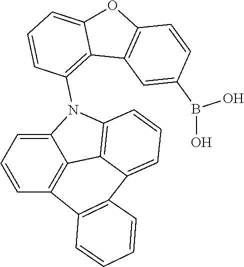 [SMILES: OB(O)c1ccc2oc3cccc(-n4c5cccc6c7ccccc7c7cccc4c7c65)c3c2c1] | 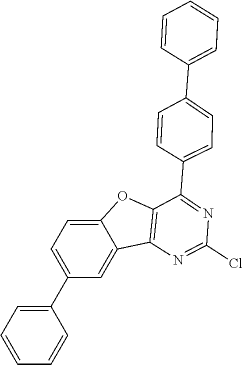 [SMILES: Clc1nc(-c2ccc(-c3ccccc3)cc2)c2oc3ccc(-c4ccccc4)cc3c2n1] | 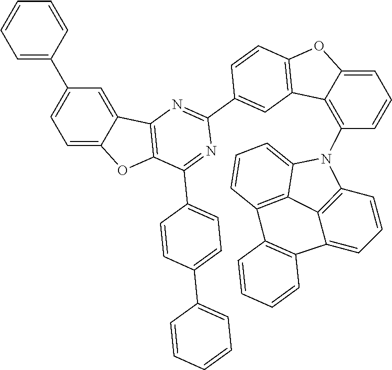 E24 | 61% |
| 15j | 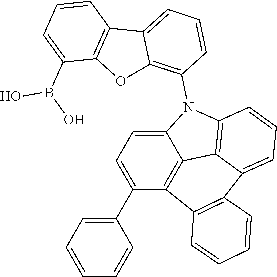 [SMILES: OB(O)c1cccc2c1oc1c(-n3c4cccc5c6ccccc6c6c(-c7ccccc7)ccc3c6c54)cccc12] | 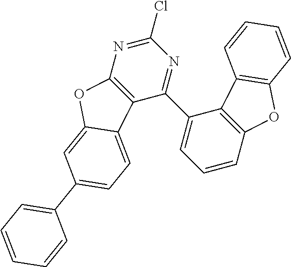 [SMILES: Clc1nc(-c2cccc3oc4ccccc4c23)c2c(n1)oc1cc(-c3ccccc3)ccc12] | 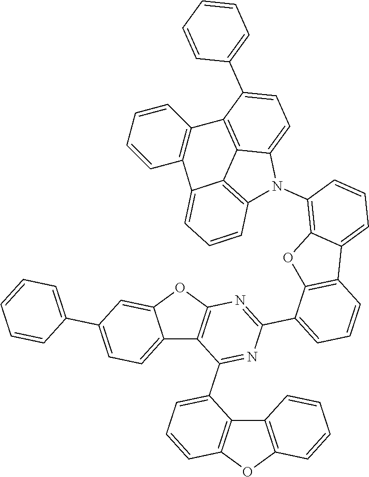 | 58% |
| 16j | 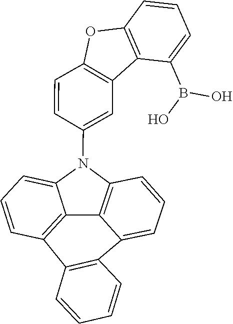 [SMILES: OB(O)c1cccc2oc3ccc(-n4c5cccc6c7ccccc7c7cccc4c7c65)cc3c12] | 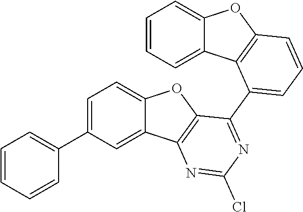 [SMILES: Clc1nc(-c2cccc3oc4ccccc4c23)c2oc3ccc(-c4ccccc4)cc3c2n1] | 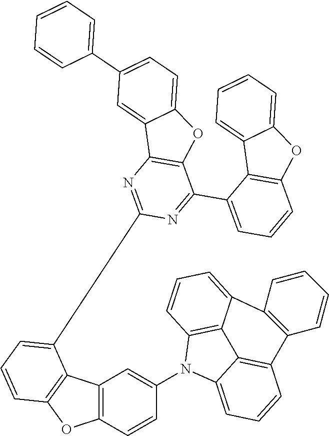 | 64% |
| 17j | 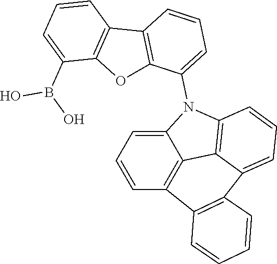 [SMILES: OB(O)c1cccc2c1oc1c(-n3c4cccc5c6ccccc6c6cccc3c6c54)cccc12] | 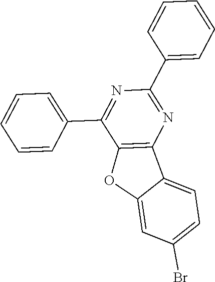 [SMILES: Brc1ccc2c(c1)oc1c(-c3ccccc3)nc(-c3ccccc3)nc12] | 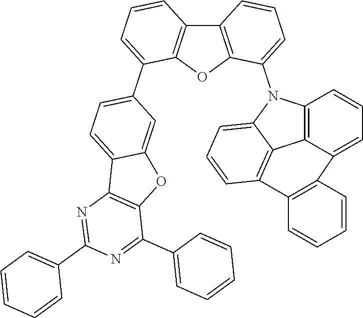 E25 | 75% |
| 18j | 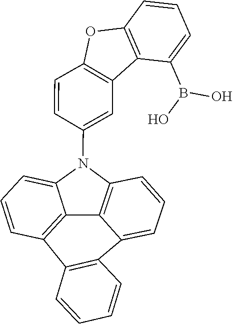 [SMILES: OB(O)c1cccc2oc3ccc(-n4c5cccc6c7ccccc7c7cccc4c7c65)cc3c12] | 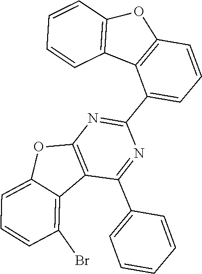 [SMILES: Brc1cccc2oc3nc(-c4cccc5oc6ccccc6c45)nc(-c4ccccc4)c3c12] | 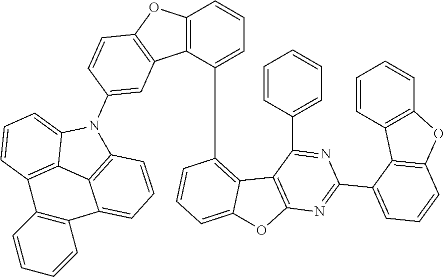 | 67% |
| 19j | 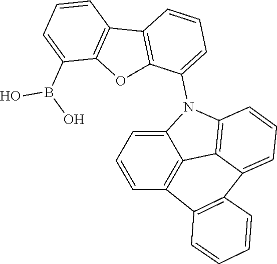 [SMILES: OB(O)c1cccc2c1oc1c(-n3c4cccc5c6ccccc6c6cccc3c6c54)cccc12] | 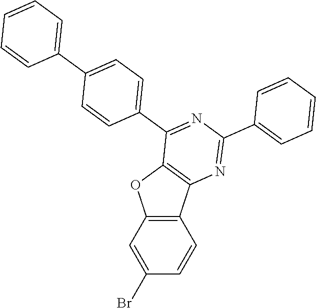 [SMILES: Brc1ccc2c(c1)oc1c(-c3ccc(-c4ccccc4)cc3)nc(-c3ccccc3)nc12] | 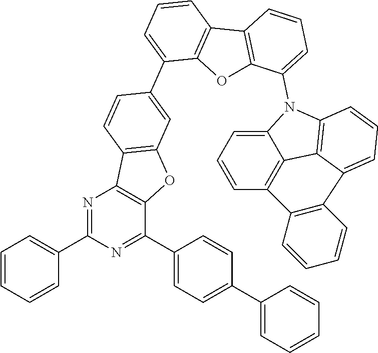 E26 | 72% |
| 20j | 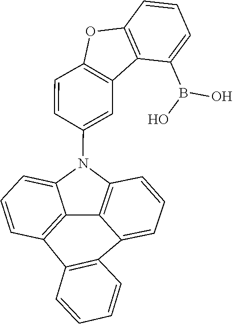 [SMILES: OB(O)c1cccc2oc3ccc(-n4c5cccc6c7ccccc7c7cccc4c7c65)cc3c12] | 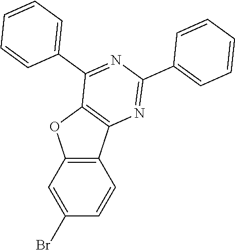 [SMILES: Brc1ccc2c(c1)oc1c(-c3ccccc3)nc(-c3ccccc3)nc12] | 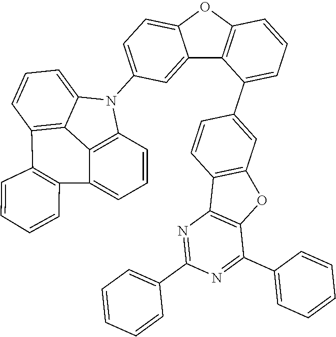 | 74% |
| 21j | 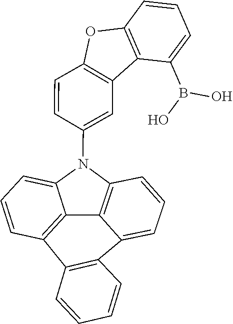 [SMILES: OB(O)c1cccc2oc3ccc(-n4c5cccc6c7ccccc7c7cccc4c7c65)cc3c12] | 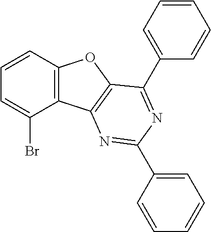 [SMILES: Brc1cccc2oc3c(-c4ccccc4)nc(-c4ccccc4)nc3c12] | 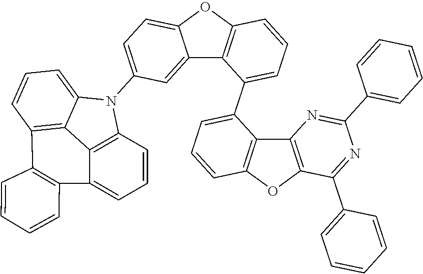 E27 | 70% |
| 22j | 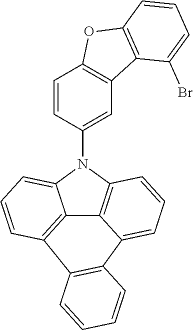 [SMILES: Brc1cccc2oc3ccc(-n4c5cccc6c7ccccc7c7cccc4c7c65)cc3c12] | 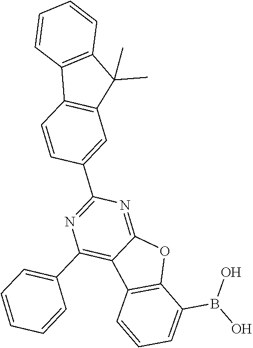 [SMILES: CC1(C)c2ccccc2-c2ccc(-c3nc(-c4ccccc4)c4c(n3)oc3c(B(O)O)cccc34)cc21] [2162151-99-5] | 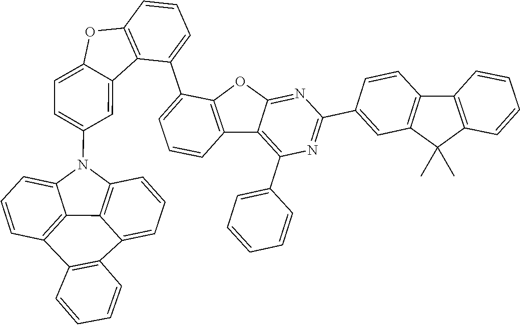 | 64% |
| 23j | 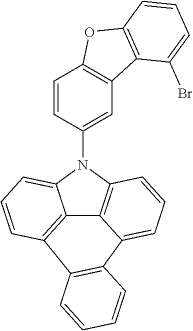 [SMILES: Brc1cccc2oc3ccc(-n4c5cccc6c7ccccc7c7cccc4c7c65)cc3c12] | 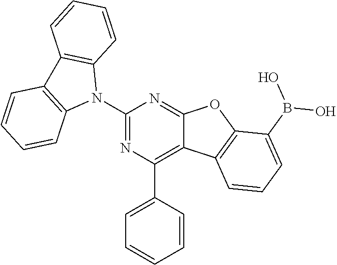 [SMILES: OB(O)c1cccc2c1oc1nc(-n3c4ccccc4c4ccccc43)nc(-c3ccccc3)c12] [2162151-78-0] | 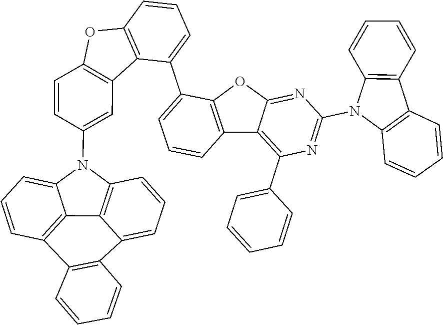 | 67% |
| 24j | 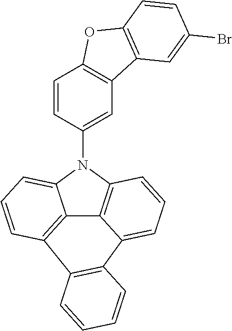 [SMILES: Brc1ccc2oc3ccc(-n4c5cccc6c7ccccc7c7cccc4c7c65)cc3c2c1] | 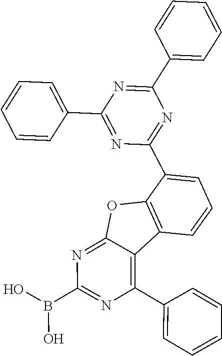 [SMILES: OB(O)c1nc(-c2ccccc2)c2c(n1)oc1c(-c3nc(-c4ccccc4)nc(-c4ccccc4)n3)cccc12] [2588220-26-0] | 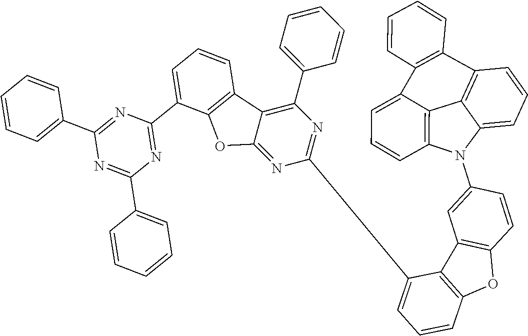 | 59% |
| 25j | 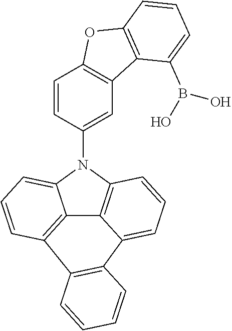 [SMILES: OB(O)c1cccc2oc3ccc(-n4c5cccc6c7ccccc7c7cccc4c7c65)cc3c12] | 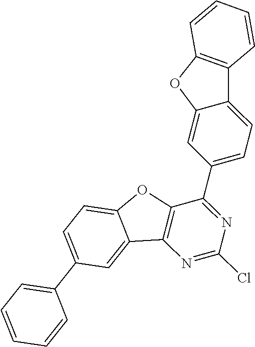 [SMILES: Clc1nc(-c2ccc3c(c2)oc2ccccc23)c2oc3ccc(-c4ccccc4)cc3c2n1] | 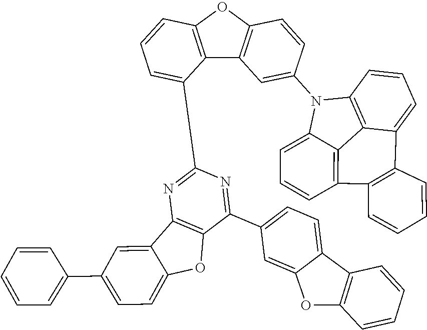 | 61% |
| 26j | 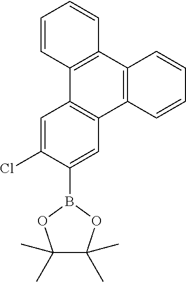 [SMILES: CC1(C)OB(c2cc3c4ccccc4c4ccccc4c3cc2Cl)OC1(C)C] [2485777-31-7] | 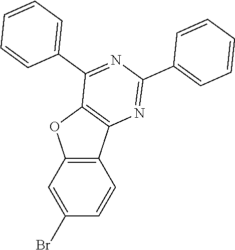 [SMILES: Brc1ccc2c(c1)oc1c(-c3ccccc3)nc(-c3ccccc3)nc12] | 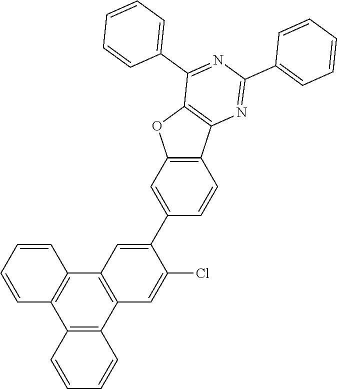 | 55% |
| 27j | 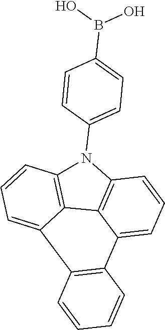 [SMILES: OB(O)c1ccc(-n2c3cccc4c5ccccc5c5cccc2c5c43)cc1] | 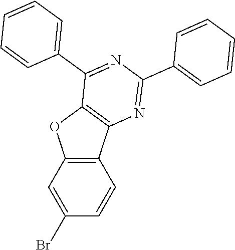 [SMILES: Brc1ccc2c(c1)oc1c(-c3ccccc3)nc(-c3ccccc3)nc12] | 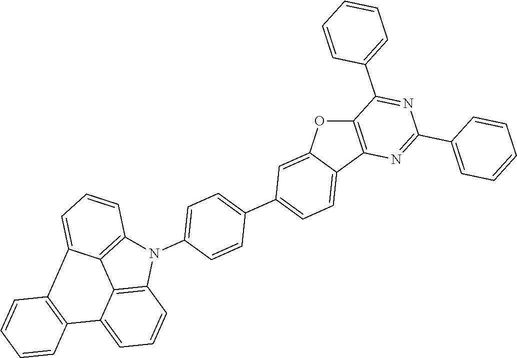 E36 | 65% |

### k) 4-(2,4-diphenylbenzofuro[3,2-d]pyrimidin-8-yl)4H-naphtho[1,2,3,4-def]carbazole-d₂₃

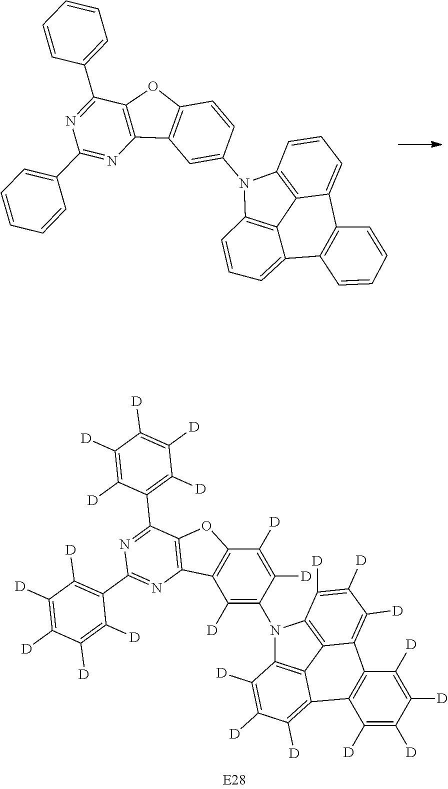 [SMILES: c1ccc(-c2nc(-c3ccccc3)c3oc4ccc(-n5c6cccc7c8ccccc8c8cccc5c8c76)cc4c3n2)cc1 ; [2H]c1c([2H])c([2H])c(-c2nc(-c3c([2H])c([2H])c([2H])c([2H])c3[2H])c3oc4c([2H])c([2H])c(-n5c6c([2H])c([2H])c([2H])c7c8c([2H])c([2H])c([2H])c([2H])c8c8c([2H])c([2H])c([2H])c5c8c76)c([2H])c4c3n2)c([2H])c1[2H]]

26.3 g (47.0 mmol; 1.00 eq) of 4-(2,4-diphenylbenzofuro[3,2-d]pyrimidin-8-yl)4H-naphtho[1,2,3,4-def]carbazole is suspended in 640 ml (120 eq) of toluene-d8 [CAS 2037-26-5]. Added to this mixture while cooling is 16.6 ml (6.00 eq.) of trifluoromethanesulfonic acid. The reaction mixture is stirred at room temperature for 6 hours. Thereafter, 120 ml (130 eq) of deuterium oxide [CAS 7789-20-0] is added dropwise at 0 °C. Neutralization with a potassium sulfate solution is followed by extraction with toluene and washing of the combined organic phases with saline solution and drying over sodium sulfate. After filtration, the solvent is removed under reduced pressure. 16.1 g (27 mmol, 59% of theory) of the product shown above is obtained in a mixture with fractions of H/D isotopomers and H/D isotopologues after purification by chromatography, and finally sublimed under high vacuum (p=5×10⁻⁷ mbar) (99.9% purity).

The following products are obtained in an analogous manner:

| | Reactant 1 | Product | Yield |
|---|---|---|---|
| 1k | 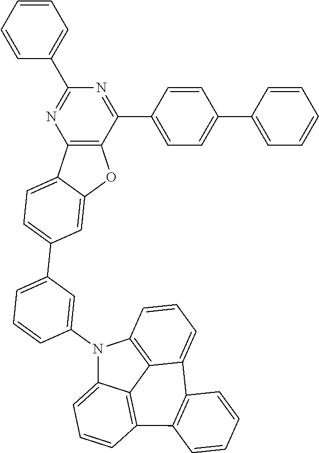 [SMILES: c1ccc(-c2ccc(-c3nc(-c4ccccc4)nc4c3oc3cc(-c5cccc(-n6c7cccc8c9ccccc9c9cccc6c9c87)c5)ccc34)cc2)cc1] | 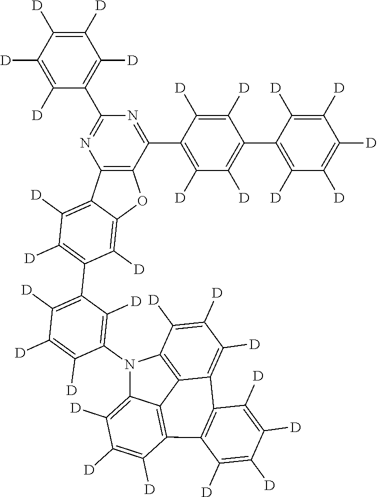 E29 | 59% |
| 2k | 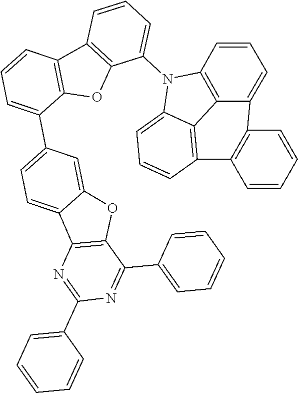 [SMILES: c1ccc(-c2nc(-c3ccccc3)c3oc4cc(-c5cccc6c5oc5c(-n7c8cccc9c%10ccccc%10c%10cccc7c%10c98)cccc56)ccc4c3n2)cc1] | 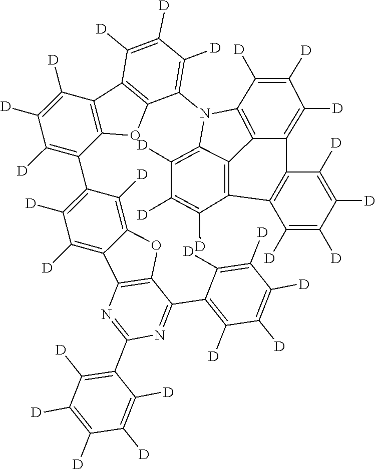 E30 | 55% |
| 3k | 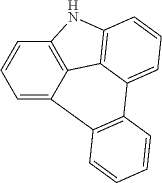 [SMILES: c1ccc2c(c1)c1cccc3[nH]c4cccc2c4c31] [109606-75-9] | 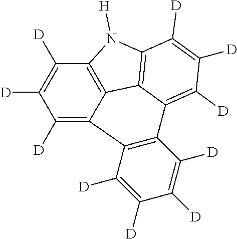 | 80% |

### l) 2-(3-Fluorotriphenylen-2-yl)-4,4,5,5-tetramethyl-1,3,2-dioxaborolane

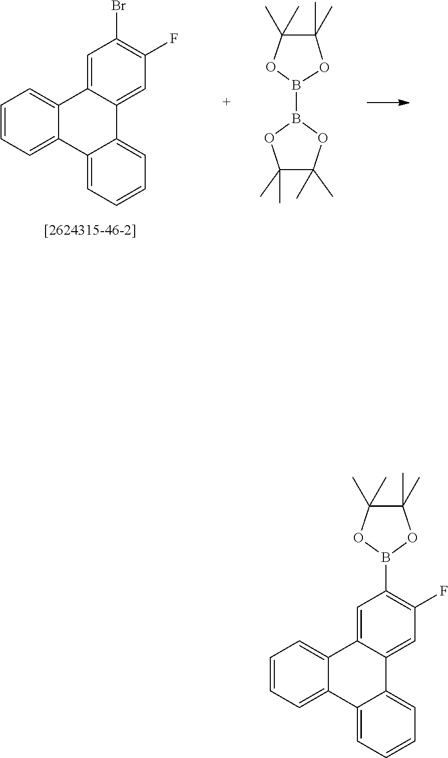

An initial charge of 40 g (123 mmol) of 2-bromo-3-fluorotriphenylene, 60 g (236 mmol) of bis(pinacolato)diboron and 35 g in 800 ml of 1,4-dioxane is inertized with argon for 30 min. Subsequently, 3.5 (3.8 mmol) of tris(dibenzylideneacetone)dipalladium (97%) and 4 (14 mmol) of tricyclohexylphosphine are added and the mixture is stirred under reflux for 24 h. After cooling, the solvent is removed on a rotary evaporator, and the residue is worked up by extraction with dichloromethane and water. The combined organic phases are dried over Na₂SO₄, ethanol (150 ml) is added, and the dichloromethane is drawn off on a rotary evaporator. The precipitated solids are filtered off with suction and dried in a vacuum drying cabinet. The crude product is used in the next stage without further purification.

Yield: 30.7 g (82 mmol, 67%), purity 94% by ¹H-NMR.

### m) 8-(3-Fluorotriphenylen-2-yl)-2,4-diphenylbenzofuro[3,2-d]pyrimidine

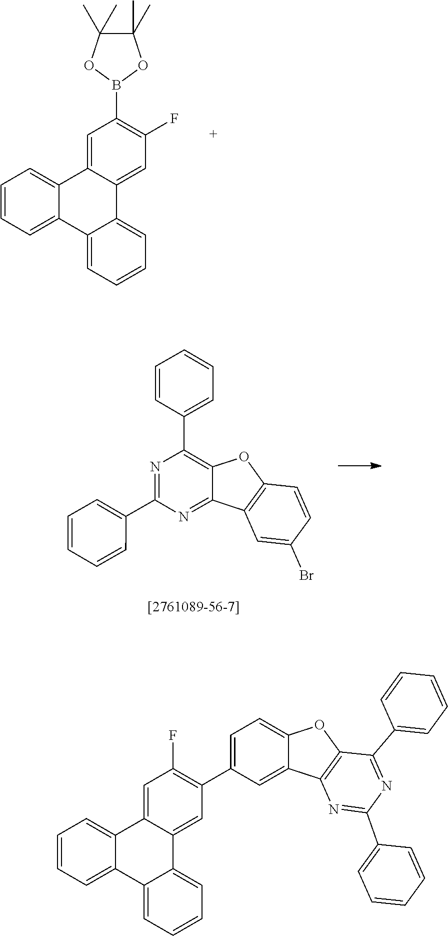 [SMILES: CC1(C)OB(c2cc3c4ccccc4c4ccccc4c3cc2F)OC1(C)C ; Brc1ccc2oc3c(-c4ccccc4)nc(-c4ccccc4)nc3c2c1 ; Fc1cc2c3ccccc3c3ccccc3c2cc1-c1ccc2oc3c(-c4ccccc4)nc(-c4ccccc4)nc3c2c1]

63 g (156 mmol) of 8-bromo-2,4-diphenylbenzofuro[3,2-d]pyrimidine, 50 g (170 mmol) of 2-(3-fluorotriphenylen-2-yl)-4,4,5,5-tetramethyl-1,3,2-dioxaborolane and 36 g (340 mmol) of sodium carbonate are suspended in 1000 ml of ethylene glycol diamine ether and 280 ml of water. 1.8 g (1.5 mmol) of tetrakis(triphenylphosphine)palladium(0) is added to this suspension, and the reaction mixture is heated under reflux for 16 h. After cooling, the organic phase is removed, filtered through silica gel and then concentrated to dryness. The product is purified via column chromatography on silica gel with toluene/heptane (1:2) and finally sublimed under high vacuum (p=5×10⁻⁷ mbar) (99.9% purity). The yield is 68 g (120 mmol), corresponding to 71% of theory.

The following compounds are prepared in an analogous manner:

| | Reactant 1 | Reactant 2 | Product | Yield |
|---|---|---|---|---|
| 1m | 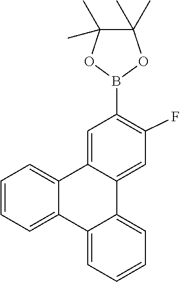 [SMILES: CC1(C)OB(c2cc3c4ccccc4c4ccccc4c3cc2F)OC1(C)C] | 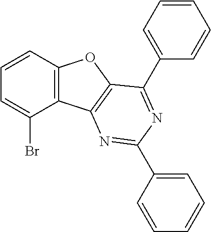 [SMILES: Brc1cccc2oc3c(-c4ccccc4)nc(-c4ccccc4)nc3c12] | 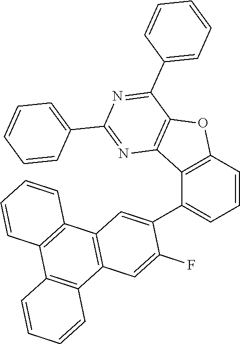 [SMILES: Fc1cc2c3ccccc3c3ccccc3c2cc1-c1cccc2oc3c(-c4ccccc4)nc(-c4ccccc4)nc3c12] | 67% |
| 2m | 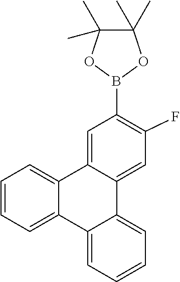 [SMILES: CC1(C)OB(c2cc3c4ccccc4c4ccccc4c3cc2F)OC1(C)C] | 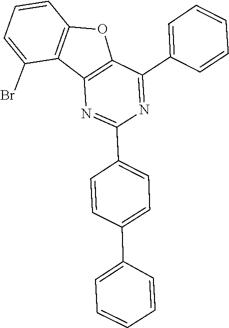 [SMILES: Brc1cccc2oc3c(-c4ccccc4)nc(-c4ccc(-c5ccccc5)cc4)nc3c12] | 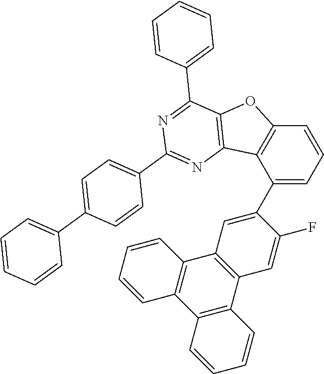 [SMILES: Fc1cc2c3ccccc3c3ccccc3c2cc1-c1cccc2oc3c(-c4ccccc4)nc(-c4ccc(-c5ccccc5)cc4)nc3c12] | 65% |
| 3m | 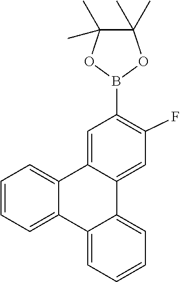 [SMILES: CC1(C)OB(c2cc3c4ccccc4c4ccccc4c3cc2F)OC1(C)C] | 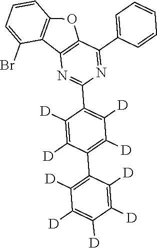 [SMILES: [2H]c1c([2H])c([2H])c(-c2c([2H])c([2H])c(-c3nc(-c4ccccc4)c4oc5cccc(Br)c5c4n3)c([2H])c2[2H])c([2H])c1[2H]] | 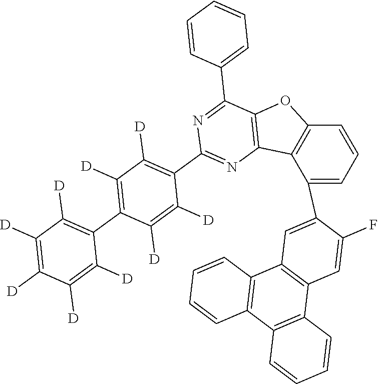 [SMILES: [2H]c1c([2H])c([2H])c(-c2c([2H])c([2H])c(-c3nc(-c4ccccc4)c4oc5cccc(-c6cc7c8ccccc8c8ccccc8c7cc6F)c5c4n3)c([2H])c2[2H])c([2H])c1[2H]] | 64% |
| 4m | 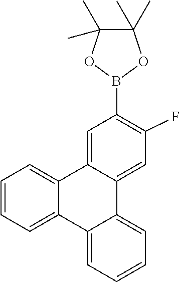 [SMILES: CC1(C)OB(c2cc3c4ccccc4c4ccccc4c3cc2F)OC1(C)C] | 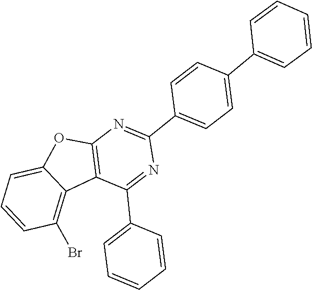 [SMILES: Brc1cccc2oc3nc(-c4ccc(-c5ccccc5)cc4)nc(-c4ccccc4)c3c12] | 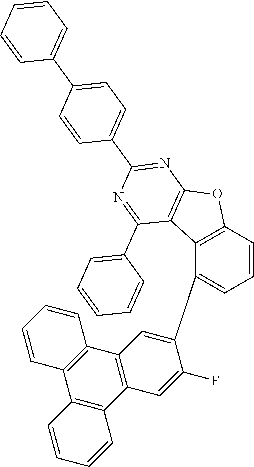 [SMILES: Fc1cc2c3ccccc3c3ccccc3c2cc1-c1cccc2oc3nc(-c4ccc(-c5ccccc5)cc4)nc(-c4ccccc4)c3c12] | 68% |
| 5m | 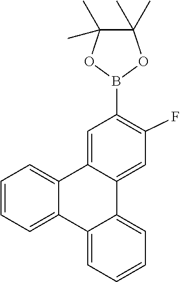 [SMILES: CC1(C)OB(c2cc3c4ccccc4c4ccccc4c3cc2F)OC1(C)C] | 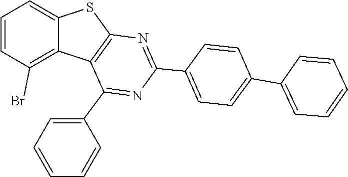 [SMILES: Brc1cccc2sc3nc(-c4ccc(-c5ccccc5)cc4)nc(-c4ccccc4)c3c12] | 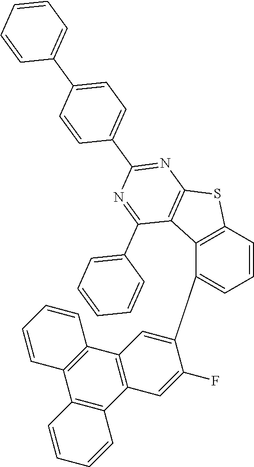 [SMILES: Fc1cc2c3ccccc3c3ccccc3c2cc1-c1cccc2sc3nc(-c4ccc(-c5ccccc5)cc4)nc(-c4ccccc4)c3c12] | 66% |
| 6m | 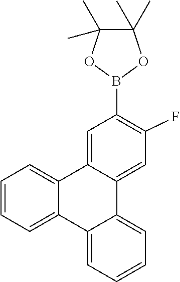 [SMILES: CC1(C)OB(c2cc3c4ccccc4c4ccccc4c3cc2F)OC1(C)C] | 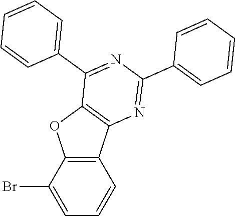 [SMILES: Brc1cccc2c1oc1c(-c3ccccc3)nc(-c3ccccc3)nc12] | 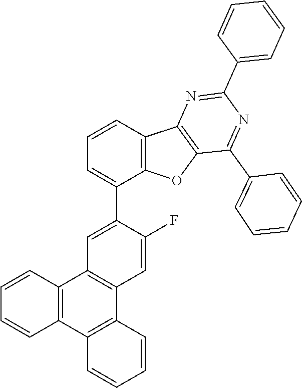 [SMILES: Fc1cc2c3ccccc3c3ccccc3c2cc1-c1cccc2c1oc1c(-c3ccccc3)nc(-c3ccccc3)nc12] | 65% |
| 7m | 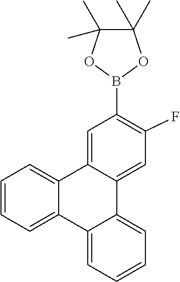 [SMILES: CC1(C)OB(c2cc3c4ccccc4c4ccccc4c3cc2F)OC1(C)C] | 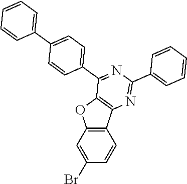 [SMILES: Brc1ccc2c(c1)oc1c(-c3ccc(-c4ccccc4)cc3)nc(-c3ccccc3)nc12] | 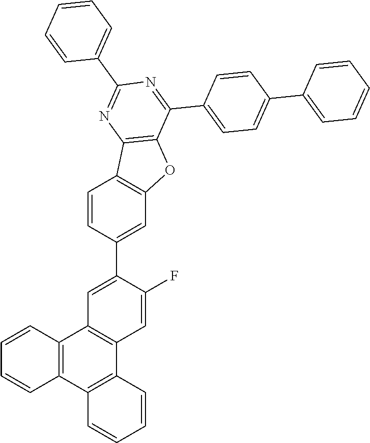 [SMILES: Fc1cc2c3ccccc3c3ccccc3c2cc1-c1ccc2c(c1)oc1c(-c3ccc(-c4ccccc4)cc3)nc(-c3ccccc3)nc12] | 61% |
| 8m | 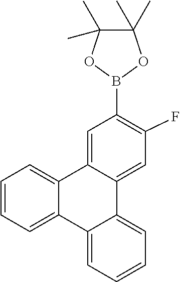 [SMILES: CC1(C)OB(c2cc3c4ccccc4c4ccccc4c3cc2F)OC1(C)C] | 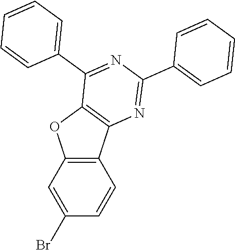 [SMILES: Brc1ccc2c(c1)oc1c(-c3ccccc3)nc(-c3ccccc3)nc12] | 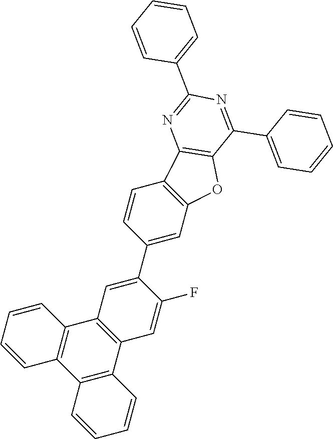 [SMILES: Fc1cc2c3ccccc3c3ccccc3c2cc1-c1ccc2c(c1)oc1c(-c3ccccc3)nc(-c3ccccc3)nc12] | 69% |
| 9m | 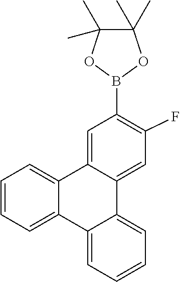 [SMILES: CC1(C)OB(c2cc3c4ccccc4c4ccccc4c3cc2F)OC1(C)C] | 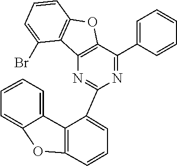 [SMILES: Brc1cccc2oc3c(-c4ccccc4)nc(-c4cccc5oc6ccccc6c45)nc3c12] | 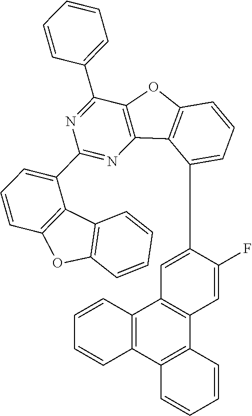 [SMILES: Fc1cc2c3ccccc3c3ccccc3c2cc1-c1cccc2oc3c(-c4ccccc4)nc(-c4cccc5oc6ccccc6c45)nc3c12] | 58% |
| 10m | 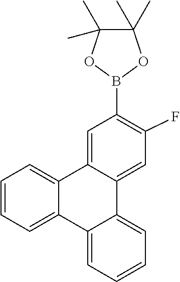 [SMILES: CC1(C)OB(c2cc3c4ccccc4c4ccccc4c3cc2F)OC1(C)C] | 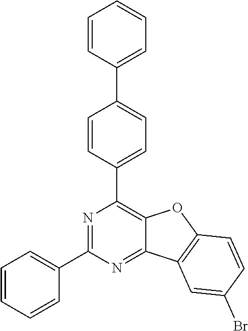 [SMILES: Brc1ccc2oc3c(-c4ccc(-c5ccccc5)cc4)nc(-c4ccccc4)nc3c2c1] [2765540-35-8] | 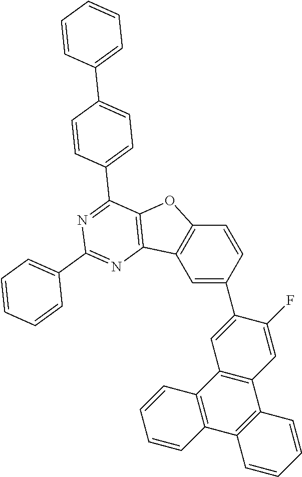 [SMILES: Fc1cc2c3ccccc3c3ccccc3c2cc1-c1ccc2oc3c(-c4ccc(-c5ccccc5)cc4)nc(-c4ccccc4)nc3c2c1] | 65% |
| 11m | 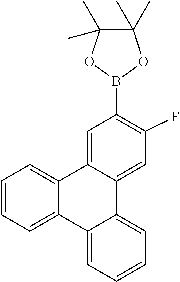 [SMILES: CC1(C)OB(c2cc3c4ccccc4c4ccccc4c3cc2F)OC1(C)C] | 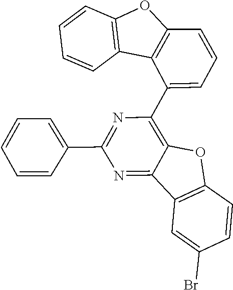 [SMILES: Brc1ccc2oc3c(-c4cccc5oc6ccccc6c45)nc(-c4ccccc4)nc3c2c1] [2922316-45-6] | 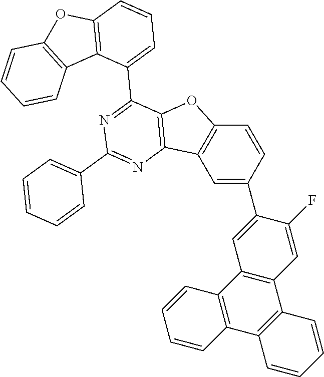 [SMILES: Fc1cc2c3ccccc3c3ccccc3c2cc1-c1ccc2oc3c(-c4cccc5oc6ccccc6c45)nc(-c4ccccc4)nc3c2c1] | 57% |
| 12m | 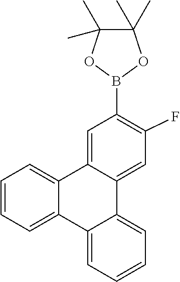 [SMILES: CC1(C)OB(c2cc3c4ccccc4c4ccccc4c3cc2F)OC1(C)C] | 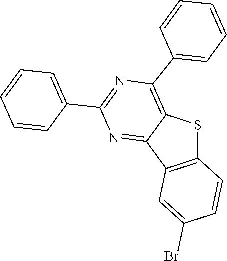 [SMILES: Brc1ccc2sc3c(-c4ccccc4)nc(-c4ccccc4)nc3c2c1] [1678536-28-1] | 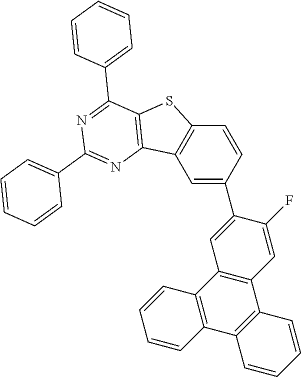 [SMILES: Fc1cc2c3ccccc3c3ccccc3c2cc1-c1ccc2sc3c(-c4ccccc4)nc(-c4ccccc4)nc3c2c1] | 62% |
| 13m | 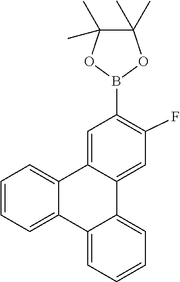 [SMILES: CC1(C)OB(c2cc3c4ccccc4c4ccccc4c3cc2F)OC1(C)C] | 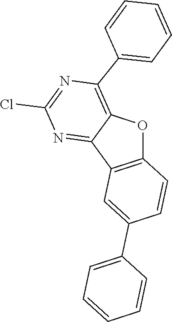 [SMILES: Clc1nc(-c2ccccc2)c2oc3ccc(-c4ccccc4)cc3c2n1] [2765540-21-2] | 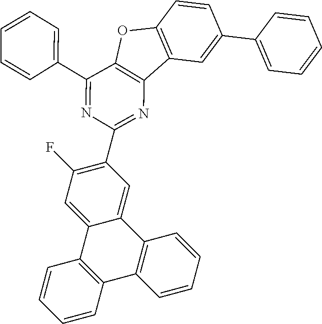 [SMILES: Fc1cc2c3ccccc3c3ccccc3c2cc1-c1nc(-c2ccccc2)c2oc3ccc(-c4ccccc4)cc3c2n1] | 60% |
| 14m | 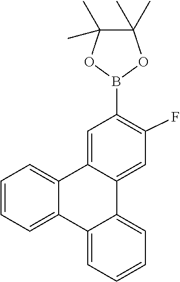 [SMILES: CC1(C)OB(c2cc3c4ccccc4c4ccccc4c3cc2F)OC1(C)C] | 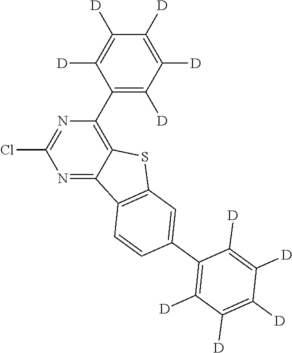 [SMILES: [2H]c1c([2H])c([2H])c(-c2ccc3c(c2)sc2c(-c4c([2H])c([2H])c([2H])c([2H])c4[2H])nc(Cl)nc23)c([2H])c1[2H]] [2201127-97-9] | 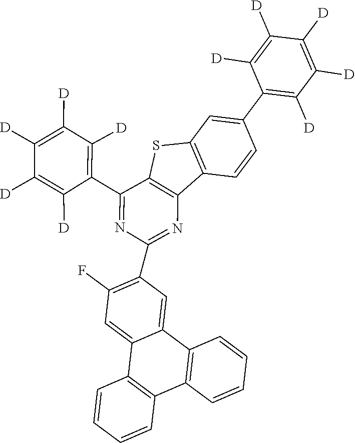 [SMILES: [2H]c1c([2H])c([2H])c(-c2ccc3c(c2)sc2c(-c4c([2H])c([2H])c([2H])c([2H])c4[2H])nc(-c4cc5c6ccccc6c6ccccc6c5cc4F)nc23)c([2H])c1[2H]] | 58% |
| 15m | 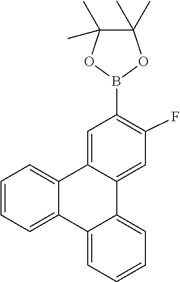 [SMILES: CC1(C)OB(c2cc3c4ccccc4c4ccccc4c3cc2F)OC1(C)C] | 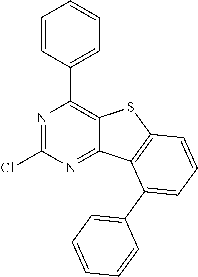 [SMILES: Clc1nc(-c2ccccc2)c2sc3cccc(-c4ccccc4)c3c2n1] [2765540-20-1] | 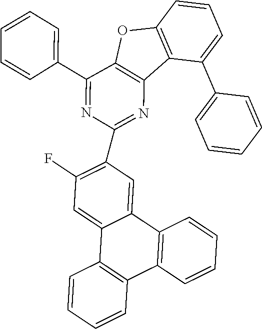 [SMILES: Fc1cc2c3ccccc3c3ccccc3c2cc1-c1nc(-c2ccccc2)c2oc3cccc(-c4ccccc4)c3c2n1] | 55% |
| 16m | 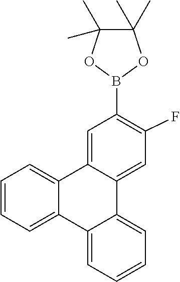 [SMILES: CC1(C)OB(c2cc3c4ccccc4c4ccccc4c3cc2F)OC1(C)C] | 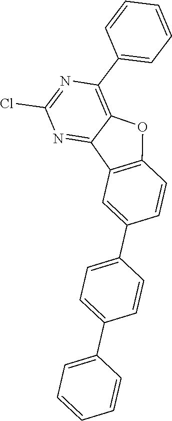 [SMILES: Clc1nc(-c2ccccc2)c2oc3ccc(-c4ccc(-c5ccccc5)cc4)cc3c2n1] [2765540-33-6] | 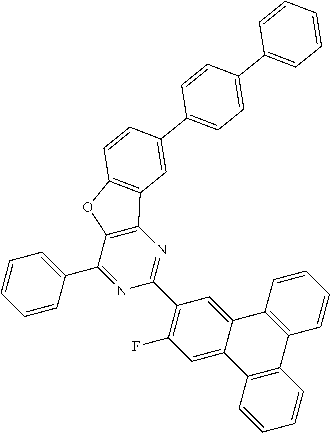 [SMILES: Fc1cc2c3ccccc3c3ccccc3c2cc1-c1nc(-c2ccccc2)c2oc3ccc(-c4ccc(-c5ccccc5)cc4)cc3c2n1] | 67% |
| 17m | 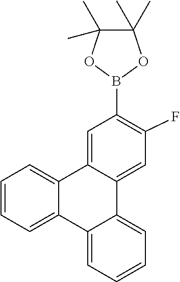 [SMILES: CC1(C)OB(c2cc3c4ccccc4c4ccccc4c3cc2F)OC1(C)C] | 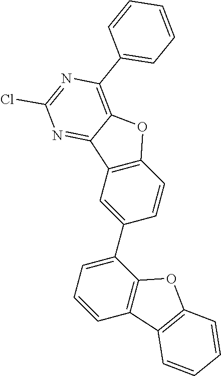 [SMILES: Clc1nc(-c2ccccc2)c2oc3ccc(-c4cccc5c4oc4ccccc45)cc3c2n1] [2268732-49-4] | 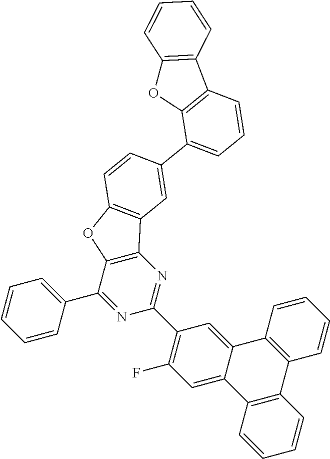 [SMILES: Fc1cc2c3ccccc3c3ccccc3c2cc1-c1nc(-c2ccccc2)c2oc3ccc(-c4cccc5c4oc4ccccc45)cc3c2n1] | 69% |
| 18m | 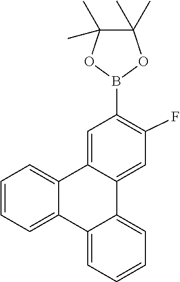 [SMILES: CC1(C)OB(c2cc3c4ccccc4c4ccccc4c3cc2F)OC1(C)C] | 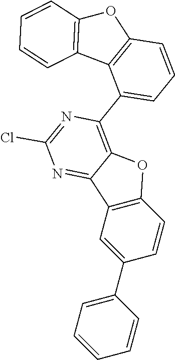 [SMILES: Clc1nc(-c2cccc3oc4ccccc4c23)c2oc3ccc(-c4ccccc4)cc3c2n1] [2768983-33-9] | 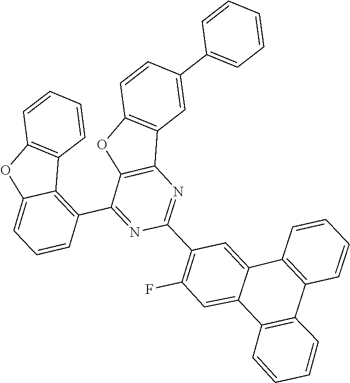 [SMILES: Fc1cc2c3ccccc3c3ccccc3c2cc1-c1nc(-c2cccc3oc4ccccc4c23)c2oc3ccc(-c4ccccc4)cc3c2n1] | 67% |
| 19m | 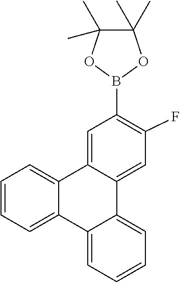 [SMILES: CC1(C)OB(c2cc3c4ccccc4c4ccccc4c3cc2F)OC1(C)C] | 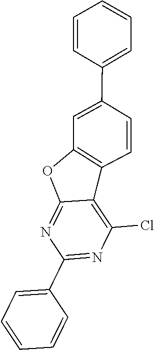 [SMILES: Clc1nc(-c2ccccc2)nc2oc3cc(-c4ccccc4)ccc3c12] [2765540-45-0] | 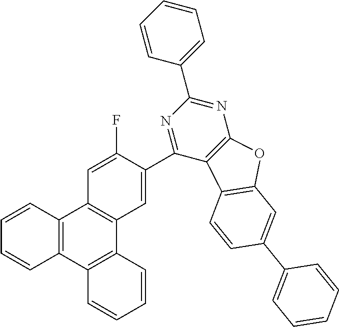 [SMILES: Fc1cc2c3ccccc3c3ccccc3c2cc1-c1nc(-c2ccccc2)nc2oc3cc(-c4ccccc4)ccc3c12] | 53% |
| 20m | 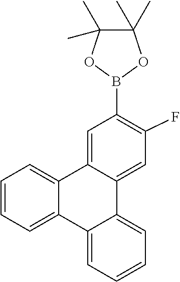 [SMILES: CC1(C)OB(c2cc3c4ccccc4c4ccccc4c3cc2F)OC1(C)C] | 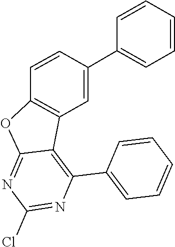 [SMILES: Clc1nc(-c2ccccc2)c2c(n1)oc1ccc(-c3ccccc3)cc12] [765540-17-6] | 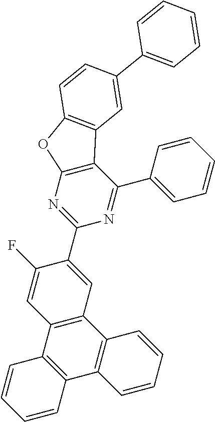 [SMILES: Fc1cc2c3ccccc3c3ccccc3c2cc1-c1nc(-c2ccccc2)c2c(n1)oc1ccc(-c3ccccc3)cc12] | 64% |
| 21m | 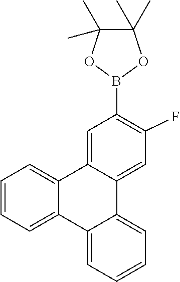 [SMILES: CC1(C)OB(c2cc3c4ccccc4c4ccccc4c3cc2F)OC1(C)C] | 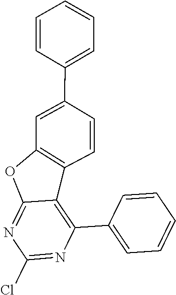 [SMILES: Clc1nc(-c2ccccc2)c2c(n1)oc1cc(-c3ccccc3)ccc12] [2346543-21-1] | 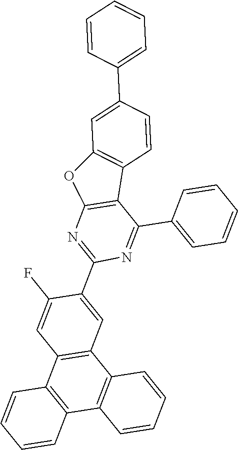 [SMILES: Fc1cc2c3ccccc3c3ccccc3c2cc1-c1nc(-c2ccccc2)c2c(n1)oc1cc(-c3ccccc3)ccc12] | 67% |
| 22m | 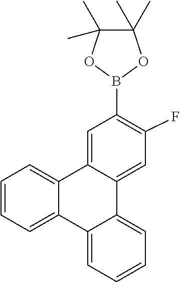 [SMILES: CC1(C)OB(c2cc3c4ccccc4c4ccccc4c3cc2F)OC1(C)C] | 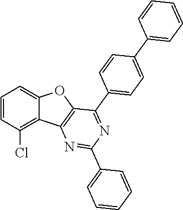 [SMILES: Clc1cccc2oc3c(-c4ccc(-c5ccccc5)cc4)nc(-c4ccccc4)nc3c12] | 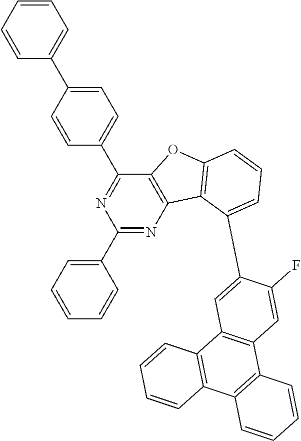 [SMILES: Fc1cc2c3ccccc3c3ccccc3c2cc1-c1cccc2oc3c(-c4ccc(-c5ccccc5)cc4)nc(-c4ccccc4)nc3c12] | 60% |
| 23m | 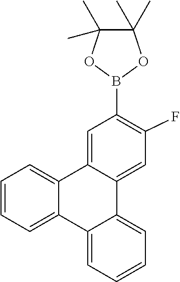 [SMILES: CC1(C)OB(c2cc3c4ccccc4c4ccccc4c3cc2F)OC1(C)C] | 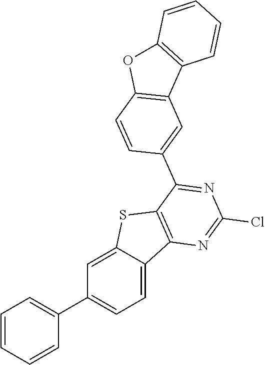 [SMILES: Clc1nc(-c2ccc3oc4ccccc4c3c2)c2sc3cc(-c4ccccc4)ccc3c2n1] | 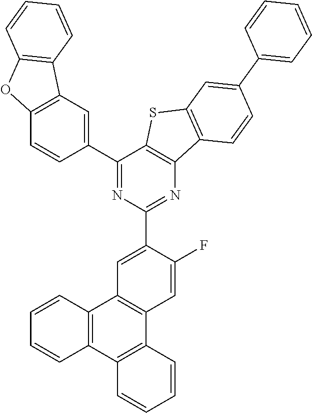 [SMILES: Fc1cc2c3ccccc3c3ccccc3c2cc1-c1nc(-c2ccc3oc4ccccc4c3c2)c2sc3cc(-c4ccccc4)ccc3c2n1] | 66% |
| 24m | 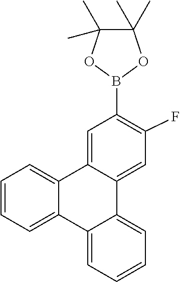 [SMILES: CC1(C)OB(c2cc3c4ccccc4c4ccccc4c3cc2F)OC1(C)C] | 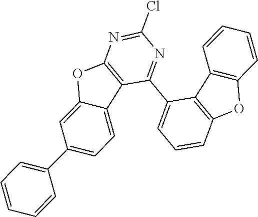 [SMILES: Clc1nc(-c2cccc3oc4ccccc4c23)c2c(n1)oc1cc(-c3ccccc3)ccc12] | 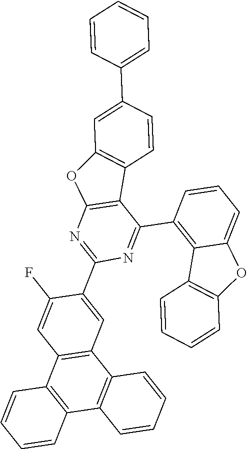 [SMILES: Fc1cc2c3ccccc3c3ccccc3c2cc1-c1nc(-c2cccc3oc4ccccc4c23)c2c(n1)oc1cc(-c3ccccc3)ccc12] | 65% |
| 25m | 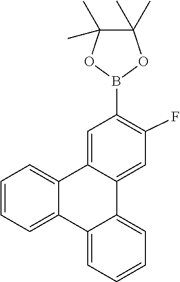 [SMILES: CC1(C)OB(c2cc3c4ccccc4c4ccccc4c3cc2F)OC1(C)C] | 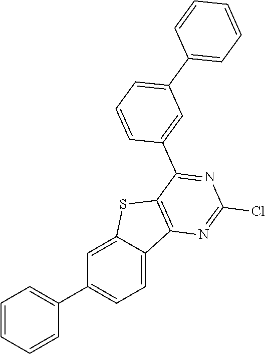 [SMILES: Clc1nc(-c2cccc(-c3ccccc3)c2)c2sc3cc(-c4ccccc4)ccc3c2n1] | 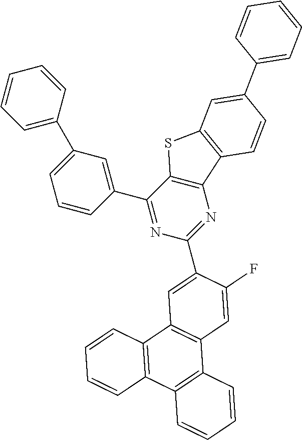 [SMILES: Fc1cc2c3ccccc3c3ccccc3c2cc1-c1nc(-c2cccc(-c3ccccc3)c2)c2sc3cc(-c4ccccc4)ccc3c2n1] | 63% |
| 26m | 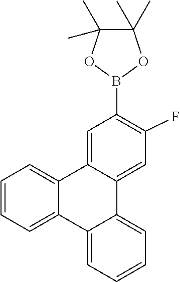 [SMILES: CC1(C)OB(c2cc3c4ccccc4c4ccccc4c3cc2F)OC1(C)C] | 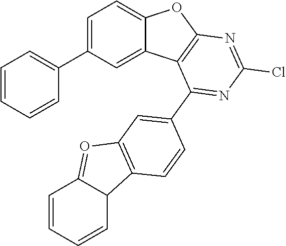 | 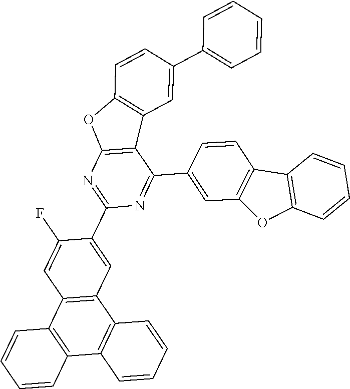 [SMILES: Fc1cc2c3ccccc3c3ccccc3c2cc1-c1nc(-c2ccc3c(c2)oc2ccccc23)c2c(n1)oc1ccc(-c3ccccc3)cc12] | 57% |
| 27m | 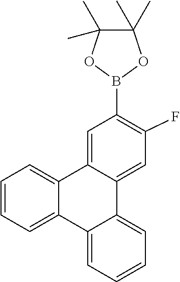 [SMILES: CC1(C)OB(c2cc3c4ccccc4c4ccccc4c3cc2F)OC1(C)C] | 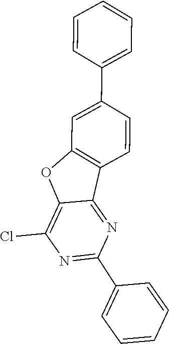 [SMILES: Clc1nc(-c2ccccc2)nc2c1oc1cc(-c3ccccc3)ccc12] [2268733-22-6] | 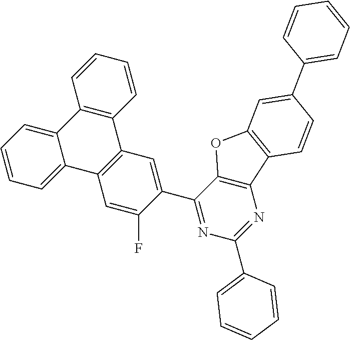 [SMILES: Fc1cc2c3ccccc3c3ccccc3c2cc1-c1nc(-c2ccccc2)nc2c1oc1cc(-c3ccccc3)ccc12] | 64% |
| 28m | 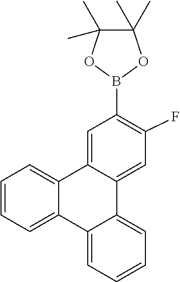 [SMILES: CC1(C)OB(c2cc3c4ccccc4c4ccccc4c3cc2F)OC1(C)C] | 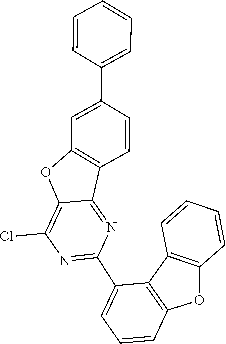 [SMILES: Clc1nc(-c2cccc3oc4ccccc4c23)nc2c1oc1cc(-c3ccccc3)ccc12] [2907762-18-7] | 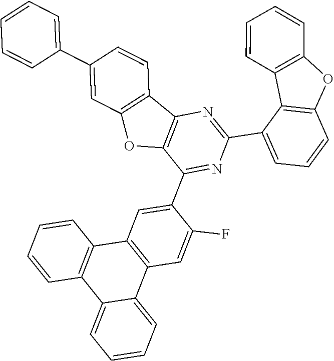 [SMILES: Fc1cc2c3ccccc3c3ccccc3c2cc1-c1nc(-c2cccc3oc4ccccc4c23)nc2c1oc1cc(-c3ccccc3)ccc12] | 67% |
| 29m | 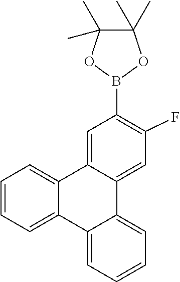 [SMILES: CC1(C)OB(c2cc3c4ccccc4c4ccccc4c3cc2F)OC1(C)C] | 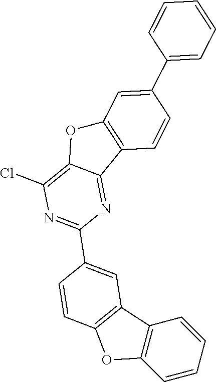 [SMILES: Clc1nc(-c2ccc3oc4ccccc4c3c2)nc2c1oc1cc(-c3ccccc3)ccc12] [2907762-22-3] | 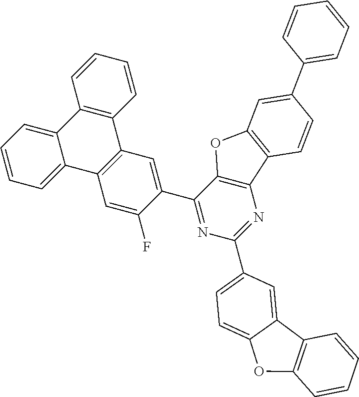 [SMILES: Fc1cc2c3ccccc3c3ccccc3c2cc1-c1nc(-c2ccc3oc4ccccc4c3c2)nc2c1oc1cc(-c3ccccc3)ccc12] | 66% |

### n) 8-(3-(4H-Naphtho[1,2,3,4-def]carbazol-4-yl)triphenylen-2-yl)-2,4-diphenylbenzofuro[3,2-d]pyrimidine

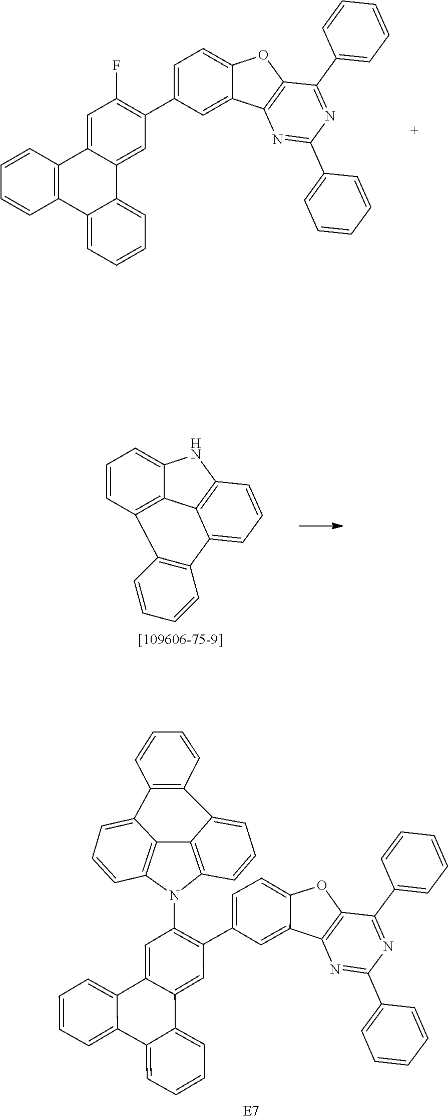 [SMILES: Fc1cc2c3ccccc3c3ccccc3c2cc1-c1ccc2oc3c(-c4ccccc4)nc(-c4ccccc4)nc3c2c1 ; c1ccc2c(c1)c1cccc3[nH]c4cccc2c4c31 ; c1ccc(-c2nc(-c3ccccc3)c3oc4ccc(-c5cc6c7ccccc7c7ccccc7c6cc5-n5c6cccc7c8ccccc8c8cccc5c8c76)cc4c3n2)cc1]

Under argon, 54 g (96 mmol) of 8-(3-fluorotriphenylen-2-yl)-2,4-diphenylbenzofuro[3,2-d]pyrimidine, 30 g (179 mmol) of carbazole and 54 g (166 mmol) of cesium carbonate are suspended in 1500 ml of N,N-dimethylformamide, and the reaction mixture is heated under reflux at 150 °C for 40 h. The reaction mixture is cooled down to room temperature, and 1500 ml of water is added. The solids that precipitate out are filtered off and washed with 300 ml ethanol and recrystallized repeatedly from xylene. After hot filtration through Alox and final sublimation under high vacuum, the purified product is obtained as a colorless solid, 58 g (81 mmol; 85%).

The following compounds are prepared in an analogous manner:

| | Reactant 1 | Reactant 2 | Product | Yield |
|---|---|---|---|---|
| 1n | 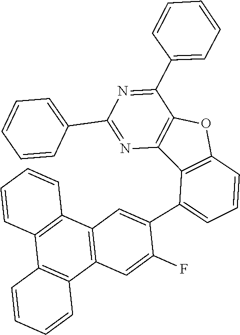 [SMILES: Fc1cc2c3ccccc3c3ccccc3c2cc1-c1cccc2oc3c(-c4ccccc4)nc(-c4ccccc4)nc3c12] | 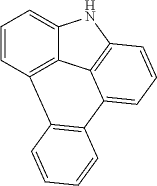 [SMILES: c1ccc2c(c1)c1cccc3[nH]c4cccc2c4c31] [109606-75-9] | 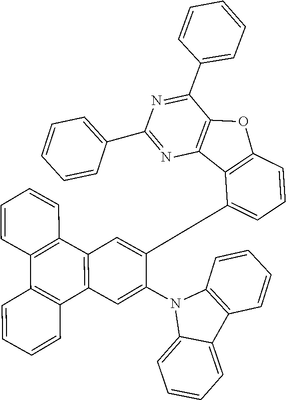 E38 | 70% |
| 2n | 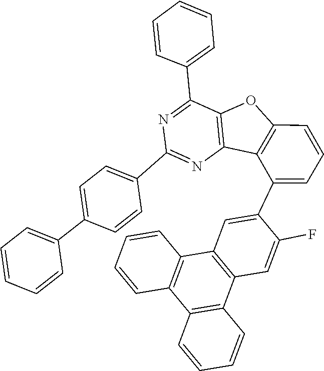 [SMILES: Fc1cc2c3ccccc3c3ccccc3c2cc1-c1cccc2oc3c(-c4ccccc4)nc(-c4ccc(-c5ccccc5)cc4)nc3c12] | 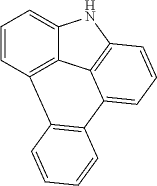 [SMILES: c1ccc2c(c1)c1cccc3[nH]c4cccc2c4c31] [109606-75-9] | 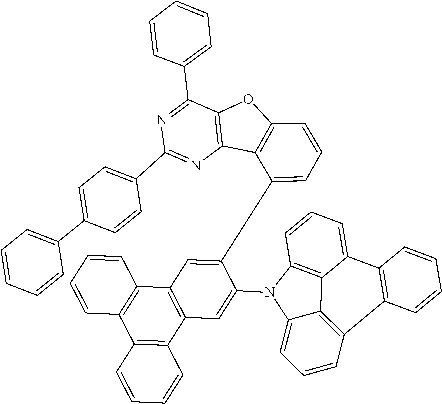 | 62% |
| 3n | 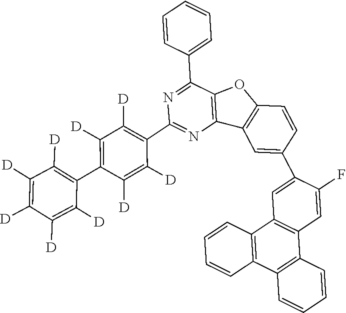 [SMILES: [2H]c1c([2H])c([2H])c(-c2c([2H])c([2H])c(-c3nc(-c4ccccc4)c4oc5ccc(-c6cc7c8ccccc8c8ccccc8c7cc6F)cc5c4n3)c([2H])c2[2H])c([2H])c1[2H]] | 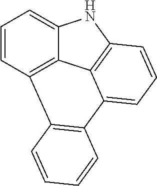 [SMILES: c1ccc2c(c1)c1cccc3[nH]c4cccc2c4c31] [109606-75-9] | 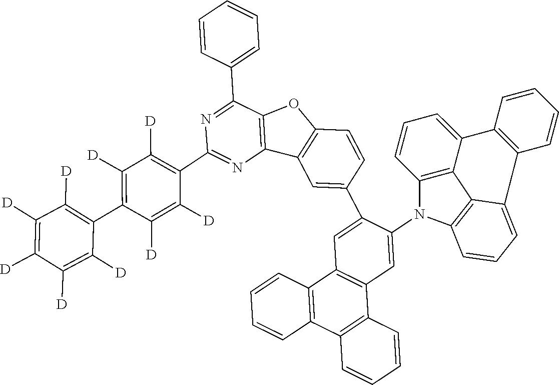 | 62% |
| 4n | 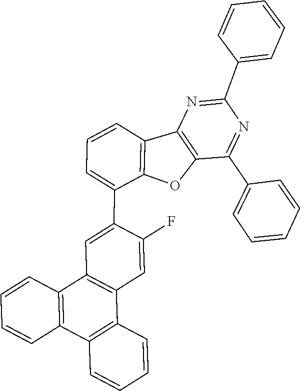 [SMILES: Fc1cc2c3ccccc3c3ccccc3c2cc1-c1cccc2c1oc1c(-c3ccccc3)nc(-c3ccccc3)nc12] | 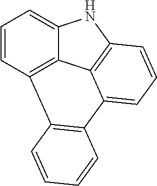 [SMILES: c1ccc2c(c1)c1cccc3[nH]c4cccc2c4c31] [109606-75-9] | 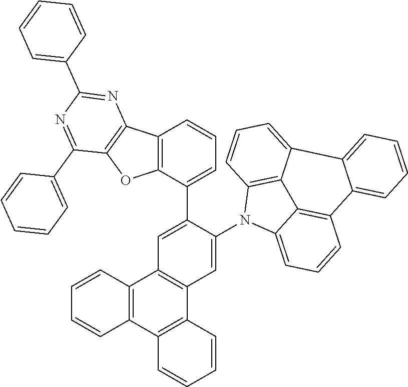 E39 | 65% |
| 5n | 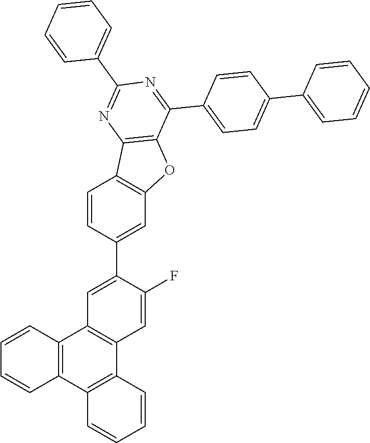 [SMILES: Fc1cc2c3ccccc3c3ccccc3c2cc1-c1ccc2c(c1)oc1c(-c3ccc(-c4ccccc4)cc3)nc(-c3ccccc3)nc12] | 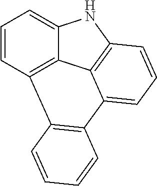 [SMILES: c1ccc2c(c1)c1cccc3[nH]c4cccc2c4c31] [109606-75-9] | 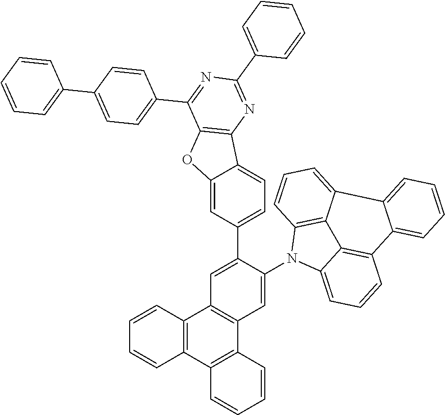 E40 | 67% |
| 6n | 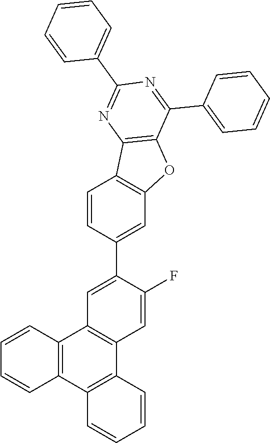 [SMILES: Fc1cc2c3ccccc3c3ccccc3c2cc1-c1ccc2c(c1)oc1c(-c3ccccc3)nc(-c3ccccc3)nc12] | 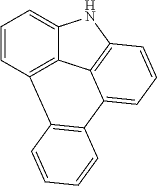 [SMILES: c1ccc2c(c1)c1cccc3[nH]c4cccc2c4c31] [109606-75-9] | 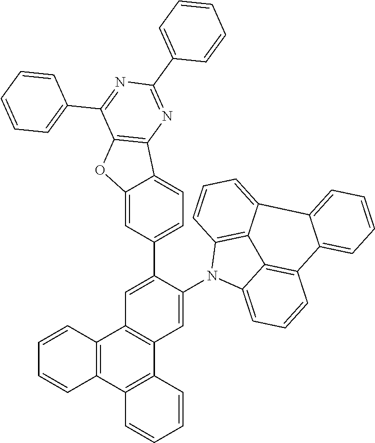 E33 | 63% |
| 7n | 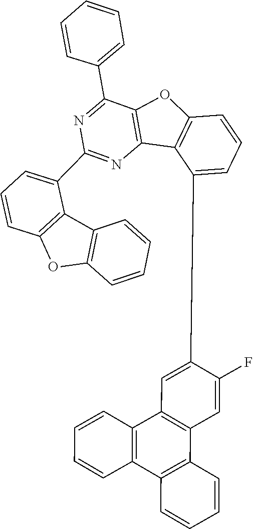 [SMILES: Fc1cc2c3ccccc3c3ccccc3c2cc1-c1cccc2oc3c(-c4ccccc4)nc(-c4cccc5oc6ccccc6c45)nc3c12] | 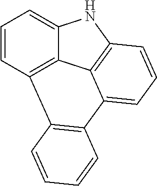 [SMILES: c1ccc2c(c1)c1cccc3[nH]c4cccc2c4c31] [109606-75-9] | 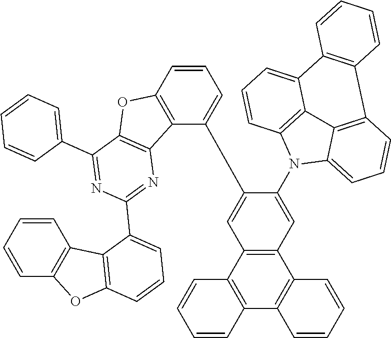 | 57% |
| 8n | 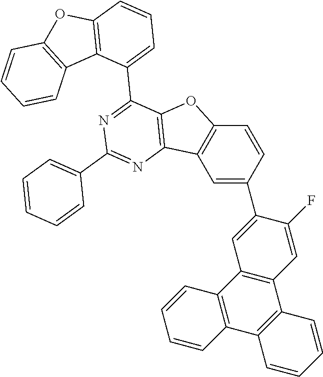 [SMILES: Fc1cc2c3ccccc3c3ccccc3c2cc1-c1ccc2oc3c(-c4cccc5oc6ccccc6c45)nc(-c4ccccc4)nc3c2c1] | 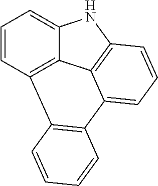 [SMILES: c1ccc2c(c1)c1cccc3[nH]c4cccc2c4c31] [109606-75-9] | 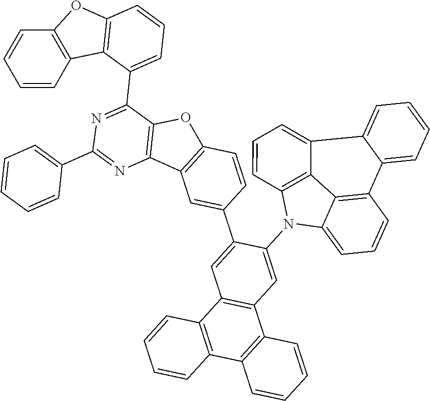 | 59% |
| 9n | 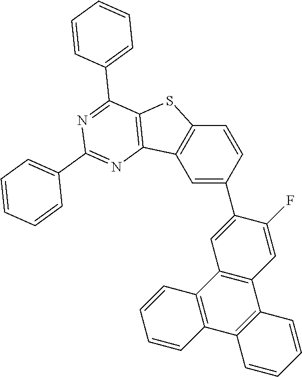 [SMILES: Fc1cc2c3ccccc3c3ccccc3c2cc1-c1ccc2sc3c(-c4ccccc4)nc(-c4ccccc4)nc3c2c1] | 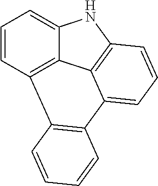 [SMILES: c1ccc2c(c1)c1cccc3[nH]c4cccc2c4c31] [109606-75-9] | 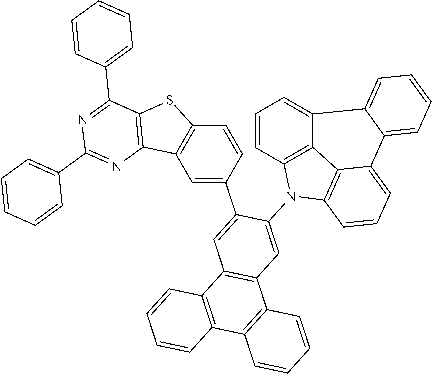 | 63% |
| 10n | 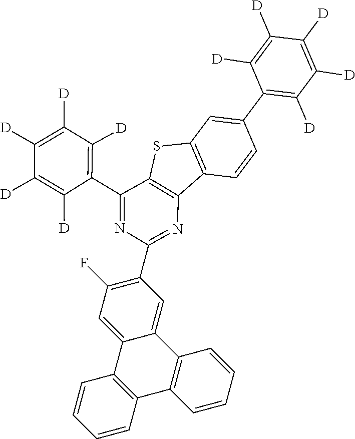 [SMILES: [2H]c1c([2H])c([2H])c(-c2ccc3c(c2)sc2c(-c4c([2H])c([2H])c([2H])c([2H])c4[2H])nc(-c4cc5c6ccccc6c6ccccc6c5cc4F)nc23)c([2H])c1[2H]] | 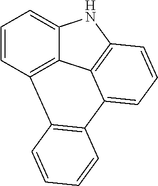 [SMILES: c1ccc2c(c1)c1cccc3[nH]c4cccc2c4c31] [109606-75-9] | 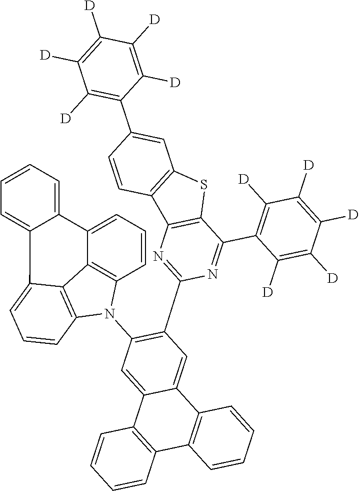 | 62% |
| 11n | 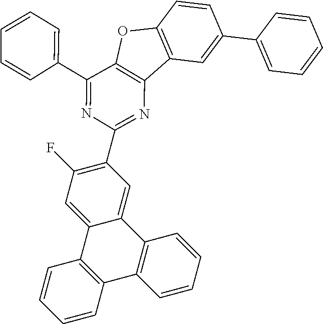 [SMILES: Fc1cc2c3ccccc3c3ccccc3c2cc1-c1nc(-c2ccccc2)c2oc3ccc(-c4ccccc4)cc3c2n1] | 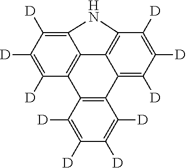 [SMILES: [2H]c1c([2H])c([2H])c2c(c1[2H])c1c([2H])c([2H])c([2H])c3[nH]c4c([2H])c([2H])c([2H])c2c4c31] | 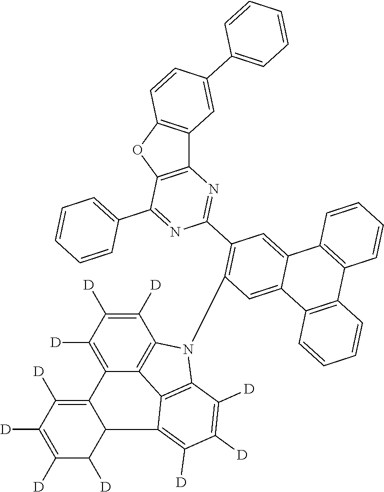 E41 | 58% |
| 12n | 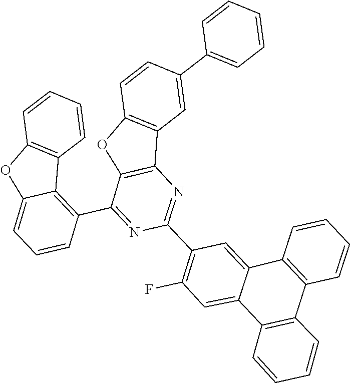 [SMILES: Fc1cc2c3ccccc3c3ccccc3c2cc1-c1nc(-c2cccc3oc4ccccc4c23)c2oc3ccc(-c4ccccc4)cc3c2n1] | 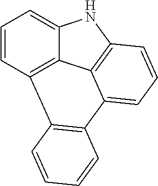 [SMILES: c1ccc2c(c1)c1cccc3[nH]c4cccc2c4c31] [109606-75-9] | 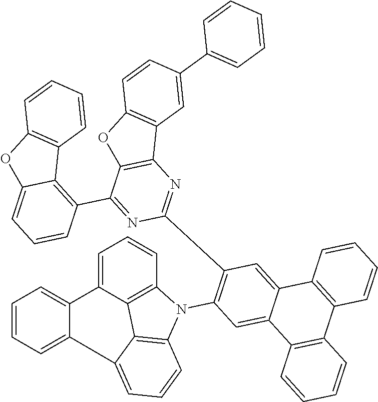 | 60% |
| 13n | 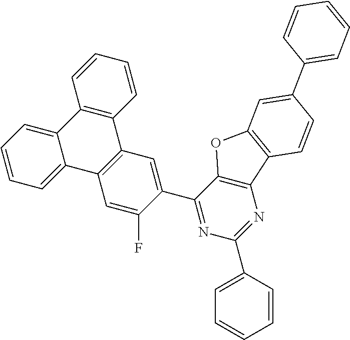 [SMILES: Fc1cc2c3ccccc3c3ccccc3c2cc1-c1nc(-c2ccccc2)nc2c1oc1cc(-c3ccccc3)ccc12] | 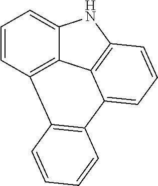 [SMILES: c1ccc2c(c1)c1cccc3[nH]c4cccc2c4c31] [109606-75-9] | 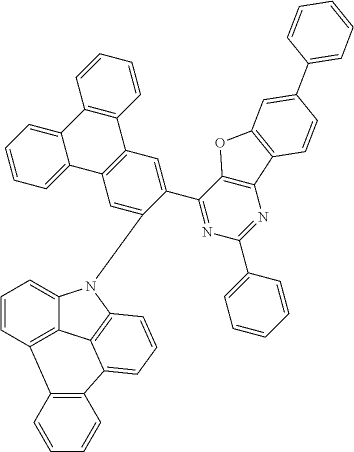 E42 | 62% |
| 14n | 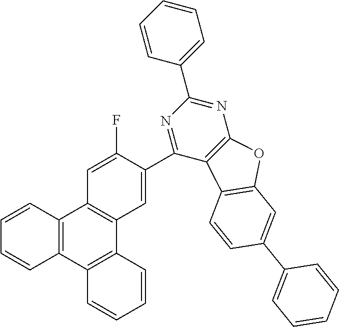 [SMILES: Fc1cc2c3ccccc3c3ccccc3c2cc1-c1nc(-c2ccccc2)nc2oc3cc(-c4ccccc4)ccc3c12] | 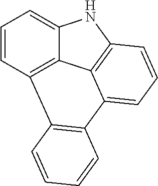 [SMILES: c1ccc2c(c1)c1cccc3[nH]c4cccc2c4c31] [109606-75-9] | 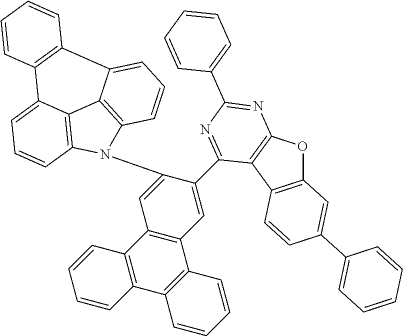 | 67% |
| 15n | 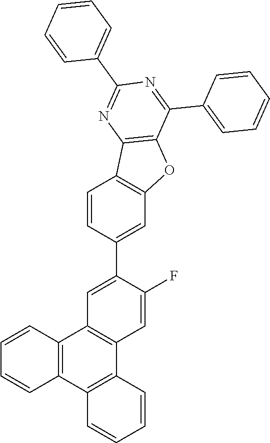 [SMILES: Fc1cc2c3ccccc3c3ccccc3c2cc1-c1ccc2c(c1)oc1c(-c3ccccc3)nc(-c3ccccc3)nc12] | 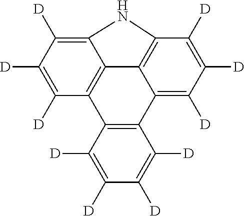 [SMILES: [2H]c1c([2H])c([2H])c2c(c1[2H])c1c([2H])c([2H])c([2H])c3[nH]c4c([2H])c([2H])c([2H])c2c4c31] | 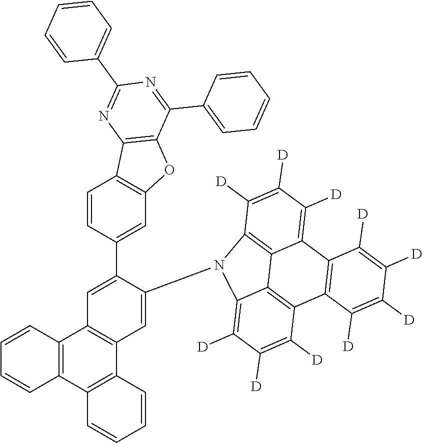 E44 | 64% |
| 16n | 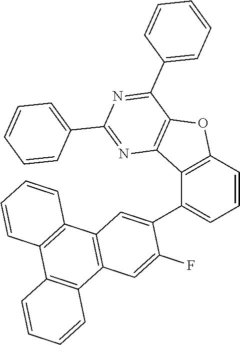 [SMILES: Fc1cc2c3ccccc3c3ccccc3c2cc1-c1cccc2oc3c(-c4ccccc4)nc(-c4ccccc4)nc3c12] | 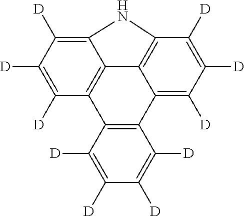 [SMILES: [2H]c1c([2H])c([2H])c2c(c1[2H])c1c([2H])c([2H])c([2H])c3[nH]c4c([2H])c([2H])c([2H])c2c4c31] | 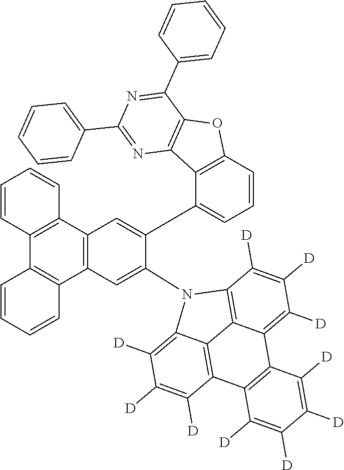 E43 | 63% |

### o) 8-(3-(4H-Naphtho[1,2,3,4-def]carbazol-4-yl-d10)triphenylen-2-yl-1,4,5,6,7,8,9,10,11,12-d10)-2,4-bis(phenyl-d5)benzofuro[3,2-d]pyrimidine-6,7,9-d₃

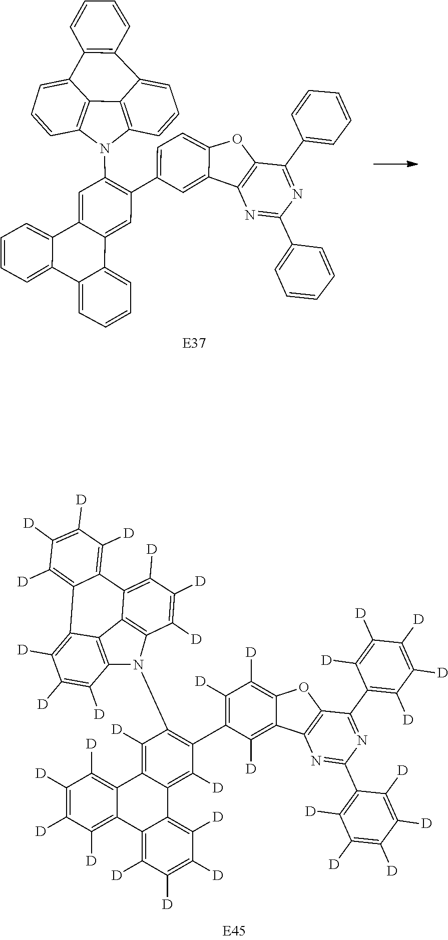 [SMILES: c1ccc(-c2nc(-c3ccccc3)c3oc4ccc(-c5cc6c7ccccc7c7ccccc7c6cc5-n5c6cccc7c8ccccc8c8cccc5c8c76)cc4c3n2)cc1 ; [2H]c1c([2H])c([2H])c(-c2nc(-c3c([2H])c([2H])c([2H])c([2H])c3[2H])c3oc4c([2H])c([2H])c(-c5c(-n6c7c([2H])c([2H])c([2H])c8c9c([2H])c([2H])c([2H])c([2H])c9c9c([2H])c([2H])c([2H])c6c9c87)c([2H])c6c7c([2H])c([2H])c([2H])c([2H])c7c7c([2H])c([2H])c([2H])c([2H])c7c6c5[2H])c([2H])c4c3n2)c([2H])c1[2H]]

34.2 g (48.0 mmol; 1.0 eq) of 2,4-diphenyl-8-(3-(4a,4b,8a,9a-tetrahydro-9H-carbazol-9-yl)triphenylen-2-yl)benzofuro[3,2-d]pyrimidine is suspended in 640 ml of (120 eq) of toluene-d8 [CAS 2037-26-5]. Added to this mixture while cooling is 16.6 ml (6.00 eq.) of trifluoromethanesulfonic acid. The reaction mixture is stirred at room temperature for 6 hours. Thereafter, 120 ml (130 eq) of deuterium oxide [CAS 7789-20-0] is added dropwise at 0 °C. Neutralization with a potassium sulfate solution is followed by extraction with toluene and washing of the combined organic phases with saline solution and drying over sodium sulfate. After filtration, the solvent is removed under reduced pressure. 25 g (33.5 mmol, 70% of theory) of the product shown above is obtained in a mixture with fractions of H/D isotopomers and H/D isotopologues after purification by chromatography, and finally sublimed under high vacuum (p=5×10⁻⁷ mbar) (99.9% purity).

### p) 4H-Naphtho[1,2,3,4-def]carbazole-d₁₀

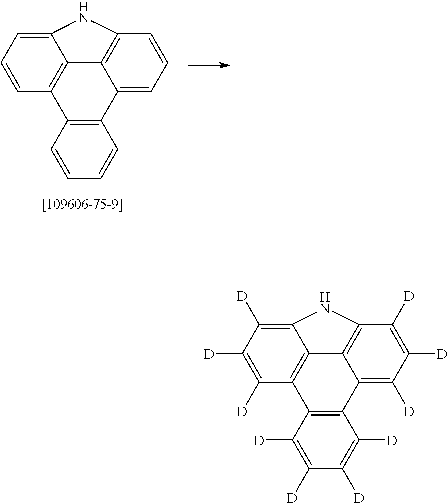

3.7 g (15.5 mmol; 1.00 eq) of 4H-naphtho[1,2,3,4-det]carbazole and 20.0 g of Pt 5% on activated carbon are suspended in 400 g (502 mmol; 1.00 eq) of deuterium oxide [CAS 7789-20-0] and 200 g (778 mmol; 1.55 eq) of toluene-d8 [CAS 2037-26-5]. The reaction mixture is stirred at 165 °C and elevated autogenous pressure for 5 hours. Cooling is followed by two extractions with tetrahydrofuran and washing of the combined organic phases with saline solution and drying over sodium sulfate. After filtration, the solvent is removed under reduced pressure. The product shown above, in a mixture with fractions of H/D isotopomers and H/D isotopologues, is obtained after further purification by extraction, recrystallization and sublimation.

The yield is 1.7 g (6.9 mmol), corresponding to 47% of theory.

### Production of the OLEDs

In examples V1 to V8 and Ex1 to Ex8 which follow (see tables 7 and 8), the data of various OLEDs are presented.

Examples Ex1 to Ex8 show data for OLEDs of the invention. Substrates used for the OLEDs in table 7 are glass plates coated with structured ITO (indium tin oxide) of thickness 50 nm.

The exact structure of the OLEDs can be found in table 7. The materials required for production of the OLEDs are shown in table 9 if not described above.

All materials are applied by thermal vapor deposition in a vacuum chamber. In this case, the emission layer always consists of at least one matrix material (also host material) and an emitting dopant (emitter) which is added to the matrix material(s) in a particular proportion by volume by co-evaporation. Details reported in the form SdT1:H2:TEG1 (32%:60%:8%) 30 nm indicate the presence of material SdT1 in a proportion by volume of 32% as host material 1, the compound H2 as host material 2 in a proportion of 60% and TEG1 in a proportion of 8% in a 30 nm thick layer. In an analogous manner, the hole injection layer (HIL) and the electron transport layer (ETL) may also consist of a mixture of two materials, for example.

The OLEDs are characterized in a standard manner. For this purpose, the electroluminescence spectra and current-voltage-luminance characteristics (IUL characteristics) are measured; these are used to calculate the EQE. The calculation is based on the assumption of Lambertian emission characteristics. Electroluminescence spectra are determined at a luminance of 1000 cd/m², and these are used to calculate the CIE 1931 x and y color coordinates. The parameter U10 in table 8 here denotes the voltage which is required for a current density of 10 mA/cm². EQE10 denotes the external quantum efficiency at a current density of 10 mA/cm².

The lifetime LT is defined as the time after which the luminance drops from a starting luminance L0 (in cd/m²) to a certain proportion L1 (in cd/m²) in the course of operation with constant current density j₀ in mA/cm². A figure of L1/L0=90% in table 8 means that the lifetime reported in the LT column corresponds to the time (in h) after which the luminance falls to 90% of its starting value (L0).

### Use of Mixtures of the Invention in OLEDs

The compounds or material combinations of the invention can be used in the emission layer in phosphorescent green OLEDs.

The data for the various OLEDs are collated in table 8. Examples V1 to V8 are comparative examples according to the prior art; examples Ex1 to Ex8 show data of OLEDs of the invention. The inventive examples show a clear benefit in particular in the lifetime of the device.

TABLE 7
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| V1 | SpMA1:PD1 | SpMA1 | SpMA2 | SdT1:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| Ex1 | SpMA1:PD1 | SpMA1 | SpMA2 | E1:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| V2-1 | SpMA1:PD1 | SpMA1 | SpMA2 | SdT2:H10:TEG2 | AT1 | ST1:LiQ | LiQ |
| | (95%:5%) | 50 nm | 20 nm | (22%:70%:8%) | 5 nm | (50%:50%) | 1 nm |
| | 20 nm | | | 40 nm | | 30 nm | |
| V2-2 | SpMA1:PD1 | SpMA1 | SpMA2 | SdT3:H10:TEG2 | AT1 | ST1:LiQ | LiQ |
| | (95%:5%) | 50 nm | 20 nm | (22%:70%:8%) | 5 nm | (50%:50%) | 1 nm |
| | 20 nm | | | 40 nm | | 30 nm | |
| Ex2 | SpMA1:PD1 | SpMA1 | SpMA2 | E1:H10:TEG2 | AT1 | ST1:LiQ | LiQ |
| | (95%:5%) | 50 nm | 20 nm | (22%:70%:8%) | 5 nm | (50%:50%) | 1 nm |
| | 20 nm | | | 40 nm | | 30 nm | |
| V3-1 | SpMA1:PD1 | SpMA1 | SpMA2 | SdT8:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| V3-2 | SpMA1:PD1 | SpMA1 | SpMA2 | SdT10:H10:TEG2 | AT1 | ST1:LiQ | LiQ |
| | (95%:5%) | 50 nm | 20 nm | (22%:70%:8%) | 5 nm | (50%:50%) | 1 nm |
| | 20 nm | | | 40 nm | | 30 nm | |
| Ex3 | SpMA1:PD1 | SpMA1 | SpMA2 | E34:H10:TEG2 | AT1 | ST1:LiQ | LiQ |
| | (95%:5%) | 50 nm | 20 nm | (22%:70%:8%) | 5 nm | (50%:50%) | 1 nm |
| | 20 nm | | | 40 nm | | 30 nm | |
| V4 | SpMA1:PD1 | SpMA1 | SpMA2 | SdT11:H10:TEG2 | AT1 | ST1:LiQ | LiQ |
| | (95%:5%) | 50 nm | 20 nm | (22%:70%:8%) | 5 nm | (50%:50%) | 1 nm |
| | 20 nm | | | 40 nm | | 30 nm | |
| Ex4- | SpMA1:PD1 | SpMA1 | SpMA2 | E36:H10:TEG2 | AT1 | ST1:LiQ | LiQ |
| 1 | (95%:5%) | 50 nm | 20 nm | (22%:70%:8%) | 5 nm | (50%:50%) | 1 nm |
| | 20 nm | | | 40 nm | | 30 nm | |
| Ex4- | SpMA1:PD1 | SpMA1 | SpMA2 | E8:H10:TEG2 | AT1 | ST1:LiQ | LiQ |
| 2 | (95%:5%) | 50 nm | 20 nm | (22%:70%:8%) | 5 nm | (50%:50%) | 1 nm |
| | 20 nm | | | 40 nm | | 30 nm | |
| V5 | SpMA1:PD1 | SpMA1 | SpMA2 | SdT- | AT1 | ST1:LiQ | LiQ |
| | (95%:5%) | 50 nm | 20 nm | 11:H21:TEG2 | 5 nm | (50%:50%) | 1 nm |
| | 20 nm | | | (42%:50%:8%) | | 30 nm | |
| | | | | 40 nm | | | |
| Ex5 | SpMA1:PD1 | SpMA1 | SpMA2 | E36:H21:TEG2 | AT1 | ST1:LiQ | LiQ |
| | (95%:5%) | 50 nm | 20 nm | (42%:50%:8%) | 5 nm | (50%:50%) | 1 nm |
| | 20 nm | | | 40 nm | | 30 nm | |
| V6 | SpMA1:PD1 | SpMA1 | SpMA2 | SdT2:H1:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E4:H10:TEG2 | AT1 | ST1:LiQ | LiQ |
| 1 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E2:H13:TEG2 | AT1 | ST1:LiQ | LiQ |
| 2 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E4:H15:TEG2 | AT1 | ST1:LiQ | LiQ |
| 3 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E9:H28:TEG3 | AT1 | ST1:LiQ | LiQ |
| 4 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E10:H3:TEG3 | AT1 | ST1:LiQ | LiQ |
| 5 | (95%:5%) | 50 nm | 20 nm | (22%:70%:8%) | 5 nm | (50%:50%) | 1 nm |
| | 20 nm | | | 40 nm | | 30 nm | |
| Ex6- | SpMA1:PD1 | SpMA1 | SpMA2 | E12:H18:TEG2 | AT1 | ST1:LiQ | LiQ |
| 7 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E18:H11:TEG2 | AT1 | ST1:LIQ | LiQ |
| 8 | (95%:5%) | 50 nm | 20 nm | (22%:70%:8%) | 5 nm | (50%:50%) | 1 nm |
| | 20 nm | | | 40 nm | | 30 nm | |
| Ex6- | SpMA1:PD1 | SpMA1 | SpMA2 | E22:H11:TEG3 | AT1 | ST1:LiQ | LiQ |
| 9 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E25:H12:TEG2 | AT1 | ST1:LiQ | LiQ |
| 10 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E26:H8:TEG2 | AT1 | ST1:LiQ | LiQ |
| 11 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E14:H2:TEG2 | AT1 | ST1:LiQ | LiQ |
| 12 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E19:H26:TEG2 | AT1 | ST1:LiQ | LiQ |
| 13 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E30:H13:TEG2 | AT1 | ST1:LiQ | LiQ |
| 14 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E28:H9:TEG2 | AT1 | ST1:LiQ | LiQ |
| 15 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E27:H11:TEG2 | AT1 | ST1:LiQ | LiQ |
| 16 | (95%:5%) | 50 nm | 20 nm | (22%:70%:8%) | 5 nm | (50%:50%) | 1 nm |
| | 20 nm | | | 40 nm | | 30 nm | |
| Ex6- | SpMA1:PD1 | SpMA1 | SpMA2 | E7:H1:TEG2 | AT1 | ST1:LiQ | LiQ |
| 17 | (95%:5%) | 50 nm | 20 nm | (42%:50%:8%) | 5 nm | (50%:50%) | 1 nm |
| | 20 nm | | | 40 nm | | 30 nm | |
| Ex6- | SpMA1:PD1 | SpMA1 | SpMA2 | E33:H10:TEG2(3 | AT1 | ST1:LiQ | LiQ |
| 18 | (95%:5%) | 50 nm | 20 nm | 2%:60%:8%) | 5 nm | (50%:50%) | 1 nm |
| | 20 nm | | | 40 nm | | 30 nm | |
| Ex6- | SpMA1:PD1 | SpMA1 | SpMA2 | E37:H11:TEG2 | AT1 | ST1:LiQ | LiQ |
| 19 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E44:H1:TEG2 | AT1 | ST1:LiQ | LiQ |
| 20 | (95%:5%) | 50 nm | 20 nm | (32%:60%:8%) | 5 nm | (50%:50%) | 1 nm |
| | 20 nm | | | 40 nm | | 30 nm | |
| Ex6- | SpMA1:PD1 | SpMA1 | SpMA2 | E45:H25:TEG2 | AT1 | ST1:LiQ | LiQ |
| 21 | (95%:5%) | 50 nm | 20 nm | (32%:60%:8%) | 5 nm | (50%:50%) | 1 nm |
| | 20 nm | | | 40 nm | | 30 nm | |
| V7-1 | SpMA1:PD1 | SpMA1 | SpMA2 | SdT4:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| V7-2 | SpMA1:PD1 | SpMA1 | SpMA2 | SdT5:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| V7-3 | SpMA1:PD1 | SpMA1 | SpMA2 | SdT6:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| V7-4 | SpMA1:PD1 | SpMA1 | SpMA2 | SdT7:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| Ex7- | SpMA1:PD1 | SpMA1 | SpMA2 | E15:H10:TEG2 | AT1 | ST1:LiQ | LiQ |
| 1 | (95%:5%) | 50 nm | 20 nm | (32%:60%:8%) | 5 nm | (50%:50%) | 1 nm |
| | 20 nm | | | 40 nm | | 30 nm | |
| Ex7- | SpMA1:PD1 | SpMA1 | SpMA2 | E17:H10:TEG2 | AT1 | ST1:LiQ | LiQ |
| 2 | (95%:5%) | 50 nm | 20 nm | (32%:60%:8%) | 5 nm | (50%:50%) | 1 nm |
| | 20 nm | | | 40 nm | | 30 nm | |
| Ex7- | SpMA1:PD1 | SpMA1 | SpMA2 | E17:H29:TEG2 | AT1 | ST1:LiQ | LiQ |
| 3 | (95%:5%) | 50 nm | 20 nm | (32%:60%:8%) | 5 nm | (50%:50%) | 1 nm |
| | 20 nm | | | 40 nm | | 30 nm | |
| Ex7- | SpMA1:PD1 | SpMA1 | SpMA2 | E16:H33:TEG2(3 | AT1 | ST1:LiQ | LiQ |
| 4 | (95%:5%) | 50 nm | 20 nm | 2%:60%:8%) | 5 nm | (50%:50%) | 1 nm |
| | 20 nm | | | 40 nm | | 30 nm | |
| V8 | SpMA1:PD1 | SpMA1 | SpMA2 | SdT9:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |
| Ex8 | SpMA1:PD1 | SpMA1 | SpMA2 | E35:H10:TEG2 | AT1 | ST1:LiQ | LiQ |
| | (95%:5%) | 50 nm | 20 nm | (32%:60%:8%) | 5 nm | (50%:50%) | 1 nm |
| | 20 nm | | | 40 nm | | 30 nm | |

TABLE 8

| | U10 | EQE10 | CIE x/y at | jo | L1/L0 | LT |
|---|---|---|---|---|---|---|
| Ex. | (V) | (%) | 1000 cd/m² | (mA/cm²) | (%) | (h) |
| V1 | 4.2 | 25.2 | 0.35/0.63 | 50 | 90 | 224 |
| Ex1 | 4.3 | 24.8 | 0.35/0.62 | 50 | 90 | 291 |
| V2-1 | 4.3 | 23.7 | 0.35/0.63 | 50 | 90 | 185 |
| V2-2 | 4.3 | 24.9 | 0.35/0.62 | 50 | 90 | 160 |
| Ex2 | 4.5 | 24.3 | 0.35/0.62 | 50 | 90 | 342 |
| V3-1 | 4.4 | 21.2 | 0.34/0.63 | 50 | 90 | 227 |
| V3-2 | 4.4 | 20.8 | 0.34/0.63 | 50 | 90 | 190 |
| Ex3 | 4.4 | 21.1 | 0.34/0.63 | 50 | 90 | 336 |
| V4 | 3.9 | 23.9 | 0.35/0.63 | 50 | 90 | 220 |
| Ex4-1 | 4.1 | 23.6 | 0.34/0.63 | 50 | 90 | 278 |
| Ex4-2 | 4.0 | 23.4 | 0.35/0.63 | 50 | 90 | 311 |
| V5 | 3.7 | 24.8 | 0.35/0.63 | 50 | 90 | 122 |
| Ex5 | 3.9 | 24.8 | 0.35/0.63 | 50 | 90 | 158 |
| V6 | 4.1 | 24.9 | 0.35/0.62 | 50 | 90 | 173 |
| Ex6-1 | 4.5 | 24.9 | 0.35/0.63 | 50 | 90 | 278 |
| Ex6-2 | 4.3 | 24.7 | 0.34/0.63 | 50 | 90 | 351 |
| Ex6-3 | 4.6 | 24.4 | 0.35/0.63 | 50 | 90 | 289 |
| Ex6-4 | 4.2 | 24.6 | 0.35/0.62 | 50 | 90 | 313 |
| Ex6-5 | 4.4 | 30.2 | 0.34/0.63 | 50 | 90 | 282 |
| Ex6-7 | 4.2 | 24.7 | 0.35/0.62 | 50 | 90 | 260 |
| Ex6-8 | 4.4 | 25.0 | 0.34/0.63 | 50 | 90 | 285 |
| Ex6-9 | 4.3 | 30.4 | 0.35/0.63 | 50 | 90 | 273 |
| Ex6-10 | 4.6 | 24.5 | 0.35/0.63 | 50 | 90 | 265 |
| Ex6-11 | 4.5 | 24.7 | 0.34/0.63 | 50 | 90 | 296 |
| Ex6-12 | 4.3 | 24.1 | 0.34/0.63 | 50 | 90 | 245 |
| Ex6-13 | 4.3 | 24.3 | 0.35/0.63 | 50 | 90 | 298 |
| Ex6-14 | 4.4 | 24.6 | 0.35/0.63 | 50 | 90 | 313 |
| Ex6-15 | 4.7 | 24.4 | 0.35/0.62 | 50 | 90 | 278 |
| Ex6-16 | 4.2 | 24.7 | 0.35/0.63 | 50 | 90 | 296 |
| Ex6-17 | 4.5 | 24.5 | 0.35/0.62 | 50 | 90 | 245 |
| Ex6-18 | 4.4 | 24.6 | 0.34/0.63 | 50 | 90 | 291 |
| Ex6-19 | 4.1 | 24.6 | 0.35/0.62 | 50 | 90 | 265 |
| Ex6-20 | 4.3 | 24.5 | 0.35/0.62 | 50 | 90 | 276 |
| Ex6-21 | 4.2 | 24.5 | 0.34/0.63 | 50 | 90 | 300 |
| V7-1 | 4.4 | 24.4 | 0.35/0.63 | 50 | 90 | 147 |
| V7-2 | 4.7 | 13.2 | 0.34/0.63 | 50 | 90 | 78 |
| V7-3 | 4.7 | 15.2 | 0.34/0.63 | 50 | 90 | 93 |
| V7-4 | 4.8 | 18.1 | 0.34/0.63 | 50 | 90 | 107 |
| Ex7-1 | 4.5 | 24.2 | 0.35/0.63 | 50 | 90 | 182 |
| Ex7-2 | 4.4 | 24.3 | 0.34/0.63 | 50 | 90 | 202 |
| Ex7-3 | 4.3 | 24.2 | 0.35/0.63 | 50 | 90 | 218 |
| Ex7-4 | 4.5 | 24.2 | 0.35/0.62 | 50 | 90 | 171 |
| V8 | 4.5 | 22.2 | 0.34/0.63 | 50 | 90 | 113 |
| Ex8 | 4.5 | 22.8 | 0.34/0.63 | 50 | 90 | 145 |

TABLE 9
Materials used, if not described above 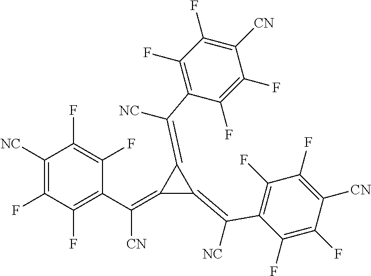 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]
PD1 (CAS Reg. No. 1224447-88-4)
Bisfluorenylamine SpMA1 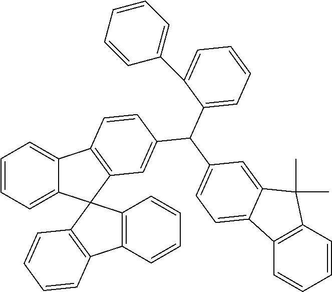 [SMILES: CC1(C)c2ccccc2-c2ccc(C(c3ccc4c(c3)C3(c5ccccc5-c5ccccc53)c3ccccc3-4)c3ccccc3-c3ccccc3)cc21]
SpMA2 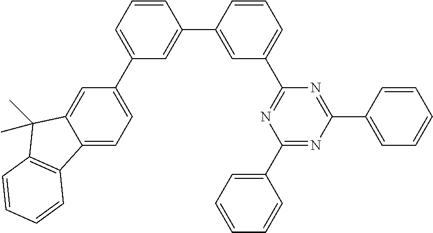 [SMILES: CC1(C)c2ccccc2-c2ccc(-c3cccc(-c4cccc(-c5nc(-c6ccccc6)nc(-c6ccccc6)n5)c4)c3)cc21]
AT1 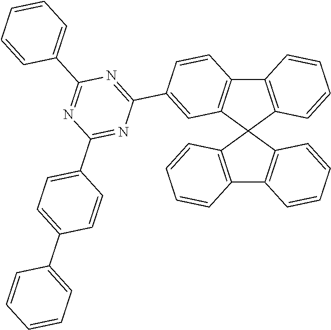 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]
ST1 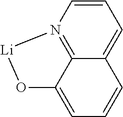
LiQ 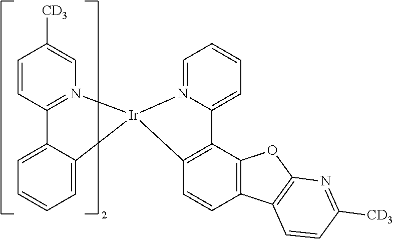
TEG2 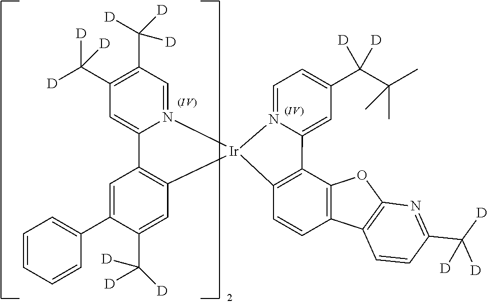
TEG3 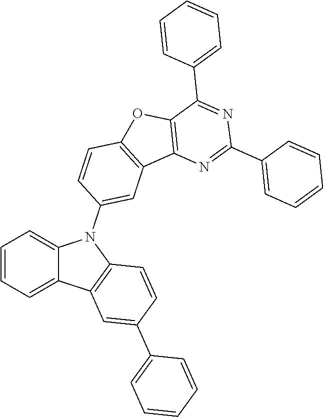 [SMILES: c1ccc(-c2ccc3c(c2)c2ccccc2n3-c2ccc3oc4c(-c5ccccc5)nc(-c5ccccc5)nc4c3c2)cc1]
SdT1 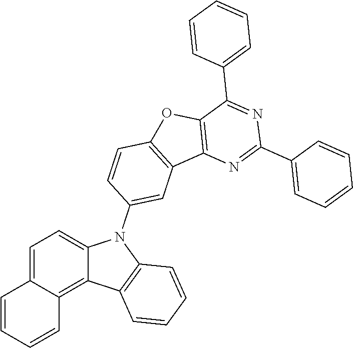 [SMILES: c1ccc(-c2nc(-c3ccccc3)c3oc4ccc(-n5c6ccccc6c6c7ccccc7ccc65)cc4c3n2)cc1]
SdT2 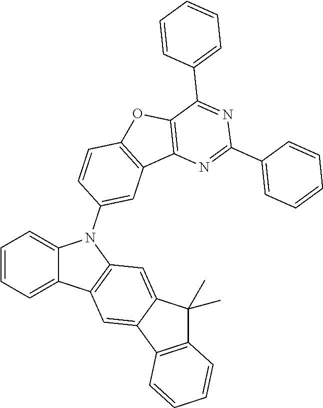 [SMILES: CC1(C)c2ccccc2-c2cc3c4ccccc4n(-c4ccc5oc6c(-c7ccccc7)nc(-c7ccccc7)nc6c5c4)c3cc21]
SdT3 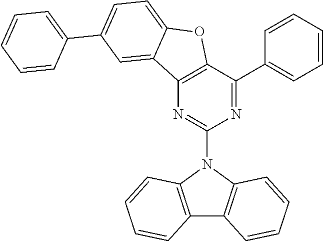 [SMILES: c1ccc(-c2ccc3oc4c(-c5ccccc5)nc(-n5c6ccccc6c6ccccc65)nc4c3c2)cc1]
SdT4 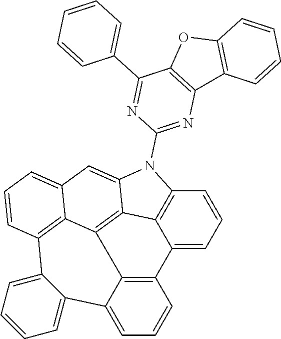 [SMILES: c1ccc(-c2nc(-n3c4cccc5c6cccc7c6c6c8c(cccc8cc3c6c54)-c3ccccc3-7)nc3c2oc2ccccc23)cc1]
SdT5 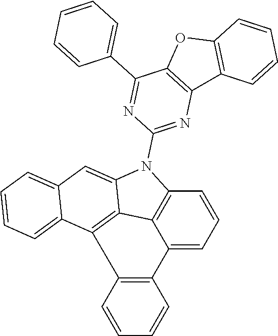 [SMILES: c1ccc(-c2nc(-n3c4cccc5c6ccccc6c6c7ccccc7cc3c6c54)nc3c2oc2ccccc23)cc1]
SdT6 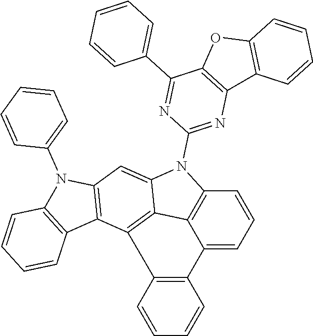 [SMILES: c1ccc(-c2nc(-n3c4cccc5c6ccccc6c6c7c8ccccc8n(-c8ccccc8)c7cc3c6c54)nc3c2oc2ccccc23)cc1]
SdT7 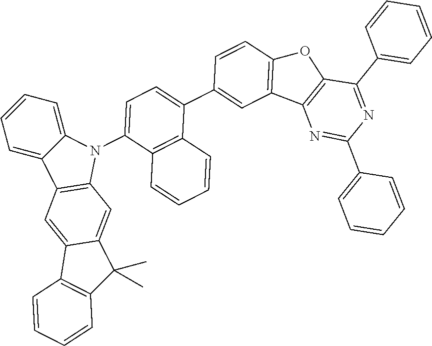 [SMILES: CC1(C)c2ccccc2-c2cc3c4ccccc4n(-c4ccc(-c5ccc6oc7c(-c8ccccc8)nc(-c8ccccc8)nc7c6c5)c5ccccc45)c3cc21]
SdT8 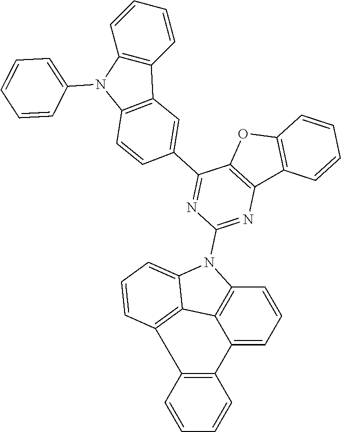 [SMILES: c1ccc(-n2c3ccccc3c3cc(-c4nc(-n5c6cccc7c8ccccc8c8cccc5c8c76)nc5c4oc4ccccc45)ccc32)cc1]
SdT9 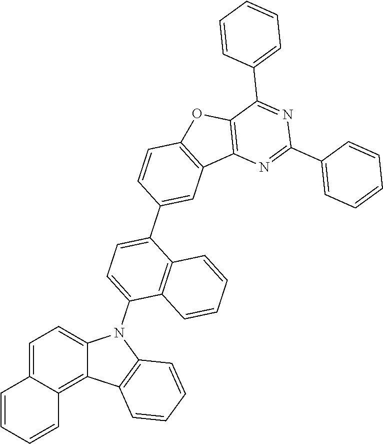 [SMILES: c1ccc(-c2nc(-c3ccccc3)c3oc4ccc(-c5ccc(-n6c7ccccc7c7c8ccccc8ccc76)c6ccccc56)cc4c3n2)cc1]
SdT10 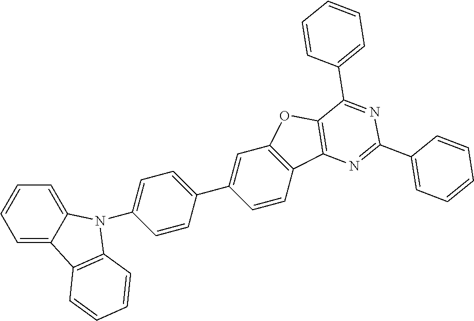 [SMILES: c1ccc(-c2nc(-c3ccccc3)c3oc4cc(-c5ccc(-n6c7ccccc7c7ccccc76)cc5)ccc4c3n2)cc1]
SdT11

1. A compound of formula (1) 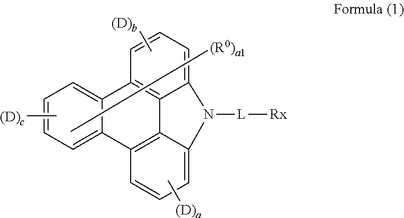
where the symbols and indices used are as follows:
L is the same or different at each instance and is a single bond or an aromatic or heteroaromatic ring system which has 5 to 40 ring atoms and may be substituted by one or more R¹ radicals;
R⁰ is the same or different at each instance and is selected from the group consisting of F, Cl, Br, I, CN, NO₂, C(═O)R², P(═O)(Ar)₂, P(Ar)₂, B(Ar)₂, Si(Ar)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR², and where one or more hydrogen atoms may be replaced by D, F, Cl, Br, I, CN or NO₂, an aromatic or heteroaromatic ring system which has 5 to 40 ring atoms and may be substituted in each case by one or more R² radicals, an aryloxy or heteroaryloxy group which has 5 to 40 ring atoms and may be substituted by one or more R² radicals, or an aralkyl or heteroaralkyl group which has 5 to 40 ring atoms and may be substituted by one or more R² radicals;
R¹ is the same or different at each instance and is selected from the group consisting of D, F, Cl, Br, I, CN, NO₂, C(═O)R², P(═O)(Ar)₂, P(Ar)₂, B(Ar)₂, Si(Ar)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR², and where one or more hydrogen atoms may be replaced by D, F, Cl, Br, I, CN or NO₂, an aromatic or heteroaromatic ring system which has 5 to 40 ring atoms and may be substituted in each case by one or more R² radicals, an aryloxy or heteroaryloxy group which has 5 to 40 ring atoms and may be substituted by one or more R² radicals, or an aralkyl or heteroaralkyl group which has 5 to 40 ring atoms and may be substituted by one or more R² radicals;
Rx conforms to one of the formulae (1-1), (1-2), (1-3) or (1-4) 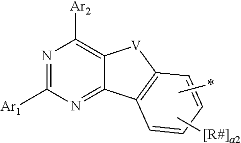
Formula (1-1) 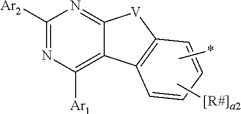
Formula (1-2) 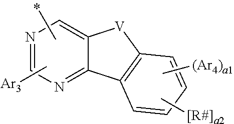
Formula (1-3) 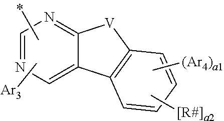
Formula (1-4)
* denotes the bond to L-Rx,
V is O or S;
R# at each instance is independently D, F, CN or phenyl that may be substituted by one or more R² radicals;
[R#]ₐ₂ represents a monosubstitution, a disubstitution, the maximum permissible substitution or no substitution by R#;
Ar is the same or different at each instance and is an aromatic or heteroaromatic ring system which has 5 to 40 ring atoms and may be substituted by one or more R² radicals;
Ar₁, Ar₂ are independently an aromatic or heteroaromatic ring system which has 5 to 40 ring atoms and may be substituted by one or more R² radicals;
Ar₃ is an aromatic ring system which has 6 to 40 ring atoms and may be substituted by one or more R² radicals, or a heteroaromatic ring system which has 5 to 40 ring atoms and may be substituted by one or more R² radicals, and where the heteroaromatic ring system contains one or more heteroatoms selected from O, S, Se or Si;
Ar₄ is the same or different at each instance and is an aromatic or heteroaromatic ring system which has 5 to 40 ring atoms and may be substituted by one or more R² radicals;
R² is the same or different at each instance and is selected from the group consisting of D, F, CN, a straight-chain alkyl group having 1 to 20 carbon atoms or a branched or cyclic alkyl group having 3 to 20 carbon atoms, where one or more nonadjacent CH₂ groups may be replaced by O or S and where one or more hydrogen atoms may be replaced by D, F, or CN;
(D)ₐ, (D)_{b}, (D)_{c} represent a monosubstitution, a disubstitution, a trisubstitution, the maximum permissible substitution or no substitution by deuterium; and
a1 is 0, 1 or 2.

2. A compound according to claim 1, where L is a bond or conforms to one of the formulae L-1 to L-34, which may be substituted by one or more R¹ radicals, where R¹ has a definition given in claim 1, 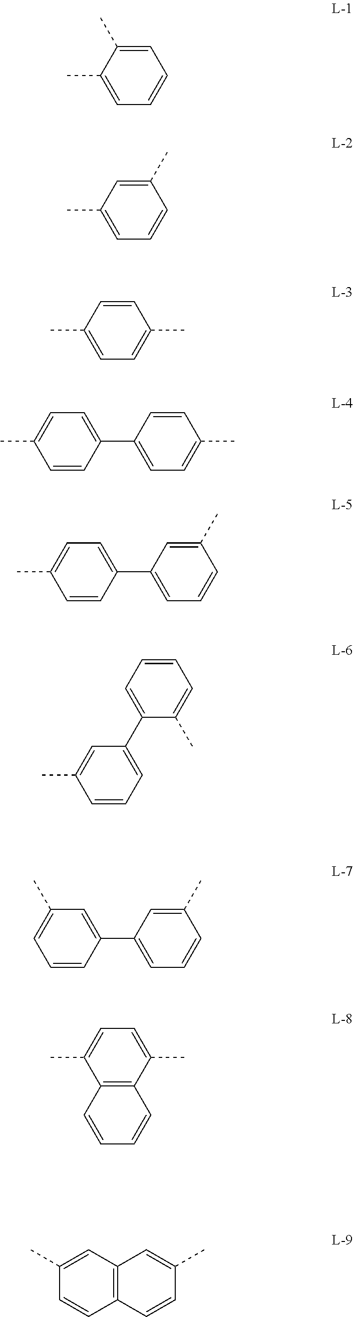 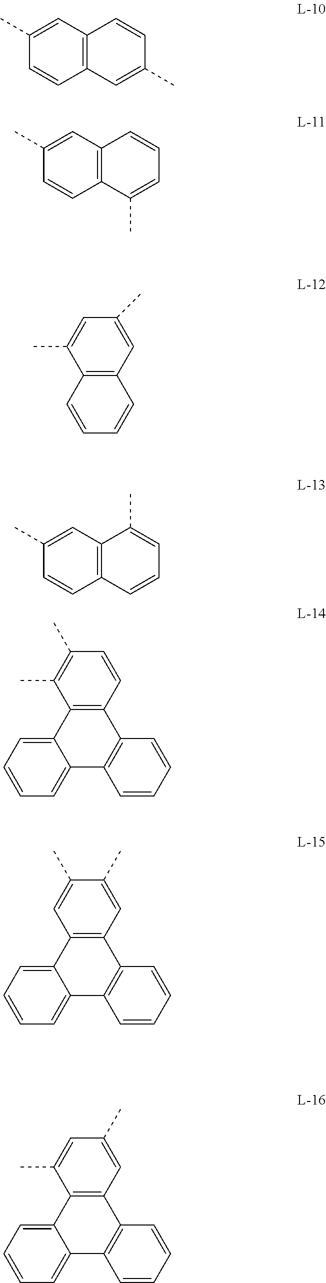 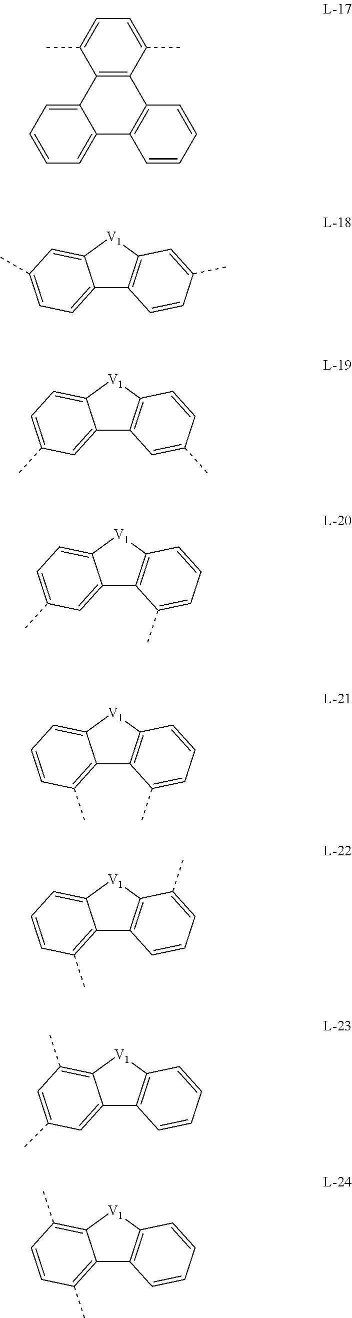 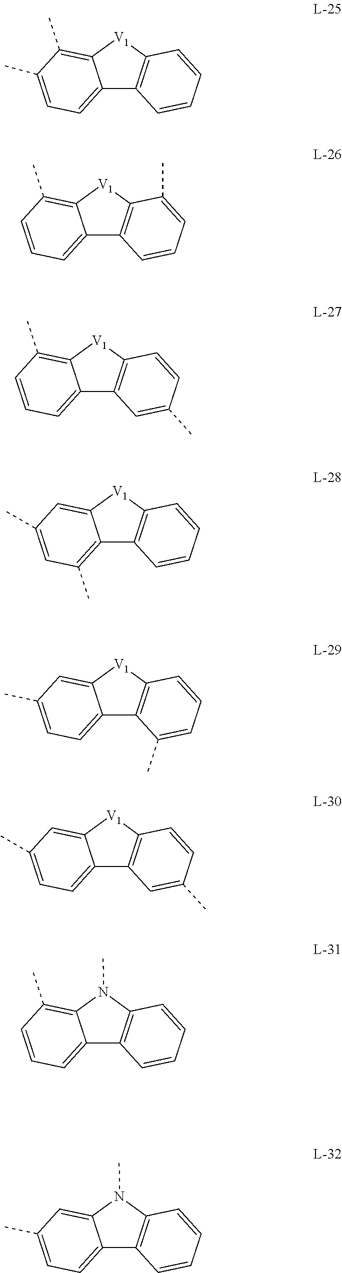 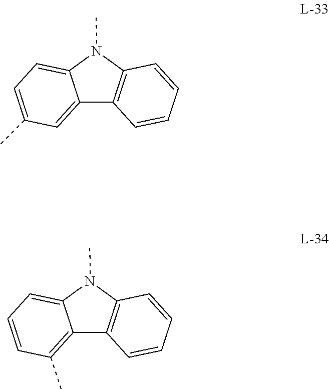
where
V₁ is O, S or Se and the dashed lines denote the attachment to the rest of the formula (1).

3. A compound according to claim 1, where V is O.

4. A compound according to claim 1, where Rx conforms to the formula (1-1) or formula (1-2).

5. A mixture comprising at least one compound according to claim 1 and at least one further compound selected from the group of the matrix materials, phosphorescent emitters, fluorescent emitters and/or emitters that exhibit TADF (thermally activated delayed fluorescence).

6. A formulation comprising at least one compound according to claim 1 and at least one solvent.

7. An organic electronic device comprising an anode, a cathode and at least one organic layer comprising at least one compound according to claim 1.

8. The organic electronic device according to claim 7, wherein the electronic device is an electroluminescent device.

9. The organic electronic device according to claim 7, wherein the organic layer contains at least one light-emitting layer containing the at least one compound.

10. The organic electronic device according to claim 9, characterized in that the light-emitting layer contains a further matrix material.

11. The organic electronic device according to claim 10, characterized in that the further matrix material corresponds to a compound of the formulae (HH-1), (HH-2), (HH-3), (HH-4), (HH-5) or (HH-6): 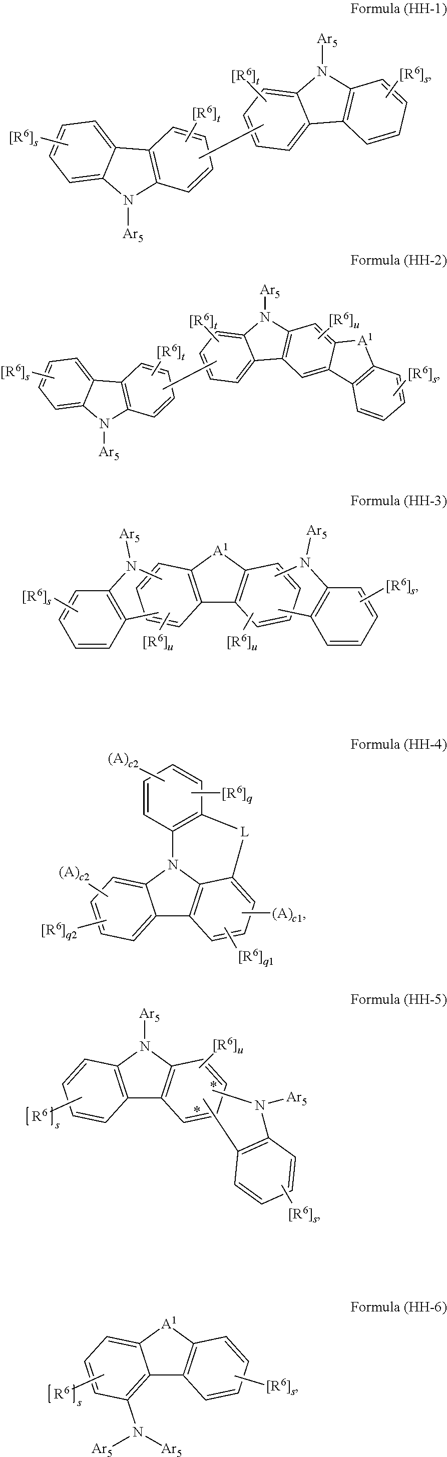
where the symbols and indices used are as follows:
A¹ is C(R⁷)₂, NR⁷, O or S;
L is a bond, O, S, C(R⁷)₂ or NR⁷;
A at each instance is independently a group of the formula (HH-4-1) or (HH-4-2), 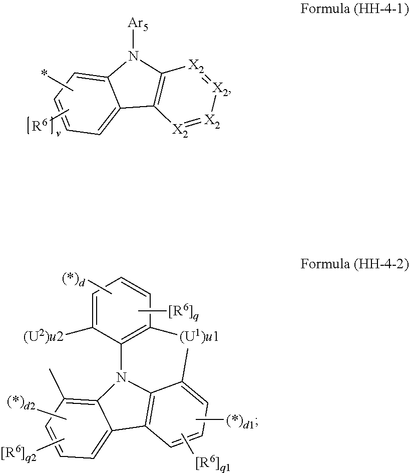
X₂ is the same or different at each instance and is CH, CR⁶ or N, where not more than 2 symbols X₂ can be N;
* indicates the binding site to the formula (HH-4);
U¹, U² where they occur are a bond, O, S, C(R⁷)₂ or NR⁷;
R⁶ at each instance is the same or different and is D, F, CN, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals and where one or more nonadjacent CH₂ groups may be replaced by Si(R⁷)₂, C═O, NR⁷, O, S or CONR⁷, or an aromatic or heteroaromatic ring system which has 5 to 60 ring atoms and may be substituted in each case by one or more R⁷ radicals; optionally where two R⁶ radicals together form an aromatic, heteroaromatic, aliphatic or heteroaliphatic ring system;
Ar₅ is the same or different at each instance and is independently an aromatic or heteroaromatic ring system which has 5 to 40 ring atoms and may be substituted by one or more R⁷ radicals;
R⁷ is the same or different at each instance and is D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals, where one or more nonadjacent CH₂ groups may be replaced by Si(R⁸)₂, C═O, NR⁸, O, S or CONR⁸, or an aromatic or heteroaromatic ring system which has 5 to 40 ring atoms and may be substituted in each case by one or more R⁸ radicals, where R⁸ is not H; at the same time, two or more R⁷ radicals together optionally form an aromatic, heteroaromatic, aliphatic or heteroaliphatic ring system;
R⁸ is the same or different at each instance and is H, D, F or an aliphatic, aromatic or heteroaromatic organic radical having 1 to 20 carbon atoms, in which one or more hydrogen atoms may also be replaced by F;
c, c1, c2 at each instance are each independently 0 or 1, where the sum total of the indices at each instance c+c1+c2=1;
d, d1, d2 at each instance are each independently 0 or 1, where the sum total of the indices at each instance d+d1+d2=1;
q, q1, q2 at each instance are each independently 0, 1, 2, 3 or 4;
s is the same or different at each instance and is 0, 1, 2, 3 or 4;
t is the same or different at each instance and is 0, 1, 2 or 3;
u is the same or different at each instance and is 0, 1 or 2;
u1, u2 at each instance are each independently 0 or 1, where the sum total u1+u2=1; and
v is 0, 1, 2 or 3.

12. The organic electronic device according to claim 9, characterized in that the light-emitting layer contains a phosphorescent emitter.

13. The organic electronic device according to claim 7, characterized in that it is an electroluminescent device selected from organic light-emitting transistors (OLETs), organic field quench devices (OFQDs), organic light-emitting electrochemical cells (OLECs, LECs, LEECs), organic laser diodes (O-lasers) and organic light-emitting diodes (OLEDs).

14. A process for producing a device according to claim 7, characterized in that the organic layer is applied by gas phase deposition or from solution.

15. A process for producing a device according to claim 9, characterized in that the light-emitting layer of the organic layer is applied by gas phase deposition, wherein the at least one compound of the formula (1) is deposited from the gas phase together with at least one further matrix material as premix, successively or simultaneously with the light-emitting materials selected from the group of the phosphorescent emitters, of the fluorescent emitters and/or of the emitters that exhibit TADF (thermally activated delayed fluorescence).

16. A formulation comprising a mixture according to claim 5 and at least one solvent.

17. The organic electronic device according to claim 11, wherein in the formulae (HH-1), (HH-2), (HH-3), (HH-4), (HH-5) or (HH-6), R⁷ has a definition given in claim 11, where two or more R⁷ radicals together do not form an aromatic, heteroaromatic, aliphatic or heteroaliphatic ring system.

18. The organic electronic device according to claim 11, wherein in the formulae (HH-1), (HH-2), (HH-3), (HH-4), (HH-5) or (HH-6), R⁸ has a definition given in claim 11, where at least one instance of R⁸ is a hydrocarbyl radical, having 1 to 20 carbon atoms, in which one or more hydrogen atoms may be replaced by F.
